(19) United States
(12) Patent Application Publication
(10) Pub. No.: US 2011/0224208 A1
(43) Pub. Date: Sep. 15, 2011

(54) NOVEL INHIBITORS OF FLAVIVIRUS REPLICATION

(75) Inventors: Dorothée Bardiot, Heverlee (BE); Emilie Blanche, Heverlee (BE); Patrick Chaltin, Zetrud-Lumay (BE); Mohamed Koukni, Anderlecht (BE); Pieter Leyssen, Tienen (BE); Johan Neyts, Kessel-Lo (BE); Arnaud Marchand, Korbeek-Lo (BE); Inge Vliegen, Holleweg (BE)

(73) Assignee: KATHOLIEKE UNIVERSITEIT LEUVEN, K.U. LEUVEN R&D, Leuven (BE)

(21) Appl. No.: 13/129,311

(22) PCT Filed: Nov. 16, 2009
(86) PCT No.: PCT/EP2009/065256

§ 371 Date: May 13, 2011

(30) Foreign Application Priority Data
Nov. 14, 2008 (GB) 0820856.3

(51) Int. Cl. A61K 31/4725; C07D 409/04; C07D 409/14; C07D 405/04; C07D 217/22; A61K 31/472; C07D 401/04; C07D 413/14; A61K 31/5377; C07D 417/04; A61K 31/506; A61K 31/497; A61P 31/14

(52) U.S. Cl. 514/235.2; 546/141; 514/309; 544/128; 544/328; 514/256; 544/405; 514/255.05

(57) ABSTRACT

The present invention relates to a series of Isoquinolone derivatives which are suitable to treat infections with viruses belonging to the family of the Flaviviridae and more preferably infections with Hepatitis C virus (HCV). The present invention also relates to Isoquinolone compounds for use as a medicine for the prevention or treatment of viral infections, preferably infections with viruses belonging to the family of the Flaviviridae.

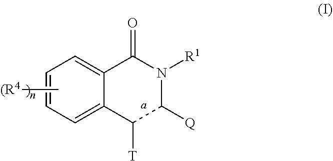

### FIELD OF THE INVENTION

The present invention relates to a series of novel compounds, methods to prevent or treat viral infections by using the novel compounds, processes for preparation of the compounds, their use to treat or prevent viral infections and their use to manufacture a medicine to treat or prevent viral infections, particularly infections with viruses belonging to the family of the Flaviviridae and more preferably infections with Hepatitis C virus (HCV). The present invention also relates to the novel compounds for use as a medicine, more preferably for use as a medicine for the prevention or treatment of viral infections, preferably infections with viruses belonging to the family of the Flaviviridae and more particularly infections with HCV. The present invention furthermore relates to pharmaceutical compositions or combination preparations of the novel compounds, to the compositions or preparations for use as a medicine, more preferably for the prevention or treatment of viral infections, preferably infections with viruses belonging to the family of the Flaviviridae and more particularly infections with HCV

### BACKGROUND OF THE INVENTION

The family of the Flaviviridae consists of 3 genera, the pestiviruses, the flaviviruses and the hepaciviruses and also contains the hepatitis G virus (HGV/GBV-C) that has not yet been assigned to a genus.

The World Health Organization estimates that world-wide 170 million people (3% of the world's population) are chronically infected with HCV. These chronic carriers are at risk of developing cirrhosis and/or liver cancer. In studies with a 10 to 20 year follow-up, cirrhosis developed in 20-30% of the patients, 1 to 5% of whom may develop liver cancer during the next then years. The only treatment option available today is the use of interferon α-2 (or its pegylated from) either alone or combined with ribavirin. However, sustained response is only observed in about 40% of the patients and treatment is associated with serious adverse effects. There is thus an urgent need for potent and selective inhibitors of the replication of the HCV in order to treat infections with HCV. Furthermore, the study of specific inhibitors of HCV replication has been hampered by the fact that it is not possible to propagate HCV (efficiently) in cell culture.

Some isoquinolinone derivatives have been described in the prior art WO2006/097323 for their use in the treatment of cancer. The compounds were identified as ligands of HDM2. The inventors further hypothesise that the compounds could inhibit the replication of HIV due to the interaction between Tat (a HIV transactivator) and HDM2. However, nothing in the prior art directs the person skilled in the art to the compounds of the present invention as Flavivirus replication inhibitors.

The present invention provides novel compounds which show activity against Flaviviridae, more specifically against HCV. There is a clear need in the field for alternative antiviral compounds, furthermore with a good activity vs toxicity profile and this specifically for the viruses of the family of the Flaviviridae, more specifically for Hepatitis C Virus. The prior art does not lead a person skilled in the art to the compounds of the present invention and to their use as antiviral compounds.

### SUMMARY OF THE INVENTION

In the present invention, new selective anti-viral compounds are provided. The compounds are isoquinolinone derivatives and it has been shown that they possess an antiviral activity against the Hepatitis C virus. The present invention demonstrates that the compounds surprisingly inhibit the replication of HCV. Therefore, these isoquinolinone derivatives constitute a new potent class of antiviral compounds that can be used in the treatment and prevention of viral infections in animals, mammals and humans, more specifically for the treatment and prevention of RNA viruses, yet more specifically of Flaviviridae, still more preferably of HCV.

The present invention relates to novel isoquinolinone derivatives. The invention further relates to compounds having antiviral activity against RNA viruses, more specifically to isoquinolinone derivatives that inhibit the replication of viruses. Most particularly, the invention relates to isoquinolinone derivatives which inhibit the replication of viruses of the family of the Flaviviridae and yet more specifically to compounds that inhibit the replication of HCV (Hepatitis C Virus). The present invention furthermore relates to the use of the compounds as a medicine and more specifically to use the compounds for the prevention or treatment of an infection orf an animal, mammal or human with a vrus. The present invention also relates to the compounds for use as a medicine. The invention also relates to methods for preparation of all such compounds and pharmaceutical compositions comprising them. The invention further relates to the use of said compounds in the manufacture of a medicament useful for the treatment of HCV infections, as well as for treatment of other Flaviviral infections. The present invention also relates to a method of treatment of viral infections, by using said compounds.

A first aspect of the present invention is the provision of isoquinolin-1-one derivatives, namely compounds of formula (I)

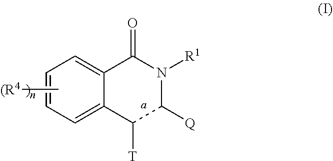

wherein,

the dotted line “a” is selected from a single bond or a double bond,

each of R¹, R² and R³ is independently selected from hydrogen or a C₁₋₁₈-hydrocarbyl group which optionally includes one or more heteroatoms, said heteroatoms being selected from the heteroatoms consisting of O, S, P, and N, wherein said hydrocarbyl group can be unsubstituted or substituted,

each R⁴ is independently selected from halogen, —OH, C₁₋₁₈-alkoxy, —SH, C₁₋₁₈-thioalkoxy, trifluoromethyl, trifluoromethoxy, nitro, -cyano, —NH—SO₂—C₁₋₆-alkyl, —COOH, —COO—C₁₋₆-alkyl, —COO—C₂₋₆-alkenyl, —COO—C₂₋₆-alkynyl, amino, C₁₋₁₈-heteroalkyl, C₁₋₁₈-alkyl, C₂₋₁₈-alkenyl, C₂₋₁₈-alkynyl, aryl, heterocycle, aryl-C₁₋₁₈-alkyl, aryl-C₂₋₁₈-alkenyl, aryl-C₂₋₁₈-alkynyl, heterocycle-C₁₋₁₈-alkyl, heterocycle-C₂₋₁₈-alkenyl, heterocycle-C₂₋₁₈-alkynyl,

n is selected from 0, 1, 2, 3 or 4,

Q is -L¹R² or —R²,

T is -L²R³ or —R³,

each of L¹ and L² is independently selected from C₁₋₆alkyl, C₁₋₆alkenyl or C₁₋₆alkynyl, wherein each of said C₁₋₆alkyl, C₁₋₆alkenyl or C₁₋₆alkynyl optionally includes one or more heteroatoms, said heteroatoms being selected from the heteroatoms consisting of O, S, P, and N, and wherein each of said C₁₋₆alkyl, C₁₋₆alkenyl or C₁₋₆alkynyl can be unsubstituted or substituted,

and isomers (in particular stereo-isomers or tautomers), solvates, hydrates, salts (in particular pharmaceutically acceptable salts) or prodrugs thereof.

In a particular embodiment, the compounds of the invention have a formula according to the formulae (Ia), (Ib), (Ic) or (Id):

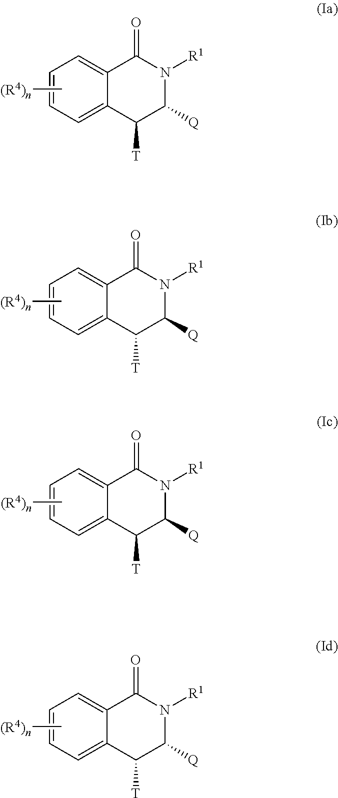

wherein each of R¹, R², R³, R⁴, n, Q and T are as for formula (I).

In yet another particular embodiment, the compounds of the invention have a structure according to formula (I), (Ia), (Ib), (Ic), or (Id), wherein,

the dotted line “a” is selected from a single bond or a double bond,

each of R¹ and R² is independently selected from alkyl, alkenyl, alkynyl, aryl, heterocycle, arylalkyl, arylalkenyl, arylalkynyl, heterocycle-alkyl, heterocycle-alkenyl or heterocycle-alkynyl, and wherein said alkyl, alkenyl, alkynyl, aryl, heterocycle, arylalkyl, arylalkenyl, arylalkynyl, heterocycle-alkyl, heterocycle-alkenyl or heterocycle-alkynyl optionally include one or more heteroatoms, said heteroatom selected from O, S, P and N, and wherein said alkyl, alkenyl, alkynyl, aryl, heterocycle, arylalkyl, arylalkenyl, arylalkynyl, heterocycle-alkyl, heterocycle-alkenyl or heterocycle-alkynyl can be unsubstituted or substituted with one or more Z¹,

R³ is selected from alkyl, alkenyl, alkynyl, heterocycle, aryl, arylalkyl, arylalkenyl, arylalkynyl, heterocycle-alkyl, heterocycle-alkenyl or heterocycle-alkynyl, wherein said alkyl, alkenyl, alkynyl, arylalkyl, arylalkenyl, arylalkynyl, heterocycle-alkyl, heterocycle-alkenyl or heterocycle-alkynyl optionally include one or more heteroatoms, said heteroatom selected from O, S, P and N, and wherein said alkyl, alkenyl, alkynyl, aryl, heterocycle, arylalkyl, arylalkenyl, arylalkynyl, heterocycle-alkyl, heterocycle-alkenyl or heterocycle-alkynyl is optionally substituted with one or more of Z¹⁶,

each of R⁴ is independently selected from the group consisting of halogen, —OH, alkoxy, —SH, thioalkoxy, trifluoromethyl, trifluoromethoxy, nitro, cyano, —COOH, —COO-alkyl, —COO-alkenyl, —COO-alkynyl, amino, heteroalkyl, alkyl, alkenyl, alkynyl, aryl, heterocycle, arylalkyl, arylalkenyl, arylalkynyl, heterocycle-alkyl, heterocycle-alkenyl, and heterocycle-alkynyl.

n is selected from 0, 1, 2, 3 or 4;

Q is —R²;

T is -L²R³ or —R³;

L² is selected from —CH₂NZ⁶—, —CH₂NH—, —CH₂O—, —CO—NZ⁶—, —COO— or —CONH—

each of Z¹ is independently selected from the group consisting of hydrogen, halogen, hydroxyl, sulfhydryl, alkoxy, thioalkoxy, ═O, ═S, trifluoromethyl, trifluoromethoxy, nitro, amino, cyano, —COOH, —COO-alkyl, —COO-alkenyl, —COO-alkynyl, —CONH₂, alkyl, alkenyl, alkynyl, aryl, heterocycle, arylalkyl, arylalkenyl, arylalkynyl, heterocycle-alkyl, heterocycle-alkenyl, heterocycle-alkynyl;

Z⁶ is independently selected from alkyl, alkenyl, alkynyl, heteroalkyl, heterocycle, heterocycle-alkyl, arylalkyl and aryl;

each of Z¹⁶ is independently selected from the group consisting of hydrogen, halogen, hydroxyl, sulfhydryl, alkoxy, thioalkoxy, ═O, ═S, trifluoromethyl, trifluoromethoxy, nitro, amino, cyano, alkyl, alkenyl, alkynyl, aryl, heterocycle, arylalkyl, arylalkenyl, arylalkynyl, heterocycle-alkyl, heterocycle-alkenyl, and heterocycle-alkynyl, wherein said alkoxy, thioalkoxy, alkyl, alkenyl, alkynyl, aryl, heterocycle, arylalkyl, arylalkenyl, arylalkynyl, heterocycle-alkyl, heterocycle-alkenyl, and heterocycle-alkynyl group optionally include one or more heteroatoms, said heteroatom selected from O, S, P and N, and wherein said alkoxy, thioalkoxy, alkyl, alkenyl, alkynyl, aryl, heterocycle, arylalkyl, arylalkenyl, arylalkynyl, heterocycle-alkyl, heterocycle-alkenyl, and heterocycle-alkynyl group is optionally substituted with one or more Z¹⁷;

each of Z¹⁷ is independently selected from halogen, —OH, alkoxy, —SH, thioalkoxy, trifluoromethyl, trifluoromethoxy, ═O, ═S, nitro, cyano, —CONH₂, —COCH₃, —COOH, —COO— alkyl, —COO-alkenyl, —COO-alkynyl, amino, heteroalkyl, alkyl, alkenyl, alkynyl, aryl, heterocycle, arylalkyl, arylalkenyl, arylalkynyl, heterocycle-alkyl, heterocycle-alkenyl, and heterocycle-alkynyl.

In a yet still further embodiment, the compounds of the invention have a structure according to formula (I), (Ia), (Ib), (Ic), or (Id), wherein,

the dotted line “a” is selected from a single bond or a double bond,

R¹ and R² are independently selected from C₁₋₁₈-alkyl, C₂₋₁₈-alkenyl, C₂₋₁₈-alkynyl, aryl, heterocycle, aryl-C₁₋₁₈-alkyl, aryl-C₂₋₁₈-alkenyl, aryl-C₂₋₁₈-alkynyl, heterocycle-C₁₋₁₈-alkyl, heterocycle-C₂₋₁₈-alkenyl or heterocycle-C₂₋₁₈-alkynyl, and wherein said C₁₋₁₈-alkyl, C₂₋₁₈-alkenyl, C₂₋₁₈-alkynyl, aryl, heterocycle, aryl-C₁₋₁₈alkyl, aryl-C₂₋₁₈-alkenyl, aryl-C₂₋₁₈-alkynyl, heterocycle-C₁₋₁₈-alkyl, heterocycle-C₂₋₁₈-alkenyl or heterocycle-C₂₋₁₈-alkynyl optionally include one or more heteroatoms, said heteroatom selected from O, S, P and N, and wherein said C₁₋₁₈-alkyl, C₂₋₁₈-alkenyl, C₂₋₁₈-alkynyl, aryl, heterocycle, aryl-C₁₋₁₈-alkyl, aryl-C₂₋₁₈-alkenyl, aryl-C₂₋₁₈-alkynyl, heterocycle-C₁₋₁₈-alkyl, heterocycle-C₂₋₁₈-alkenyl or heterocycle-C₂₋₁₈-alkynyl can be unsubstituted or substituted with one or more Z¹,

R³ is an C₁₋₁₈-alkyl, heterocyclic or aryl group optionally substituted with one or more of Z¹⁶,

each of R⁴ is independently selected from the group consisting of halogen, —OH, C₁₋₁₈-alkoxy, —SH, C₁₋₁₈-thioalkoxy, trifluoromethyl, trifluoromethoxy, nitro, -cyano, —NH—SO₂—C₁₋₆-alkyl, —COOH, —COO—C₁₋₆-alkyl, —COO—C₂₋₆-alkenyl, —COO—C₂₋₆-alkynyl, amino, C₁₋₁₈-heteroalkyl, C₁₋₁₈-alkyl, C₂₋₁₈-alkenyl, C₂₋₁₈-alkynyl, aryl and heterocycle.

n is selected from 0, 1, 2, 3 or 4,

Q is —R²,

T is -L²R³ or —R³,

L² is selected from —CH₂NZ⁶—, CH₂NH—, —CO—NZ⁶—, —CH₂O—, —COO—, —C(═O)— or —CONH

each of Z¹ is independently selected from the group consisting of hydrogen, halogen, hydroxyl, sulfhydryl, C₁₋₁₈-alkoxy, C₁₋₁₈-thioalkoxy, ═O, ═S, trifluoromethyl, trifluoromethoxy, nitro, -amino, C₁₋₁₈-alkyl, C₂₋₁₈-alkenyl, C₂₋₁₈-alkynyl, aryl, heterocycle, aryl-C₁₋₁₈-alkyl, aryl-C₂₋₁₈-alkenyl, aryl-C₂₋₁₈-alkynyl, heterocycle-C₁₋₁₈-alkyl, heterocycle-C₂₋₁₈-alkenyl, heterocycle-C₂₋₁₈-alkynyl,

Z⁶ is independently selected from alkyl, heteroalkyl, aryl,

each of Z¹⁶ is independently selected from the group consisting of hydrogen, halogen, hydroxyl, sulfhydryl, C₁₋₁₈-alkoxy, C₁₋₁₈-thioalkoxy, ═O, ═S, trifluoromethyl, trifluoromethoxy, nitro, -amino, cyano, —NH—SO₂—C₁₋₈-alkyl, —COOH, —COO—C₁₋₈-alkyl, —COO—C₂₋₈-alkenyl, —COO—C₂₋₈-alkynyl, —CONH₂, C₁₋₁₈-alkyl, C₂₋₁₈-alkenyl, C₂₋₁₈-alkynyl, aryl, heterocycle, aryl-C₁₋₁₈-alkyl, aryl-C₂₋₁₈-alkenyl, aryl-C₂₋₁₈-alkynyl, heterocycle-C₁₋₁₈-alkyl, heterocycle-C₂₋₁₈-alkenyl, heterocycle-C₂₋₁₈-alkynyl, wherein said C₁₋₁₈-alkoxy, C₁₋₁₈-thioalkoxy, C₁₋₁₈-alkyl, C₂₋₁₈-alkenyl, C₁₋₁₈-alkynyl, aryl, heterocycle, aryl-C₁₋₁₈-alkyl, aryl-C₂₋₁₈-alkenyl, aryl-C₂₋₁₈-alkynyl, heterocycle-C₁₋₁₈-alkyl, heterocycle-C₂₋₁₈-alkenyl and heterocycle-C₂₋₁₈-alkynyl group is optionally substituted with a heterocyclic or carbocyclic group,

and isomers (in particular stereo-isomers or tautomers), solvates, hydrates, salts (in particular pharmaceutically acceptable salts) or prodrugs thereof.

In another particular embodiment, the compounds of the invention have a structure according to formula (II)

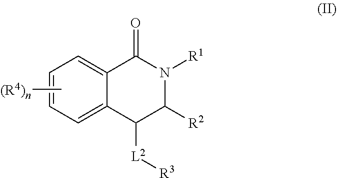

wherein,

R¹ is C₁₋₁₈-alkyl, C₁₋₁₈-heteroalkyl or trialkylsilyl, which can be unsubstituted or substituted with one or more Z¹,

each of R² and R³ is independently selected from aryl, heterocycle, aryl-C₁₋₁₈-alkyl, aryl-C₂₋₁₈-alkenyl, aryl-C₂₋₁₈-alkynyl, heterocycle-C₁₋₁₈-alkyl, heterocycle-C₂₋₁₈-alkenyl or heterocycle-C₂₋₁₈-alkynyl, wherein said aryl, heterocycle, aryl-C₁₋₁₈-alkyl, aryl-C₂₋₁₈-alkenyl, aryl-C₂₋₁₈-alkynyl, heterocycle-C₁₋₁₈-alkyl, heterocycle-C₂₋₁₈-alkenyl or heterocycle-C₂₋₁₈-alkynyl optionally include one or more heteroatoms, said heteroatom selected from O, S, P and N, and wherein said aryl, heterocycle, aryl-C₁₋₁₈-alkyl, aryl-C₂₋₁₈-alkenyl, aryl-C₂₋₁₈-alkynyl, heterocycle-C₁₋₁₈-alkyl, heterocycle-C₂₋₁₈-alkenyl or heterocycle-C₂₋₁₈-alkynyl can be unsubstituted or substituted with one or more Z¹,

L² is selected from —CH₂NZ⁶—, CH₂NH—, —CH₂O—, —CO—NZ⁶—, or —CONH—,

each of R⁴ is independently selected from the group consisting of halogen, —OH, C₁₋₁₈-alkoxy, —SH, C₁₋₁₈-thioalkoxy, trifluoromethyl, trifluoromethoxy, nitro, -cyano, —NHSO₂⁻C₁₋₆-alkyl, —COOH, —COO—C₁₋₆-alkyl, —COO—C₂₋₆-alkenyl, —COO—C₂₋₆-alkynyl, amino, C₁₋₁₈-heteroalkyl, C₁₋₁₈-alkyl, C₂₋₁₈-alkenyl, C₂₋₁₈-alkynyl, aryl and heteroaryl,

n is selected from 0, 1, 2, 3 or 4,

Z¹ is independently selected from the group consisting of halogen, hydroxyl, C₁₋₁₈-alkoxy, C₁₋₁₈-thioalkoxy, trifluoromethyl, trifluoromethoxy, nitro, amino, cyano, —NH—SO₂—C₁₋₆-alkyl, —COOH, —COO—C₁₋₆-alkyl, —COO—C₂₋₆-alkenyl, —COO—C₂₋₆-alkynyl, C₁₋₁₈-alkyl, C₂₋₁₈-alkenyl,

C₂₋₁₈-alkynyl and aryl groups,

Z⁶ is independently selected from C₁₋₁₈-alkyl, C₁₋₁₈-heteroalkyl, aryl, and isomers (in particular stereo-isomers or tautomers), solvates, hydrates, salts (in particular pharmaceutically acceptable salts) or prodrugs thereof.

In a particularly preferred embodiment of the compounds having the structure according to formula (II) R³ is an aryl group substituted with a halogen with Cl being preferred, a —NH—SO₂—C₁₋₆-alkyl group or a C₁₋₆-alkoxy group

In yet another particular embodiment, the compounds of the invention have a structure according to formula (IIIa),

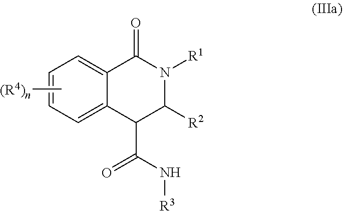

wherein,
R¹ is a C₁₋₁₈-heteroalkyl group or an C₁₋₁₈-alkyl group optionally substituted with a halogen, hydroxyl, carboxy, —COO—C₁₋₆-alkyl, —COO—C₂₋₆-alkenyl, —COO—C₂₋₆-alkynyl, aryl or an optionally C₁₋₁₈-alkyl group-substituted heterocyclic group, R² is an C₁₋₁₈-alkyl, aryl or C₁₋₁₈-heteroaryl group optionally substituted with a halogen or C₁₋₁₈-alkyl group, R³ is an C₁₋₁₈-alkyl, heterocyclic group or aryl group optionally substituted with one or more Z¹, R⁴ is a halogen atom, trifluoromethyl, trifluoromethoxy, —NH—SO₂—C₁₋₆-alkyl, an C₁₋₁₈-alkyl group, a C₁₋₁₈-alkoxy group, a nitro group, a cyano group or an amino group, n is 0, 1, 2, 3 or 4 and Z¹ is independently selected from the group consisting of halogen, hydroxyl, C₁₋₁₈-alkoxy, C₁₋₁₈-thioalkoxy, trifluoromethyl, trifluoromethoxy, nitro, amino, cyano, —NH—SO₂—C₁₋₆-alkyl, —COOH, —COO—C₁₋₆-alkyl, —COO—C₂₋₆-alkenyl, —COO—C₂₋₆-alkynyl, C₁₋₁₈-alkyl, C₂₋₁₈-alkenyl, C₂₋₁₈-alkynyl and aryl groups, and isomers (in particular stereo-isomers or tautomers), solvates, hydrates, salts (in particular pharmaceutically acceptable salts) or prodrugs thereof.

In a preferred embodiment of compounds according to formula (IIIa) R¹ is a C₁₋₆-heteroalkyl group or a C₁₋₆-alkyl group optionally substituted with a halogen, hydroxyl, carboxy, —COO—C₁₋₆-alkyl, —COO—C₂₋₆-alkenyl, —COO—C₂₋₆-alkynyl, aryl or an optionally C₁₋₆-alkyl group-substituted heterocyclic group, R² is an aryl or heteroaryl group optionally substituted with a halogen or C₁₋₆-alkyl group, R³ is an aryl, heterecyclic or C₁₋₆-alkyl group optionally substituted with one of more Z¹, Z¹ is independently selected from the group consisting of halogen, hydroxyl, C₁₋₆-alkoxy, C₁₋₆-thioalkoxy, trifluoromethyl, trifluoromethoxy, nitro, amino, cyano, —NH—SO₂—C₁₋₆-alkyl, —COO—C₁₋₆-alkyl, —COO—C₂₋₆-alkenyl, —COO—C₂₋₆-alkynyl, C₁₋₆-alkyl, C₂₋₆-alkenyl, C₂₋₆-alkynyl and aryl groups and n=0 i.e. wherein n is preferably 0 i.e. the four free positions of the benzene ring are unsubstituted.

In a particularly preferred embodiment of the compounds according to formula (IIIa) R¹ is a C₁₋₆-heteroalkyl group or a C₁₋₆-alkyl group optionally substituted with an optionally alkyl group-substituted heterocyclic group, R³ is an aryl or heterocyclic group optionally substituted with one of more Z¹, Z¹ is independently selected from the group consisting of halogen, hydroxyl, alkoxy, thioalkoxy, trifluoromethyl, trifluoromethoxy, nitro, amino, cyano, —NH—SO₂—C₁₋₆-alkyl, —COO—C₁₋₆-alkyl, —COO—C₂₋₆-alkenyl, —COO—C₂₋₆-alkynyl, C₁₋₆-alkyl, C₂₋₆-alkenyl, C₂₋₆-alkynyl and aryl groups and n=0 i.e. the four free hydrogen atoms of the benzene ring are unsubstituted.

In still another particular embodiment, the compounds of the invention have a structure according to formula (IVa):

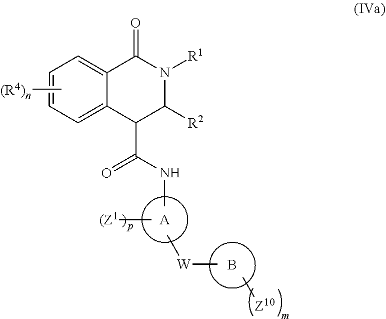

wherein

R¹ is selected from alkyl, alkenyl, alkynyl, aryl, heterocycle, arylalkyl, arylalkenyl, arylalkynyl, heterocycle-alkyl, heterocycle-alkenyl or heterocycle-alkynyl, and wherein said alkyl, alkenyl, alkynyl, aryl, heterocycle, arylalkyl, arylalkenyl, arylalkynyl, heterocycle-alkyl, heterocycle-alkenyl or heterocycle-alkynyl optionally include one or more heteroatoms, said heteroatom selected from O, S, P and N, and wherein said alkyl, alkenyl, alkynyl, aryl, heterocycle, arylalkyl, arylalkenyl, arylalkynyl, heterocycle-alkyl, heterocycle-alkenyl or heterocycle-alkynyl can be unsubstituted or substituted with one or more Z¹,

R² is selected from aryl, heterocycle, arylalkyl, arylalkenyl, arylalkynyl, heterocycle-alkyl, heterocycle-alkenyl or heterocycle-alkynyl, and wherein said arylalkyl, arylalkenyl, arylalkynyl, heterocycle-alkyl, heterocycle-alkenyl or heterocycle-alkynyl optionally include one or more heteroatoms, said heteroatom selected from O, S, P and N, and wherein said aryl, heterocycle, arylalkyl, arylalkenyl, arylalkynyl, heterocycle-alkyl, heterocycle-alkenyl or heterocycle-alkynyl can be unsubstituted or substituted with one or more Z¹,

each of R⁴ is independently selected from the group consisting of halogen, —OH, alkoxy, —SH, thioalkoxy, trifluoromethyl, trifluoromethoxy, nitro, cyano, —NH—SO₂—C₁₋₆-alkyl, —COOH, —COO-alkyl, —COO-alkenyl, —COO-alkynyl, amino, heteroalkyl, alkyl, alkenyl, alkynyl, aryl, heterocycle, arylalkyl, arylalkenyl, arylalkynyl, heterocycle-alkyl, heterocycle-alkenyl, and heterocycle-alkynyl.

n is selected from 0, 1, 2, 3 or 4,

each of cycle A and cycle B is independently selected from aryl or heterocycle,

“—W-” is a single bond, —O—, —S—, an alkylene group, a —C(═O)O— group, a —NH—C(═O)— group, a —C(═O)— group or a —C(═O)—NH— group, wherein said alkylene optionally includes one or more heteroatoms, said heteroatom selected from O, S, P and N,

each of Z¹ and Z¹⁰ are independently selected from the group consisting of hydrogen, halogen, hydroxyl, sulfhydryl, alkoxy, thioalkoxy, ═O, ═S, trifluoromethyl, trifluoromethoxy, nitro, amino, cyano, —NH—SO₂—C₁₋₆-alkyl, —COOH, —COO-alkyl, —COO-alkenyl, —COO-alkynyl, —CONH₂, alkyl, alkenyl, alkynyl, aryl, heterocycle, arylalkyl, arylalkenyl, arylalkynyl, heterocycle-alkyl, heterocycle-alkenyl, heterocycle-alkynyl,

m is 0, 1, 2, 3, or 4,

n is 0, 1, 2, 3, or 4,

p is 0, 1, 2, or 3,

and isomers (in particular stereo-isomers or tautomers), solvates, hydrates, salts (in particular pharmaceutically acceptable salts) or prodrugs thereof.

According to a preferred embodiment of the compounds according to formula (IVa):

R¹ is a C₁₋₁₈-heteroalkyl group or a C₁₋₁₈-alkyl group optionally substituted with an optionally C₁₋₆-alkyl group-substituted heterocyclic group,

R² is a C₁₋₁₈-alkyl, aryl or heteroaryl group optionally substituted with a halogen or C₁₋₁₈-alkyl group,

each of cycle A and cycle B is independently selected from aryl or heterocyclic groups,

—W— is a single bond, —O—, —S—, a C₁₋₆-alkylene group, a —C(═O)O— group, a —NH—C(═O)— group, a —C(═O)— group or a —C(═O)—NH— group, wherein said C₁₋₆-alkylene optionally includes one or more heteroatoms, said heteroatom selected from O, S, P and N,

R⁴ is a halogen atom, an C₁₋₁₈-alkyl group, a C₁₋₁₈-alkoxy group, a nitro group or an amino group,

Z¹ and Z¹⁰ are independently selected from the group consisting of halogen, hydroxyl, C₁₋₆-alkoxy, C₁₋₆-thioalkoxy, trifluoromethyl, trifluoromethoxy, nitro, amino, cyano, —NH—SO₂—C₁₋₆-alkyl, —COO—C₁₋₆-alkyl, —COO—C₂₋₆-alkenyl, —COO—C₂₋₆-alkynyl, C₁₋₆-alkyl, C₂₋₆-alkenyl, C₂₋₆-alkynyl and aryl groups,

m is 0, 1, 2, 3 or 4, n is 0, 1, 2, 3 or 4, p=0, 1, 2 or 3, wherein n is preferably 0 i.e. the four free positions of the benzene ring are unsubstituted, and isomers (in particular stereo-isomers or tautomers), solvates, hydrates, salts (in particular pharmaceutically acceptable salts) or prodrugs thereof.

In a particularly preferred embodiment of the compounds according to formula (IVa) A is an aryl group and B is a heterocyclic group.

In a further particularly preferred embodiment of the compounds according to formula (IVa), R² is exclusive of indolyl and phenyl groups.

In an especially preferred embodiment of the compounds according to formula (IVa) the compounds are exclusive of one or more of the following compounds:
4-Isoquinolinecarboxamide, 1,2,3,4-tetrahydro-2-methyl-1-oxo-3-phenyl-N-[4-(1H-1,2,4-triazol-1-ylmethyl)phenyl]-,
4-Isoquinolinecarboxamide, N-(5-cyclopropyl-1,3,4-thiadiazol-2-yl)-1,2,3,4-tetrahydro-2-methyl-1-oxo-3-phenyl-,
4-Isoquinolinecarboxamide, 1,2,3,4-tetrahydro-2-methyl-1-oxo-3-phenyl-N-[5-(phenylmethyl)-1,3,4-thiadiazol-2-yl]-,
4-Isoquinolinecarboxamide, 2-cyclopropyl-1,2,3,4-tetrahydro-3-(3-methoxyphenyl)-1-oxo-N-[1-(phenylmethyl)-4-piperidinyl]-,
4-Isoquinolinecarboxamide, 1,2,3,4-tetrahydro-2-(2-methoxyethyl)-1-oxo-3-phenyl-N-[1-(phenylmethyl)-4-piperidinyl]-,
4-Isoquinolinecarboxamide, 2-cyclopropyl-1,2,3,4-tetrahydro-3-(3-methylphenyl)-1-oxo-N-[1-(phenylmethyl)-4-piperidinyl]-,
4-Isoquinolinecarboxamide, 1,2,3,4-tetrahydro-1-oxo-N-[1-(phenylmethyl)-4-piperidinyl]-2-propyl-3-(3,4,5-trimethoxyphenyl)-,
4-Isoquinolinecarboxamide, 1,2,3,4-tetrahydro-2-methyl-N-[2-(4-methyl-1-piperazinyl)phenyl]-1-oxo-3-(2-thienyl)-,
4-Isoquinolinecarboxamide, 1,2,3,4-tetrahydro-2-methyl-N-[4-[(4-methyl-1-piperidinyl)methyl]phenyl]-1-oxo-3-(2-thienyl)-,
4-Isoquinolinecarboxamide, 1,2,3,4-tetrahydro-3-(1H-indol-3-yl)-2-(2-methoxyethyl)-1-oxo-N-[1-(phenylmethyl)-4-piperidinyl]-,
4-Isoquinolinecarboxamide, 1,2,3,4-tetrahydro-2-(2-methoxyethyl)-3-(1-methyl-1H-indol-3-yl)-N-[2-(4-methyl-1-piperazinyl)phenyl]-1-oxo-,
4-Isoquinolinecarboxamide, 1,2,3,4-tetrahydro-3-(1H-indol-3-yl)-2-(2-methoxyethyl)-N-[2-(4-methyl-1-piperazinyl)phenyl]-1-oxo-,
4-Isoquinolinecarboxamide, 1,2,3,4-tetrahydro-2-methyl-3-(1-methyl-1H-indol-3-yl)-N-[2-(4-methyl-1-piperazinyl)phenyl]-1-oxo-,
4-Isoquinolinecarboxamide, 3-(2-fluorophenyl)-1,2,3,4-tetrahydro-6,7-dimethoxy-2-methyl-N-[2-(4-methyl-1-piperazinyl)phenyl]-1-oxo-,
4-Isoquinolinecarboxamide, N-[4-[(4-ethyl-1-piperazinyl)methyl]phenyl]-1,2,3,4-tetrahydro-2-(2-methoxyethyl)-1-oxo-3-(2-thienyl)-,
4-Isoquinolinecarboxamide, 1,2,3,4-tetrahydro-2-(2-methoxyethyl)-N-[4-[(4-methyl-1-piperidinyl)methyl]phenyl]-1-oxo-3-(2-thienyl)-,
4-Isoquinolinecarboxamide, 1,2,3,4-tetrahydro-2-(2-methoxyethyl)-N-[2-(4-methyl-1-piperazinyl)phenyl]-1-oxo-3-(2-thienyl)-,
4-Isoquinolinecarboxamide, 1,2,3,4-tetrahydro-2-(2-methoxyethyl)-1-oxo-N-[1-(phenylmethyl)-4-piperidinyl]-3-(2-thienyl)-,
4-Isoquinolinecarboxamide, 2-cyclopentyl-N-[4-[(4-ethyl-1-piperazinyl)methyl]phenyl]-1,2,3,4-tetrahydro-1-oxo-3-(2-thienyl)-,
4-Isoquinolinecarboxamide, 2-cyclopentyl-1,2,3,4-tetrahydro-N-[4-[(4-methyl-1-piperidinyl)methyl]phenyl]-1-oxo-3-(2-thienyl)-,
4-Isoquinolinecarboxamide, 2-cyclopentyl-1,2,3,4-tetrahydro-N-[2-(4-methyl-1-piperazinyl)phenyl]-1-oxo-3-(2-thienyl)-,
4-Isoquinolinecarboxamide, 2-cyclopentyl-1,2,3,4-tetrahydro-1-oxo-N-[1-(phenylmethyl)-4-piperidinyl]-3-(2-thienyl)-,
4-Isoquinolinecarboxamide, N-[4-[(4-ethyl-1-piperazinyl)methyl]phenyl]-1,2,3,4-tetrahydro-2-methyl-1-oxo-3-(2-thienyl)-,
4-Isoquinolinecarboxamide, 1,2,3,4-tetrahydro-2-methyl-1-oxo-N-[4-(1-piperidinylsulfonyl)phenyl]-3-(2-thienyl)-,
4-Isoquinolinecarboxamide, N-[4-[(4-ethyl-1-piperazinyl)methyl]phenyl]-1,2,3,4-tetrahydro-2-(2-methylpropyl)-1-oxo-3-(2-thienyl)-,
4-Isoquinolinecarboxamide, 1,2,3,4-tetrahydro-N-[4-[(4-methyl-1-piperidinyl)methyl]phenyl]-2-(2-methylpropyl)-1-oxo-3-(2-thienyl)-,
4-Isoquinolinecarboxamide, 1,2,3,4-tetrahydro-2-(2-methylpropyl)-1-oxo-N-[4-(1-piperidinylsulfonyl)phenyl]-3-(2-thienyl)-,
4-Isoquinolinecarboxamide, 1,2,3,4-tetrahydro-N-[2-(4-methyl-1-piperazinyl)phenyl]-2-(2-methylpropyl)-1-oxo-3-(2-thienyl)-,
4-Isoquinolinecarboxamide, 1,2,3,4-tetrahydro-2-(2-methylpropyl)-1-oxo-N-[1-(phenylmethyl)-4-piperidinyl]-3-(2-thienyl)-,
4-Isoquinolinecarboxamide, 1,2,3,4-tetrahydro-2-(2-methoxyethyl)-3-(1-methyl-1H-indol-3-yl)-1-oxo-N-(4-phenoxyphenyl)-,
4-Isoquinolinecarboxamide, 1,2,3,4-tetrahydro-2-methyl-3-(1-methyl-1H-indol-3-yl)-1-oxo-N-(4-phenoxyphenyl)-,
4-Isoquinolinecarboxamide, 1,2,3,4-tetrahydro-6,7-dimethoxy-2-methyl-3-[4-(methylthio)phenyl]-1-oxo-N-(4-phenoxyphenyl)-,
4-Isoquinolinecarboxamide, 1,2,3,4-tetrahydro-6,7-dimethoxy-3-(4-methoxyphenyl)-2-methyl-1-oxo-N-(4-phenoxyphenyl)-,
4-Isoquinolinecarboxamide, 1,2,3,4-tetrahydro-2-methyl-1-oxo-N-(4-phenoxyphenyl)-3-(2-thienyl)-,
4-Isoquinolinecarboxamide, 3-(2-fluorophenyl)-1,2,3,4-tetrahydro-6,7-dimethoxy-2-methyl-1-oxo-N-(4-phenoxyphenyl)-,
4-Isoquinolinecarboxamide, 1,2,3,4-tetrahydro-6,7-dimethoxy-2-methyl-1-oxo-N-(4-phenoxyphenyl)-3-(2-thienyl)-,
4-Isoquinolinecarboxamide, 1,2,3,4-tetrahydro-6,7-dimethoxy-2-methyl-1-oxo-N-(4-phenoxyphenyl)-3-(3-pyridinyl)-,
4-Isoquinolinecarboxamide, 1,2,3,4-tetrahydro-3-(4-methoxyphenyl)-2-methyl-1-oxo-N-(4-phenoxyphenyl)-,
4-Isoquinolinecarboxamide, 3-(3,4-dimethoxyphenyl)-1,2,3,4-tetrahydro-6,7-dimethoxy-2-methyl-1-oxo-N-(4-phenoxyphenyl)-,
4-Isoquinolinecarboxamide, 3-(4-fluorophenyl)-1,2,3,4-tetrahydro-2-(2-methoxyethyl)-1-oxo-N-(4-phenoxyphenyl)-,
4-Isoquinolinecarboxamide, 2-cyclohexyl-1,2,3,4-tetrahydro-3-(4-methoxyphenyl)-1-oxo-N-(4-phenoxyphenyl)-,
4-Isoquinolinecarboxamide, 1,2,3,4-tetrahydro-2-(2-methoxyethyl)-3-(1-methyl-1H-indol-3-yl)-N-[2-(4-methyl-1-piperazinyl)phenyl]-1-oxo-, (3R,4R)-rel-,
4-Isoquinolinecarboxamide, 1,2,3,4-tetrahydro-2-(2-methoxyethyl)-3-(1-methyl-1H-indol-3-yl)-1-oxo-N-(4-phenoxyphenyl)-, (3R,4R)-rel-,
4-Isoquinolinecarboxamide, 1,2,3,4-tetrahydro-3-(1H-indol-3-yl)-2-(2-methoxyethyl)-N-[2-(4-methyl-1-piperazinyl)phenyl]-1-oxo-, (3R,4R)-rel-,
4-Isoquinolinecarboxamide, N-[1-(3-chlorophenyl)-4-piperidinyl]-1,2,3,4-tetrahydro-3-(1H-indol-3-yl)-2-(2-methoxyethyl)-1-oxo-, (3R,4R)-rel-,
4-Isoquinolinecarboxamide, 1,2,3,4-tetrahydro-3-(1H-indol-3-yl)-2-(2-methoxyethyl)-1-oxo-N-[1-(phenylmethyl)-4-piperidinyl]-, (3R,4R)-rel-,
4-Isoquinolinecarboxamide, 1,2,3,4-tetrahydro-3-(1H-indol-3-yl)-2-(2-methoxyethyl)-1-oxo-N-(4-phenoxyphenyl)-, (3R,4R)-rel-,
4-Isoquinolinecarboxamide, 1,2,3,4-tetrahydro-3-(1H-indol-3-yl)-2-methyl-1-oxo-N-(4-phenoxyphenyl)-, (3R,4R)-rel-,
4-Isoquinolinecarboxamide, 1,2,3,4-tetrahydro-2-methyl-3-(1-methyl-1H-indol-3-yl)-1-oxo-N-[1-(phenylmethyl)-4-piperidinyl]-, (3R,4R)-rel-,
4-Isoquinolinecarboxamide, 1,2,3,4-tetrahydro-2-methyl-3-(1-methyl-1H-indol-3-yl)-1-oxo-N-(4-phenoxyphenyl)-, (3R,4R)-rel-,
4-Isoquinolinecarboxamide, 1,2,3,4-tetrahydro-2-methyl-3-(1-methyl-1H-indol-3-yl)-1-oxo-N-[1-(phenylmethyl)-4-piperidinyl]-,
4-Isoquinolinecarboxamide, 1,2,3,4-tetrahydro-3-(1H-indol-3-yl)-2-(2-methoxyethyl)-1-oxo-N-(4-phenoxyphenyl)-,
4-Isoquinolinecarboxamide, 1,2,3,4-tetrahydro-3-(1H-indol-3-yl)-2-methyl-1-oxo-N-(4-phenoxyphenyl)-,
4-Isoquinolinecarboxamide, 3-[3,5-bis(trifluoromethyl)phenyl]-N-(5,8-diphenyl-1,2,4-triazocin-3-yl)-1,2,3,4-tetrahydro-2-methyl-1-oxo-,
2(1H)-Isoquinolinepropanamide, 3-[3,5-bis(trifluoromethyl)phenyl]-3,4-dihydro-1-oxo-4-[[[1-(phenylmethyl)-3-pyrrolidinyl]amino]carbonyl]-,
2(1H)-Isoquinolinepropanamide, 3-[3,5-bis(trifluoromethyl)phenyl]-3,4-dihydro-1-oxo-4-[[[1-(phenylmethyl)-4-piperidinyl]amino]carbonyl]-.

In still another particular embodiment, the compounds of the invention have a structure according to formula (V),

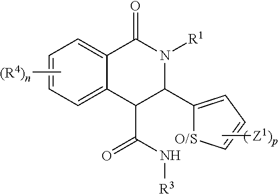

wherein,

R¹ is selected from heteroalkyl which can be unsubstituted or substituted with one or more Z¹,

R³ is selected from heterocycle, and aryl, wherein said aryl and heterocycle is optionally substituted with one or more of Z¹⁶,

each of R⁴ is independently selected from the group consisting of halogen, —OH, alkoxy, —SH, thioalkoxy, trifluoromethyl, trifluoromethoxy, nitro, cyano, —COOH, COO-alkyl, —COO-alkenyl, —COO-alkynyl, amino, heteroalkyl, alkyl, alkenyl, alkynyl, aryl, heterocycle, arylalkyl, arylalkenyl, arylalkynyl, heterocycle-alkyl, heterocycle-alkenyl, and heterocycle-alkynyl,

n is selected from 0, 1, 2, 3 or 4,

p is selected from 0, 1, 2, or 3,

each of Z¹ is independently selected from the group consisting of hydrogen, halogen, hydroxyl, sulfhydryl, alkoxy, thioalkoxy, ═O, ═S, trifluoromethyl, trifluoromethoxy, nitro, amino, cyano, —COOH, —COO-alkyl, —COO-alkenyl, —COO-alkynyl, —CONH₂, alkyl, alkenyl, alkynyl, aryl, heterocycle, arylalkyl, arylalkenyl, arylalkynyl, heterocycle-alkyl, heterocycle-alkenyl, heterocycle-alkynyl,

each of Z¹⁶ is independently selected from the group consisting of hydrogen, halogen, hydroxyl, sulfhydryl, alkoxy, thioalkoxy, ═O, ═S, trifluoromethyl, trifluoromethoxy, nitro, amino, cyano, alkyl, alkenyl, alkynyl, aryl, heterocycle, arylalkyl, arylalkenyl, arylalkynyl, heterocycle-alkyl, heterocycle-alkenyl, and heterocycle-alkynyl, wherein said alkoxy, thioalkoxy, alkyl, alkenyl, alkynyl, aryl, heterocycle, arylalkyl, arylalkenyl, arylalkynyl, heterocycle-alkyl, heterocycle-alkenyl, and heterocycle-alkynyl group optionally include one or more heteroatoms, said heteroatom selected from O, S, P and N, and wherein said alkoxy, thioalkoxy, alkyl, alkenyl, alkynyl, aryl, heterocycle, arylalkyl, arylalkenyl, arylalkynyl, heterocycle-alkyl, heterocycle-alkenyl, and heterocycle-alkynyl group is optionally substituted with one or more Z¹⁷,

each of Z¹⁷ is independently selected from halogen, —OH, alkoxy, —SH, thioalkoxy, trifluoromethyl, trifluoromethoxy, ═O, ═S, nitro, cyano, —COOH, —COO-alkyl, —COO-alkenyl, —COO-alkynyl, amino, heteroalkyl, alkyl, alkenyl, alkynyl, aryl, heterocycle, arylalkyl, arylalkenyl, arylalkynyl, heterocycle-alkyl, heterocycle-alkenyl, and heterocycle-alkynyl. and isomers (in particular stereo-isomers or tautomers), solvates, hydrates, salts (in particular pharmaceutically acceptable salts) or prodrugs thereof.

According to a preferred embodiment of the compounds with a structure according to formula (V): R¹ is a C₁₋₁₈-heteroalkyl group or a C₁₋₁₈-alkyl group optionally substituted with an optionally C₁₋₆-alkyl group-substituted heterocyclic group, R³ is an aryl, C₁₋₆-alkyl or a heterocyclic group optionally substituted with one of more Z¹, R⁴ is a halogen atom, a C₁₋₁₈-alkyl group, a C₁₋₁₈-alkoxy group, a nitro group or an amino group, Z¹ is independently selected from the group consisting of halogen, hydroxyl, C₁₋₆-alkoxy, C₁₋₆-thioalkoxy, trifluoromethyl, trifluoromethoxy, nitro, amino, cyano, —NH—SO₂—C₁₋₆-alkyl, —COO—C₁₋₆-alkyl, —COO—C₂₋₆-alkenyl, —COO—C₂₋₆-alkynyl, C₁₋₆-alkyl, C₂₋₆-alkenyl, C₂₋₆-alkynyl and aryl groups, n is 0, 1, 2, 3 or 4, p=0, 1, 2 or 3, wherein n is preferably 0 i.e. the four free positions of the benzene ring are unsubstituted, and isomers (in particular stereo-isomers or tautomers), solvates, hydrates, salts (in particular pharmaceutically acceptable salts) or prodrugs thereof.

According to a particularly preferred embodiment of the compounds with the structure according to formula (V), the compounds are exclusive of one or more of:
4-Isoquinolinecarboxamide, 1,2,3,4-tetrahydro-2-(2-methoxyethyl)-N-(2-methylcyclohexyl)-1-oxo-3-(2-thienyl)-,
4-Isoquinolinecarboxamide, 1,2,3,4-tetrahydro-2-(2-methoxyethyl)-N-(4-methylcyclohexyl)-1-oxo-3-(2-thienyl)-,
4-Isoquinolinecarboxamide, 1,2,3,4-tetrahydro-2-(2-methoxyethyl)-1-oxo-N-(1,2,3,4-tetrahydro-1-naphthalenyl)-3-(2-thienyl)-,
4-Isoquinolinecarboxamide, N-(2,3-dimethylcyclohexyl)-1,2,3,4-tetrahydro-2-(2-methoxyethyl)-1-oxo-3-(2-thienyl)-,
4-Isoquinolinecarboxamide, N-(3,5-dimethylphenyl)-1,2,3,4-tetrahydro-2-(2-methoxyethyl)-1-oxo-3-(2-thienyl)-,
Benzoic acid, 4-[[[1,2,3,4-tetrahydro-2-(2-methoxyethyl)-1-oxo-3-(2-thienyl)-4-isoquinolinyl]carbonyl]amino]-, methyl ester,
4-Isoquinolinecarboxamide, N-[4-[(4-ethyl-1-piperazinyl)methyl]phenyl]-1,2,3,4-tetrahydro-2-(2-methoxyethyl)-1-oxo-3-(2-thienyl)-,
4-Isoquinolinecarboxamide, 1,2,3,4-tetrahydro-2-(2-methoxyethyl)-N-[4-[(4-methyl-1-piperidinyl)methyl]phenyl]-1-oxo-3-(2-thienyl)-,
4-Isoquinolinecarboxamide, 1,2,3,4-tetrahydro-2-(2-methoxyethyl)-N-(4-methyl-2-oxo-2H-1-benzopyran-7-yl)-1-oxo-3-(2-thienyl)-,
4-Isoquinolinecarboxamide, N-(3-acetylphenyl)-1,2,3,4-tetrahydro-2-(2-methoxyethyl)-1-oxo-3-(2-thienyl)-,
Benzoic acid, 4-[[[1,2,3,4-tetrahydro-2-(2-methoxyethyl)-1-oxo-3-(2-thienyl)-4-isoquinolinyl]carbonyl]amino]-, ethyl ester,
4-Isoquinolinecarboxamide, N-(4-chlorophenyl)-1,2,3,4-tetrahydro-2-(2-methoxyethyl)-1-oxo-3-(2-thienyl)-,
4-Isoquinolinecarboxamide, N-(2-chlorophenyl)-1,2,3,4-tetrahydro-2-(2-methoxyethyl)-1-oxo-3-(2-thienyl)-,
4-Isoquinolinecarboxamide, N-(2-cyanophenyl)-1,2,3,4-tetrahydro-2-(2-methoxyethyl)-1-oxo-3-(2-thienyl)-,
4-Isoquinolinecarboxamide, N-(2-carbamoylphenyl)-1,2,3,4-tetrahydro-2-(2-methoxyethyl)-1-oxo-3-(2-thienyl)-,
4-Isoquinolinecarboxamide, 1,2,3,4-tetrahydro-2-(2-methoxyethyl)-N-(4-methoxyphenyl)-1-oxo-3-(2-thienyl)-,
4-Isoquinolinecarboxamide, N-(2-chloro-3-pyridinyl)-1,2,3,4-tetrahydro-2-(2-methoxyethyl)-1-oxo-3-(2-thienyl)-,
4-Isoquinolinecarboxamide, N-(4-acetylphenyl)-1,2,3,4-tetrahydro-2-(2-methoxyethyl)-1-oxo-3-(2-thienyl)-,
4-Isoquinolinecarboxamide, N-(2-ethylphenyl)-1,2,3,4-tetrahydro-2-(2-methoxyethyl)-1-oxo-3-(2-thienyl)-,
4-Isoquinolinecarboxamide, 1,2,3,4-tetrahydro-2-(2-methoxyethyl)-N-(2-methylphenyl)-1-oxo-3-(2-thienyl)-,
4-Isoquinolinecarboxamide, 1,2,3,4-tetrahydro-2-(2-methoxyethyl)-N-[2-(4-methyl-1-piperazinyl)phenyl]-1-oxo-3-(2-thienyl)-,
4-Isoquinolinecarboxamide, 1,2,3,4-tetrahydro-2-(2-methoxyethyl)-1-oxo-N-3-pyridinyl-3-(2-thienyl)-,
4-Isoquinolinecarboxamide, N-(5-chloro-2-pyridinyl)-1,2,3,4-tetrahydro-2-(2-methoxyethyl)-1-oxo-3-(2-thienyl)-,
4-Isoquinolinecarboxamide, 1,2,3,4-tetrahydro-2-(2-methoxyethyl)-N-(4-methyl-2-pyridinyl)-1-oxo-3-(2-thienyl)-,
4-Isoquinolinecarboxamide, N-cyclopentyl-1,2,3,4-tetrahydro-2-(2-methoxyethyl)-1-oxo-3-(2-thienyl)-,
4-Isoquinolinecarboxamide, 1,2,3,4-tetrahydro-2-(2-methoxyethyl)-N-[3-(methylthio)phenyl]-1-oxo-3-(2-thienyl)-,
4-Isoquinolinecarboxamide, 1,2,3,4-tetrahydro-2-(2-methoxyethyl)-N-(5-methyl-2-pyridinyl)-1-oxo-3-(2-thienyl)-,
4-Isoquinolinecarboxamide, N-(3-ethylphenyl)-1,2,3,4-tetrahydro-2-(2-methoxyethyl)-1-oxo-3-(2-thienyl)-,
4-Isoquinolinecarboxamide, N-(3-cyanophenyl)-1,2,3,4-tetrahydro-2-(2-methoxyethyl)-1-oxo-3-(2-thienyl)-,
4-Isoquinolinecarboxamide, 1,2,3,4-tetrahydro-2-(2-methoxyethyl)-1-oxo-N-[1-(phenylmethyl)-4-piperidinyl]-3-(2-thienyl)-,
4-Isoquinolinecarboxamide, N-cyclopropyl-1,2,3,4-tetrahydro-2-(2-methoxyethyl)-1-oxo-3-(2-thienyl)-,
4-Isoquinolinecarboxamide, N-(2-chloro-4-methylphenyl)-1,2,3,4-tetrahydro-2-(2-methoxyethyl)-1-oxo-3-(2-thienyl)-,
1-Piperidinecarboxylic acid, 4-[[[1,2,3,4-tetrahydro-2-(2-methoxyethyl)-1-oxo-3-(2-thienyl)-4-isoquinolinyl]carbonyl]amino]-, ethyl ester,
4-Isoquinolinecarboxamide, N-(4-chloro-2-methoxy-5-methylphenyl)-1,2,3,4-tetrahydro-2-(2-methoxyethyl)-1-oxo-3-(2-thienyl)-,
4-Isoquinolinecarboxamide, 1,2,3,4-tetrahydro-2-(2-methoxyethyl)-N-[4-(1-methylethyl)phenyl]-1-oxo-3-(2-thienyl)-,
4-Isoquinolinecarboxamide, N-(4-ethylphenyl)-1,2,3,4-tetrahydro-2-(2-methoxyethyl)-1-oxo-3-(2-thienyl)-,
4-Isoquinolinecarboxamide, N-(2-ethoxyphenyl)-1,2,3,4-tetrahydro-2-(2-methoxyethyl)-1-oxo-3-(2-thienyl)-,
Benzoic acid, 2-[[[1,2,3,4-tetrahydro-2-(2-methoxyethyl)-1-oxo-3-(2-thienyl)-4-isoquinolinyl]carbonyl]amino]-, ethyl ester,
4-Isoquinolinecarboxamide, 1,2,3,4-tetrahydro-2-(2-methoxyethyl)-N-(2-methoxy-5-nitrophenyl)-1-oxo-3-(2-thienyl)-,
4-Isoquinolinecarboxamide, N-(2,5-dimethoxyphenyl)-1,2,3,4-tetrahydro-2-(2-methoxyethyl)-1-oxo-3-(2-thienyl)-,
4-Isoquinolinecarboxamide, N-(4-ethoxyphenyl)-1,2,3,4-tetrahydro-2-(2-methoxyethyl)-1-oxo-3-(2-thienyl)-,
Benzoic acid, 2-[[[1,2,3,4-tetrahydro-2-(2-methoxyethyl)-1-oxo-3-(2-thienyl)-4-isoquinolinyl]carbonyl]amino]-, methyl ester,
4-Isoquinolinecarboxamide, N-(2,3-dihydro-1,4-benzodioxin-6-yl)-1,2,3,4-tetrahydro-2-(2-methoxyethyl)-1-oxo-3-(2-thienyl)-,
4-Isoquinolinecarboxamide, N-(5-chloro-2,4-dimethoxyphenyl)-1,2,3,4-tetrahydro-2-(2-methoxyethyl)-1-oxo-3-(2-thienyl)-,
4-Isoquinolinecarboxamide, 1,2,3,4-tetrahydro-2-(2-methoxyethyl)-N-(3-methoxyphenyl)-1-oxo-3-(2-thienyl)-,
4-Isoquinolinecarboxamide, N-(2-chloro-4-fluorophenyl)-1,2,3,4-tetrahydro-2-(2-methoxyethyl)-1-oxo-3-(2-thienyl)-,
4-Isoquinolinecarboxamide, N-(5-chloro-2-methoxyphenyl)-1,2,3,4-tetrahydro-2-(2-methoxyethyl)-1-oxo-3-(2-thienyl)-,
4-Isoquinolinecarboxamide, N-(3,4-dimethoxyphenyl)-1,2,3,4-tetrahydro-2-(2-methoxyethyl)-1-oxo-3-(2-thienyl)-,
4-Isoquinolinecarboxamide, N-(2,4-dimethoxyphenyl)-1,2,3,4-tetrahydro-2-(2-methoxyethyl)-1-oxo-3-(2-thienyl)-,
4-Isoquinolinecarboxamide, N-(3-chloro-4-methoxyphenyl)-1,2,3,4-tetrahydro-2-(2-methoxyethyl)-1-oxo-3-(2-thienyl)-,
4-Isoquinolinecarboxamide, N-(3-chloro-4-fluorophenyl)-1,2,3,4-tetrahydro-2-(2-methoxyethyl)-1-oxo-3-(2-thienyl)-,
4-Isoquinolinecarboxamide, N-cyclohexyl-1,2,3,4-tetrahydro-2-(2-methoxyethyl)-1-oxo-3-(2-thienyl)-,
4-Isoquinolinecarboxamide, 1,2,3,4-tetrahydro-2-(2-methoxyethyl)-1-oxo-N-2-pyridinyl-3-(2-thienyl)-,
4-Isoquinolinecarboxamide, N-(2-bromo-4-methylphenyl)-1,2,3,4-tetrahydro-2-(2-methoxyethyl)-1-oxo-3-(2-thienyl)-,
4-Isoquinolinecarboxamide, 1,2,3,4-tetrahydro-2-(2-methoxyethyl)-1-oxo-3-(2-thienyl)-N-[3-(trifluoromethyl)phenyl]-,
4-Isoquinolinecarboxamide, N-(3-chlorophenyl)-1,2,3,4-tetrahydro-2-(2-methoxyethyl)-1-oxo-3-(2-thienyl)-,
4-Isoquinolinecarboxamide, N-(4-fluorophenyl)-1,2,3,4-tetrahydro-2-(2-methoxyethyl)-1-oxo-3-(2-thienyl)-,
4-Isoquinolinecarboxamide, 1,2,3,4-tetrahydro-2-(2-methoxyethyl)-N-(2-methoxyphenyl)-1-oxo-3-(2-thienyl)-,
4-Isoquinolinecarboxamide, N-2-benzothiazolyl-1,2,3,4-tetrahydro-2-(2-methoxyethyl)-1-oxo-3-(2-thienyl)-, and
4-Isoquinolinecarboxamide, 1,2,3,4-tetrahydro-2-(2-methoxyethyl)-N-(6-methyl-2-pyridinyl)-1-oxo-3-(2-thienyl)-.

According to a further preferred embodiment of the compounds with the structure according to formula (V), the compound has a structure according to formula (Va)

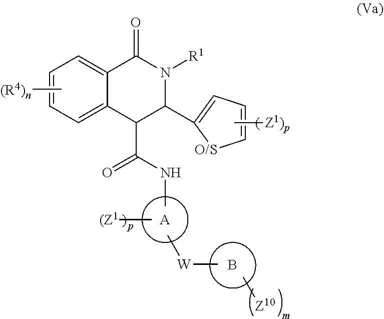

wherein,

each of cycle A and cycle B is independently selected from aryl or heterocycle,

“—W—” is a single bond, —O—, —S—, an alkylene group, a —C(═O)O— group, a —NH—C(═O)— group, a —C(═O)— group or a —C(═O)—NH— group, wherein said alkylene optionally includes one or more heteroatoms, said heteroatom selected from O, S, P and N,

each of R⁴ is independently selected from the group consisting of halogen, —OH, alkoxy, —SH, thioalkoxy, trifluoromethyl, trifluoromethoxy, nitro, cyano, —COOH, —COO-alkyl, —COO-alkenyl, —COO-alkynyl, amino, heteroalkyl, alkyl, alkenyl, alkynyl, aryl, heterocycle, arylalkyl, arylalkenyl, arylalkynyl, heterocycle-alkyl, heterocycle-alkenyl, and heterocycle-alkynyl.

each of Z¹ and Z¹⁰ are independently selected from the group consisting of hydrogen, halogen, hydroxyl, sulfhydryl, alkoxy, thioalkoxy, ═O, ═S, trifluoromethyl, trifluoromethoxy, nitro, amino, cyano, —COOH, —COO-alkyl, —COO-alkenyl, —COO-alkynyl, —CONH₂, alkyl, alkenyl, alkynyl, aryl, heterocycle, arylalkyl, arylalkenyl, arylalkynyl, heterocycle-alkyl, heterocycle-alkenyl, heterocycle-alkynyl,

m is 0, 1, 2, 3, or 4,

n is 0, 1, 2, 3, or 4,

p is 0, 1, 2, or 3,

and isomers (in particular stereo-isomers or tautomers), solvates, hydrates, salts (in particular pharmaceutically acceptable salts) or prodrugs thereof.

In a preferred embodiment, the compounds having a structure according to formula (Va):

R¹ is a C₁₋₁₈-heteroalkyl group or a C₁₋₁₈-alkyl group optionally substituted with an optionally C₁₋₆-alkyl group-substituted heterocyclic group,

each of cycle A and cycle B is independently selected from aryl or heterocyclic groups,

—W— is a single bond, —O—, —S—, a C₁₋₆-alkylene group, a —C(═O)O— group, a —NH—C(═O)— group, a —C(═O)— group or a —C(═O)—NH— group, wherein said C₁₋₆-alkylene group optionally includes one or more heteroatoms, said heteroatom selected from O, S, P and N,

R⁴ is a halogen atom, a C₁₋₁₈-alkyl group, a C₁₋₁₈-alkoxy group, a nitro group or an amino group,

Z¹ and Z¹⁰ are independently selected from the group consisting of halogen, hydroxyl, C₁₋₆-alkoxy, C₁₋₆-thioalkoxy, trifluoromethyl, trifluoromethoxy, nitro, amino, cyano, —NH—SO₂—C₁₋₆-alkyl, —COO—C₁₋₆-alkyl, —COO—C₂₋₆-alkenyl, —COO—C₂₋₆-alkynyl, amino, C₁₋₆-alkyl, C₂₋₆-alkenyl, C₂₋₆-alkynyl and aryl groups,

m is 0, 1, 2, 3 or 4, n is 0, 1, 2, 3 or 4, p=0, 1, 2 or 3, wherein n is preferably 0 i.e. the four free positions of the benzene ring are unsubstituted,
and isomers (in particular stereo-isomers or tautomers), solvates, hydrates, salts (in particular pharmaceutically acceptable salts) or prodrugs thereof.

In a particularly preferred embodiment of compounds according to formula (Va) the compounds are exclusive of 4-isoquinolinecarboxamide, N-[3-[4-(2,5-dimethylphenyl)-1-piperazinyl]propyl]-1,2,3,4-tetrahydro-2-(2-methoxyethyl)-1-oxo-3-(2-thienyl)-, 4-isoquinolinecarboxamide, N-[4-[(4-ethyl-1-piperazinyl)methyl]phenyl]-1,2,3,4-tetrahydro-2-(2-methoxyethyl)-1-oxo-3-(2-thienyl)-, 4-isoquinolinecarboxamide, 1,2,3,4-tetrahydro-2-(2-methoxyethyl)-N-[4-[(4-methyl-1-piperidinyl)methyl]phenyl]-1-oxo-3-(2-thienyl)-, 4-isoquinolinecarboxamide, N-[3-[4-(5-chloro-2-methylphenyl)-1-piperazinyl]propyl]-1,2,3,4-tetrahydro-2-(2-methoxyethyl)-1-oxo-3-(2-thienyl)-, 4-isoquinolinecarboxamide, 1,2,3,4-tetrahydro-2-(2-methoxyethyl)-N-[2-(4-methyl-1-piperazinyl)phenyl]-1-oxo-3-(2-thienyl)-, 4-isoquinolinecarboxamide, 1,2,3,4-tetrahydro-2-(2-methoxyethyl)-1-oxo-N-[3-[4-(phenylmethyl)-1-piperazinyl]propyl]-3-(2-thienyl)-, 4-isoquinolinecarboxamide, 1,2,3,4-tetrahydro-2-(2-methoxyethyl)-N-[[1-[(3-methylphenyl)methyl]-4-piperidinyl]methyl]-1-oxo-3-(2-thienyl)-, 4-isoquinolinecarboxamide, 1,2,3,4-tetrahydro-2-(2-methoxyethyl)-1-oxo-N-[1-(phenylmethyl)-4-piperidinyl]-3-(2-thienyl)-, and 4-isoquinolinecarboxamide, 1,2,3,4-tetrahydro-2-(2-methoxyethyl)-N-[2-[4-(4-methoxyphenyl)-1-piperazinyl]ethyl]-1-oxo-3-(2-thienyl)-.

In still another particular embodiment, the compounds of the invention have a structure according to formula (Vb)

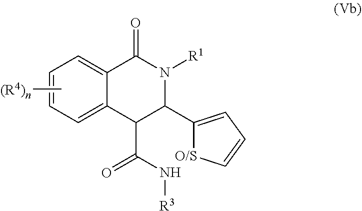

wherein,
R¹ is a C₁₋₁₈-heteroalkyl group or a C₁₋₁₈-alkyl group optionally substituted with an optionally C₁₋₆-alkyl group-substituted heterocyclic group, R³ is an aryl, C₁₋₁₈-alkyl, heterocyclic group optionally substituted with one of more Z¹, R⁴ is a halogen atom, a C₁₋₆-alkyl group, a C₁₋₆-alkoxy group, a nitro group or an amino group, Z¹ is independently selected from the group consisting of halogen, hydroxyl, C₁₋₆-alkoxy, C₁₋₆-thioalkoxy, trifluoromethyl, trifluoromethoxy, nitro, amino, cyano, —NH—SO₂—C₁₋₆-alkyl, —COO—C₁₋₆-alkyl, —COO—C₂₋₆-alkenyl, —COO—C₂₋₆-alkynyl, C₁₋₆-alkyl, C₂₋₆-alkenyl, C₂₋₆-alkynyl and aryl groups,
n is 0, 1, 2, 3 or 4, wherein n is preferably 0 i.e. the four free positions of the benzene ring are unsubstituted, and isomers (in particular stereo-isomers or tautomers), solvates, hydrates, salts (in particular pharmaceutically acceptable salts) or prodrugs thereof.

In a preferred embodiment of the compounds according to formulae (V) and (Vb), R¹ is a C₁₋₆ heteroalkyl group or a C₁₋₆ alkyl group optionally substituted with an optionally alkyl group-substituted heterocyclic group, R³ is an optionally substituted aryl, heterecyclic or C₁₋₆-alkyl group with one or more of Z¹, Z¹ is independently selected from the group consisting of halogen, hydroxyl, C₁₋₆-alkoxy, C₁₋₆-thioalkoxy, trifluoromethyl, trifluoromethoxy, nitro, amino, cyano, —NH—SO₂—C₁₋₆-alkyl, —COO—C₁₋₆-alkyl, —COO—C₂₋₆-alkenyl, —COO—C₂₋₆-alkynyl, C₁₋₆-alkyl, C₂₋₆-alkenyl, C₂₋₆-alkynyl and aryl groups, p if applicable is 0 or 1 and n=0 i.e. the four free hydrogen atoms of the benzene ring are unsubstituted.

In a particularly preferred embodiment of the compounds according to formula (V) and (Vb) R¹ is a C₁₋₆ heteroalkyl group or a C₁₋₆ alkyl group optionally substituted with an optionally C₁₋₆-alkyl group-substituted heterocyclic group, R³ is an aryl or heterocyclic group optionally substituted with one or more of Z¹, Z¹ is independently selected from the group consisting of halogen, hydroxyl, C₁₋₆-alkoxy, C₁₋₆-thioalkoxy, trifluoromethyl, trifluoromethoxy, nitro, amino, cyano, —NH—SO₂—C₁₋₆-alkyl, —COO—C₁₋₆-alkyl, —COO—C₂₋₆-alkenyl, —COO—C₂₋₆-alkynyl, C₁₋₆-alkyl, C₂₋₆-alkenyl, C₂₋₆-alkynyl and aryl groups, p if applicable is 0 and n=0 i.e. the four free hydrogen atoms of the benzene ring are unsubstituted.

A very particular embodiment relates to the compounds selected from:
(3R,4R)-2-(2-methoxyethyl)-N-(3-methoxyphenyl)-1-oxo-3-(thiophen-2-yl)-1,2,3,4-tetrahydroisoquinoline-4-carboxamide
(3S,4R)-2-(2-methoxyethyl)-N-(3-methoxyphenyl)-1-oxo-3-(thiophen-2-yl)-1,2,3,4-tetrahydroisoquinoline-4-carboxamide
2-(2-methoxyethyl)-N-(3-methoxyphenyl)-7-nitro-1-oxo-3-(thiophen-2-yl)-1,2,3,4-tetrahydroisoquinoline-4-carboxamide
2-(furan-2-ylmethyl)-N-(3-methoxyphenyl)-1-oxo-3-(thiophen-2-yl)-1,2,3,4-tetrahydroisoquinoline-4-carboxamide
2-ethyl-N-(3-methoxyphenyl)-1-oxo-3-(thiophen-2-yl)-1,2,3,4-tetrahydroisoquinoline-4-carboxamide
3-(furan-2-yl)-2-(2-methoxyethyl)-N-(3-methoxyphenyl)-1-oxo-1,2,3,4-tetrahydroisoquinoline-4-carboxamide
3-(2-fluorophenyl)-2-(2-methoxyethyl)-N-(3-methoxyphenyl)-1-oxo-1,2,3,4-tetrahydroisoquinoline-4-carboxamide
3-(3-fluorophenyl)-2-(2-methoxyethyl)-N-(3-methoxyphenyl)-1-oxo-1,2,3,4-tetrahydroisoquinoline-4-carboxamide
3-(4-fluorophenyl)-2-(2-methoxyethyl)-N-(3-methoxyphenyl)-1-oxo-1,2,3,4-tetrahydroisoquinoline-4-carboxamide
3-(2,6-difluorophenyl)-2-(2-methoxyethyl)-N-(3-methoxyphenyl)-1-oxo-1,2,3,4-tetrahydroisoquinoline-4-carboxamide
2-(2-methoxyethyl)-N-(3-methoxyphenyl)-1-oxo-3-(thiophen-3-yl)-1,2,3,4-tetrahydroisoquinoline-4-carboxamide
2-(2-methoxyethyl)-N-(3-methoxyphenyl)-3-(5-methylthiophen-2-yl)-1-oxo-1,2,3,4-tetrahydroisoquinoline-4-carboxamide
N-(3-methoxyphenyl)-2-(3-methoxypropyl)-1-oxo-3-(thiophen-2-yl)-1,2,3,4-tetrahydroisoquinoline-4-carboxamide
methyl 2-(4-(3-methoxyphenylcarbamoyl)-1-oxo-3-(thiophen-2-yl)-3,4-dihydroisoquinolin-2(1H)-yl)acetate
2-(2-(dimethylamino)ethyl)-N-(3-methoxyphenyl)-1-oxo-3-(thiophen-2-yl)-1,2,3,4-tetrahydroisoquinoline-4-carboxamide
2-butyl-N-(3-methoxyphenyl)-1-oxo-3-(thiophen-2-yl)-1,2,3,4-tetrahydroisoquinoline-4-carboxamide
3-(3-chlorothiophen-2-yl)-2-(2-methoxyethyl)-N-(3-methoxyphenyl)-1-oxo-1,2,3,4-tetrahydroisoquinoline-4-carboxamide
2-(2-methoxyethyl)-1-oxo-N-phenyl-3-(thiophen-2-yl)-1,2,3,4-tetrahydroisoquinoline-4-carboxamide
2-(2-methoxyethyl)-1-oxo-3-(thiophen-2-yl)-N-(4-(trifluoromethyl)phenyl)-1,2,3,4-tetrahydroisoquinoline-4-carboxamide
N-(4-cyanophenyl)-2-(2-methoxyethyl)-1-oxo-3-(thiophen-2-yl)-1,2,3,4-tetrahydroisoquinoline-4-carboxamide
N-(biphenyl-3-yl)-2-(2-methoxyethyl)-1-oxo-3-(thiophen-2-yl)-1,2,3,4-tetrahydroisoquinoline-4-carboxamide
2-(cyclohexylmethyl)-N-(3-methoxyphenyl)-1-oxo-3-(thiophen-2-yl)-1,2,3,4-tetrahydroisoquinoline-4-carboxamide
2-(2-hydroxyethyl)-N-(3-methoxyphenyl)-1-oxo-3-(thiophen-2-yl)-1,2,3,4-tetrahydroisoquinoline-4-carboxamide
8-Chloro-2-(2-methoxyethyl)-N-(3-methoxyphenyl)-1-oxo-3-(thiophen-2-yl)-1,2,3,4-tetrahydroisoquinoline-4-carboxamide
2-cyclohexyl-N-(3-methoxyphenyl)-1-oxo-3-(thiophen-2-yl)-1,2,3,4-tetrahydroisoquinoline-4-carboxamide
2-(2-methoxyethyl)-N-(3-methoxyphenyl)-1-oxo-3-(pyridin-2-yl)-1,2,3,4-tetrahydroisoquinoline-4-carboxamide
3-(benzo[b]thiophen-2-yl)-2-(2-methoxyethyl)-N-(3-methoxyphenyl)-1-oxo-1,2,3,4-tetrahydroisoquinoline-4-carboxamide
2-(2-methoxyethyl)-N-(3-methoxyphenyl)-1-oxo-3-phenyl-1,2,3,4-tetrahydroisoquinoline-4-carboxamide
2-(2-methoxyethyl)-N-(3-methoxyphenyl)-1-oxo-3-(1H-pyrrol-2-yl)-1,2,3,4-tetrahydroisoquinoline-4-carboxamide
2-(2-methoxyethyl)-N-(3-methoxyphenyl)-1-oxo-3-(pyridin-4-yl)-1,2,3,4-tetrahydroisoquinoline-4-carboxamide
N-(3-methoxyphenyl)-1-oxo-2-((tetrahydrofuran-2-yl)methyl)-3-(thiophen-2-yl)-1,2,3,4-tetrahydroisoquinoline-4-carboxamide
2-(2-methoxyethyl)-N-(3-methoxyphenyl)-1-oxo-3-(thiophen-2-yl)-1,2-dihydroisoquinoline-4-carboxamide
4-(hydroxymethyl)-2-(2-methoxyethyl)-3-(thiophen-2-yl)-3,4-dihydroisoquinolin-1(2H)-one
2-(2-methoxyethyl)-1-oxo-N-(2-phenoxyethyl)-3-(thiophen-2-yl)-1,2,3,4-tetrahydroisoquinoline-4-carboxamide
3-(1H-imidazol-2-yl)-2-(2-methoxyethyl)-N-(3-methoxyphenyl)-1-oxo-1,2,3,4-tetrahydroisoquinoline-4-carboxamide
2-(2-methoxyethyl)-1-oxo-3-(thiophen-2-yl)-1,2,3,4-tetrahydroisoquinoline-4-carboxylic acid
2-benzyl-N-(3-methoxyphenyl)-1-oxo-3-(thiophen-2-yl)-1,2,3,4-tetrahydroisoquinoline-4-carboxamide
3-(furan-2-yl)-2-(2-methoxyethyl)-N-(3-methoxyphenyl)-1-oxo-1,2-dihydroisoquinoline-4-carboxamide
2-(2-methoxyethyl)-N-(3-methoxyphenyl)-5-methyl-1-oxo-3-(thiophen-2-yl)-1,2,3,4-tetrahydroisoquinoline-4-carboxamide
2-(2-methoxyethyl)-N-(3-methoxyphenyl)-6-methyl-1-oxo-3-(thiophen-2-yl)-1,2,3,4-tetrahydroisoquinoline-4-carboxamide
2-(2-methoxyethyl)-N-(3-methoxyphenyl)-7-methyl-1-oxo-3-(thiophen-2-yl)-1,2,3,4-tetrahydroisoquinoline-4-carboxamide
2-(2-methoxyethyl)-N-(3-methoxyphenyl)-8-methyl-1-oxo-3-(thiophen-2-yl)-1,2,3,4-tetrahydroisoquinoline-4-carboxamide
7-chloro-2-(2-methoxyethyl)-N-(3-methoxyphenyl)-1-oxo-3-(thiophen-2-yl)-1,2,3,4-tetrahydroisoquinoline-4-carboxamide
2-(2-methoxyethyl)-N-(2-(1-methyl-1H-indol-3-yl)ethyl)-1-oxo-3-(thiophen-2-yl)-1,2,3,4-tetrahydroisoquinoline-4-carboxamide
methyl 4-(2-(2-methoxyethyl)-1-oxo-3-(thiophen-2-yl)-1,2,3,4-tetrahydroisoquinoline-4-carboxamido)benzoate
ethyl 3-(2-(2-methoxyethyl)-1-oxo-3-(thiophen-2-yl)-1,2,3,4-tetrahydroisoquinoline-4-carboxamido)benzoate
N-(3-fluorophenyl)-2-(2-methoxyethyl)-1-oxo-3-(thiophen-2-yl)-1,2,3,4-tetrahydroisoquinoline-4-carboxamide
N-(3,4-dimethoxyphenyl)-2-(2-methoxyethyl)-1-oxo-3-(thiophen-2-yl)-1,2,3,4-tetrahydroisoquinoline-4-carboxamide
N-(4-chlorophenyl)-2-(2-methoxyethyl)-1-oxo-3-(thiophen-2-yl)-1,2,3,4-tetrahydroisoquinoline-4-carboxamide
7-methoxy-2-(2-methoxyethyl)-N-(3-methoxyphenyl)-1-oxo-3-(thiophen-2-yl)-1,2,3,4-tetrahydroisoquinoline-4-carboxamide
8-fluoro-2-(2-methoxyethyl)-N-(3-methoxyphenyl)-1-oxo-3-(thiophen-2-yl)-1,2,3,4-tetrahydroisoquinoline-4-carboxamide
7-fluoro-2-(2-methoxyethyl)-N-(3-methoxyphenyl)-1-oxo-3-(thiophen-2-yl)-1,2,3,4-tetrahydroisoquinoline-4-carboxamide
2-(2-methoxyethyl)-N-(3-methoxyphenyl)-1-oxo-3-(pyridin-3-yl)-1,2,3,4-tetrahydroisoquinoline-4-carboxamide
2-(2-methoxyethyl)-N-(3-methoxyphenyl)-3-(4-methyl-1H-imidazol-5-yl)-1-oxo-1,2,3,4-tetrahydroisoquinoline-4-carboxamide
6-chloro-2-(2-methoxyethyl)-N-(3-methoxyphenyl)-1-oxo-3-(thiophen-2-yl)-1,2,3,4-tetrahydroisoquinoline-4-carboxamide
7-iodo-2-(2-methoxyethyl)-N-(3-methoxyphenyl)-1-oxo-3-(thiophen-2-yl)-1,2,3,4-tetrahydroisoquinoline-4-carboxamide
3-tert-butyl-2-(2-methoxyethyl)-N-(3-methoxyphenyl)-1-oxo-1,2,3,4-tetrahydroisoquinoline-4-carboxamide
3-ethyl-2-(2-methoxyethyl)-N-(3-methoxyphenyl)-1-oxo-1,2,3,4-tetrahydroisoquinoline-4-carboxamide
N-(3-ethoxyphenyl)-2-(2-methoxyethyl)-1-oxo-3-(thiophen-2-yl)-1,2,3,4-tetrahydroisoquinoline-4-carboxamide
N-(3-chlorophenyl)-2-(2-methoxyethyl)-1-oxo-3-(thiophen-2-yl)-1,2,3,4-tetrahydroisoquinoline-4-carboxamide
5-fluoro-2-(2-methoxyethyl)-N-(3-methoxyphenyl)-1-oxo-3-(thiophen-2-yl)-1,2,3,4-tetrahydroisoquinoline-4-carboxamide
N-(3-methoxyphenyl)-1-oxo-3-(thiophen-2-yl)-2-(2,2,2-trifluoroethyl)-1,2,3,4-tetrahydroisoquinoline-4-carboxamide
N-(3-methoxyphenyl)-2-(2-(methylthio)ethyl)-1-oxo-3-(thiophen-2-yl)-1,2,3,4-tetrahydroisoquinoline-4-carboxamide
N-(3-methoxyphenyl)-2-(3-morpholinopropyl)-1-oxo-3-(thiophen-2-yl)-1,2,3,4-tetrahydroisoquinoline-4-carboxamide
2-(2-ethoxyethyl)-N-(3-methoxyphenyl)-1-oxo-3-(thiophen-2-yl)-1,2,3,4-tetrahydroisoquinoline-4-carboxamide
2-(2-isopropoxyethyl)-N-(3-methoxyphenyl)-1-oxo-3-(thiophen-2-yl)-1,2,3,4-tetrahydroisoquinoline-4-carboxamide
2-((1-ethylpyrrolidin-2-ylmethyl)-N-(3-methoxyphenyl)-1-oxo-3-(thiophen-2-yl)-1,2,3,4-tetrahydroisoquinoline-4-carboxamide
3-benzyl-2-(2-methoxyethyl)-N-(3-methoxyphenyl)-1-oxo-1,2,3,4-tetrahydroisoquinoline-4-carboxamide
2-(2-methoxyethyl)-N-(3-methoxyphenyl)-3-methyl-1-oxo-1,2,3,4-tetrahydroisoquinoline-4-carboxamide
2-(2-methoxyethyl)-N-(3-methoxyphenyl)-1-oxo-3-((trimethylsilyl)ethynyl)-1,2,3,4-tetrahydroisoquinoline-4-carboxamide
2-(2-methoxyethyl)-N-(3-methoxyphenyl)-1-oxo-3-(2,2,2-trifluoroethyl)-1,2,3,4-tetrahydroisoquinoline-4-carboxamide
2-(2-methoxyethyl)-N-(naphthalen-1-ylmethyl)-1-oxo-3-(thiophen-2-yl)-1,2,3,4-tetrahydroisoquinoline-4-carboxamide
2-(2-methoxyethyl)-4-((3-methoxyphenylamino)methyl)-3-(thiophen-2-yl)-3,4-dihydroisoquinolin-1(2H)-one
2-(((S)-1-ethylpyrrolidin-2-yl)methyl)-N-(3-methoxyphenyl)-1-oxo-3-(thiophen-2-yl)-1,2,3,4-tetrahydroisoquinoline-4-carboxamide
6,7-dimethoxy-2-(2-methoxyethyl)-N-(3-methoxyphenyl)-1-oxo-3-(thiophen-2-yl)-1,2,3,4-tetrahydroisoquinoline-4-carboxamide
4-(benzyloxymethyl)-2-(2-methoxyethyl)-3-(thiophen-2-yl)-3,4-dihydroisoquinolin-1(2H)-one
6-fluoro-2-(2-methoxyethyl)-N-(3-methoxyphenyl)-1-oxo-3-(thiophen-2-yl)-1,2,3,4-tetrahydroisoquinoline-4-carboxamide
4-((4-chlorophenylamino)methyl)-2-(2-methoxyethyl)-3-(thiophen-2-yl)-3,4-dihydroisoquinolin-1(2H)-one
N-(4-methoxybenzyl)-2-(2-methoxyethyl)-1-oxo-3-(thiophen-2-yl)-1,2,3,4-tetrahydroisoquinoline-4-carboxamide
2-(2-methoxyethyl)-1-oxo-N-(pyridin-4-ylmethyl)-3-(thiophen-2-yl)-1,2,3,4-tetrahydroisoquinoline-4-carboxamide
N-(benzo[d]thiazol-5-yl)-2-(2-methoxyethyl)-1-oxo-3-(thiophen-2-yl)-1,2,3,4-tetrahydroisoquinoline-4-carboxamide
2-(2-methoxyethyl)-N-(3-methoxyphenyl)-3-(methylthiomethyl)-1-oxo-1,2,3,4-tetrahydroisoquinoline-4-carboxamide
3-ethynyl-2-(2-methoxyethyl)-N-(3-methoxyphenyl)-1-oxo-1,2,3,4-tetrahydroisoquinoline-4-carboxamide
N-(biphenyl-2-yl)-2-(2-methoxyethyl)-1-oxo-3-(thiophen-2-yl)-1,2,3,4-tetrahydroisoquinoline-4-carboxamide
2-(2-methoxyethyl)-1-oxo-3-(thiophen-2-yl)-N-m-tolyl-1,2,3,4-tetrahydroisoquinoline-4-carboxamide
2-(2-methoxyethyl)-1-oxo-3-(thiophen-2-yl)-N-p-tolyl-1,2,3,4-tetrahydroisoquinoline-4-carboxamide
N-(3,4-dichlorophenyl)-2-(2-methoxyethyl)-1-oxo-3-(thiophen-2-yl)-1,2,3,4-tetrahydroisoquinoline-4-carboxamide
N-(3,4-difluorophenyl)-2-(2-methoxyethyl)-1-oxo-3-(thiophen-2-yl)-1,2,3,4-tetrahydroisoquinoline-4-carboxamide
N-(3-benzylphenyl)-2-(2-methoxyethyl)-1-oxo-3-(thiophen-2-yl)-1,2,3,4-tetrahydroisoquinoline-4-carboxamide
N-(4-benzylphenyl)-2-(2-methoxyethyl)-1-oxo-3-(thiophen-2-yl)-1,2,3,4-tetrahydroisoquinoline-4-carboxamide
5,8-difluoro-2-(2-methoxyethyl)-N-(3-methoxyphenyl)-1-oxo-3-(thiophen-2-yl)-1,2,3,4-tetrahydroisoquinoline-4-carboxamide
2-(2-methoxyethyl)-N-(3-methoxyphenyl)-1-oxo-3-(thiophen-2-yl)-7-(trifluoromethyl)-1,2,3,4-tetrahydroisoquinoline-4-carboxamide
8-fluoro-2-(2-methoxyethyl)-N-(3-methoxyphenyl)-7-methyl-1-oxo-3-(thiophen-2-yl)-1,2,3,4-tetrahydroisoquinoline-4-carboxamide
N-(3-chloro-4-fluorophenyl)-2-(2-methoxyethyl)-1-oxo-3-(thiophen-2-yl)-1,2,3,4-tetrahydroisoquinoline-4-carboxamide
N-(4-fluorophenyl)-2-(2-methoxyethyl)-1-oxo-3-(thiophen-2-yl)-1,2,3,4-tetrahydroisoquinoline-4-carboxamide
3-(2,4-dimethylthiazol-5-yl)-2-(2-methoxyethyl)-N-(3-methoxyphenyl)-1-oxo-1,2,3,4-tetrahydroisoquinoline-4-carboxamide
2-(2-methoxyethyl)-N-(3-methoxyphenyl)-1-oxo-3-((tetrahydro-2H-pyran-4-ylmethyl)-1,2,3,4-tetrahydroisoquinoline-4-carboxamide
2-(2-methoxyethyl)-N-(3-methoxyphenyl)-3-(2-methylthiazol-4-yl)-1-oxo-1,2,3,4-tetrahydroisoquinoline-4-carboxamide
3-(3,5-dimethylisoxazol-4-yl)-2-(2-methoxyethyl)-N-(3-methoxyphenyl)-1-oxo-1,2,3,4-tetrahydroisoquinoline-4-carboxamide
3-(1,3-dimethyl-1H-pyrazol-5-yl)-2-(2-methoxyethyl)-N-(3-methoxyphenyl)-1-oxo-1,2,3,4-tetrahydroisoquinoline-4-carboxamide
3-(1,5-dimethyl-1H-pyrazol-3-yl)-2-(2-methoxyethyl)-N-(3-methoxyphenyl)-1-oxo-1,2,3,4-tetrahydroisoquinoline-4-carboxamide
2-(2-methoxyethyl)-N-(3-methoxyphenyl)-1-oxo-3-(thiazol-2-yl)-1,2,3,4-tetrahydroisoquinoline-4-carboxamide
2-(2-methoxyethyl)-N-(3-methoxyphenyl)-3-(5-methylisoxazol-3-yl)-1-oxo-1,2,3,4-tetrahydroisoquinoline-4-carboxamide
2-(2-methoxyethyl)-N-(3-methoxyphenyl)-3-(3-methylisoxazol-5-yl)-1-oxo-1,2,3,4-tetrahydroisoquinoline-4-carboxamide
2-(2-methoxyethyl)-N-(3-methoxyphenyl)-3-(1-methyl-1H-pyrazol-5-yl)-1-oxo-1,2,3,4-tetrahydroisoquinoline-4-carboxamide
2-(2-methoxyethyl)-N-(3-methoxyphenyl)-3-(1-methyl-1H-pyrrol-2-yl)-1-oxo-1,2,3,4-tetrahydroisoquinoline-4-carboxamide
2-(2-methoxyethyl)-1-oxo-N-(4-(piperidin-1-yl)phenyl)-3-(thiophen-2-yl)-1,2,3,4-tetrahydroisoquinoline-4-carboxamide
N-(3-acetamidophenyl)-2-(2-methoxyethyl)-1-oxo-3-(thiophen-2-yl)-1,2,3,4-tetrahydroisoquinoline-4-carboxamide
N-(1,3-dimethyl-1H-pyrazol-5-yl)-2-(2-methoxyethyl)-1-oxo-3-(thiophen-2-yl)-1,2,3,4-tetrahydroisoquinoline-4-carboxamide
2-(2-methoxyethyl)-1-oxo-3-(thiophen-2-yl)-N-(3-(trifluoromethyl)phenyl)-1,2,3,4-tetrahydroisoquinoline-4-carboxamide
N-(4-chloro-3-methoxyphenyl)-2-(2-methoxyethyl)-1-oxo-3-(thiophen-2-yl)-1,2,3,4-tetrahydroisoquinoline-4-carboxamide
2-(2-methoxyethyl)-1-oxo-3-(thiophen-2-yl)-N-(3-(trifluoromethoxy)phenyl)-1,2,3,4-tetrahydroisoquinoline-4-carboxamide
2-(2-methoxyethyl)-1-oxo-3-(thiophen-2-yl)-N-(4-(trifluoromethoxy)phenyl)-1,2,3,4-tetrahydroisoquinoline-4-carboxamide
methyl 2-methoxy-4-(2-(2-methoxyethyl)-1-oxo-3-(thiophen-2-yl)-1,2,3,4-tetrahydroisoquinoline-4-carboxamido)benzoate
N-(3-(1H-pyrrol-1-yl)phenyl)-2-(2-methoxyethyl)-1-oxo-3-(thiophen-2-yl)-1,2,3,4-tetrahydroisoquinoline-4-carboxamide
N-(4-(1H-pyrrol-1-yl)phenyl)-2-(2-methoxyethyl)-1-oxo-3-(thiophen-2-yl)-1,2,3,4-tetrahydroisoquinoline-4-carboxamide
N-(2,3-dihydrobenzo[b][1,4]dioxin-6-yl)-2-(2-methoxyethyl)-1-oxo-3-(thiophen-2-yl)-1,2,3,4-tetrahydroisoquinoline-4-carboxamide
3-cyclopentyl-2-(2-methoxyethyl)-N-(3-methoxyphenyl)-1-oxo-1,2,3,4-tetrahydroisoquinoline-4-carboxamide
2-(2-methoxyethyl)-N-(3-methoxyphenyl)-1-oxo-3-(phenylethynyl)-1,2,3,4-tetrahydroisoquinoline-4-carboxamide
2-(2-methoxyethyl)-N-(3-methoxyphenyl)-3-(2-methylprop-1-enyl)-1-oxo-1,2,3,4-tetrahydroisoquinoline-4-carboxamide
2-(2-methoxyethyl)-N-(4-morpholinophenyl)-1-oxo-3-(thiophen-2-yl)-1,2,3,4-tetrahydroisoquinoline-4-carboxamide
2-(2-methoxyethyl)-1-oxo-N-(3-(pyrrolidin-1-yl)phenyl)-3-(thiophen-2-yl)-1,2,3,4-tetrahydroisoquinoline-4-carboxamide
2-(2-methoxyethyl)-N-(1-methyl-1H-indol-5-yl)-1-oxo-3-(thiophen-2-yl)-1,2,3,4-tetrahydroisoquinoline-4-carboxamide
2-(2-methoxyethyl)-N-((1r,4R)-4-methylcyclohexyl)-1-oxo-3-(thiophen-2-yl)-1,2,3,4-tetrahydroisoquinoline-4-carboxamide
N-(5-ethyl-1,3,4-oxadiazol-2-yl)-2-(2-methoxyethyl)-1-oxo-3-(thiophen-2-yl)-1,2,3,4-tetrahydroisoquinoline-4-carboxamide
2-(2-methoxyethyl)-N-(5-methylisoxazol-3-yl)-1-oxo-3-(thiophen-2-yl)-1,2,3,4-tetrahydroisoquinoline-4-carboxamide
2-(2-methoxyethyl)-N-(3-methylisoxazol-5-yl)-1-oxo-3-(thiophen-2-yl)-1,2,3,4-tetrahydroisoquinoline-4-carboxamide
8-fluoro-7-methoxy-2-(2-methoxyethyl)-N-(3-methoxyphenyl)-1-oxo-3-(thiophen-2-yl)-1,2,3,4-tetrahydroisoquinoline-4-carboxamide
N-(4-acetamidophenyl)-2-(2-methoxyethyl)-1-oxo-3-(thiophen-2-yl)-1,2,3,4-tetrahydroisoquinoline-4-carboxamide
N-(3,5-dimethoxyphenyl)-2-(2-methoxyethyl)-1-oxo-3-(thiophen-2-yl)-1,2,3,4-tetrahydroisoquinoline-4-carboxamide
2-(2-methoxyethyl)-N-(2-methyl-1H-indol-5-yl)-1-oxo-3-(thiophen-2-yl)-1,2,3,4-tetrahydroisoquinoline-4-carboxamide
N-(1H-indazol-5-yl)-2-(2-methoxyethyl)-1-oxo-3-(thiophen-2-yl)-1,2,3,4-tetrahydroisoquinoline-4-carboxamide
2-(2-methoxyethyl)-N,3-bis(3-methoxyphenyl)-1-oxo-1,2,3,4-tetrahydroisoquinoline-4-carboxamide
2-(2-methoxyethyl)-3-(2-methoxyphenyl)-N-(3-methoxyphenyl)-1-oxo-1,2,3,4-tetrahydroisoquinoline-4-carboxamide
3-(5-chlorothiophen-2-yl)-2-(2-methoxyethyl)-N-(3-methoxyphenyl)-1-oxo-1,2,3,4-tetrahydroisoquinoline-4-carboxamide
ethyl 1-(2-(2-methoxyethyl)-1-oxo-3-(thiophen-2-yl)-1,2,3,4-tetrahydroisoquinoline-4-carbonyl)piperidine-4-carboxylate
N-(4-(benzylcarbamoyl)phenyl)-2-(2-methoxyethyl)-1-oxo-3-(thiophen-2-yl)-1,2,3,4-tetrahydroisoquinoline-4-carboxamide
3-(benzyloxymethyl)-2-(2-methoxyethyl)-N-(3-methoxyphenyl)-1-oxo-1,2,3,4-tetrahydroisoquinoline-4-carboxamide
N-(4-(dimethylcarbamoyl)phenyl)-2-(2-methoxyethyl)-1-oxo-3-(thiophen-2-yl)-1,2,3,4-tetrahydroisoquinoline-4-carboxamide
3-(hydroxymethyl)-2-(2-methoxyethyl)-N-(3-methoxyphenyl)-1-oxo-1,2,3,4-tetrahydroisoquinoline-4-carboxamide
2-(2-methoxyethyl)-N-(4-(methylcarbamoyl)phenyl)-1-oxo-3-(thiophen-2-yl)-1,2,3,4-tetrahydroisoquinoline-4-carboxamide
N-(5-tert-butylisoxazol-3-yl)-2-(2-methoxyethyl)-1-oxo-3-(thiophen-2-yl)-1,2,3,4-tetrahydroisoquinoline-4-carboxamide
3-cyano-2-(2-methoxyethyl)-N-(3-methoxyphenyl)-1-oxo-1,2,3,4-tetrahydroisoquinoline-4-carboxamide
N-(2-amino-4-chlorophenyl)-2-(2-methoxyethyl)-1-oxo-3-(thiophen-2-yl)-1,2,3,4-tetrahydroisoquinoline-4-carboxamide
N-(2-amino-5-chlorophenyl)-2-(2-methoxyethyl)-1-oxo-3-(thiophen-2-yl)-1,2,3,4-tetrahydroisoquinoline-4-carboxamide
2-(2-methoxyethyl)-N4-(3-methoxyphenyl)-1-oxo-1,2,3,4-tetrahydroisoquinoline-3,4-dicarboxamide
7-amino-2-(2-methoxyethyl)-N-(3-methoxyphenyl)-1-oxo-3-(thiophen-2-yl)-1,2,3,4-tetrahydroisoquinoline-4-carboxamide
5-methoxy-2-(2-methoxyethyl)-N-(3-methoxyphenyl)-1-oxo-3-(thiophen-2-yl)-1,2,3,4-tetrahydroisoquinoline-4-carboxamide
2-(2-methoxyethyl)-N-(3-methoxyphenyl)-3-(methylsulfonylmethyl)-1-oxo-1,2,3,4-tetrahydroisoquinoline-4-carboxamide
N-(4-(1H-pyrrol-1-yl)phenyl)-8-fluoro-7-methoxy-2-(2-methoxyethyl)-1-oxo-3-(thiophen-2-yl)-1,2,3,4-tetrahydroisoquinoline-4-carboxamide
2-(2-methoxyethyl)-N-(3-methoxyphenyl)-N-methyl-1-oxo-3-(thiophen-2-yl)-1,2,3,4-tetrahydroisoquinoline-4-carboxamide
N-(4-(1H-pyrrol-1-yl)phenyl)-3-ethynyl-2-(2-methoxyethyl)-1-oxo-1,2,3,4-tetrahydroisoquinoline-4-carboxamide
N-(4-(1H-pyrrol-1-yl)phenyl)-3-(5-chlorothiophen-2-yl)-2-(2-methoxyethyl)-1-oxo-1,2,3,4-tetrahydroisoquinoline-4-carboxamide
2-(2-methoxyethyl)-N-(4-methyloxazol-2-yl)-1-oxo-3-(thiophen-2-yl)-1,2,3,4-tetrahydroisoquinoline-4-carboxamide
N-(4-cyano-3-methylisoxazol-5-yl)-2-(2-methoxyethyl)-1-oxo-3-(thiophen-2-yl)-1,2,3,4-tetrahydroisoquinoline-4-carboxamide
N-(4,5-dimethylthiazol-2-yl)-2-(2-methoxyethyl)-1-oxo-3-(thiophen-2-yl)-1,2,3,4-tetrahydroisoquinoline-4-carboxamide
2-(2-methoxyethyl)-N-(4-(2-methylthiazol-4-yl)phenyl)-1-oxo-3-(thiophen-2-yl)-1,2,3,4-tetrahydroisoquinoline-4-carboxamide
2-(2-methoxyethyl)-1-oxo-N-(6-phenoxypyridin-3-yl)-3-(thiophen-2-yl)-1,2,3,4-tetrahydroisoquinoline-4-carboxamide
N-(4-(furan-2-yl)phenyl)-2-(2-methoxyethyl)-1-oxo-3-(thiophen-2-yl)-1,2,3,4-tetrahydroisoquinoline-4-carboxamide
N-(5-(4-fluorophenyl)isoxazol-3-yl)-2-(2-methoxyethyl)-1-oxo-3-(thiophen-2-yl)-1,2,3,4-tetrahydroisoquinoline-4-carboxamide
N-(3-methoxyphenyl)-1-oxo-3-(thiophen-2-yl)-1,2,3,4-tetrahydroisoquinoline-4-carboxamide
2-(2-methoxyethyl)-1-oxo-N-(pyrimidin-4-yl)-3-(thiophen-2-yl)-1,2,3,4-tetrahydroisoquinoline-4-carboxamide
2-(2-methoxyethyl)-1-oxo-N-(pyrazin-2-yl)-3-(thiophen-2-yl)-1,2,3,4-tetrahydroisoquinoline-4-carboxamide
N-(benzo[d]thiazol-5-yl)-2-(2-methoxyethyl)-1-oxo-3-(thiophen-2-yl)-1,2,3,4-tetrahydroisoquinoline-4-carboxamide
N-(4-isopropylphenyl)-2-(2-methoxyethyl)-1-oxo-3-(thiophen-2-yl)-1,2,3,4-tetrahydroisoquinoline-4-carboxamide
N-(3-(hydroxymethyl)phenyl)-2-(2-methoxyethyl)-1-oxo-3-(thiophen-2-yl)-1,2,3,4-tetrahydroisoquinoline-4-carboxamide
N-(4-(1H-1,2,4-triazol-1-yl)phenyl)-2-(2-methoxyethyl)-1-oxo-3-(thiophen-2-yl)-1,2,3,4-tetrahydroisoquinoline-4-carboxamide
2-(2-methoxyethyl)-N-(2-methylquinolin-6-yl)-1-oxo-3-(thiophen-2-yl)-1,2,3,4-tetrahydroisoquinoline-4-carboxamide
N-(3,5-dimethylisoxazol-4-0)-2-(2-methoxyethyl)-1-oxo-3-(thiophen-2-yl)-1,2,3,4-tetrahydroisoquinoline-4-carboxamide
N-(benzo[d]thiazol-6-yl)-2-(2-methoxyethyl)-1-oxo-3-(thiophen-2-yl)-1,2,3,4-tetrahydroisoquinoline-4-carboxamide
2-(2-methoxyethyl)-1-oxo-N-(quinoxalin-6-yl)-3-(thiophen-2-yl)-1,2,3,4-tetrahydroisoquinoline-4-carboxamide
N-(3-(1H-pyrazol-1-yl)phenyl)-2-(2-methoxyethyl)-1-oxo-3-(thiophen-2-yl)-1,2,3,4-tetrahydroisoquinoline-4-carboxamide
2-(2-methoxyethyl)-1-oxo-N-(4-(pyridin-3-yl)phenyl)-3-(thiophen-2-yl)-1,2,3,4-tetrahydroisoquinoline-4-carboxamide
N-(4-((1H-pyrazol-1-ylmethyl)phenyl)-2-(2-methoxyethyl)-1-oxo-3-(thiophen-2-yl)-1,2,3,4-tetrahydroisoquinoline-4-carboxamide
2-(2-methoxyethyl)-1-oxo-N-(4-(pyrimidin-2-yl)phenyl)-3-(thiophen-2-yl)-1,2,3,4-tetrahydroisoquinoline-4-carboxamide
2-(2-methoxyethyl)-N-(1-methylindolin-5-yl)-1-oxo-3-(thiophen-2-yl)-1,2,3,4-tetrahydroisoquinoline-4-carboxamide
N-(4-tert-butylthiazol-2-yl)-2-(2-methoxyethyl)-1-oxo-3-(thiophen-2-yl)-1,2,3,4-tetrahydroisoquinoline-4-carboxamide
2-(2-methoxyethyl)-N-(4-(oxazol-5-yl)phenyl)-1-oxo-3-(thiophen-2-yl)-1,2,3,4-tetrahydroisoquinoline-4-carboxamide
2-(2-methoxyethyl)-1-oxo-3-(thiophen-2-yl)-N-(3-(trifluoromethyl)-1,2,4-thiadiazol-5-yl)-1,2,3,4-tetrahydroisoquinoline-4-carboxamide
2-(2-methoxyethyl)-N-(1-methyl-1H-pyrazol-3-yl)-1-oxo-3-(thiophen-2-yl)-1,2,3,4-tetrahydroisoquinoline-4-carboxamide
N-(4-(hydroxymethyl)phenyl)-2-(2-methoxyethyl)-1-oxo-3-(thiophen-2-yl)-1,2,3,4-tetrahydroisoquinoline-4-carboxamide
2-(2-methoxyethyl)-1-oxo-N-(5-phenyl-1H-pyrazol-3-yl)-3-(thiophen-2-yl)-1,2,3,4-tetrahydroisoquinoline-4-carboxamide
N-(5-(furan-2-yl)-1H-pyrazol-3-yl)-2-(2-methoxyethyl)-1-oxo-3-(thiophen-2-yl)-1,2,3,4-tetrahydroisoquinoline-4-carboxamide
2-(2-methoxyethyl)-1-oxo-N-(1H-pyrazol-3-yl)-3-(thiophen-2-yl)-1,2,3,4-tetrahydroisoquinoline-4-carboxamide
N-(5-tert-butyl-1H-pyrazol-3-yl)-2-(2-methoxyethyl)-1-oxo-3-(thiophen-2-yl)-1,2,3,4-tetrahydroisoquinoline-4-carboxamide
2-(2-methoxyethyl)-1-oxo-N-(6-phenylpyridin-3-yl)-3-(thiophen-2-yl)-1,2,3,4-tetrahydroisoquinoline-4-carboxamide
2-(2-methoxyethyl)-N-(4-(5-methyl-1,2,4-oxadiazol-3-yl)phenyl)-1-oxo-3-(thiophen-2-yl)-1,2,3,4-tetrahydroisoquinoline-4-carboxamide
2-(2-methoxyethyl)-N-(4-(morpholinomethyl)phenyl)-1-oxo-3-(thiophen-2-yl)-1,2,3,4-tetrahydroisoquinoline-4-carboxamide
N-(1,3-dimethyl-1H-pyrazol-5-yl)-2-(2-methoxyethyl)-1-oxo-3-(thiophen-2-yl)-1,2,3,4-tetrahydroisoquinoline-4-carboxamide
N-(1,3-dihydroisobenzofuran-5-yl)-2-(2-methoxyethyl)-1-oxo-3-(thiophen-2-yl)-1,2,3,4-tetrahydroisoquinoline-4-carboxamide
2-(2-methoxyethyl)-N-(4-(6-methylpyrazin-2-yloxy)phenyl)-1-oxo-3-(thiophen-2-yl)-1,2,3,4-tetrahydroisoquinoline-4-carboxamide
2-(2-methoxyethyl)-1-oxo-N-(4-phenoxyphenyl)-3-(thiophen-2-yl)-1,2,3,4-tetrahydroisoquinoline-4-carboxamide
ethyl 5-(2-(2-methoxyethyl)-1-oxo-3-(thiophen-2-yl)-1,2,3,4-tetrahydroisoquinoline-4-carboxamido)-1,3,4-oxadiazole-2-carboxylate
2-(2-methoxyethyl)-1-oxo-N-(3-phenyl-1,2,4-thiadiazol-5-yl)-3-(thiophen-2-yl)-1,2,3,4-tetrahydroisoquinoline-4-carboxamide
N-(benzofuran-5-yl)-2-(2-methoxyethyl)-1-oxo-3-(thiophen-2-yl)-1,2,3,4-tetrahydroisoquinoline-4-carboxamide
2-(2-methoxyethyl)-N-(5-methyl-1H-pyrazol-3-yl)-1-oxo-3-(thiophen-2-yl)-1,2,3,4-tetrahydroisoquinoline-4-carboxamide
N-(3-cyclopropyl-1-methyl-1H-pyrazol-5-yl)-2-(2-methoxyethyl)-1-oxo-3-(thiophen-2-yl)-1,2,3,4-tetrahydroisoquinoline-4-carboxamide
N-(1,1-dioxobenzo[b]thiophen-6-yl)-2-(2-methoxyethyl)-1-oxo-3-(thiophen-2-yl)-1,2,3,4-tetrahydroisoquinoline-4-carboxamide
N-(6-fluorobenzo[d]thiazol-2-yl)-2-(2-methoxyethyl)-1-oxo-3-(thiophen-2-yl)-1,2,3,4-tetrahydroisoquinoline-4-carboxamide
N-(3-(1H-1,2,4-triazol-1-yl)phenyl)-2-(2-methoxyethyl)-1-oxo-3-(thiophen-2-yl)-1,2,3,4-tetrahydroisoquinoline-4-carboxamide
N-(4-(3,5-dimethyl-1H-pyrazol-1-yl)phenyl)-2-(2-methoxyethyl)-1-oxo-3-(thiophen-2-yl)-1,2,3,4-tetrahydroisoquinoline-4-carboxamide
N-(6-chlorobenzo[d]thiazol-2-yl)-2-(2-methoxyethyl)-1-oxo-3-(thiophen-2-yl)-1,2,3,4-tetrahydroisoquinoline-4-carboxamide
2-(2-methoxyethyl)-N-(4-(1-methyl-1H-pyrazol-3-yl)phenyl)-1-oxo-3-(thiophen-2-yl)-1,2,3,4-tetrahydroisoquinoline-4-carboxamide
N-(1,3-dimethyl-1H-pyrazol-5-yl)-2-(2-methoxyethyl)-1-oxo-3-(thiophen-2-yl)-1,2,3,4-tetrahydroisoquinoline-4-carboxamide
2-(2-methoxyethyl)-N-(4-(5-methyl-1,3,4-oxadiazol-2-yl)phenyl)-1-oxo-3-(thiophen-2-yl)-1,2,3,4-tetrahydroisoquinoline-4-carboxamide
2-(2-methoxyethyl)-N-(1-methyl-2-oxoindolin-5-yl)-1-oxo-3-(thiophen-2-yl)-1,2,3,4-tetrahydroisoquinoline-4-carboxamide
5-amino-3-(2-(2-methoxyethyl)-1-oxo-3-(thiophen-2-yl)-1,2,3,4-tetrahydroisoquinoline-4-carbonyl)benzo[d]oxazol-2(3H)-one
2-(2-methoxyethyl)-N-(1-methyl-3-phenyl-1H-pyrazol-5-yl)-1-oxo-3-(thiophen-2-yl)-1,2,3,4-tetrahydroisoquinoline-4-carboxamide
methyl 4-(2-(2-methoxyethyl)-1-oxo-3-(thiophen-2-yl)-1,2,3,4-tetrahydroisoquinoline-4-carboxamido)-1-methyl-1H-pyrrole-2-carboxylate
2-(2-methoxyethyl)-N-(3-(oxazol-5-yl)phenyl)-1-oxo-3-(thiophen-2-yl)-1,2,3,4-tetrahydroisoquinoline-4-carboxamide
2-(2-methoxyethyl)-N-(4-(2-methyl-1H-imidazol-1-yl)phenyl)-1-oxo-3-(thiophen-2-yl)-1,2,3,4-tetrahydroisoquinoline-4-carboxamide
N-(3-((1H-imidazol-1-yl)methyl)phenyl)-2-(2-methoxyethyl)-1-oxo-3-(thiophen-2-yl)-1,2,3,4-tetrahydroisoquinoline-4-carboxamide
2-(2-methoxyethyl)-N-(4-methyl-3-(methylsulfonamido)phenyl)-1-oxo-3-(thiophen-2-yl)-1,2,3,4-tetrahydroisoquinoline-4-carboxamide
2-(2-methoxyethyl)-N-(4-(4-methyl-4H-1,2,4-triazol-3-yl)phenyl)-1-oxo-3-(thiophen-2-yl)-1,2,3,4-tetrahydroisoquinoline-4-carboxamide
N-(4-((1H-imidazol-1-yl)methyl)phenyl)-2-(2-methoxyethyl)-1-oxo-3-(thiophen-2-yl)-1,2,3,4-tetrahydroisoquinoline-4-carboxamide
2-(2-methoxyethyl)-1-oxo-N-(4-(piperidin-1-ylmethyl)phenyl)-3-(thiophen-2-yl)-1,2,3,4-tetrahydroisoquinoline-4-carboxamide
N-(4-((1H-1,2,4-triazol-1-yl)methyl)phenyl)-2-(2-methoxyethyl)-1-oxo-3-(thiophen-2-yl)-1,2,3,4-tetrahydroisoquinoline-4-carboxamide
2-(2-methoxyethyl)-1-oxo-N-(4-(tetrahydro-2H-pyran-4-yloxy)phenyl)-3-(thiophen-2-yl)-1,2,3,4-tetrahydroisoquinoline-4-carboxamide
N-(benzo[d]oxazol-6-yl)-2-(2-methoxyethyl)-1-oxo-3-(thiophen-2-yl)-1,2,3,4-tetrahydroisoquinoline-4-carboxamide
N-(3,4-dimethylisoxazol-5-yl)-2-(2-methoxyethyl)-1-oxo-3-(thiophen-2-yl)-1,2,3,4-tetrahydroisoquinoline-4-carboxamide
N-(4-cyano-3-(trifluoromethyl)phenyl)-2-(2-methoxyethyl)-1-oxo-3-(thiophen-2-yl)-1,2,3,4-tetrahydroisoquinoline-4-carboxamide
N-(3-cyano-4-(1H-pyrrol-1-yl)phenyl)-2-(2-methoxyethyl)-1-oxo-3-(thiophen-2-yl)-1,2,3,4-tetrahydroisoquinoline-4-carboxamide
N-(5-chloropyridin-2-yl)-2-(2-methoxyethyl)-1-oxo-3-(thiophen-2-yl)-1,2,3,4-tetrahydroisoquinoline-4-carboxamide
2-(2-methoxyethyl)-4-((3-methoxyphenylamino)methyl)-3-(thiophen-2-yl)isoquinolin-1(2H)-one
2-cyclohexyl-N-(3-methoxyphenyl)-3-(4-methoxyphenyl)-1-oxo-1,2,3,4-tetrahydroisoquinoline-4-carboxamide
3-(4-fluorophenyl)-N-(6-methoxybenzo[d]thiazol-2-yl)-2-(2-methoxyethyl)-1-oxo-1,2,3,4-tetrahydroisoquinoline-4-carboxamide
3-(4-fluorophenyl)-2-(2-methoxyethyl)-1-oxo-N-(quinolin-3-yl)-1,2,3,4-tetrahydroisoquinoline-4-carboxamide
3-(4-fluorophenyl)-N-(furan-2-ylmethyl)-2-(2-methoxyethyl)-1-oxo-1,2,3,4-tetrahydroisoquinoline-4-carboxamide
N-((1-ethylpyrrolidin-2-ylmethyl)-3-(4-fluorophenyl)-2-(2-methoxyethyl)-1-oxo-1,2,3,4-tetrahydroisoquinoline-4-carboxamide
3-(4-fluorophenyl)-2-(2-methoxyethyl)-1-oxo-N-(pyridin-2-ylmethyl)-1,2,3,4-tetrahydroisoquinoline-4-carboxamide
3-(3,4-dimethoxyphenyl)-N-(3-methoxyphenyl)-2-methyl-1-oxo-1,2,3,4-tetrahydroisoquinoline-4-carboxamide
2-(4-fluorobenzyl)-N-(3-methoxyphenyl)-3-(4-methoxyphenyl)-1-oxo-1,2,3,4-tetrahydroisoquinoline-4-carboxamide
3-(3,4-dimethoxyphenyl)-6,7-dimethoxy-N-(3-methoxyphenyl)-2-methyl-1-oxo-1,2,3,4-tetrahydroisoquinoline-4-carboxamide
6,7-dimethoxy-N-(3-methoxyphenyl)-3-(4-methoxyphenyl)-2-methyl-1-oxo-1,2,3,4-tetrahydroisoquinoline-4-carboxamide
6,7-dimethoxy-N-(3-methoxyphenyl)-2-methyl-1-oxo-3-(thiophen-2-yl)-1,2,3,4-tetrahydroisoquinoline-4-carboxamide
3-(2-fluorophenyl)-6,7-dimethoxy-N-(3-methoxyphenyl)-2-methyl-1-oxo-1,2,3,4-tetrahydroisoquinoline-4-carboxamide
N-(3-methoxyphenyl)-2-methyl-1-oxo-3-(thiophen-2-yl)-1,2,3,4-tetrahydroisoquinoline-4-carboxamide
2-(2-methoxyethyl)-N-(2-methoxyphenyl)-1-oxo-3-(thiophen-2-yl)-1,2,3,4-tetrahydroisoquinoline-4-carboxamide
2-(2-methoxyethyl)-1-oxo-N-(pyridin-2-yl)-3-(thiophen-2-yl)-1,2,3,4-tetrahydroisoquinoline-4-carboxamide
2-(2-methoxyethyl)-1-oxo-N-((tetrahydrofuran-2-yl)methyl)-3-(thiophen-2-yl)-1,2,3,4-tetrahydroisoquinoline-4-carboxamide
N,2-bis(2-methoxyethyl)-1-oxo-3-(thiophen-2-yl)-1,2,3,4-tetrahydroisoquinoline-4-carboxamide
N-(furan-2-ylmethyl)-2-(2-methoxyethyl)-1-oxo-3-(thiophen-2-yl)-1,2,3,4-tetrahydroisoquinoline-4-carboxamide
N-((1-ethylpyrrolidin-2-yl)methyl)-2-(2-methoxyethyl)-1-oxo-3-(thiophen-2-yl)-1,2,3,4-tetrahydroisoquinoline-4-carboxamide
2-(2-methoxyethyl)-1-oxo-N-(pyridin-2-ylmethyl)-3-(thiophen-2-yl)-1,2,3,4-tetrahydroisoquinoline-4-carboxamide
2-(2-methoxyethyl)-1-oxo-N-(pyridin-3-ylmethyl)-3-(thiophen-2-yl)-1,2,3,4-tetrahydroisoquinoline-4-carboxamide
ethyl 2-(2-(2-methoxyethyl)-1-oxo-3-(thiophen-2-yl)-1,2,3,4-tetrahydroisoquinoline-4-carboxamido)benzoate
N-(3-cyanophenyl)-2-(2-methoxyethyl)-1-oxo-3-(thiophen-2-yl)-1,2,3,4-tetrahydroisoquinoline-4-carboxamide
N-(3-methoxybenzyl)-2-(2-methoxyethyl)-1-oxo-3-(thiophen-2-yl)-1,2,3,4-tetrahydroisoquinoline-4-carboxamide
N-(2-chloropyridin-3-yl)-2-(2-methoxyethyl)-1-oxo-3-(thiophen-2-yl)-1,2,3,4-tetrahydroisoquinoline-4-carboxamide
2-(2-methoxyethyl)-N-(4-methoxyphenyl)-1-oxo-3-(thiophen-2-yl)-1,2,3,4-tetrahydroisoquinoline-4-carboxamide
N-(2-cyanophenyl)-2-(2-methoxyethyl)-1-oxo-3-(thiophen-2-yl)-1,2,3,4-tetrahydroisoquinoline-4-carboxamide
N-(2-chlorophenyl)-2-(2-methoxyethyl)-1-oxo-3-(thiophen-2-yl)-1,2,3,4-tetrahydroisoquinoline-4-carboxamide
2-(2-methoxyethyl)-4-(4-(3-methoxyphenyl)piperazine-1-carbonyl)-3-(thiophen-2-yl)-3,4-dihydroisoquinolin-1(2H)-one
N-(3-acetylphenyl)-2-(2-methoxyethyl)-1-oxo-3-(thiophen-2-yl)-1,2,3,4-tetrahydroisoquinoline-4-carboxamide
N-benzyl-2-(2-methoxyethyl)-1-oxo-3-(thiophen-2-yl)-1,2,3,4-tetrahydroisoquinoline-4-carboxamide
2-isobutyl-N-(3-methoxyphenyl)-1-oxo-3-(thiophen-2-yl)-1,2,3,4-tetrahydroisoquinoline-4-carboxamide
2-cyclopentyl-1-oxo-N-(pyridin-3-yl)-3-(thiophen-2-yl)-1,2,3,4-tetrahydroisoquinoline-4-carboxamide
4-(2-(2-methoxyethyl)-1-oxo-3-(thiophen-2-yl)-1,2,3,4-tetrahydroisoquinoline-4-carboxamido)benzoic acid
N-cyclohexyl-2-(2-methoxyethyl)-1-oxo-3-(thiophen-2-yl)-1,2,3,4-tetrahydroisoquinoline-4-carboxamide
2-(2-methoxyethyl)-4-(4-methylpiperidine-1-carbonyl)-3-(thiophen-2-yl)-3,4-dihydroisoquinolin-1(2H)-one
2-(2-methoxyethyl)-N-(3-(4-methylpiperidin-1-yl)propyl)-1-oxo-3-(thiophen-2-yl)-1,2,3,4-tetrahydroisoquinoline-4-carboxamide
N-(2,5-dimethylphenyl)-N-ethyl-2-(2-methoxyethyl)-1-oxo-3-(thiophen-2-yl)-1,2,3,4-tetrahydroisoquinoline-4-carboxamide
2-(2-methoxyethyl)-1-oxo-N-(pyridin-3-yl)-3-(thiophen-2-yl)-1,2,3,4-tetrahydroisoquinoline-4-carboxamide
N-(2-carbamoylphenyl)-2-(2-methoxyethyl)-1-oxo-3-(thiophen-2-yl)-1,2,3,4-tetrahydroisoquinoline-4-carboxamide
2-cyclopentyl-N-(3-methoxyphenyl)-1-oxo-3-(thiophen-2-yl)-1,2,3,4-tetrahydroisoquinoline-4-carboxamide
N-(2-methoxybenzyl)-2-(2-methoxyethyl)-1-oxo-3-(thiophen-2-yl)-1,2,3,4-tetrahydroisoquinoline-4-carboxamide
N-(2-ethoxybenzyl)-2-(2-methoxyethyl)-1-oxo-3-(thiophen-2-yl)-1,2,3,4-tetrahydroisoquinoline-4-carboxamide
2-isobutyl-N-isopropyl-1-oxo-3-(thiophen-2-yl)-1,2,3,4-tetrahydroisoquinoline-4-carboxamide
2-isobutyl-N-(2-morpholinoethyl)-1-oxo-3-(thiophen-2-yl)-1,2,3,4-tetrahydroisoquinoline-4-carboxamide
2-isobutyl-N-(4-methylbenzyl)-1-oxo-3-(thiophen-2-yl)-1,2,3,4-tetrahydroisoquinoline-4-carboxamide
2-cyclopentyl-N-(3-morpholinopropyl)-1-oxo-3-(thiophen-2-yl)-1,2,3,4-tetrahydroisoquinoline-4-carboxamide
2-cyclopentyl-N-(2-(4-methylpiperazin-1-yl)ethyl)-1-oxo-3-(thiophen-2-yl)-1,2,3,4-tetrahydroisoquinoline-4-carboxamide
   and isomers (in particular stereo-isomers or tautomers), solvates, hydrates, salts (in particular pharmaceutically acceptable salts) or prodrugs thereof.

In still another embodiment the compounds of the invention have a structure according to formula (VI):

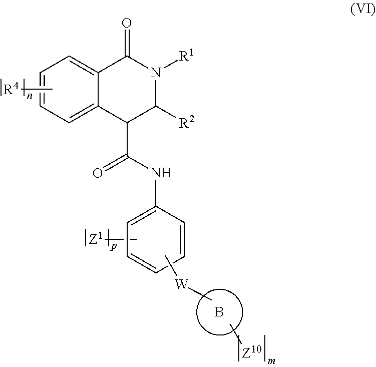

In still another embodiment the compounds of the invention have a structure according to formula (VII):

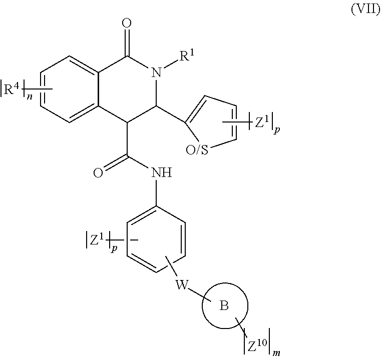

In still another embodiment the compounds of the invention have a structure according to formula (VIII):

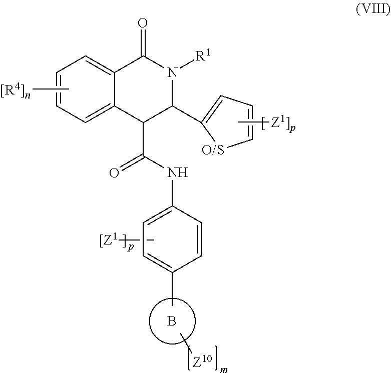

An especially particular embodiment relates to the compounds selected from:
2-(2-methoxyethyl)-N-(3-methoxyphenyl)-7-nitro-1-oxo-3-(thiophen-2-yl)-1,2,3,4-tetrahydroisoquinoline-4-carboxamide,
N-(3-methoxyphenyl)-2-(3-methoxypropyl)-1-oxo-3-(thiophen-2-yl)-1,2,3,4-tetrahydroisoquinoline-4-carboxamide,
2-(2-methoxyethyl)-N-(3-methoxyphenyl)-1-oxo-3-(thiophen-2-yl)-1,2,3,4-tetrahydroisoquinoline-4-carboxamide,
Methyl 2-(4-(3-methoxyphenylcarbamoyl)-1-oxo-3-(thiophen-2-yl)-3,4-dihydroisoquinolin-2(1H)-yl)acetate,
2-(2-(Dimethylamino)ethyl)-N-(3-methoxyphenyl)-1-oxo-3-(thiophen-2-yl)-1,2,3,4-tetrahydroisoquinoline-4-carboxamide,
2-Butyl-N-(3-methoxyphenyl)-1-oxo-3-(thiophen-2-yl)-1,2,3,4-tetrahydroisoquinoline-4-carboxamide,
2-(2-Methoxyethyl)-N-(3-methoxyphenyl)-5-methyl-1-oxo-3-(thiophen-2-yl)-1,2,3,4-tetrahydroisoquinoline-4-carboxamide,
2-(2-Methoxyethyl)-N-(3-methoxyphenyl)-6-methyl-1-oxo-3-(thiophen-2-yl)-1,2,3,4-tetrahydroisoquinoline-4-carboxamide,
2-(2-Methoxyethyl)-N-(3-methoxyphenyl)-7-methyl-1-oxo-3-(thiophen-2-yl)-1,2,3,4-tetrahydroisoquinoline-4-carboxamide,
2-(2-Methoxyethyl)-N-(3-methoxyphenyl)-8-methyl-1-oxo-3-(thiophen-2-yl)-1,2,3,4-tetrahydroisoquinoline-4-carboxamide,
7-Chloro-2-(2-methoxyethyl)-N-(3-methoxyphenyl)-1-oxo-3-(thiophen-2-yl)-1,2,3,4-tetrahydroisoquinoline-4-carboxamide,
8-Chloro-2-(2-methoxyethyl)-N-(3-methoxyphenyl)-1-oxo-3-(thiophen-2-yl)-1,2,3,4-tetrahydroisoquinoline-4-carboxamide,
7-Iodo-2-(2-methoxyethyl)-N-(3-methoxyphenyl)-1-oxo-3-(thiophen-2-yl)-1,2,3,4-tetrahydroisoquinoline-4-carboxamide,
2-(2-Methoxyethyl)-1-oxo-N-phenyl-3-(thiophen-2-yl)-1,2,3,4-tetrahydroisoquinoline-4-carboxamide,
2-(2-Methoxyethyl)-1-oxo-3-(thiophen-2-yl)-N-(4-(trifluoromethyl)phenyl)-1,2,3,4-tetrahydroisoquinoline-4-carboxamide,
N-(4-cyanophenyl)-2-(2-methoxyethyl)-1-oxo-3-(thiophen-2-yl)-1,2,3,4-tetrahydroisoquinoline-4-carboxamide,
N-(biphenyl-3-yl)-2-(2-methoxyethyl)-1-oxo-3-(thiophen-2-yl)-1,2,3,4-tetrahydroisoquinoline-4-carboxamide,
2-(2-Methoxyethyl)-1-oxo-N-(2-phenoxyethyl)-3-(thiophen-2-yl)-1,2,3,4-tetrahydroisoquinoline-4-carboxamide,
2-(2-Methoxyethyl)-N-(2-(1-methyl-1H-indol-3-yl)ethyl)-1-oxo-3-(thiophen-2-yl)-1,2,3,4-tetrahydroisoquinoline-4-carboxamide,
Methyl 4-(2-(2-methoxyethyl)-1-oxo-3-(thiophen-2-yl)-1,2,3,4-tetrahydroisoquinoline-4-carboxamido)benzoate,
Ethyl 3-(2-(2-methoxyethyl)-1-oxo-3-(thiophen-2-yl)-1,2,3,4-tetrahydroisoquinoline-4-carboxamido)benzoate,
N-(3-fluorophenyl)-2-(2-methoxyethyl)-1-oxo-3-(thiophen-2-yl)-1,2,3,4-tetrahydroisoquinoline-4-carboxamide,
N-(3,4-dimethoxyphenyl)-2-(2-methoxyethyl)-1-oxo-3-(thiophen-2-yl)-1,2,3,4-tetrahydroisoquinoline-4-carboxamide,
N-(4-chlorophenyl)-2-(2-methoxyethyl)-1-oxo-3-(thiophen-2-yl)-1,2,3,4-tetrahydroisoquinoline-4-carboxamide,
2-(Furan-2-ylmethyl)-N-(3-methoxyphenyl)-1-oxo-3-(thiophen-2-yl)-1,2,3,4-tetrahydroisoquinoline-4-carboxamide,
2-Ethyl-N-(3-methoxyphenyl)-1-oxo-3-(thiophen-2-yl)-1,2,3,4-tetrahydroisoquinoline-4-carboxamide,
2-(Cyclohexylmethyl)-N-(3-methoxyphenyl)-1-oxo-3-(thiophen-2-yl)-1,2,3,4-tetrahydroisoquinoline-4-carboxamide,
2-(2-Hydroxyethyl)-N-(3-methoxyphenyl)-1-oxo-3-(thiophen-2-yl)-1,2,3,4-tetrahydroisoquinoline-4-carboxamide,
2-Cyclohexyl-N-(3-methoxyphenyl)-1-oxo-3-(thiophen-2-yl)-1,2,3,4-tetrahydroisoquinoline-4-carboxamide,
N-(3-methoxyphenyl)-1-oxo-2-((tetrahydrofuran-2-ylmethyl)-3-(thiophen-2-yl)-1,2,3,4-tetrahydroisoquinoline-4-carboxamide,
2-Benzyl-N-(3-methoxyphenyl)-1-oxo-3-(thiophen-2-yl)-1,2,3,4-tetrahydroisoquinoline-4-carboxamide,
7-Methoxy-2-(2-methoxyethyl)-N-(3-methoxyphenyl)-1-oxo-3-(thiophen-2-yl)-1,2,3,4-tetrahydroisoquinoline-4-carboxamide,
8-Fluoro-2-(2-methoxyethyl)-N-(3-methoxyphenyl)-1-oxo-3-(thiophen-2-yl)-1,2,3,4-tetrahydroisoquinoline-4-carboxamide,
7-Fluoro-2-(2-methoxyethyl)-N-(3-methoxyphenyl)-1-oxo-3-(thiophen-2-yl)-1,2,3,4-tetrahydroisoquinoline-4-carboxamide,
5-Fluoro-2-(2-methoxyethyl)-N-(3-methoxyphenyl)-1-oxo-3-(thiophen-2-yl)-1,2,3,4-tetrahydroisoquinoline-4-carboxamide,
N-(3-methoxyphenyl)-1-oxo-3-(thiophen-2-yl)-2-(2,2,2-trifluoroethyl)-1,2,3,4-tetrahydroisoquinoline-4-carboxamide,
N-(3-methoxyphenyl)-2-(2-(methylthio)ethyl)-1-oxo-3-(thiophen-2-yl)-1,2,3,4-tetrahydroisoquinoline-4-carboxamide,
N-(3-methoxyphenyl)-2-(3-morpholinopropyl)-1-oxo-3-(thiophen-2-yl)-1,2,3,4-tetrahydroisoquinoline-4-carboxamide,
N-(3-ethoxyphenyl)-2-(2-methoxyethyl)-1-oxo-3-(thiophen-2-yl)-1,2,3,4-tetrahydroisoquinoline-4-carboxamide,
N-(3-chlorophenyl)-2-(2-methoxyethyl)-1-oxo-3-(thiophen-2-yl)-1,2,3,4-tetrahydroisoquinoline-4-carboxamide,
2-(2-Ethoxyethyl)-N-(3-methoxyphenyl)-1-oxo-3-(thiophen-2-yl)-1,2,3,4-tetrahydroisoquinoline-4-carboxamide,
2-(2-Isopropoxyethyl)-N-(3-methoxyphenyl)-1-oxo-3-(thiophen-2-yl)-1,2,3,4-tetrahydroisoquinoline-4-carboxamide,
2-((1-ethylpyrrolidin-2-ylmethyl)-N-(3-methoxyphenyl)-1-oxo-3-(thiophen-2-yl)-1,2,3,4-tetrahydroisoquinoline-4-carboxamide,
2-(((S)-1-ethylpyrrolidin-2-ylmethyl)-N-(3-methoxyphenyl)-1-oxo-3-(thiophen-2-yl)-1,2,3,4-tetrahydroisoquinoline-4-carboxamide,
6,7-dimethoxy-2-(2-methoxyethyl)-N-(3-methoxyphenyl)-1-oxo-3-(thiophen-2-yl)-1,2,3,4-tetrahydroisoquinoline-4-carboxamide,
3-(furan-2-yl)-2-(2-methoxyethyl)-N-(3-methoxyphenyl)-1-oxo-1,2,3,4-tetrahydroisoquinoline-4-carboxamide,
3-(2-fluorophenyl)-2-(2-methoxyethyl)-N-(3-methoxyphenyl)-1-oxo-1,2,3,4-tetrahydroisoquinoline-4-carboxamide,
3-(3-Fluorophenyl)-2-(2-methoxyethyl)-N-(3-methoxyphenyl)-1-oxo-1,2,3,4-tetrahydroisoquinoline-4-carboxamide,
3-(4-Fluorophenyl)-2-(2-methoxyethyl)-N-(3-methoxyphenyl)-1-oxo-1,2,3,4-tetrahydroisoquinoline-4-carboxamide,
3-(2,6-Difluorophenyl)-2-(2-methoxyethyl)-N-(3-methoxyphenyl)-1-oxo-1,2,3,4-tetrahydroisoquinoline-4-carboxamide,
2-(2-Methoxyethyl)-N-(3-methoxyphenyl)-1-oxo-3-(thiophen-3-yl)-1,2,3,4-tetrahydroisoquinoline-4-carboxamide,
2-(2-Methoxyethyl)-N-(3-methoxyphenyl)-3-(5-methylthiophen-2-yl)-1-oxo-1,2,3,4-tetrahydroisoquinoline-4-carboxamide,
3-(3-Chlorothiophen-2-yl)-2-(2-methoxyethyl)-N-(3-methoxyphenyl)-1-oxo-1,2,3,4-tetrahydroisoquinoline-4-carboxamide,
2-(2-Methoxyethyl)-N-(3-methoxyphenyl)-1-oxo-3-(pyridin-2-yl)-1,2,3,4-tetrahydroisoquinoline-4-carboxamide,
2-(2-Methoxyethyl)-N-(3-methoxyphenyl)-1-oxo-3-(pyridin-4-0)-1,2,3,4-tetrahydroisoquinoline-4-carboxamide,
2-(2-Methoxyethyl)-N-(3-methoxyphenyl)-1-oxo-3-phenyl-1,2,3,4-tetrahydroisoquinoline-4-carboxamide,
2-(2-Methoxyethyl)-N-(3-methoxyphenyl)-1-oxo-3-(1H-pyrrol-2-yl)-1,2,3,4-tetrahydroisoquinoline-4-carboxamide,
3-(1H-imidazol-2-yl)-2-(2-methoxyethyl)-N-(3-methoxyphenyl)-1-oxo-1,2,3,4-tetrahydroisoquinoline-4-carboxamide,
2-(2-Methoxyethyl)-N-(3-methoxyphenyl)-1-oxo-3-(pyridin-3-yl)-1,2,3,4-tetrahydroisoquinoline-4-carboxamide,
2-(2-Methoxyethyl)-N-(3-methoxyphenyl)-3-(4-methyl-1H-imidazol-5-yl)-1-oxo-1,2,3,4-tetrahydroisoquinoline-4-carboxamide,
3-(Benzo[b]thiophen-2-yl)-2-(2-methoxyethyl)-N-(3-methoxyphenyl)-1-oxo-1,2,3,4-tetrahydroisoquinoline-4-carboxamide,
3-tert-Butyl-2-(2-methoxyethyl)-N-(3-methoxyphenyl)-1-oxo-1,2,3,4-tetrahydroisoquinoline-4-carboxamide,
3-Ethyl-2-(2-methoxyethyl)-N-(3-methoxyphenyl)-1-oxo-1,2,3,4-tetrahydroisoquinoline-4-carboxamide,
3-Benzyl-2-(2-methoxyethyl)-N-(3-methoxyphenyl)-1-oxo-1,2,3,4-tetrahydroisoquinoline-4-carboxamide,
2-(2-methoxyethyl)-N-(3-methoxyphenyl)-3-methyl-1-oxo-1,2,3,4-tetrahydroisoquinoline-4-carboxamide,
2-(2-methoxyethyl)-N-(3-methoxyphenyl)-1-oxo-3-((trimethylsilyl)ethynyl)-1,2,3,4-tetrahydroisoquinoline-4-carboxamide,
2-(2-methoxyethyl)-4-((3-methoxyphenylamino)methyl)-3-(thiophen-2-yl)isoquinolin-1(2H)-one,
2-(2-methoxyethyl)-1-oxo-3-(thiophen-2-yl)-1,2,3,4-tetrahydroisoquinoline-4-carboxylic acid,
2-(2-methoxyethyl)-N-(3-methoxyphenyl)-1-oxo-3-(thiophen-2-yl)-1,2-dihydroisoquinoline-4-carboxamide,
3-(furan-2-yl)-2-(2-methoxyethyl)-N-(3-methoxyphenyl)-1-oxo-1,2-dihydroisoquinoline-4-carboxamide,
4-(hydroxymethyl)-2-(2-methoxyethyl)-3-(thiophen-2-yl)-3,4-dihydroisoquinolin-1(2H)-one,
4-(benzyloxymethyl)-2-(2-methoxyethyl)-3-(thiophen-2-yl)-3,4-dihydroisoquinolin-1(2H)-one,
6-fluoro-2-(2-methoxyethyl)-N-(3-methoxyphenyl)-1-oxo-3-(thiophen-2-yl)-1,2,3,4-tetrahydroisoquinoline-4-carboxamide,
2-(2-methoxyethyl)-4-((3-methoxyphenylamino)methyl)-3-(thiophen-2-yl)-3,4-dihydroisoquinolin-1(2H)-one,
4-((4-chlorophenylamino)methyl)-2-(2-methoxyethyl)-3-(thiophen-2-yl)-3,4-dihydroisoquinolin-1(2H)-one.
N-cyclohexyl-2-(2-methoxyethyl)-1-oxo-3-(thiophen-2-yl)-1,2,3,4-tetrahydroisoquinoline-4-carboxamide,
2-(2-methoxyethyl)-4-(4-methylpiperidine-1-carbonyl)-3-(thiophen-2-yl)-3,4-dihydroisoquinolin-1(2H)-one,
2-(2-methoxyethyl)-N-(3-(4-methylpiperidin-1-yl)propyl)-1-oxo-3-(thiophen-2-yl)-1,2,3,4-tetrahydroisoquinoline-4-carboxamide,
N-(2,5-dimethylphenyl)-N-ethyl-2-(2-methoxyethyl)-1-oxo-3-(thiophen-2-yl)-1,2,3,4-tetrahydroisoquinoline-4-carboxamide,
2-(2-methoxyethyl)-1-oxo-N-(pyridin-3-yl)-3-(thiophen-2-yl)-1,2,3,4-tetrahydroisoquinoline-4-carboxamide,
2-cyclohexyl-N-(3-methoxyphenyl)-3-(4-methoxyphenyl)-1-oxo-1,2,3,4-tetrahydroisoquinoline-4-carboxamide,
3-(4-fluorophenyl)-N-(6-methoxybenzo[d]thiazol-2-yl)-2-(2-methoxyethyl)-1-oxo-1,2,3,4-tetrahydroisoquinoline-4-carboxamide,
3-(4-fluorophenyl)-2-(2-methoxyethyl)-1-oxo-N-(quinolin-3-yl)-1,2,3,4-tetrahydroisoquinoline-4-carboxamide,
3-(4-fluorophenyl)-N-(furan-2-ylmethyl)-2-(2-methoxyethyl)-1-oxo-1,2,3,4-tetrahydroisoquinoline-4-carboxamide,
N-((1-ethylpyrrolidin-2-yl)methyl)-3-(4-fluorophenyl)-2-(2-methoxyethyl)-1-oxo-1,2,3,4-tetrahydroisoquinoline-4-carboxamide,
3-(4-fluorophenyl)-2-(2-methoxyethyl)-1-oxo-N-(pyridin-2-ylmethyl)-1,2,3,4-tetrahydroisoquinoline-4-carboxamide,
3-(3,4-dimethoxyphenyl)-N-(3-methoxyphenyl)-2-methyl-1-oxo-1,2,3,4-tetrahydroisoquinoline-4-carboxamide,
2-(4-fluorobenzyl)-N-(3-methoxyphenyl)-3-(4-methoxyphenyl)-1-oxo-1,2,3,4-tetrahydroisoquinoline-4-carboxamide,
3-(3,4-dimethoxyphenyl)-6,7-dimethoxy-N-(3-methoxyphenyl)-2-methyl-1-oxo-1,2,3,4-tetrahydroisoquinoline-4-carboxamide,
6,7-dimethoxy-N-(3-methoxyphenyl)-3-(4-methoxyphenyl)-2-methyl-1-oxo-1,2,3,4-tetrahydroisoquinoline-4-carboxamide,
6,7-dimethoxy-N-(3-methoxyphenyl)-2-methyl-1-oxo-3-(thiophen-2-yl)-1,2,3,4-tetrahydroisoquinoline-4-carboxamide,
3-(2-fluorophenyl)-6,7-dimethoxy-N-(3-methoxyphenyl)-2-methyl-1-oxo-1,2,3,4-tetrahydroisoquinoline-4-carboxamide,
N-(3-methoxyphenyl)-2-methyl-1-oxo-3-(thiophen-2-yl)-1,2,3,4-tetrahydroisoquinoline-4-carboxamide,
2-(2-methoxyethyl)-1-oxo-N-((tetrahydrofuran-2-yl)methyl)-3-(thiophen-2-yl)-1,2,3,4-tetrahydroisoquinoline-4-carboxamide,
N,2-bis(2-methoxyethyl)-1-oxo-3-(thiophen-2-yl)-1,2,3,4-tetrahydroisoquinoline-4-carboxamide,
N-(furan-2-ylmethyl)-2-(2-methoxyethyl)-1-oxo-3-(thiophen-2-yl)-1,2,3,4-tetrahydroisoquinoline-4-carboxamide,
N-((1-ethylpyrrolidin-2-yl)methyl)-2-(2-methoxyethyl)-1-oxo-3-(thiophen-2-yl)-1,2,3,4-tetrahydroisoquinoline-4-carboxamide,
2-(2-methoxyethyl)-1-oxo-N-(pyridin-2-ylmethyl)-3-(thiophen-2-yl)-1,2,3,4-tetrahydroisoquinoline-4-carboxamide,
N-(2-chloropyridin-3-yl)-2-(2-methoxyethyl)-1-oxo-3-(thiophen-2-yl)-1,2,3,4-tetrahydroisoquinoline-4-carboxamide,
N-(2-carbamoylphenyl)-2-(2-methoxyethyl)-1-oxo-3-(thiophen-2-yl)-1,2,3,4-tetrahydroisoquinoline-4-carboxamide,
2-(2-methoxyethyl)-4-(4-(3-methoxyphenyl)piperazine-1-carbonyl)-3-(thiophen-2-yl)-3,4-dihydroisoquinolin-1(2H)-one,
N-(3-acetylphenyl)-2-(2-methoxyethyl)-1-oxo-3-(thiophen-2-yl)-1,2,3,4-tetrahydroisoquinoline-4-carboxamide,
N-benzyl-2-(2-methoxyethyl)-1-oxo-3-(thiophen-2-yl)-1,2,3,4-tetrahydroisoquinoline-4-carboxamide,
2-isobutyl-N-(3-methoxyphenyl)-1-oxo-3-(thiophen-2-yl)-1,2,3,4-tetrahydroisoquinoline-4-carboxamide,
2-cyclopentyl-N-(3-methoxyphenyl)-1-oxo-3-(thiophen-2-yl)-1,2,3,4-tetrahydroisoquinoline-4-carboxamide, and their isomers (i.e. cis or trans), salts, solvates, hydrates and prodrugs.

A second aspect of the present invention relates to the compounds described in the first aspect and all embodiments thereof for use as a medicine.

According to an embodiment of the second aspect of the present invention, a compound according to formula (II),

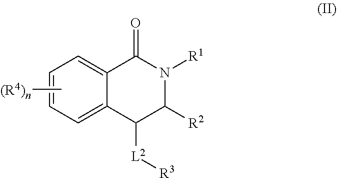

wherein

R¹ is selected from alkyl, alkenyl, alkynyl, aryl, heterocycle, arylalkyl, arylalkenyl, arylalkynyl, heterocycle-alkyl, heterocycle-alkenyl or heterocycle-alkynyl, and wherein said alkyl, alkenyl, alkynyl, aryl, heterocycle, arylalkyl, arylalkenyl, arylalkynyl, heterocycle-alkyl, heterocycle-alkenyl or heterocycle-alkynyl optionally include one or more heteroatoms, said heteroatom selected from O, S, P and N, and wherein said alkyl, alkenyl, alkynyl, aryl, heterocycle, arylalkyl, arylalkenyl, arylalkynyl, heterocycle-alkyl, heterocycle-alkenyl or heterocycle-alkynyl can be unsubstituted or substituted with one or more Z¹,

R² is selected from thienyl, or furanyl, wherein said thienyl or furanyl can be unsubstituted or substituted with one or more Z¹,

R³ is selected from alkyl, alkenyl, alkynyl, heterocycle, aryl, arylalkyl, arylalkenyl, arylalkynyl, heterocycle-alkyl, heterocycle-alkenyl or heterocycle-alkynyl, wherein said alkyl, alkenyl, alkynyl, arylalkyl, arylalkenyl, arylalkynyl, heterocycle-alkyl, heterocycle-alkenyl or heterocycle-alkynyl optionally include one or more heteroatoms, said heteroatom selected from O, S, P and N, and wherein said alkyl, alkenyl, alkynyl, aryl, heterocycle, arylalkyl, arylalkenyl, arylalkynyl, heterocycle-alkyl, heterocycle-alkenyl or heterocycle-alkynyl is optionally substituted with one or more of Z¹⁶,

L² is selected from —CH₂NZ⁶—, CH₂NH—, —CH₂O—, —CO—NZ⁶—, or —CONH—,

each of R⁴ is independently selected from the group consisting of halogen, —OH, alkoxy, —SH, thioalkoxy, trifluoromethyl, trifluoromethoxy, nitro, cyano, —COOH, COO-alkyl, —COO-alkenyl, —COO-alkynyl, amino, heteroalkyl, alkyl, alkenyl, alkynyl, aryl, heterocycle, arylalkyl, arylalkenyl, arylalkynyl, heterocycle-alkyl, heterocycle-alkenyl, and heterocycle-alkynyl,

n is selected from 0, 1, 2, 3 or 4,

each of Z¹ is independently selected from the group consisting of hydrogen, halogen, hydroxyl, sulfhydryl, alkoxy, thioalkoxy, ═O, ═S, trifluoromethyl, trifluoromethoxy, nitro, amino, cyano, —COOH, —COO-alkyl, —COO-alkenyl, —COO-alkynyl, —CONH₂, alkyl, alkenyl, alkynyl, aryl, heterocycle, arylalkyl, arylalkenyl, arylalkynyl, heterocycle-alkyl, heterocycle-alkenyl, heterocycle-alkynyl,

Z⁶ is independently selected from alkyl, alkenyl, alkynyl, heteroalkyl, heterocycle, and aryl,

each of Z¹⁶ is independently selected from the group consisting of hydrogen, halogen, hydroxyl, sulfhydryl, alkoxy, thioalkoxy, ═O, ═S, trifluoromethyl, trifluoromethoxy, nitro, amino, cyano, alkyl, alkenyl, alkynyl, aryl, heterocycle, arylalkyl, arylalkenyl, arylalkynyl, heterocycle-alkyl, heterocycle-alkenyl, and heterocycle-alkynyl, wherein said alkoxy, thioalkoxy, alkyl, alkenyl, alkynyl, aryl, heterocycle, arylalkyl, arylalkenyl, arylalkynyl, heterocycle-alkyl, heterocycle-alkenyl, and heterocycle-alkynyl group optionally include one or more heteroatoms, said heteroatom selected from O, S, P and N, and wherein said alkoxy, thioalkoxy, alkyl, alkenyl, alkynyl, aryl, heterocycle, arylalkyl, arylalkenyl, arylalkynyl, heterocycle-alkyl, heterocycle-alkenyl, and heterocycle-alkynyl group is optionally substituted with one or more Z¹⁷,

each of Z¹⁷ is independently selected from halogen, —OH, alkoxy, —SH, thioalkoxy, trifluoromethyl, trifluoromethoxy, ═O, ═S, nitro, cyano, —COOH, —COO-alkyl, —COO-alkenyl, —COO-alkynyl, amino, heteroalkyl, alkyl, alkenyl, alkynyl, aryl, heterocycle, arylalkyl, arylalkenyl, arylalkynyl, heterocycle-alkyl, heterocycle-alkenyl, and heterocycle-alkynyl and isomers (in particular stereo-isomers or tautomers), solvates, hydrates, salts (in particular pharmaceutically acceptable salts) or prodrugs thereof, for use as a medicine,

with exclusion of one or more of the following compounds being preferred:
2-[(4-chlorophenyl)methyl]-3-(5-chloro-2-thienyl)-N-[2-(dimethylamino)ethyl]-1,2,3,4-tetrahydro-1-oxo-4-isoquinolinecarboxamide;
4-Isoquinolinecarboxamide, 2-[(4-chlorophenyl)methyl]-3-(5-chloro-2-thienyl)-1,2,3,4-tetrahydro-N-(2-methoxyethyl)-1-oxo-;
4-Isoquinolinecarboxamide, N-[(1-ethyl-2-pyrrolidinyl)methyl]-1,2,3,4-tetrahydro-6,7-dimethoxy-2-methyl-1-oxo-3-(2-thienyl)-;
4-Isoquinolinecarboxamide, 1,2,3,4-tetrahydro-6,7-dimethoxy-2-methyl-N-(1-methylpropyl)-1-oxo-3-(2-thienyl)-;
4-Isoquinolinecarboxamide, 2-cyclopentyl-1,2,3,4-tetrahydro-1-oxo-N-[3-(4-phenyl-1-piperazinyl)propyl]-3-(2-thienyl)-;
4-Isoquinolinecarboxamide, 2-cyclopentyl-N-[3-[cyclopentyl(2-furanylmethyl)amino]propyl]-1,2,3,4-tetrahydro-1-oxo-3-(2-thienyl)-;
4-Isoquinolinecarboxamide, 2-cyclopentyl-N-[3-(3,4-dihydro-2(1H)-isoquinolinyl)propyl]-1,2,3,4-tetrahydro-1-oxo-3-(2-thienyl)-;
4-Isoquinolinecarboxamide, 2-cyclopentyl-N-[3-(ethylphenylamino)propyl]-1,2,3,4-tetrahydro-1-oxo-3-(2-thienyl)-;
4-Isoquinolinecarboxamide, 1,2,3,4-tetrahydro-N-[3-[(1-methylethyl)(phenylmethyl)amino]propyl]-2-(2-methylpropyl)-1-oxo-3-(2-thienyl)-;
4-Isoquinolinecarboxamide, 1,2,3,4-tetrahydro-6,7-dimethoxy-2-methyl-N-[2-(4-morpholinyl)ethyl]-1-oxo-3-(2-thienyl)-;
4-Isoquinolinecarboxamide, N-[3-(diethylamino)propyl]-1,2,3,4-tetrahydro-6,7-dimethoxy-2-methyl-1-oxo-3-(2-thienyl)-;
4-Isoquinolinecarboxamide, N-(2-furanylmethyl)-1,2,3,4-tetrahydro-6,7-dimethoxy-2-methyl-1-oxo-3-(2-thienyl)-;
4-Isoquinolinecarboxamide, N-[3-(dipropylamino)propyl]-1,2,3,4-tetrahydro-6,7-dimethoxy-2-methyl-1-oxo-3-(2-thienyl)-;
2(1H)-Isoquinolineheptanamide, 3,4-dihydro-3-[5-[(4-methoxyphenyl)methyl]-2-furanyl]-1-oxo-4-[[(tricyclo[3.3.1.13.7]dec-1-ylmethyl)amino]carbonyl]-;
L-Tryptophan, N-[[(3R,4R)-3-(2-furanyl)-1,2,3,4-tetrahydro-1-oxo-2-(phenylmethyl)-4-isoquinolinyl]carbonyl]-, methyl ester.

A third aspect of the present invention relates to the use of the compounds herein described for the manufacture of a medicament for the prevention or treatment of an infection of an animal, mammal or human with a virus. In a particular embodiment said medicament is for the prevention or treatment of a RNA virus, yet more particularly a Flavivirus, still more particularly the Hepatitis C virus.

A fourth aspect of the present invention relates to the compounds described herein for the prevention or treatment of an infection of an animal, mammal or human with a virus. In a particular embodiment, the viral infection is caused by a RNA virus, yet more particular by a Flavivirus, yet more preferably by the Hepatitis C virus.

A fifth aspect of the present invention relates to a pharmaceutical composition comprising the compounds described herein above and all embodiments thereof in combination with a pharmaceutically acceptable carrier.

A sixth aspect of the present invention relates to a method for the prevention or treatment of a viral infection in an animal, mammal or human comprising administering to an animal, mammal or human in need for such prevention or treatment an effective dose of the compounds of the first aspect and the embodiments thereof.

A seventh aspect of the present invention relates to a method for the preparation of 1-oxo-1,2,3,4-tetrahydroisoquinoline-4-carboxylic acids, intermediates in the preparation of the compounds of present invention, comprising the steps of

reacting an aromatic aldehyde with a primary amine to obtain an azomethine,

reacting a homophthalic acid optionally substituted in the benzene ring with an anhydride to obtain the corresponding homophthalic anhydride, and

reacting the homophthalic anhydride with the azomethine in a polar or apolar solvent to obtain a 1-oxo-1,2,3,4-tetrahydroisoquinoline-4-carboxylic acid.

In a preferred embodiment of the seventh aspect of the present invention, the benzene ring of the homophthalic acid is substituted with at least one substituent from the group consisting of C₁₋₁₈-alkyl, C₁₋₁₈-alkoxy, C₁₋₁₈-thioalkoxy, halogen, nitro and amino groups.

An eighth aspect of the present invention relates to a method for the preparation of compounds according to the present invention comprising the steps of:

preparing an 1-oxo-1,2,3,4-tetrahydroisoquinoline-4-carboxylic acid according to the above-described method,

converting the carboxy group into a —CH₂OH group, and

converting said —CH₂OH group into an ether, or —CH₂-amino group.

The above and other characteristics, features and advantages of the present invention will become apparent from the following detailed description. This description is given for the sake of example only, without limiting the scope of the invention.

### DETAILED DESCRIPTION OF THE INVENTION

The present invention will be described with respect to particular embodiments but the invention is not limited thereto but only by the claims. Furthermore, the terms first, second, third and the like in the description and in the claims, are used for distinguishing between similar elements and not necessarily for describing a sequence, either temporally, spatially, in ranking or in any other manner. It is to be understood that the terms so used are interchangeable under appropriate circumstances and that the embodiments of the invention described herein are capable of operation in other sequences than described or illustrated herein.

It is to be noticed that the term “comprising”, used in the claims, should not be interpreted as being restricted to the means listed thereafter, it does not exclude other elements or steps. It is thus to be interpreted as specifying the presence of the stated features, integers, steps or components as referred to, but does not preclude the presence or addition of one or more other features, integers, steps or components, or groups thereof.

Reference throughout this specification to “one embodiment” or “an embodiment” means that a particular feature, structure or characteristic described in connection with the embodiment is included in at least one embodiment of the present invention. Thus, appearances of the phrases “in one embodiment” or “in an embodiment” in various places throughout this specification are not necessarily all referring to the same embodiment, but may. Furthermore, the particular features, structures or characteristics may be combined in any suitable manner, as would be apparent to one of ordinary skill in the art from this disclosure, in one or more embodiments.

Similarly it should be appreciated that in the description of exemplary embodiments of the invention, various features of the invention are sometimes grouped together in a single embodiment, figure, or description thereof for the purpose of streamlining the disclosure and aiding in the understanding of one or more of the various inventive aspects. This method of disclosure, however, is not to be interpreted as reflecting an intention that the claimed invention requires more features than are expressly recited in each claim. Rather, as the following claims reflect, inventive aspects lie in less than all features of a single foregoing disclosed embodiment. Thus, the claims following the detailed description are hereby expressly incorporated into this detailed description, with each claim standing on its own as a separate embodiment of this invention.

Furthermore, while some embodiments described herein include some but not other features included in other embodiments, combinations of features of different embodiments are meant to be within the scope of the invention, and form different embodiments, as would be understood by those in the art. For example, in the following claims, any of the claimed embodiments can be used in any combination.

In the description provided herein, numerous specific details are set forth. However, it is understood that embodiments of the invention may be practiced without these specific details. In other instances, well-known methods, structures and techniques have not been shown in detail in order not to obscure an understanding of this description.

The following terms are provided solely to aid in the understanding of the invention.

### DEFINITIONS

In each of the following definitions, the number of carbon atoms represents the maximum number of carbon atoms generally optimally present in the substituent or linker, it is understood that where otherwise indicated in the present application, the number of carbon atoms represents the optimal maximum number of carbon atoms for that particular substituent or linker.

Formulae I, Ia, Ib, Ic and Id include the groups Q and T respectively, where Q represents -L¹R² or —R² and T represents -L²R³ or —R³. Such representations are fully equivalent to representations of Q and T as —XR² and —YR³ respectively in which X and Y are optionally not present, since one skilled in the art would realise that two part bonds correspond to a single bond and X and Y correspond to L¹ and L² respectively.

The term “hydrocarbyl group” or “C₁₋₁₈ hydrocarbyl group” as used herein refers to C₁₋₁₈ normal, secondary, tertiary, ethylenically and acetylenically unsaturated or saturated acyclic or cyclic, (including aromatic), hydrocarbons and combinations thereof. This term therefore comprises alkyl, alkenyl, alkynyl, cycloalkyl, cycloalkenyl, cycloalkynyl, aryl, arylalkyl, arylalkenyl, arylakynyl, alkyl-5-alkyl, dialkylamino-alkyl among others.

The term C₁₋₁₈ represents groups with 1, 2, 3, 4, 5, 6, 7, 8, 9, 10, 11, 12, 13, 14, 15, 16 17 and 18 carbon atoms, except in the case of heteroalkyl in which one or more of these carbon atoms can be replaced by a heteroatom such as O, S, N, Si and P.

The term C₂₋₁₈ represents groups with 2, 3, 4, 5, 6, 7, 8, 9, 10, 11, 12, 13, 14, 15, 16 17 and 18 carbon atoms.

The term C₁₋₆ represents groups with 1, 2, 3, 4, 5 and 6 carbon atoms.

The term C₂₋₆ represents groups with 2, 3, 4, 5 and 6 carbon atoms.

The term “hydrocarbyl group which optionally includes one or more heteroatoms, said heteroatoms being selected from the atoms consisting of O, S, Si and N” as used herein, refers to a hydrocarbyl group where one or more carbon atoms are replaced by an oxygen, nitrogen, silicon or sulphur atom and thus includes heteroalkyl, heteroalkenyl, heteroalkynyl, cycloheteroalkyl, cycloheteroalkenyl, cycloheteroalkynyl, heteroaryl, arylheteroalkyl, heteroarylalkyl, heteroarylheteroalkyl, arylheteroalkenyl, heteroarylalkenyl, heteroarylheteroalkenyl, heteroarylheteroalkenyl, arylheteroalkynyl, heteroarylalkynyl, heteroarylheteroalkynyl, among others. This term therefore comprises as an example trialkylsilyl, alkoxy, alkenyloxy, alkyl-O-alkyl, alkenyl-O-alkyl, arylalkoxy, benzyloxy, heterocycle, heterocycle-alkyl, heterocycle-alkoxy, among others.

The term “alkyl” as used herein means C₁-C₁₈ normal, secondary, or tertiary, linear or cyclic hydrocarbon with no site of unsaturation. Examples are methyl, ethyl, 1-propyl, 2-propyl, 1-butyl, 2-methyl-1-propyl(i-Bu), 2-butyl (s-Bu) 2-methyl-2-propyl (t-Bu), 1-pentyl (n-pentyl), 2-pentyl, 3-pentyl, 2-methyl-2-butyl, 3-methyl-2-butyl, 3-methyl-1-butyl, 2-methyl-1-butyl, 1-hexyl, 2-hexyl, 3-hexyl, 2-methyl-2-pentyl, 3-methyl-2-pentyl, 4-methyl-2-pentyl, 3-methyl-3-pentyl, 2-methyl-3-pentyl, 2,3-dimethyl-2-butyl, 3,3-dimethyl-2-butyl, cyclopropyl, cyclobutyl, cyclopentyl and cyclohexyl. As used herein and unless otherwise stated, the term “cycloalkyl” means a monocyclic saturated hydrocarbon monovalent radical having from 3 to 10 carbon atoms, such as for instance cyclopropyl, cyclobutyl, cyclopentyl, cyclohexyl, cycloheptyl, cyclooctyl and the like, or a C₇₋₁₀ polycyclic saturated hydrocarbon monovalent radical having from 7 to 10 carbon atoms such as, for instance, norbornyl, fenchyl, trimethyltricycloheptyl or adamantyl. When reference is made to “linear alkyl” or “acyclic alkyl”, this term refers to a C₁₋₁₈ normal, secondary, or tertiary, non-cyclic hydrocarbon with no site of unsaturation.

The term heteroalkyl as used herein means C₁-C₁₈ normal, secondary, or tertiary, linear or cyclic hydrocarbon with no site of unsaturation in which one or more of the carbon atoms has been replaced by a heteroatom selected from the group consisting of C, S, N and P.

The term “alkenyl” as used herein is C₂-C₁₈ normal, secondary or tertiary, linear or cyclic hydrocarbon with at least one site (usually 1 to 3, preferably 1) of unsaturation, i.e. a carbon-carbon, sp2 double bond. Examples include, but are not limited to: ethylene or vinyl (—CH═CH₂), allyl (—CH₂CH═CH₂), cyclopentenyl (—C₅H₇), cyclohenenyl (—C₆H₉) and 5-hexenyl (—CH₂CH₂CH₂CH₂CH═CH₂). The double bond may be in the cis or trans configuration.

The term “acyclic alkenyl” or “linear alkenyl” as used herein refers to C₂-C₁₈ normal, secondary or tertiary, linear, branched or straight hydrocarbon with at least one site (usually 1 to 3, preferably 1) of unsaturation, namely a carbon-carbon, sp2 double bond. Examples include, but are not limited to: ethylene or vinyl (—CH═CH₂), allyl (—CH₂CH═CH₂) and 5-hexenyl (—CH₂CH₂CH₂CH₂CH═CH₂). The double bond may be in the cis or trans configuration.

The term “cycloalkenyl” as used herein refers to a non-aromatic hydrocarbon radical having from 3 to 18 carbon atoms with at least one site (usually 1 to 3, preferably 1) of unsaturation, namely a carbon-carbon, sp2 double bond and consisting of or comprising a C₃₋₁₀ monocyclic or C₇₋₁₈ polycyclic hydrocarbon. Examples include, but are not limited to: cyclopentenyl (—C₅H₇), cyclopentenylpropylene, methylcyclohexenylene and cyclohexenyl (—C₆H₉). The double bond may be in the cis or trans configuration.

The term “alkynyl” as used herein refer respectively C₂-C₁₈ normal, secondary, tertiary, linear or cyclic hydrocarbon with at least one site (usually 1 to 3, preferably 1) of unsaturation, i.e. a carbon-carbon, sp triple bond. Examples include, but are not limited to: acetylenic (—C≡CH) and propargyl (—CH₂C≡CH).

The term “acyclic alkynyl” or “linear alkynyl” as used herein refers to C₂-C₁₈ normal, secondary, tertiary, linear, branched or straight hydrocarbon with at least one site (usually 1 to 3, preferably 1) of unsaturation, namely a carbon-carbon, sp triple bond. Examples include, but are not limited to: ethynyl (—C≡CH) and 1-propynyl (propargyl, —CH₂C≡CH).

The term “cycloalkynyl” as used herein refers to a non-aromatic hydrocarbon radical having from 3 to 18 carbon atoms with at least one site (usually 1 to 3, preferably 1) of unsaturation, namely a carbon-carbon, sp triple bond and consisting of or comprising a C₃₋₁₀ monocyclic or C₇₋₁₈ polycyclic hydrocarbon. Examples include, but are not limited to: cyclohept-1-yne, 3-ethyl-cyclohept-1-ynylene, 4-cyclohept-1-yn-methylene and ethylene-cyclohept-1-yne.

The term “C₁₋₁₈-alkylene” as used herein each refer to a saturated, branched or straight chain hydrocarbon radical of 1-18 carbon atoms, and having two monovalent radical centers derived by the removal of two hydrogen atoms from the same or two different carbon atoms of a parent alkane. Typical alkylene radicals include, but are not limited to: methylene (—CH₂—) 1,2-ethyl (—CH₂CH₂—), 1,3-propyl (—CH₂CH₂CH₂—), 1,4-butyl (—CH₂CH₂CH₂CH₂—), and the like.

The term “alkenylene” as used herein each refer to a branched or straight chain hydrocarbon radical of 2-18 carbon atoms (more in particular C₂₋₁₂ or C₂₋₆-carbon atoms) with at least one site (usually 1 to 3, preferably 1) of unsaturation, namely a carbon-carbon, sp2 double bond, and having two monovalent radical centers derived by the removal of two hydrogen atoms from the same or two different carbon atoms of a parent alkene.

The term “alkynylene” as used herein each refer to a branched or straight chain hydrocarbon of 2-18 carbon atoms (more in particular C₂₋₁₂ or C₂₋₆ carbon atoms) with at least one site (usually 1 to 3, preferably 1) of unsaturation, namely a carbon-carbon, sp triple bond, and having two monovalent radical centers derived by the removal of two hydrogen atoms from the same or two different carbon atoms of a parent alkyne.

The term “aryl” as used herein means a aromatic hydrocarbon of 6-20 carbon atoms derived by the removal of hydrogen from a carbon atom of a parent aromatic ring system. Typical aryl groups include, but are not limited to 1 ring, or 2 or 3 rings fused together, radicals derived from benzene, naphthalene, spiro, anthracene, biphenyl, and the like.

The term “arylalkyl” or “arylalkyl-” as used herein refers to an alkyl in which one of the hydrogen atoms bonded to a carbon atom, typically a terminal or sp3 carbon atom, is replaced with an aryl radical. Typical arylalkyl groups include, but are not limited to, benzyl, 2-phenylethan-1-yl, 2-phenylethen-1-yl, naphthylmethyl, 2-naphthylethan-1-yl, 2-naphthylethen-1-yl, naphthobenzyl, 2-naphthophenylethan-1-yl and the like. The arylalkyl group comprises 6 to 20 carbon atoms, e.g. the alkyl moiety, including alkanyl, alkenyl or alkynyl groups, of the arylalkyl group is 1 to 6 carbon atoms and the aryl moiety is 5 to 14 carbon atoms.

The term “arylalkenyl” or “arylalkenyl-” as used herein refers to an acyclic alkenyl radical in which one of the hydrogen atoms bonded to a carbon atom, is replaced with an aryl radical. The arylalkenyl group comprises 6 to 20 carbon atoms, e.g. the alkenyl moiety of the arylalkenyl group is 1 to 6 carbon atoms and the aryl moiety is 6 to 14 carbon atoms.

The term “arylalkynyl” or “arylalkynyl-” as used herein refers to an acyclic alkynyl radical in which one of the hydrogen atoms bonded to a carbon atom, is replaced with an aryl radical. The arylalkynyl group comprises 6 to 20 carbon atoms, e.g. the alkynyl moiety of the arylalkynyl group is 1 to 6 carbon atoms and the aryl moiety is 6 to 14 carbon atoms.

The term “heterocycle” as used herein means a saturated, unsaturated or aromatic ring system including at least one N, O, S, or P. Heterocycle thus include heteroaryl groups. Heterocycle as used herein includes by way of example and not limitation these heterocycles described in Paquette, Leo A., “Principles of Modern Heterocyclic Chemistry” (W. A. Benjamin, New York, 1968), particularly Chapters 1, 3, 4, 6, 7, and 9, “The Chemistry of Heterocyclic Compounds, A series of Monographs” (John Wiley & Sons, New York, 1950 to present), in particular Volumes 13, 14, 16, 19, and 28, Katritzky, Alan R., Rees, C. W. and Scriven, E. “Comprehensive Heterocyclic Chemistry” (Pergamon Press, 1996), and J. Am. Chem. Soc. (1960) 82:5566. In a particular embodiment, the term means pyridyl, dihydropyridyl, tetrahydropyridyl(piperidyl), thiazolyl, tetrahydrothiophenyl, sulfur oxidized tetrahydrothiophenyl, furanyl, thienyl, pyrrolyl, pyrazolyl, imidazolyl, tetrazolyl, benzofuranyl, thianaphthalenyl, indolyl, indolenyl, quinolinyl, isoquinolinyl, benzimidazolyl, piperidinyl, 4-piperidonyl, pyrrolidinyl, 2-pyrrolidonyl, pyrrolinyl, tetrahydrofuranyl, bis-tetrahydrofuranyl, tetrahydropyranyl, bis-tetrahydropyranyl, tetrahydroquinolinyl, tetrahydroisoquinolinyl, decahydroquinolinyl, octahydroisoquinolinyl, azocinyl, triazinyl, 6H-1,2,5-thiadiazinyl, 2H,6H-1,5,2-dithiazinyl, thianthrenyl, pyranyl, isobenzofuranyl, chromenyl, xanthenyl, phenoxathinyl, 2H-pyrrolyl, isothiazolyl, isoxazolyl, pyrazinyl, pyridazinyl, indolizinyl, isoindolyl, 3H-indolyl, 1H-indazolyl, purinyl, 4H-quinolizinyl, phthalazinyl, naphthyridinyl, quinoxalinyl, quinazolinyl, cinnolinyl, pteridinyl, 4aH-carbazolyl, carbazolyl, β-carbolinyl, phenanthridinyl, acridinyl, pyrimidinyl, phenanthrolinyl, phenazinyl, phenothiazinyl, furazanyl, phenoxazinyl, isochromanyl, chromanyl, imidazolidinyl, imidazolinyl, pyrazolidinyl, pyrazolinyl, piperazinyl, indolinyl, isoindolinyl, quinuclidinyl, morpholinyl, oxazolidinyl, benzotriazolyl, benzisoxazolyl, oxindolyl, benzoxazolinyl, benzothienyl, benzothiazolyl and isatinoyl.

The term heteroaryl, as used herein, means pyridyl, pyridazinyl, pyrimidinyl, pyrazinyl, s-triazinyl, oxazolyl, imidazolyl, thiazolyl, isoxazolyl, pyrazolyl, isothiazolyl, furanyl, thiofuranyl, thienyl, and pyrrolyl.

The term “non-aromatic heterocycle” as used herein means a saturated or unsaturated non-aromatic ring system of 3 to 18 atoms including at least one N, O, S, or P.

The term heterocyclic group includes both “heteroaryl groups” and “non-aromatic heterocycles” e.g. dihydropyridyl.

The term carbocyclic group includes both aryl groups and non-aryl carbocyclic groups e.g. cyclopropyl, cyclobutyl, cyclopentyl, cyclohexyl and unsaturated variants thereof which are not aromatic.

The term “heterocycle-alkyl” or “heterocycle-alkyl-” as used herein refers to an acyclic alkyl radical in which one of the hydrogen atoms bonded to a carbon atom, typically a terminal or sp3 carbon atom, is replaced with a heterocyle radical. An example of a heterocycle-alkyl group is 2-pyridyl-methylene. The heterocycle-alkyl group comprises 6 to 20 atoms, e.g. the alkyl moiety of the heterocycle-alkyl group is 1 to 6 carbon atoms and the heterocycle moiety is 3 to 14 atoms.

The term “heterocycle-alkenyl” or “heterocycle-alkenyl-” as used herein refers to an acyclic alkenyl radical in which one of the hydrogen atoms bonded to a carbon atom, is replaced with an heterocycle radical. The heterocycle-alkenyl group comprises 6 to 20 atoms, e.g. the alkenyl moiety of the heterocycle-alkenyl group is 1 to 6 carbon atoms and the heterocycle moiety is 3 to 14 atoms.

The term “heterocycle-alkynyl” or “heterocycle-alkynyl-” as used herein refers to an acyclic alkynyl radical in which one of the hydrogen atoms bonded to a carbon atom, is replaced with a heterocycle radical. The heterocycle-alkynyl group comprises 6 to 20 atoms, e.g. the alkynyl moiety of the heterocycle-alkynyl group is 1 to 6 carbon atoms and the heterocycle moiety is 3 to 14 atoms.

The term “heteroaryl-alkyl” or “heteroaryl-alkyl-” as used herein refers to an acyclic alkyl radical in which one of the hydrogen atoms bonded to a carbon atom, typically a terminal or sp3 carbon atom, is replaced with a heteraryl radical. An example of a heteroaryl-alkyl group is 2-pyridyl-methylene. The heteroaryl-alkyl group comprises 6 to 20 atoms, e.g. the alkyl moiety of the heteroaryl-alkyl group is 1 to 6 carbon atoms and the heteroaryl moiety is 5 to 14 atoms.

The term “heteroaryl-alkenyl” or “heteroaryl-alkenyl-” as used herein refers to an acyclic alkenyl radical in which one of the hydrogen atoms bonded to a carbon atom, is replaced with an heteroaryl radical. The heteroaryl-alkenyl group comprises 6 to 20 atoms, e.g. the alkenyl moiety of the heteroaryl-alkenyl group is 1 to 6 carbon atoms and the heteroaryl moiety is 5 to 14 atoms.

The term “heteroaryl-alkynyl” or “heteroaryl-alkynyl-” as used herein refers to an acyclic alkynyl radical in which one of the hydrogen atoms bonded to a carbon atom, is replaced with a heteroaryl radical. The heteroaryl-alkynyl group comprises 6 to 20 atoms, e.g. the alkynyl moiety of the heteroaryl-alkynyl group is 1 to 6 carbon atoms and the heteroaryl moiety is 5 to 14 atoms.

As used herein the term amino means a —NH₂ group, but also amino groups in which one or more of the hydrogen atoms has been substituted by an alkyl or aryl group.

As used herein the term ester means an alkyl, heteroalkyl, aryl and heterocyclic ester of a carboxylic acid.

O/S as used in formulae herein means either an oxygen atom or a sulfur atom.

As used herein and unless otherwise stated, the terms “C₁₋₁₈ alkoxy”, “C₃₋₁₀ cycloalkoxy”, “aryloxy”, “arylalkyloxy”, “oxyheterocyclic ring”, “thio C₁₋₇ alkyl”, “thio C₃₋₁₀ cycloalkyl”, “arylthio”, “arylalkylthio” and “thioheterocyclic ring” refer to substituents wherein a C₁₋₁₈ alkyl radical, respectively a C₃₋₁₀ cycloalkyl, aryl, arylalkyl or heterocyclic ring radical (each of them such as defined herein), are attached to an oxygen atom or a sulfur atom through a single bond, such as but not limited to methoxy, ethoxy, propoxy, butoxy, thioethyl, thiomethyl, phenyloxy, benzyloxy, mercaptobenzyl and the like.

As used herein and unless otherwise stated, the term halogen means any atom selected from the group consisting of fluorine, chlorine, bromine and iodine.

As used herein with respect to a substituting group, and unless otherwise stated, the terms “substituted” such as in “substituted alkyl”, “substituted alkenyl”, substituted alkynyl”, “substituted aryl”, “substituted heterocycle”, “substituted arylalkyl”, “substituted heterocycle-alkyl” and the like refer to the chemical structures defined herein, and wherein the said hydrocarbyl, heterohydrocarbyl group and/or the said aryl or heterocycle may be optionally substituted with one or more substituents (preferable 1, 2, 3, 4, 5 or 6), meaning that one or more hydrogen atoms are each independently replaced with a substituent. Typical substituents include, but are not limited to and in a particular embodiment said substituents are independently selected from the group consisting of halogen, amino, hydroxyl, sulfhydryl, alkyl, alkoxy, alkenyl, alkenyloxy, alkynyl, alkynyloxy, cycloalkyl, cycloalkenyl, cycloalkynyl, aryl, heterocycle, arylalkyl, arylalkenyl, arylalkynyl, heterocycle-alkyl, heterocycle-alkenyl and heterocycle-alkynyl, —X¹, —Z¹⁰⁰, —O⁻, —OZ¹⁰⁰, ═O, —SZ¹⁰⁰, —S⁻, ═S, —NZ¹⁰⁰₂, —N⁺Z¹⁰⁰₃, ═NZ¹⁰⁰, ═N—OZ¹⁰⁰, —CX¹₃ (e.g. trifluoromethyl), —ON, —OCN, —SCN, —N═C═O, —N═C═S, —NO, —NO₂, ═N₂, —N₃,

—NZ¹¹¹C(O)Z¹¹¹, —NZ¹¹¹C(S)Z¹¹¹, —NZ¹¹¹C(O)O⁻, —NZ¹¹¹C(O)OZ¹¹¹, —NZ¹¹¹C(S)OZ¹¹¹, —NZ¹¹¹C(O)NZ¹¹¹Z¹¹¹, NZ¹¹¹C(NZ¹¹¹)Z¹¹¹, NZ¹¹¹C(NZ¹¹¹)NZ¹¹¹Z¹¹¹, C(O)NZ¹¹¹Z¹¹¹, —C(NZ¹¹¹)Z¹¹¹, —S(O)₂O⁻, —S(O)₂OZ¹¹¹, —S(O)₂Z¹¹¹, —OS(O)₂OZ¹¹¹, —OS(O)₂Z¹¹¹, —OS(O)₂O⁻, —S(O)₂NZ¹¹¹, —S(O)Z¹¹¹,
—OP(O)(OZ¹¹¹)₂, —P(O)(OZ¹¹¹)₂, —P(O)(O′)₂, —P(O)(OZ¹¹¹)(O′), —P(O)(OH)₂, —C(O)Z¹¹¹, —C(O)X¹, —C(S)Z¹¹¹, —C(O)OZ¹¹¹, —C(O)O′, —C(S)OZ¹¹¹, —C(O)SZ¹¹¹, —C(S)SZ¹¹¹, —C(O)NZ¹¹¹Z¹¹¹, —C(S)NZ¹¹¹Z¹¹¹, —C(NZ¹¹¹)NZ¹¹¹Z¹¹¹, —OC(O)Z¹¹¹, —OC(S)Z¹¹¹, —OC(O)O′, —OC(O)OZ¹¹¹, —OC(S)OZ¹¹¹, wherein each X¹ is independently a halogen selected from F, Cl, Br, or I, and each Z¹⁰⁰ is independently —H, alkyl, alkenyl, alkynyl, aryl, heterocycle, protecting group or prodrug moiety, while two Z¹¹¹ bonded to a nitrogen atom can be taken together with the nitrogen atom to which they are bonded to form a heterocycle. Alkyl(ene), alkenyl(ene), and alkynyl(ene) groups may also be similarly substituted. The substituent groups alkyl, alkenyl and alkynyl may also include one or more heteroatoms in their chain, said heteroatoms being selected from the heteroatoms consisting of O, S, P, and N.

The term “therapeutically suitable pro-drug” is defined herein as “a compound modified in such a way as to be transformed in vivo to the therapeutically active form, whether by way of a single or by multiple biological transformations, when in contact with the tissues of the animal, mammal or human to which the pro-drug has been administered, and without undue toxicity, irritation, or allergic response, and achieving the intended therapeutic outcome”.

The term “prodrug”, as used herein, relates to an inactive or significantly less active derivative of a compound such as represented by the structural formula (I), which undergoes spontaneous or enzymatic transformation within the body in order to release the pharmacologically active form of the compound. For a comprehensive review, reference is made to Rautio J. et al. (“Prodrugs: design and clinical applications” Nature Reviews Drug Discovery, 2008, doi: 10.1038/nrd2468).

By way of example, carbon bonded heterocyclic rings are bonded at position 2, 3, 4, 5, or 6 of a pyridine, position 3, 4, 5, or 6 of a pyridazine, position 2, 4, 5, or 6 of a pyrimidine, position 2, 3, 5, or 6 of a pyrazine, position 2, 3, 4, or 5 of a furan, tetrahydrofuran, thiofuran, thiophene, pyrrole or tetrahydropyrrole, position 2, 4, or 5 of an oxazole, imidazole or thiazole, position 3, 4, or 5 of an isoxazole, pyrazole, or isothiazole, position 2 or 3 of an aziridine, position 2, 3, or 4 of an azetidine, position 2, 3, 4, 5, 6, 7, or 8 of a quinoline or position 1, 3, 4, 5, 6, 7, or 8 of an isoquinoline. Still more typically, carbon bonded heterocycles include 2-pyridyl, 3-pyridyl, 4-pyridyl, 5-pyridyl, 6-pyridyl, 3-pyridazinyl, 4-pyridazinyl, 5-pyridazinyl, 6-pyridazinyl, 2-pyrimidinyl, 4-pyrimidinyl, 5-pyrimidinyl, 6-pyrimidinyl, 2-pyrazinyl, 3-pyrazinyl, 5-pyrazinyl, 6-pyrazinyl, 2-thiazolyl, 4-thiazolyl, or 5-thiazolyl.

By way of example, nitrogen bonded heterocyclic rings are bonded at position 1 of an aziridine, azetidine, pyrrole, pyrrolidine, 2-pyrroline, 3-pyrroline, imidazole, imidazolidine, 2-imidazoline, 3-imidazoline, pyrazole, pyrazoline, 2-pyrazoline, 3-pyrazoline, piperidine, piperazine, indole, indoline, 1H-indazole, position 2 of a isoindole, or isoindoline, position 4 of a morpholine, and position 9 of a carbazole, or β-carboline. Still more typically, nitrogen bonded heterocycles include 1-aziridyl, 1-azetedyl, 1-pyrrolyl, 1-imidazolyl, 1-pyrazolyl, and 1-piperidinyl.

Any substituent designation that is found in more than one site in a compound of this invention shall be independently selected.

Substituents optionally are designated with or without bonds. Regardless of bond indications, if a substituent is polyvalent (based on its position in the structure referred to), then any and all possible orientations of the substituent are intended.

It is to be understood that although preferred embodiments, specific constructions and configurations, as well as materials, have been discussed herein for devices according to the present invention, various changes or modifications in form and detail may be made without departing from the scope and spirit of this invention. For example, any formulas given above are merely representative of procedures that may be used. Functionality may be added or deleted from the block diagrams and operations may be interchanged among functional blocks. Steps may be added or deleted to methods described within the scope of the present invention.

One aspect of the present invention is the provision of isoquinolin-1-one derivatives, namely compounds of formula (I)

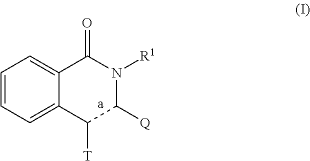

wherein,

the dotted line “a” is selected from a single bond or a double bond,

each of R¹, R² and R³ is independently selected from hydrogen or a hydrocarbyl group which optionally includes one or more heteroatoms, said heteroatoms being selected from the heteroatoms consisting of O, S, P, and N, wherein said hydrocarbyl group can be unsubstituted or substituted,

each R⁴ is independently selected from hydrogen, halogen, —OH, —OR⁵, —SH, —SR⁵, —S(O)R⁶, —S(O)₂R⁶, —SO₂NR⁷R⁸, trifluoromethyl, trifluoromethoxy, nitro, —NR⁵C(O)R⁵, —NR⁵S(O)₂R⁵, —NR⁵C(O)NR⁷R⁸, —NR⁷R⁸, -cyano, —COOH, —COOR⁵, —C(O)NR⁷R⁸, —C(O)R⁶, alkyl, alkenyl, alkynyl, aryl, heterocycle, arylalkyl, arylalkenyl, arylalkynyl, heterocycle-alkyl, heterocycle-alkenyl, heterocycle-alkynyl,
and wherein said alkyl, alkenyl, alkynyl, aryl, heterocycle, arylalkyl, arylalkenyl, arylalkynyl, heterocycle-alkyl, heterocycle-alkenyl or heterocycle-alkynyl, optionally includes one or more heteroatoms, said heteroatoms being selected from the atoms O, S, P and N,
and wherein said alkyl, alkenyl, alkynyl, aryl, heterocycle, arylalkyl, arylalkenyl, arylalkynyl, heterocycle-alkyl, heterocycle-alkenyl or heterocycle-alkynyl can be unsubstituted or substituted with halogen, —OH, —OR⁵, —SH, —SR⁵, —S(O)R⁶, —S(O)₂R⁶, —SO₂NR⁷R⁸, trifluoromethyl, nitro, —NR⁵C(O)R⁵, —NR⁵S(O)₂R⁵, —NR⁵C(O)NR⁷R⁸, —NR⁷R⁸, -cyano, —COOH, —COOR⁵, —C(O)NR⁷R⁸, —C(O)R⁶,

n is selected from 0, 1, 2, 3 or 4,

S is L¹R² or R²,

L¹ is selected from C₁₋₆alkyl, C₁₋₆alkenyl or C₁₋₆alkynyl, wherein each of said C₁₋₆alkyl, C₁₋₆alkenyl or C₁₋₆alkynyl optionally includes one or more heteroatoms, said heteroatoms being selected from the heteroatoms consisting of O, S, P, and N, and wherein each of said C₁₋₆alkyl, C₁₋₆alkenyl or C₁₋₆alkynyl can be unsubstituted or substituted,

T is L²R³ or R³,

L² is selected from C₁₋₆alkyl, C₁₋₆alkenyl or C₁₋₆alkynyl, wherein each of said C₁₋₆alkyl, C₁₋₆alkenyl or C₁₋₆alkynyl optionally includes one or more heteroatoms, said heteroatoms being selected from the heteroatoms consisting of O, S, P, and N, and wherein each of said C₁₋₆alkyl, C₁₋₆alkenyl or C₁₋₆alkynyl can be unsubstituted or substituted,

each R⁵ is independently selected from alkyl, alkenyl, alkynyl, aryl, heterocycle, arylalkyl, arylalkenyl, arylalkynyl, heterocycle-alkyl, heterocycle-alkenyl or heterocycle-alkynyl, and wherein said alkyl, alkenyl, alkynyl, aryl, heterocycle, arylalkyl, arylalkenyl, arylalkynyl, heterocycle-alkyl, heterocycle-alkenyl or heterocycle-alkynyl optionally include one or more heteroatoms, said heteroatom selected from O, S, P and N, and wherein said alkyl, alkenyl, alkynyl, aryl, heterocycle, arylalkyl, arylalkenyl, arylalkynyl, heterocycle-alkyl, heterocycle-alkenyl or heterocycle-alkynyl can be unsubstituted or substituted,

each R⁶ is independently selected from hydroxyl, alkyl, alkenyl, alkynyl, aryl, heterocycle, arylalkyl, arylalkenyl, arylalkynyl, heterocycle-alkyl, heterocycle-alkenyl or heterocycle-alkynyl, and wherein said alkyl, alkenyl, alkynyl, aryl, heterocycle, arylalkyl, arylalkenyl, arylalkynyl, heterocycle-alkyl, heterocycle-alkenyl or heterocycle-alkynyl optionally includes one or more heteroatoms, said heteroatom selected from O, S, P and N, and wherein said alkyl, alkenyl, alkynyl, aryl, heterocycle, arylalkyl, arylalkenyl, arylalkynyl, heterocycle-alkyl, heterocycle-alkenyl or heterocycle-alkynyl can be unsubstituted or substituted,

each R⁷ and R⁸ is independently selected from hydrogen, alkyl, alkenyl, alkynyl, aryl, heterocycle, arylalkyl, arylalkenyl, arylalkynyl, heterocycle-alkyl, heterocycle-alkenyl or heterocycle-alkynyl, alkyl, alkenyl, alkynyl, aryl, heterocycle, arylalkyl, arylalkenyl, arylalkynyl, heterocycle-alkyl, heterocycle-alkenyl or heterocycle-alkynyl optionally includes one or more heteroatoms, said heteroatom selected from O, S, P and N, and wherein said alkyl, alkenyl, alkynyl, aryl, heterocycle, arylalkyl, arylalkenyl, arylalkynyl, heterocycle-alkyl, heterocycle-alkenyl or heterocycle-alkynyl can be unsubstituted or substituted with hydroxyl, halogen, —SH, trifluoromethyl, —OCH₃, —OCF₃, cyano, nitro, —COOH or NH₂, and wherein R⁷ and R⁸ can be taken together in order to form a (5-, 6-, or 7-membered) heterocycle which can be unsubstituted or substituted,

and isomers (in particular stereo-isomers or tautomers), solvates, hydrates, salts (in particular pharmaceutically acceptable salts) or prodrugs thereof,
for use as a medicament for the prevention or treatment of a RNA virus infection in an animal, a mammal (including a human).

In a particular embodiment, the compounds of the invention have a formula according to the formula (Ia), (Ib), (Ic) or (Id):

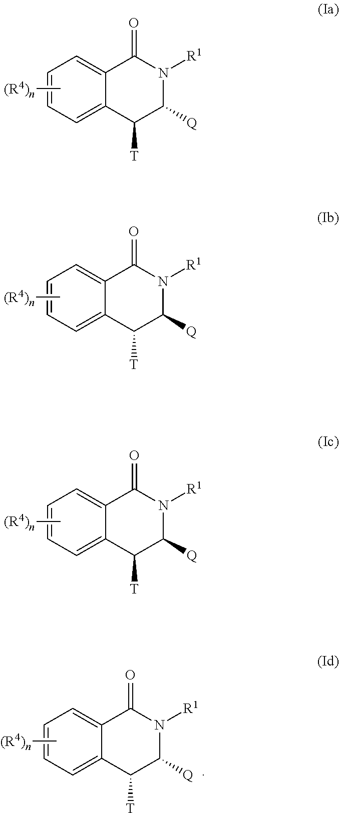

wherein each of R¹, R², R³, R⁴, n, S and T are as for formula (I).

In yet another particular embodiment, the compounds of the invention have a structure according to formula (I), (Ia), (Ib), (Ic), or (Id), wherein,

R¹ and R² are independently selected from alkyl, alkenyl, alkynyl, aryl, heterocycle, arylalkyl, arylalkenyl, arylalkynyl, heterocycle-alkyl, heterocycle-alkenyl or heterocycle-alkynyl, and wherein said alkyl, alkenyl, alkynyl, aryl, heterocycle, arylalkyl, arylalkenyl, arylalkynyl, heterocycle-alkyl, heterocycle-alkenyl or heterocycle-alkynyl optionally include one or more heteroatoms, said heteroatom selected from O, S, P and N, and wherein said alkyl, alkenyl, alkynyl, aryl, heterocycle, arylalkyl, arylalkenyl, arylalkynyl, heterocycle-alkyl, heterocycle-alkenyl or heterocycle-alkynyl can be unsubstituted or substituted with one or more Z¹⁰,

R³ is independently selected from alkyl, alkenyl, alkynyl, aryl, heterocycle, arylalkyl, arylalkenyl, arylalkynyl, heterocycle-alkyl, heterocycle-alkenyl or heterocycle-alkynyl, and wherein said alkyl, alkenyl, alkynyl, aryl, heterocycle, arylalkyl, arylalkenyl, arylalkynyl, heterocycle-alkyl, heterocycle-alkenyl or heterocycle-alkynyl optionally include one or more heteroatoms, said heteroatom selected from O, S, P and N, and wherein said alkyl, alkenyl, alkynyl, aryl, heterocycle, arylalkyl, arylalkenyl, arylalkynyl, heterocycle-alkyl, heterocycle-alkenyl or heterocycle-alkynyl can be unsubstituted or substituted with one or more Z¹,

each R⁴ is independently selected from hydrogen, halogen, —OH, —OR⁵, —SH, —SR⁵, —S(O)R⁶, —S(O)₂R⁶, —SO₂NR⁷R⁸, trifluoromethyl, nitro, —NR⁵C(O)R⁵, —NR⁵S(O)₂R⁵, —NR⁵C(O)NR⁷R⁸, —NR⁷R⁸, -cyano, —COOH, —COOR⁵, —C(O)NR⁷R⁸, —C(O)R⁶, alkyl, alkenyl, alkynyl, aryl, heterocycle, arylalkyl, arylalkenyl, arylalkynyl, heterocycle-alkyl, heterocycle-alkenyl, heterocycle-alkynyl,
and wherein said alkyl, alkenyl, alkynyl, aryl, heterocycle, arylalkyl, arylalkenyl, arylalkynyl, heterocycle-alkyl, heterocycle-alkenyl or heterocycle-alkynyl, optionally includes one or more heteroatoms, said heteroatoms being selected from the atoms O, S, P and N,
and wherein said alkyl, alkenyl, alkynyl, aryl, heterocycle, arylalkyl, arylalkenyl, arylalkynyl, heterocycle-alkyl, heterocycle-alkenyl or heterocycle-alkynyl can be unsubstituted or substituted with one or more halogen, —OH, ═O, ═S, —OR⁵, —SH, —SR⁵, —S(O)R⁶, —S(O)₂R⁶, —SO₂NR⁷R⁸, trifluoromethyl, nitro, —NR⁵C(O)R⁵, —NR⁵S(O)₂R⁵, —NR⁵C(O)NR⁷R⁸, —NR⁷R⁸, -cyano, —COOH, —COOR⁵, —C(O)NR⁷R⁸, —C(O)R⁶,

n is selected from 0, 1, 2, 3 or 4,

Q is L¹R² or R²,

L¹ is selected from C₁₋₆alkyl, C₁₋₆alkenyl or C₁₋₆alkynyl, wherein each of said C₁₋₆alkyl, C₁₋₆alkenyl or C₁₋₆alkynyl optionally includes one or more heteroatoms, said heteroatoms being selected from the heteroatoms consisting of O, S, P, and N, and wherein each of said C₁₋₆alkyl, C₁₋₆alkenyl or C₁₋₆alkynyl can be unsubstituted or substituted with one or more Z¹⁰,

T is L²R³ or R³,

L² is selected from C₁₋₆alkyl, C₁₋₆alkenyl or C₁₋₆alkynyl, wherein each of said C₁₋₆alkyl, C₁₋₆alkenyl or C₁₋₆alkynyl optionally includes one or more heteroatoms, said heteroatoms being selected from the heteroatoms consisting of O, S, P, and N, and wherein each of said C₁₋₆alkyl, C₁₋₆alkenyl or C₁₋₆alkynyl can be unsubstituted or substituted with one or more Z¹⁰,

each R⁵ is independently selected from alkyl, alkenyl, alkynyl, aryl, heterocycle, arylalkyl, arylalkenyl, arylalkynyl, heterocycle-alkyl, heterocycle-alkenyl or heterocycle-alkynyl, and wherein said alkyl, alkenyl, alkynyl, aryl, heterocycle, arylalkyl, arylalkenyl, arylalkynyl, heterocycle-alkyl, heterocycle-alkenyl or heterocycle-alkynyl optionally include one or more heteroatoms, said heteroatom selected from O, S, P and N, and wherein said alkyl, alkenyl, alkynyl, aryl, heterocycle, arylalkyl, arylalkenyl, arylalkynyl, heterocycle-alkyl, heterocycle-alkenyl or heterocycle-alkynyl can be unsubstituted or substituted with one or more Z¹⁰,

each R⁶ is independently selected from hydroxyl, alkyl, alkenyl, alkynyl, aryl, heterocycle, arylalkyl, arylalkenyl, arylalkynyl, heterocycle-alkyl, heterocycle-alkenyl or heterocycle-alkynyl, and wherein said alkyl, alkenyl, alkynyl, aryl, heterocycle, arylalkyl, arylalkenyl, arylalkynyl, heterocycle-alkyl, heterocycle-alkenyl or heterocycle-alkynyl optionally includes one or more heteroatoms, said heteroatom selected from O, S, P and N, and wherein said alkyl, alkenyl, alkynyl, aryl, heterocycle, arylalkyl, arylalkenyl, arylalkynyl, heterocycle-alkyl, heterocycle-alkenyl or heterocycle-alkynyl can be unsubstituted or substituted with one or more Z¹⁰,

each R⁷ and R⁸ is independently selected from hydrogen, alkyl, alkenyl, alkynyl, aryl, heterocycle, arylalkyl, arylalkenyl, arylalkynyl, heterocycle-alkyl, heterocycle-alkenyl or heterocycle-alkynyl, alkyl, alkenyl, alkynyl, aryl, heterocycle, arylalkyl, arylalkenyl, arylalkynyl, heterocycle-alkyl, heterocycle-alkenyl or heterocycle-alkynyl optionally includes one or more heteroatoms, said heteroatom selected from O, S, P and N, and wherein said alkyl, alkenyl, alkynyl, aryl, heterocycle, arylalkyl, arylalkenyl, arylalkynyl, heterocycle-alkyl, heterocycle-alkenyl or heterocycle-alkynyl can be unsubstituted or substituted with hydroxyl, halogen, —SH, trifluoromethyl, —OCH₃, —OCF₃, cyano, nitro, —COOH or NH₂, and wherein R⁷ and R⁸ can be taken together in order to form a (5-, 6-, or 7-membered) heterocycle which can be unsubstituted or substituted with one or more Z¹⁰, each Z¹ is independently selected from the group consisting of hydrogen, halogen, hydroxyl, sulfhydryl, —OZ², —SZ², ═O, ═S, —S(O)Z³, —S(O)₂Z³, —SO₂NZ⁴Z⁵, trifluoromethyl, trifluoromethoxy, nitro, —NZ⁴Z⁵, —NZ⁴S(O)₂Z², —NZ⁴C(O)Z², —NZ⁴C(O)NZ⁴Z⁵, cyano, —COOZ², —C(O)NZ⁴Z⁵, —C(O)Z³, —C(O)H, alkyl, alkenyl, alkynyl, aryl, heterocycle, arylalkyl, arylalkenyl, arylalkynyl, heterocycle-alkyl, heterocycle-alkenyl, heterocycle-alkynyl,
and wherein said alkyl, alkenyl, alkynyl, aryl, heterocycle, arylalkyl, arylalkenyl, arylalkynyl, heterocycle-alkyl, heterocycle-alkenyl or heterocycle-alkynyl, optionally includes one or more heteroatoms, said heteroatoms being selected from the atoms O, S, P and N,
and wherein said alkyl, alkenyl, alkynyl, aryl, heterocycle, arylalkyl, arylalkenyl, arylalkynyl, heterocycle-alkyl, heterocycle-alkenyl or heterocycle-alkynyl can be unsubstituted or substituted with one or more Z¹⁰,

each Z² is independently selected from hydrogen, alkyl, alkenyl, alkynyl, aryl, heterocycle, arylalkyl, arylalkenyl, arylalkynyl, heterocycle-alkyl, heterocycle-alkenyl or heterocycle-alkynyl, and wherein said alkyl, alkenyl, alkynyl, aryl, heterocycle, arylalkyl, arylalkenyl, arylalkynyl, heterocycle-alkyl, heterocycle-alkenyl or heterocycle-alkynyl optionally include one or more heteroatoms, said heteroatom selected from O, S, P and N, and wherein said alkyl, alkenyl, alkynyl, aryl, heterocycle, arylalkyl, arylalkenyl, arylalkynyl, heterocycle-alkyl, heterocycle-alkenyl or heterocycle-alkynyl can be unsubstituted or substituted with one or more Z¹⁰,

each Z³ is independently selected from hydroxyl, alkyl, alkenyl, alkynyl, aryl, heterocycle, arylalkyl, arylalkenyl, arylalkynyl, heterocycle-alkyl, heterocycle-alkenyl or heterocycle-alkynyl, and wherein said alkyl, alkenyl, alkynyl, aryl, heterocycle, arylalkyl, arylalkenyl, arylalkynyl, heterocycle-alkyl, heterocycle-alkenyl or heterocycle-alkynyl optionally includes one or more heteroatoms, said heteroatom selected from O, S, P and N, and wherein said alkyl, alkenyl, alkynyl, aryl, heterocycle, arylalkyl, arylalkenyl, arylalkynyl, heterocycle-alkyl, heterocycle-alkenyl or heterocycle-alkynyl can be unsubstituted or substituted with one or more Z¹⁰,

each Z⁴ and Z⁵ is independently selected from hydrogen, alkyl, alkenyl, alkynyl, aryl, heterocycle, arylalkyl, arylalkenyl, arylalkynyl, heterocycle-alkyl, heterocycle-alkenyl or heterocycle-alkynyl, alkyl, alkenyl, alkynyl, aryl, heterocycle, arylalkyl, arylalkenyl, arylalkynyl, heterocycle-alkyl, heterocycle-alkenyl or heterocycle-alkynyl optionally includes one or more heteroatoms, said heteroatom selected from O, S, P and N, and wherein said alkyl, alkenyl, alkynyl, aryl, heterocycle, arylalkyl, arylalkenyl, arylalkynyl, heterocycle-alkyl, heterocycle-alkenyl or heterocycle-alkynyl can be unsubstituted or substituted with one or more Z¹⁰, and wherein Z⁴ and Z⁵ can be taken together in order to form a (5-, 6-, or 7-membered) heterocycle which can be unsubstituted or substituted with one or more Z¹⁰,

each Z¹⁰ is independently selected from the group consisting of hydrogen, halogen, hydroxyl, sulfhydryl, —OZ¹², —SZ¹², ═O, ═S, —S(O)Z¹³, —S(O)₂Z¹³, —SO₂NZ¹⁴Z¹⁵, trifluoromethyl, trifluoromethoxy, nitro, —NZ¹⁴Z¹⁵, —NZ¹⁴S(O)₂Z¹², —NZ¹⁴C(O)Z¹², —NZ¹⁴C(O)NZ¹⁴Z¹⁵, ⁻cyano, —COOZ¹², —C(O)NZ¹⁴Z¹⁵, —C(O)Z¹³, —C(O)H, alkyl, alkenyl, alkynyl, aryl, heterocycle, arylalkyl, arylalkenyl, arylalkynyl, heterocycle-alkyl, heterocycle-alkenyl, heterocycle-alkynyl,
and wherein said alkyl, alkenyl, alkynyl, aryl, heterocycle, arylalkyl, arylalkenyl, arylalkynyl, heterocycle-alkyl, heterocycle-alkenyl or heterocycle-alkynyl, optionally includes one or more heteroatoms, said heteroatoms being selected from the atoms O, S, P and N,
and wherein said alkyl, alkenyl, alkynyl, aryl, heterocycle, arylalkyl, arylalkenyl, arylalkynyl, heterocycle-alkyl, heterocycle-alkenyl or heterocycle-alkynyl can be unsubstituted or substituted with one or more hydroxyl, halogen, —SH, trifluoromethyl, —OCH₃, —OCF₃, cyano, nitro, —COOH or NH₂,

each Z¹² is independently selected from hydrogen, alkyl, alkenyl, alkynyl, aryl, heterocycle, arylalkyl, arylalkenyl, arylalkynyl, heterocycle-alkyl, heterocycle-alkenyl or heterocycle-alkynyl, and wherein said alkyl, alkenyl, alkynyl, aryl, heterocycle, arylalkyl, arylalkenyl, arylalkynyl, heterocycle-alkyl, heterocycle-alkenyl or heterocycle-alkynyl optionally include one or more heteroatoms, said heteroatom selected from O, S, P and N, and wherein said alkyl, alkenyl, alkynyl, aryl, heterocycle, arylalkyl, arylalkenyl, arylalkynyl, heterocycle-alkyl, heterocycle-alkenyl or heterocycle-alkynyl can be unsubstituted or substituted with one or more hydroxyl, halogen, —SH, trifluoromethyl, —OCH₃, —OCF₃, cyano, nitro, —COOH or NH₂,

each Z¹³ is independently selected from hydroxyl, alkyl, alkenyl, alkynyl, aryl, heterocycle, arylalkyl, arylalkenyl, arylalkynyl, heterocycle-alkyl, heterocycle-alkenyl or heterocycle-alkynyl, and wherein said alkyl, alkenyl, alkynyl, aryl, heterocycle, arylalkyl, arylalkenyl, arylalkynyl, heterocycle-alkyl, heterocycle-alkenyl or heterocycle-alkynyl optionally includes one or more heteroatoms, said heteroatom selected from O, S, P and N, and wherein said alkyl, alkenyl, alkynyl, aryl, heterocycle, arylalkyl, arylalkenyl, arylalkynyl, heterocycle-alkyl, heterocycle-alkenyl or heterocycle-alkynyl can be unsubstituted or substituted with one or more hydroxyl, halogen, —SH, trifluoromethyl, —OCH₃, —OCF₃, cyano, nitro, —COOH or NH₂,

each Z¹⁴ and Z¹⁵ is independently selected from hydrogen, alkyl, alkenyl, alkynyl, aryl, heterocycle, arylalkyl, arylalkenyl, arylalkynyl, heterocycle-alkyl, heterocycle-alkenyl or heterocycle-alkynyl, alkyl, alkenyl, alkynyl, aryl, heterocycle, arylalkyl, arylalkenyl, arylalkynyl, heterocycle-alkyl, heterocycle-alkenyl or heterocycle-alkynyl optionally includes one or more heteroatoms, said heteroatom selected from O, S, P and N, and wherein said alkyl, alkenyl, alkynyl, aryl, heterocycle, arylalkyl, arylalkenyl, arylalkynyl, heterocycle-alkyl, heterocycle-alkenyl or heterocycle-alkynyl can be unsubstituted or substituted with one or more hydroxyl, halogen, —SH, trifluoromethyl, —OCH₃, —OCF₃, cyano, nitro, —COOH or NH₂, and wherein Z¹⁴ and Z¹⁵ can be taken together in order to form a (5-, 6-, or 7-membered) heterocycle which can be unsubstituted or substituted with one or more hydroxyl, halogen, —SH, trifluoromethyl, —OCH₃, —OCF₃, cyano, nitro, —COOH or NH₂.

In yet another particular embodiment, the compounds of the invention have a structure according to formula (I), (Ia), (Ib), (Ic), or (Id), wherein,

R¹, R² and R³ are independently selected from alkyl, alkenyl, alkynyl, aryl, heterocycle, arylalkyl, arylalkenyl, arylalkynyl, heterocycle-alkyl, heterocycle-alkenyl or heterocycle-alkynyl, and wherein said alkyl, alkenyl, alkynyl, aryl, heterocycle, arylalkyl, arylalkenyl, arylalkynyl, heterocycle-alkyl, heterocycle-alkenyl or heterocycle-alkynyl optionally include one or more heteroatoms, said heteroatom selected from O, S, P and N, and wherein said alkyl, alkenyl, alkynyl, aryl, heterocycle, arylalkyl, arylalkenyl, arylalkynyl, heterocycle-alkyl, heterocycle-alkenyl or heterocycle-alkynyl can be unsubstituted or substituted with one or more Z¹,

each R⁴ is independently selected from hydrogen, halogen, —OH, —OR⁵, —SH, —SR⁵, —S(O)R⁶, —S(O)₂R⁶, —SO₂NR⁷R⁸, trifluoromethyl, nitro, —NR⁵C(O)R⁵, —NR⁵S(O)₂R⁵, —NR⁵C(O)NR⁷R⁸, —NR⁷R⁸, -cyano, —COOH, —COOR⁵, —C(O)NR⁷R⁸, —C(O)R⁶, alkyl, alkenyl, alkynyl, aryl, heterocycle, arylalkyl, arylalkenyl, arylalkynyl, heterocycle-alkyl, heterocycle-alkenyl, heterocycle-alkynyl,
and wherein said alkyl, alkenyl, alkynyl, aryl, heterocycle, arylalkyl, arylalkenyl, arylalkynyl, heterocycle-alkyl, heterocycle-alkenyl or heterocycle-alkynyl, optionally includes one or more heteroatoms, said heteroatoms being selected from the atoms O, S, P and N,
and wherein said alkyl, alkenyl, alkynyl, aryl, heterocycle, arylalkyl, arylalkenyl, arylalkynyl, heterocycle-alkyl, heterocycle-alkenyl or heterocycle-alkynyl can be unsubstituted or substituted with one or more halogen, —OH, ═O, ═S, —OR⁵, —SH, —SR⁵, —S(O)R⁶, —S(O)₂R⁶, —SO₂NR⁷R⁸, trifluoromethyl, nitro, —NR⁵C(O)R⁵, —NR⁵S(O)₂R⁵, —NR⁵C(O)NR⁷R⁸, —NR⁷R⁸, -cyano, —COOH, —COOR⁵, —C(O)NR⁷R⁸, —C(O)R⁶,

n is selected from 0, 1, 2, 3 or 4,

Q is L¹R² or R²,

L¹ is selected from C₁₋₆alkyl, C₁₋₆alkenyl or C₁₋₆alkynyl, wherein each of said C₁₋₆alkyl, C₁₋₆alkenyl or C₁₋₆alkynyl optionally includes one or more heteroatoms, said heteroatoms being selected from the heteroatoms consisting of O, S, P, and N, and wherein each of said C₁₋₆alkyl, C₁₋₆alkenyl or C₁₋₆alkynyl can be unsubstituted or substituted with one or more Z¹,

T is L²R³ or R³,

L² is selected from C₁₋₆alkyl, C₁₋₆alkenyl or C₁₋₆alkynyl, wherein each of said C₁₋₆alkyl, C₁₋₆alkenyl or C₁₋₆alkynyl optionally includes one or more heteroatoms, said heteroatoms being selected from the heteroatoms consisting of O, S, P, and N, and wherein each of said C₁₋₆alkyl, C₁₋₆alkenyl or C₁₋₆alkynyl can be unsubstituted or substituted with one or more Z¹,

each R⁵ is independently selected from alkyl, alkenyl, alkynyl, aryl, heterocycle, arylalkyl, arylalkenyl, arylalkynyl, heterocycle-alkyl, heterocycle-alkenyl or heterocycle-alkynyl, and wherein said alkyl, alkenyl, alkynyl, aryl, heterocycle, arylalkyl, arylalkenyl, arylalkynyl, heterocycle-alkyl, heterocycle-alkenyl or heterocycle-alkynyl optionally include one or more heteroatoms, said heteroatom selected from O, S, P and N, and wherein said alkyl, alkenyl, alkynyl, aryl, heterocycle, arylalkyl, arylalkenyl, arylalkynyl, heterocycle-alkyl, heterocycle-alkenyl or heterocycle-alkynyl can be unsubstituted or substituted with one or more Z¹,

each R⁶ is independently selected from hydroxyl, alkyl, alkenyl, alkynyl, aryl, heterocycle, arylalkyl, arylalkenyl, arylalkynyl, heterocycle-alkyl, heterocycle-alkenyl or heterocycle-alkynyl, and wherein said alkyl, alkenyl, alkynyl, aryl, heterocycle, arylalkyl, arylalkenyl, arylalkynyl, heterocycle-alkyl, heterocycle-alkenyl or heterocycle-alkynyl optionally includes one or more heteroatoms, said heteroatom selected from O, S, P and N, and wherein said alkyl, alkenyl, alkynyl, aryl, heterocycle, arylalkyl, arylalkenyl, arylalkynyl, heterocycle-alkyl, heterocycle-alkenyl or heterocycle-alkynyl can be unsubstituted or substituted with one or more Z¹,

each of R⁷ and R⁸ is independently selected from hydrogen, alkyl, alkenyl, alkynyl, aryl, heterocycle, arylalkyl, arylalkenyl, arylalkynyl, heterocycle-alkyl, heterocycle-alkenyl or heterocycle-alkynyl, alkyl, alkenyl, alkynyl, aryl, heterocycle, arylalkyl, arylalkenyl, arylalkynyl, heterocycle-alkyl, heterocycle-alkenyl or heterocycle-alkynyl optionally includes one or more heteroatoms, said heteroatom selected from O, S, P and N, and wherein said alkyl, alkenyl, alkynyl, aryl, heterocycle, arylalkyl, arylalkenyl, arylalkynyl, heterocycle-alkyl, heterocycle-alkenyl or heterocycle-alkynyl can be unsubstituted or substituted with hydroxyl, halogen, —SH, trifluoromethyl, —OCH₃, —OCF₃, cyano, nitro, —COOH or NH₂, and wherein R⁷ and R⁸ can be taken together in order to form a (5-, 6-, or 7-membered) heterocycle which can be unsubstituted or substituted with one or more Z¹,

each of Z¹ is independently selected from the group consisting of hydrogen, halogen, hydroxyl, sulfhydryl, —OZ², —SZ², ═O, ═S, —S(O)Z³, —S(O)₂Z³, —SO₂NZ⁴Z⁵, trifluoromethyl, trifluoromethoxy, nitro, —NZ⁴Z⁵, —NZ⁴S(O)₂Z², —NZ⁴C(O)Z², —NZ⁴C(O)NZ⁴Z⁵, ⁻cyano, —COOZ², —C(O)NZ⁴Z⁵, —C(O)Z³, —C(O)H, alkyl, alkenyl, alkynyl, aryl, heterocycle, arylalkyl, arylalkenyl, arylalkynyl, heterocycle-alkyl, heterocycle-alkenyl, heterocycle-alkynyl,
and wherein said alkyl, alkenyl, alkynyl, aryl, heterocycle, arylalkyl, arylalkenyl, arylalkynyl, heterocycle-alkyl, heterocycle-alkenyl or heterocycle-alkynyl, optionally includes one or more heteroatoms, said heteroatoms being selected from the atoms O, S, P and N,
and wherein said alkyl, alkenyl, alkynyl, aryl, heterocycle, arylalkyl, arylalkenyl, arylalkynyl, heterocycle-alkyl, heterocycle-alkenyl or heterocycle-alkynyl can be unsubstituted or substituted with one or more hydroxyl, ═O, ═S, halogen, —SH, trifluoromethyl, —OCH₃, —O-alkyl, —OCF₃, cyano, nitro, —COOH or NH₂,

each of Z² and Z³ is independently selected from hydrogen, alkyl, alkenyl, alkynyl, aryl, heterocycle, arylalkyl, arylalkenyl, arylalkynyl, heterocycle-alkyl, heterocycle-alkenyl or heterocycle-alkynyl, and wherein said alkyl, alkenyl, alkynyl, aryl, heterocycle, arylalkyl, arylalkenyl, arylalkynyl, heterocycle-alkyl, heterocycle-alkenyl or heterocycle-alkynyl optionally include one or more heteroatoms, said heteroatom selected from O, S, P and N, and wherein said alkyl, alkenyl, alkynyl, aryl, heterocycle, arylalkyl, arylalkenyl, arylalkynyl, heterocycle-alkyl, heterocycle-alkenyl or heterocycle-alkynyl can be unsubstituted or substituted with one or more hydroxyl, ═O, ═S, halogen, —SH, trifluoromethyl, —OCH₃, —O-alkyl, —OCF₃, cyano, nitro, —COOH or NH₂,

each of Z⁴ and Z⁵ is independently selected from hydrogen, alkyl, alkenyl, alkynyl, aryl, heterocycle, arylalkyl, arylalkenyl, arylalkynyl, heterocycle-alkyl, heterocycle-alkenyl or heterocycle-alkynyl, alkyl, alkenyl, alkynyl, aryl, heterocycle, arylalkyl, arylalkenyl, arylalkynyl, heterocycle-alkyl, heterocycle-alkenyl or heterocycle-alkynyl optionally includes one or more heteroatoms, said heteroatom selected from O, S, P and N, and wherein said alkyl, alkenyl, alkynyl, aryl, heterocycle, arylalkyl, arylalkenyl, arylalkynyl, heterocycle-alkyl, heterocycle-alkenyl or heterocycle-alkynyl can be unsubstituted or substituted with one or more Z¹⁰, and wherein Z⁴ and Z⁵ can be taken together in order to form a (5-, 6-, or 7-membered) heterocycle which can be unsubstituted or substituted with one or more hydroxyl, ═O, ═S, halogen, —SH, trifluoromethyl, —OCH₃, —O-alkyl, —OCF₃, cyano, nitro, —COOH or NH₂.

In yet another particular embodiment, the compounds of the invention are according to formula (I), (Ia), (Ib), (Ic), or (Id), wherein,

each of R¹, R² and R³ is independently selected from alkyl, alkenyl, alkynyl, aryl, heterocycle, arylalkyl, arylalkenyl, arylalkynyl, heterocycle-alkyl, heterocycle-alkenyl or heterocycle-alkynyl, and wherein said alkyl, alkenyl, alkynyl, aryl, heterocycle, arylalkyl, arylalkenyl, arylalkynyl, heterocycle-alkyl, heterocycle-alkenyl or heterocycle-alkynyl optionally include one or more heteroatoms, said heteroatom selected from O, S, P and N, and wherein said alkyl, alkenyl, alkynyl, aryl, heterocycle, arylalkyl, arylalkenyl, arylalkynyl, heterocycle-alkyl, heterocycle-alkenyl or heterocycle-alkynyl can be unsubstituted or substituted with one or more Z¹ independently selected,

Q is L¹R² or R²,

R² is selected from C₁₋₆alkyl, C₁₋₆alkenyl or C₁₋₆alkynyl, wherein each of said C₁₋₆alkyl, C₁₋₆alkenyl or C₁₋₆alkynyl optionally includes one or more heteroatoms, said heteroatoms being selected from the heteroatoms consisting of O, S, P, and N, and wherein each of said C₁₋₆alkyl, C₁₋₆alkenyl or C₁₋₆alkynyl can be unsubstituted or substituted with one or more Z¹,

T is L²R³ or R³,

L² is selected from C₁₋₆alkyl, C₁₋₆alkenyl or C₁₋₆alkynyl, wherein each of said C₁₋₆alkyl, C₁₋₆alkenyl or C₁₋₆alkynyl optionally includes one or more heteroatoms, said heteroatoms being selected from the heteroatoms consisting of O, S, P, and N, and wherein each of said C₁₋₆alkyl, C₁₋₆alkenyl or C₁₋₆alkynyl can be unsubstituted or substituted with one or more Z¹,

each R⁵ is independently selected from alkyl, alkenyl, alkynyl, aryl, heterocycle, arylalkyl, arylalkenyl, arylalkynyl, heterocycle-alkyl, heterocycle-alkenyl or heterocycle-alkynyl, and wherein said alkyl, alkenyl, alkynyl, aryl, heterocycle, arylalkyl, arylalkenyl, arylalkynyl, heterocycle-alkyl, heterocycle-alkenyl or heterocycle-alkynyl optionally include one or more heteroatoms, said heteroatom selected from O, S, P and N, and wherein said alkyl, alkenyl, alkynyl, aryl, heterocycle, arylalkyl, arylalkenyl, arylalkynyl, heterocycle-alkyl, heterocycle-alkenyl or heterocycle-alkynyl can be unsubstituted or substituted with one or more Z¹,

each R⁶ is independently selected from hydroxyl, alkyl, alkenyl, alkynyl, aryl, heterocycle, arylalkyl, arylalkenyl, arylalkynyl, heterocycle-alkyl, heterocycle-alkenyl or heterocycle-alkynyl, and wherein said alkyl, alkenyl, alkynyl, aryl, heterocycle, arylalkyl, arylalkenyl, arylalkynyl, heterocycle-alkyl, heterocycle-alkenyl or heterocycle-alkynyl optionally includes one or more heteroatoms, said heteroatom selected from O, S, P and N, and wherein said alkyl, alkenyl, alkynyl, aryl, heterocycle, arylalkyl, arylalkenyl, arylalkynyl, heterocycle-alkyl, heterocycle-alkenyl or heterocycle-alkynyl can be unsubstituted or substituted with one or more Z¹,

each R⁷ and R⁸ is independently selected from hydrogen, alkyl, alkenyl, alkynyl, aryl, heterocycle, arylalkyl, arylalkenyl, arylalkynyl, heterocycle-alkyl, heterocycle-alkenyl or heterocycle-alkynyl, alkyl, alkenyl, alkynyl, aryl, heterocycle, arylalkyl, arylalkenyl, arylalkynyl, heterocycle-alkyl, heterocycle-alkenyl or heterocycle-alkynyl optionally includes one or more heteroatoms, said heteroatom selected from O, S, P and N, and wherein said alkyl, alkenyl, alkynyl, aryl, heterocycle, arylalkyl, arylalkenyl, arylalkynyl, heterocycle-alkyl, heterocycle-alkenyl or heterocycle-alkynyl can be unsubstituted or substituted with hydroxyl, halogen, —SH, trifluoromethyl, —OCH₃, —OCF₃, cyano, nitro, —COOH or NH₂, and wherein R⁷ and R⁸ can be taken together in order to form a (5-, 6-, or 7-membered) heterocycle which can be unsubstituted or substituted with one or more Z¹,

Z¹ is independently selected from the group consisting of hydrogen, halogen, hydroxyl, sulfhydryl, —OZ², —SZ², ═O, ═S, —S(O)Z³, —S(O)₂Z³, —SO₂NZ⁴Z⁵, trifluoromethyl, trifluoromethoxy, nitro, —NZ⁴Z⁵, —NZ⁴S(O)₂Z², —NZ⁴C(O)Z², —NZ⁴C(O)NZ⁴Z⁵, ⁻cyano, —COOZ², —C(O)NZ⁴Z⁵, —C(O)Z³, —C(O)H, alkyl, alkenyl, alkynyl, aryl, heterocycle, arylalkyl, arylalkenyl, arylalkynyl, heterocycle-alkyl, heterocycle-alkenyl, heterocycle-alkynyl,
and wherein said alkyl, alkenyl, alkynyl, aryl, heterocycle, arylalkyl, arylalkenyl, arylalkynyl, heterocycle-alkyl, heterocycle-alkenyl or heterocycle-alkynyl, optionally includes one or more heteroatoms, said heteroatoms being selected from the atoms O, S, P and N,
and wherein said alkyl, alkenyl, alkynyl, aryl, heterocycle, arylalkyl, arylalkenyl, arylalkynyl, heterocycle-alkyl, heterocycle-alkenyl or heterocycle-alkynyl can be unsubstituted or substituted with one or more hydroxyl, ═O, ═S, halogen, —SH, trifluoromethyl, —OCH₃, —O-alkyl, —OCF₃, cyano, nitro, —COOH or NH₂,

each of Z² and Z³ is independently selected from hydrogen, alkyl, alkenyl, alkynyl, aryl, heterocycle, arylalkyl, arylalkenyl, arylalkynyl, heterocycle-alkyl, heterocycle-alkenyl or heterocycle-alkynyl, and wherein said alkyl, alkenyl, alkynyl, aryl, heterocycle, arylalkyl, arylalkenyl, arylalkynyl, heterocycle-alkyl, heterocycle-alkenyl or heterocycle-alkynyl optionally include one or more heteroatoms, said heteroatom selected from O, S, P and N, and wherein said alkyl, alkenyl, alkynyl, aryl, heterocycle, arylalkyl, arylalkenyl, arylalkynyl, heterocycle-alkyl, heterocycle-alkenyl or heterocycle-alkynyl can be unsubstituted or substituted with one or more hydroxyl, halogen, —SH, ═O, ═S, —O-alkyl, trifluoromethyl, —OCH₃, —OCF₃, cyano, nitro, —COOH or NH₂,

each Z⁴ and Z⁵ is independently selected from hydrogen, alkyl, alkenyl, alkynyl, aryl, heterocycle, arylalkyl, arylalkenyl, arylalkynyl, heterocycle-alkyl, heterocycle-alkenyl or heterocycle-alkynyl, alkyl, alkenyl, alkynyl, aryl, heterocycle, arylalkyl, arylalkenyl, arylalkynyl, heterocycle-alkyl, heterocycle-alkenyl or heterocycle-alkynyl optionally includes one or more heteroatoms, said heteroatom selected from O, S, P and N, and wherein said alkyl, alkenyl, alkynyl, aryl, heterocycle, arylalkyl, arylalkenyl, arylalkynyl, heterocycle-alkyl, heterocycle-alkenyl or heterocycle-alkynyl can be unsubstituted or substituted with one or more hydroxyl, halogen, —SH, ═O, ═S, —O-alkyl, trifluoromethyl, —OCH₃, —OCF₃, cyano, nitro, —COOH or NH₂, and wherein Z⁴ and Z⁵ can be taken together in order to form a (5-, 6-, or 7-membered) heterocycle which can be unsubstituted or substituted with hydroxyl, halogen, —SH, ═O, ═S, —O-alkyl, trifluoromethyl, —OCH₃, —OCF₃, cyano, nitro, —COOH or —NH₂.

In yet another particular embodiment, the compounds of the invention are according to formula (I), (Ia), (Ib), (Ic), or (Id), wherein,

each of R¹, R² and R³ is independently selected from alkyl, alkenyl, alkynyl, aryl, heterocycle, arylalkyl, arylalkenyl, arylalkynyl, heterocycle-alkyl, heterocycle-alkenyl or heterocycle-alkynyl, and wherein said alkyl, alkenyl, alkynyl, aryl, heterocycle, arylalkyl, arylalkenyl, arylalkynyl, heterocycle-alkyl, heterocycle-alkenyl or heterocycle-alkynyl optionally include one or more heteroatoms, said heteroatom selected from O, S, P and N, and wherein said alkyl, alkenyl, alkynyl, aryl, heterocycle, arylalkyl, arylalkenyl, arylalkynyl, heterocycle-alkyl, heterocycle-alkenyl or heterocycle-alkynyl can be unsubstituted or substituted with one or more Z¹,

Q is L¹R² or R²,

L¹ is selected from C₁₋₆alkyl, C₁₋₆alkenyl or C₁₋₆alkynyl, wherein each of said C₁₋₆alkyl, C₁₋₆alkenyl or C₁₋₆alkynyl optionally includes one or more heteroatoms, said heteroatoms being selected from the heteroatoms consisting of O, S, P, and N, and wherein each of said C₁₋₆alkyl, C₁₋₆alkenyl or C₁₋₆alkynyl can be unsubstituted or substituted with one or more Z¹,

T is L²R³ or R³,

L² is selected from C₁₋₆alkyl, C₁₋₆alkenyl or C₁₋₆alkynyl, wherein each of said C₁₋₆alkyl, C₁₋₆alkenyl or C₁₋₆alkynyl optionally includes one or more heteroatoms, said heteroatoms being selected from the heteroatoms consisting of O, S, P, and N, and wherein each of said C₁₋₆alkyl, C₁₋₆alkenyl or C₁₋₆alkynyl can be unsubstituted or substituted with one or more Z¹,

each R⁵ is independently selected from alkyl, alkenyl, alkynyl, aryl, heterocycle, arylalkyl, arylalkenyl, arylalkynyl, heterocycle-alkyl, heterocycle-alkenyl or heterocycle-alkynyl, and wherein said alkyl, alkenyl, alkynyl, aryl, heterocycle, arylalkyl, arylalkenyl, arylalkynyl, heterocycle-alkyl, heterocycle-alkenyl or heterocycle-alkynyl optionally include one or more heteroatoms, said heteroatom selected from O, S, P and N, and wherein said alkyl, alkenyl, alkynyl, aryl, heterocycle, arylalkyl, arylalkenyl, arylalkynyl, heterocycle-alkyl, heterocycle-alkenyl or heterocycle-alkynyl can be unsubstituted or substituted with one or more Z¹,

each R⁶ is independently selected from hydroxyl, alkyl, alkenyl, alkynyl, aryl, heterocycle, arylalkyl, arylalkenyl, arylalkynyl, heterocycle-alkyl, heterocycle-alkenyl or heterocycle-alkynyl, and wherein said alkyl, alkenyl, alkynyl, aryl, heterocycle, arylalkyl, arylalkenyl, arylalkynyl, heterocycle-alkyl, heterocycle-alkenyl or heterocycle-alkynyl optionally includes one or more heteroatoms, said heteroatom selected from O, S, P and N, and wherein said alkyl, alkenyl, alkynyl, aryl, heterocycle, arylalkyl, arylalkenyl, arylalkynyl, heterocycle-alkyl, heterocycle-alkenyl or heterocycle-alkynyl can be unsubstituted or substituted with one or more Z¹,

each R⁷ and R⁸ is independently selected from hydrogen, alkyl, alkenyl, alkynyl, aryl, heterocycle, arylalkyl, arylalkenyl, arylalkynyl, heterocycle-alkyl, heterocycle-alkenyl or heterocycle-alkynyl, alkyl, alkenyl, alkynyl, aryl, heterocycle, arylalkyl, arylalkenyl, arylalkynyl, heterocycle-alkyl, heterocycle-alkenyl or heterocycle-alkynyl optionally includes one or more heteroatoms, said heteroatom selected from O, S, P and N, and wherein said alkyl, alkenyl, alkynyl, aryl, heterocycle, arylalkyl, arylalkenyl, arylalkynyl, heterocycle-alkyl, heterocycle-alkenyl or heterocycle-alkynyl can be unsubstituted or substituted with hydroxyl, halogen, —SH, trifluoromethyl, —OCH₃, —OCF₃, cyano, nitro, —COOH or NH₂, and wherein R⁷ and R⁸ can be taken together in order to form a (5-, 6-, or 7-membered) heterocycle which can be unsubstituted or substituted with one or more Z¹,

Z¹ is independently selected from the group consisting of hydrogen, halogen, hydroxyl, —SZ², —S(O)Z³, —S(O)₂Z³, —SO₂NZ⁴Z⁵, trifluoromethyl, nitro, —NZ⁴Z⁵, -cyano, —COOZ², —C(O)NZ⁴Z⁵, —C(O)Z³, alkyl, alkenyl, alkynyl, aryl, heterocycle, arylalkyl, arylalkenyl, arylalkynyl, heterocycle-alkyl, heterocycle-alkenyl, heterocycle-alkynyl,
and wherein said alkyl, alkenyl, alkynyl, aryl, heterocycle, arylalkyl, arylalkenyl, arylalkynyl, heterocycle-alkyl, heterocycle-alkenyl or heterocycle-alkynyl, optionally includes one or more heteroatoms, said heteroatoms being selected from the atoms O, S, P and N,
and wherein said alkyl, alkenyl, alkynyl, aryl, heterocycle, arylalkyl, arylalkenyl, arylalkynyl, heterocycle-alkyl, heterocycle-alkenyl or heterocycle-alkynyl can be unsubstituted or substituted with hydroxyl, halogen, —SH, trifluoromethyl, —OCH₃, —OCF₃, cyano, nitro, —COOH or NH₂,

each Z² and Z³ is independently selected from hydrogen, alkyl, alkenyl, alkynyl, aryl, heterocycle, arylalkyl, arylalkenyl, arylalkynyl, heterocycle-alkyl, heterocycle-alkenyl or heterocycle-alkynyl, and wherein said alkyl, alkenyl, alkynyl, aryl, heterocycle, arylalkyl, arylalkenyl, arylalkynyl, heterocycle-alkyl, heterocycle-alkenyl or heterocycle-alkynyl optionally include one or more heteroatoms, said heteroatom selected from O, S, P and N, and wherein said alkyl, alkenyl, alkynyl, aryl, heterocycle, arylalkyl, arylalkenyl, arylalkynyl, heterocycle-alkyl, heterocycle-alkenyl or heterocycle-alkynyl can be unsubstituted or substituted with hydroxyl, halogen, —SH, trifluoromethyl, —OCH₃, —OCF₃, cyano, nitro, —COOH or NH₂,

each Z⁴ and Z⁵ is independently selected from hydrogen, alkyl, alkenyl, alkynyl, aryl, heterocycle, arylalkyl, arylalkenyl, arylalkynyl, heterocycle-alkyl, heterocycle-alkenyl or heterocycle-alkynyl, alkyl, alkenyl, alkynyl, aryl, heterocycle, arylalkyl, arylalkenyl, arylalkynyl, heterocycle-alkyl, heterocycle-alkenyl or heterocycle-alkynyl optionally includes one or more heteroatoms, said heteroatom selected from O, S, P and N, and wherein said alkyl, alkenyl, alkynyl, aryl, heterocycle, arylalkyl, arylalkenyl, arylalkynyl, heterocycle-alkyl, heterocycle-alkenyl or heterocycle-alkynyl can be unsubstituted or substituted with hydroxyl, halogen, —SH, trifluoromethyl, —OCH₃, —OCF₃, cyano, nitro, —COOH or NH₂, and wherein Z⁴ and Z⁵ can be taken together in order to form a (5-, 6-, or 7-membered) heterocycle which can be unsubstituted or substituted with hydroxyl, halogen, —SH, trifluoromethyl, —OCH₃, —OCF₃, cyano, nitro, —COOH or —NH₂.

In yet another particular embodiment, n is selected from 2, 3 or 4. In another embodiment, the dotted line “a” is a single bond. In still a more particular embodiment, R¹ is selected from alkyl, alkenyl, or alkynyl, wherein said alkyl, alkenyl or alkynyl optionally include one or more heteroatoms, said heteroatom selected from O, S, P and N, and wherein said alkyl, alkenyl or alkynyl can be unsubstituted or substituted. In a more preferred embodiment, R¹ is selected from a linear, straight or branched, C₁₋₆-alkyl, C₂₋₆-alkenyl, or C₂₋₆-alkynyl, wherein said C₁₋₆-alkyl, C₂₋₆-alkenyl or C₂₋₆-alkynyl optionally include one or more heteroatoms, said heteroatom selected from O, S, P and N, and wherein said C₁₋₆-alkyl, C₂₋₆-alkenyl or C₂₋₆-alkynyl can be unsubstituted or substituted. In yet a more particular embodiment, R¹ is selected from —C₁₋₆ alkyl-O—C₁₋₆ alkyl or —C₁₋₆ alkyl-S—C₁₋₆ alkyl, which can be unsubstituted or substituted with one or more Z¹. Still in a more particular embodiment, R¹ is selected from —C₁₋₆ alkyl-O-Me or —C₁₋₆ alkyl-S-Me. In a further preferred embodiment, R¹ is selected from an acyclic alkyl, acyclic alkenyl, or acyclic alkynyl, wherein said acyclic alkyl, acyclic alkenyl or acyclic alkynyl optionally include one or more heteroatoms, said heteroatom selected from O, S, P and N, and wherein said acyclic alkyl, acyclic alkenyl or acyclic alkynyl can be unsubstituted or substituted.

In another more particular embodiment, Q is R² and R² is selected from aryl, heterocycle, arylalkyl, arylalkenyl, arylalkynyl, heterocycle-alkyl, heterocycle-alkenyl or heterocycle-alkynyl, wherein said aryl, heterocycle, arylalkyl, arylalkenyl, arylalkynyl, heterocycle-alkyl, heterocycle-alkenyl or heterocycle-alkynyl optionally include one or more heteroatoms, said heteroatom selected from O, S, P and N, and wherein said aryl, heterocycle, arylalkyl, arylalkenyl, arylalkynyl, heterocycle-alkyl, heterocycle-alkenyl or heterocycle-alkynyl can be unsubstituted or substituted. In yet another embodiment, R² is selected from unsubstituted or substituted thienyl or furanyl.

In another particular embodiment, L² is selected from —CH₂NZ⁶—, CH₂NH—, —CH₂O—, —CO—NZ⁶—, —CONH—, or —CH₂O—, and each Z⁶ is independently selected from alkyl, alkenyl, alkynyl, aryl, heterocycle, arylalkyl, arylalkenyl, arylalkynyl, heterocycle-alkyl, heterocycle-alkenyl or heterocycle-alkynyl, alkyl, alkenyl, alkynyl, aryl, heterocycle, arylalkyl, arylalkenyl, arylalkynyl, heterocycle-alkyl, heterocycle-alkenyl or heterocycle-alkynyl optionally includes one or more heteroatoms, said heteroatom selected from O, S, P and N, and wherein said alkyl, alkenyl, alkynyl, aryl, heterocycle, arylalkyl, arylalkenyl, arylalkynyl, heterocycle-alkyl, heterocycle-alkenyl or heterocycle-alkynyl can be unsubstituted or substituted with hydroxyl, halogen, —SH, ═O, ═S, trifluoromethyl, —OCH₃, —OCF₃, cyano, nitro, —COOH or NH₂, and wherein Z⁶ can be taken together with R³ in order to form a (5-, 6-, or 7-membered) heterocycle which can be unsubstituted or substituted with one or more Z¹. In a particular embodiment, L² is —CONH—, wherein the —CO of —CONH— is coupled to the isoquinoline and the NH— of —CONH— is coupled to R³.

In a more particular embodiment, R³ is selected from aryl, heterocycle, arylalkyl, arylalkenyl, arylalkynyl, heterocycle-alkyl, heterocycle-alkenyl or heterocycle-alkynyl, and wherein said alkyl, alkenyl, alkynyl, aryl, heterocycle, arylalkyl, arylalkenyl, arylalkynyl, heterocycle-alkyl, heterocycle-alkenyl or heterocycle-alkynyl optionally include one or more heteroatoms, said heteroatom selected from O, S, P and N, and wherein said alkyl, alkenyl, alkynyl, aryl, heterocycle, arylalkyl, arylalkenyl, arylalkynyl, heterocycle-alkyl, heterocycle-alkenyl or heterocycle-alkynyl can be unsubstituted or substituted. In yet another particular embodiment, R³ has a structure according to the formula (AB).

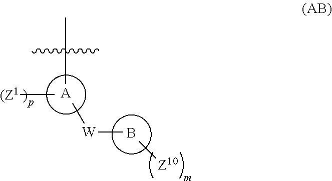

wherein,

each of cycle A and cycle B are selected from aryl or heterocycle,

each of p and m are independently selected from 0, 1, 2, 3, and 4,

—W— is selected from a single bond, —O—, —S—, -alkylene-, -alkenylene-, and -alkynylene-, wherein each of said alkyl, alkenyl or alkynyl optionally include one or more heteroatoms, said heteroatom selected from O, S, P and N, and wherein said alkyl, alkenyl or alkynyl can be unsubstituted or substituted with one or more hydroxyl, halogen, —SH, ═O, ═S, —O-alkyl, trifluoromethyl, —OCH₃, —OCF₃, cyano, nitro, —COOH or —NH₂.

Z¹ is independently selected from the group consisting of hydrogen, halogen, hydroxyl, sulfhydryl, —OZ², —SZ², ═O, ═S, —S(O)Z³, —S(O)₂Z³, —SO₂NZ⁴Z⁵, trifluoromethyl, trifluoromethoxy, nitro, —NZ⁴Z⁵, —NZ⁴S(O)₂Z², —NZ⁴C(O)Z², —NZ⁴C(O)NZ⁴Z⁵, ⁻cyano, —COOZ², —C(O)NZ⁴Z⁵, —C(O)Z³, —C(O)H, alkyl, alkenyl, alkynyl, aryl, heterocycle, arylalkyl, arylalkenyl, arylalkynyl, heterocycle-alkyl, heterocycle-alkenyl, heterocycle-alkynyl,
and wherein said alkyl, alkenyl, alkynyl, aryl, heterocycle, arylalkyl, arylalkenyl, arylalkynyl, heterocycle-alkyl, heterocycle-alkenyl or heterocycle-alkynyl, optionally includes one or more heteroatoms, said heteroatoms being selected from the atoms O, S, P and N,
and wherein said alkyl, alkenyl, alkynyl, aryl, heterocycle, arylalkyl, arylalkenyl, arylalkynyl, heterocycle-alkyl, heterocycle-alkenyl or heterocycle-alkynyl can be unsubstituted or substituted with one or more Z¹⁰,

each of Z² and Z³ is independently selected from hydrogen, alkyl, alkenyl, alkynyl, aryl, heterocycle, arylalkyl, arylalkenyl, arylalkynyl, heterocycle-alkyl, heterocycle-alkenyl or heterocycle-alkynyl, and wherein said alkyl, alkenyl, alkynyl, aryl, heterocycle, arylalkyl, arylalkenyl, arylalkynyl, heterocycle-alkyl, heterocycle-alkenyl or heterocycle-alkynyl optionally include one or more heteroatoms, said heteroatom selected from O, S, P and N, and wherein said alkyl, alkenyl, alkynyl, aryl, heterocycle, arylalkyl, arylalkenyl, arylalkynyl, heterocycle-alkyl, heterocycle-alkenyl or heterocycle-alkynyl can be unsubstituted or substituted with one or more Z¹⁰,

each Z⁴ and Z⁵ is independently selected from hydrogen, alkyl, alkenyl, alkynyl, aryl, heterocycle, arylalkyl, arylalkenyl, arylalkynyl, heterocycle-alkyl, heterocycle-alkenyl or heterocycle-alkynyl, alkyl, alkenyl, alkynyl, aryl, heterocycle, arylalkyl, arylalkenyl, arylalkynyl, heterocycle-alkyl, heterocycle-alkenyl or heterocycle-alkynyl optionally includes one or more heteroatoms, said heteroatom selected from O, S, P and N, and wherein said alkyl, alkenyl, alkynyl, aryl, heterocycle, arylalkyl, arylalkenyl, arylalkynyl, heterocycle-alkyl, heterocycle-alkenyl or heterocycle-alkynyl can be unsubstituted or substituted with one or more Z¹⁰, and wherein Z⁴ and Z⁵ can be taken together in order to form a (5-, 6-, or 7-membered) heterocycle which can be unsubstituted or substituted with one or more Z¹⁰,

Z¹⁰ is independently selected from the group consisting of hydrogen, halogen, hydroxyl, sulfhydryl, —OZ¹², —SZ¹², ═O, ═S, —S(O)Z¹³, —S(O)₂Z¹³, —SO₂NZ¹⁴Z¹⁵, trifluoromethyl, trifluoromethoxy, nitro, —NZ¹⁴Z¹⁵, —NZ¹⁴S(O)₂Z¹², —NZ¹⁴C(O)Z¹², —NZ¹⁴C(O)NZ¹⁴Z¹⁵, ⁻cyano, —COOZ¹², —C(O)NZ¹⁴Z¹⁵, —C(O)Z¹³, —C(O)H, alkyl, alkenyl, alkynyl, aryl, heterocycle, arylalkyl, arylalkenyl, arylalkynyl, heterocycle-alkyl, heterocycle-alkenyl, heterocycle-alkynyl,
and wherein said alkyl, alkenyl, alkynyl, aryl, heterocycle, arylalkyl, arylalkenyl, arylalkynyl, heterocycle-alkyl, heterocycle-alkenyl or heterocycle-alkynyl, optionally includes one or more heteroatoms, said heteroatoms being selected from the atoms O, S, P and N,
and wherein said alkyl, alkenyl, alkynyl, aryl, heterocycle, arylalkyl, arylalkenyl, arylalkynyl, heterocycle-alkyl, heterocycle-alkenyl or heterocycle-alkynyl can be unsubstituted or substituted with one or more hydroxyl, halogen, —SH, trifluoromethyl, —OCH₃, —OCF₃, cyano, nitro, —COOH or NH₂,

each Z¹² is independently selected from hydrogen, alkyl, alkenyl, alkynyl, aryl, heterocycle, arylalkyl, arylalkenyl, arylalkynyl, heterocycle-alkyl, heterocycle-alkenyl or heterocycle-alkynyl, and wherein said alkyl, alkenyl, alkynyl, aryl, heterocycle, arylalkyl, arylalkenyl, arylalkynyl, heterocycle-alkyl, heterocycle-alkenyl or heterocycle-alkynyl optionally include one or more heteroatoms, said heteroatom selected from O, S, P and N, and wherein said alkyl, alkenyl, alkynyl, aryl, heterocycle, arylalkyl, arylalkenyl, arylalkynyl, heterocycle-alkyl, heterocycle-alkenyl or heterocycle-alkynyl can be unsubstituted or substituted with one or more hydroxyl, halogen, —SH, trifluoromethyl, —OCH₃, —OCF₃, cyano, nitro, —COOH or NH₂,

each Z¹³ is independently selected from hydroxyl, alkyl, alkenyl, alkynyl, aryl, heterocycle, arylalkyl, arylalkenyl, arylalkynyl, heterocycle-alkyl, heterocycle-alkenyl or heterocycle-alkynyl, and wherein said alkyl, alkenyl, alkynyl, aryl, heterocycle, arylalkyl, arylalkenyl, arylalkynyl, heterocycle-alkyl, heterocycle-alkenyl or heterocycle-alkynyl optionally includes one or more heteroatoms, said heteroatom selected from O, S, P and N, and wherein said alkyl, alkenyl, alkynyl, aryl, heterocycle, arylalkyl, arylalkenyl, arylalkynyl, heterocycle-alkyl, heterocycle-alkenyl or heterocycle-alkynyl can be unsubstituted or substituted with one or more hydroxyl, halogen, —SH, trifluoromethyl, —OCH₃, —OCF₃, cyano, nitro, —COOH or NH₂,

each Z¹⁴ and Z¹⁵ is independently selected from hydrogen, alkyl, alkenyl, alkynyl, aryl, heterocycle, arylalkyl, arylalkenyl, arylalkynyl, heterocycle-alkyl, heterocycle-alkenyl or heterocycle-alkynyl, alkyl, alkenyl, alkynyl, aryl, heterocycle, arylalkyl, arylalkenyl, arylalkynyl, heterocycle-alkyl, heterocycle-alkenyl or heterocycle-alkynyl optionally includes one or more heteroatoms, said heteroatom selected from O, S, P and N, and wherein said alkyl, alkenyl, alkynyl, aryl, heterocycle, arylalkyl, arylalkenyl, arylalkynyl, heterocycle-alkyl, heterocycle-alkenyl or heterocycle-alkynyl can be unsubstituted or substituted with one or more hydroxyl, halogen, —SH, trifluoromethyl, —OCH₃, —OCF₃, cyano, nitro, —COOH or NH₂, and wherein Z¹⁴ and Z¹⁵ can be taken together in order to form a (5-, 6-, or 7-membered) heterocycle which can be unsubstituted or substituted with one or more hydroxyl, halogen, —SH, trifluoromethyl, —OCH₃, —OCF₃, cyano, nitro, —COOH or NH₂.

In a preferred embodiment, the compounds of the invention are according to the formulas (I), (Ia), (Ib), (Ic), or (Id), wherein

R¹ is independently selected from alkyl, alkenyl, or alkynyl, wherein said alkyl, alkenyl, or alkynyl, optionally include one or more heteroatoms, said heteroatom selected from O, S, P and N, and wherein said alkyl, alkenyl, or alkynyl, can be unsubstituted or substituted with one or more Z¹,

each of R² and R³ is independently selected from aryl, heterocycle, arylalkyl, arylalkenyl, arylalkynyl, heterocycle-alkyl, heterocycle-alkenyl or heterocycle-alkynyl, wherein said aryl, heterocycle, arylalkyl, arylalkenyl, arylalkynyl, heterocycle-alkyl, heterocycle-alkenyl or heterocycle-alkynyl optionally include one or more heteroatoms, said heteroatom selected from O, S, P and N, and wherein said aryl, heterocycle, arylalkyl, arylalkenyl, arylalkynyl, heterocycle-alkyl, heterocycle-alkenyl or heterocycle-alkynyl can be unsubstituted or substituted with one or more Z¹,

Q is R²,

L² is selected from —CH₂NZ⁶—, CH₂NH—, —CH₂O—, —CO—NZ⁶—, or —CONH—,

each R⁴ is independently selected from hydrogen, cyano, halogen, —OH, —OW, —SH, —SR⁵, —S(O)R⁶, —S(O)₂R⁶, —SO₂NR⁷R⁸, trifluoromethyl, nitro, —NR⁵C(O)R⁵, —NR⁵S(O)₂R⁵, —NR⁵C(O)NR⁷R⁸, —NR⁷R⁸, -cyano, —COOH, —COOR⁵, —C(O)NR⁷R⁸, —C(O)R⁶, alkyl, alkenyl, alkynyl, aryl, heterocycle, arylalkyl, arylalkenyl, arylalkynyl, heterocycle-alkyl, heterocycle-alkenyl, heterocycle-alkynyl,
and wherein said alkyl, alkenyl, alkynyl, aryl, heterocycle, arylalkyl, arylalkenyl, arylalkynyl, heterocycle-alkyl, heterocycle-alkenyl or heterocycle-alkynyl, optionally includes one or more heteroatoms, said heteroatoms being selected from the atoms O, S, P and N,
and wherein said alkyl, alkenyl, alkynyl, aryl, heterocycle, arylalkyl, arylalkenyl, arylalkynyl, heterocycle-alkyl, heterocycle-alkenyl or heterocycle-alkynyl can be unsubstituted or substituted with halogen, —OH, —OR⁵, —SH, —SR⁵, —S(O)R⁶, —S(O)₂R⁶, —SO₂NR⁷R⁸, trifluoromethyl, nitro, —NR⁵C(O)R⁵, —NR⁵S(O)₂R⁵, —NR⁵C(O)NR⁷R⁸, —NR⁷R⁸, -cyano, —COOH, —COOR⁵, —C(O)NR⁷R⁸, —C(O)R⁶,

n is selected from 0, 1, 2, 3 or 4,

Z¹ is independently selected from the group consisting of hydrogen, halogen, hydroxyl, —SZ², —S(O)Z³, —S(O)₂Z³, —SO₂NZ⁴Z⁵, trifluoromethyl, nitro, —NZ⁴Z⁵, ⁻cyano, —COOZ², —C(O)NZ⁴Z⁵, —C(O)Z³, alkyl, alkenyl, alkynyl, aryl, heterocycle, arylalkyl, arylalkenyl, arylalkynyl, heterocycle-alkyl, heterocycle-alkenyl, heterocycle-alkynyl,
and wherein said alkyl, alkenyl, alkynyl, aryl, heterocycle, arylalkyl, arylalkenyl, arylalkynyl, heterocycle-alkyl, heterocycle-alkenyl or heterocycle-alkynyl, optionally includes one or more heteroatoms, said heteroatoms being selected from the atoms O, S, P and N,
and wherein said alkyl, alkenyl, alkynyl, aryl, heterocycle, arylalkyl, arylalkenyl, arylalkynyl, heterocycle-alkyl, heterocycle-alkenyl or heterocycle-alkynyl can be unsubstituted or substituted with hydroxyl, halogen, —SH, trifluoromethyl, —OCH₃, —OCF₃, cyano, nitro, —COOH or NH₂,

each of Z² and Z³ is independently selected from hydrogen, alkyl, alkenyl, alkynyl, aryl, heterocycle, arylalkyl, arylalkenyl, arylalkynyl, heterocycle-alkyl, heterocycle-alkenyl or heterocycle-alkynyl, and wherein said alkyl, alkenyl, alkynyl, aryl, heterocycle, arylalkyl, arylalkenyl, arylalkynyl, heterocycle-alkyl, heterocycle-alkenyl or heterocycle-alkynyl optionally include one or more heteroatoms, said heteroatom selected from O, S, P and N, and wherein said alkyl, alkenyl, alkynyl, aryl, heterocycle, arylalkyl, arylalkenyl, arylalkynyl, heterocycle-alkyl, heterocycle-alkenyl or heterocycle-alkynyl can be unsubstituted or substituted with hydroxyl, halogen, —SH, trifluoromethyl, —OCH₃, —OCF₃, cyano, nitro, —COOH or NH₂,

each Z⁴ and Z⁵ is independently selected from hydrogen, alkyl, alkenyl, alkynyl, aryl, heterocycle, arylalkyl, arylalkenyl, arylalkynyl, heterocycle-alkyl, heterocycle-alkenyl or heterocycle-alkynyl, alkyl, alkenyl, alkynyl, aryl, heterocycle, arylalkyl, arylalkenyl, arylalkynyl, heterocycle-alkyl, heterocycle-alkenyl or heterocycle-alkynyl optionally includes one or more heteroatoms, said heteroatom selected from O, S, P and N, and wherein said alkyl, alkenyl, alkynyl, aryl, heterocycle, arylalkyl, arylalkenyl, arylalkynyl, heterocycle-alkyl, heterocycle-alkenyl or heterocycle-alkynyl can be unsubstituted or substituted with hydroxyl, halogen, —SH, trifluoromethyl, —OCH₃, —OCF₃, cyano, nitro, —COOH or NH₂, and wherein Z⁴ and Z⁵ can be taken together in order to form a (5-, 6-, or 7-membered) heterocycle which can be unsubstituted or substituted with hydroxyl, halogen, —SH, trifluoromethyl, —OCH₃, —OCF₃, cyano, nitro, —COOH or —NH₂.

each Z⁶ is independently selected from alkyl, alkenyl, alkynyl, aryl, heterocycle, arylalkyl, arylalkenyl, arylalkynyl, heterocycle-alkyl, heterocycle-alkenyl or heterocycle-alkynyl, alkyl, alkenyl, alkynyl, aryl, heterocycle, arylalkyl, arylalkenyl, arylalkynyl, heterocycle-alkyl, heterocycle-alkenyl or heterocycle-alkynyl optionally includes one or more heteroatoms, said heteroatom selected from O, S, P and N, and wherein said alkyl, alkenyl, alkynyl, aryl, heterocycle, arylalkyl, arylalkenyl, arylalkynyl, heterocycle-alkyl, heterocycle-alkenyl or heterocycle-alkynyl can be unsubstituted or substituted with hydroxyl, halogen, —SH, trifluoromethyl, —OCH₃, —OCF₃, cyano, nitro, —COOH or NH₂, and wherein Z⁶ can be taken together with R³ in order to form a (5-, 6-, or 7-membered) heterocycle which can be unsubstituted or substituted with one or more Z¹.

In another particular embodiment, the compounds of the invention have a structure according to formula (II)

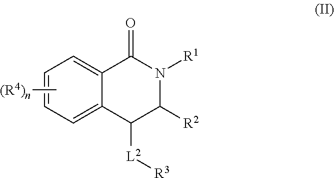

wherein,

R¹ is independently selected from alkyl, alkenyl, or alkynyl, wherein said alkyl, alkenyl, or alkynyl, optionally include one or more heteroatoms, said heteroatom selected from O, S, P and N, and wherein said alkyl, alkenyl, or alkynyl, can be unsubstituted or substituted with one or more Z¹,

each of R² and R³ is independently selected from aryl, heterocycle, arylalkyl, arylalkenyl, arylalkynyl, heterocycle-alkyl, heterocycle-alkenyl or heterocycle-alkynyl, wherein said aryl, heterocycle, arylalkyl, arylalkenyl, arylalkynyl, heterocycle-alkyl, heterocycle-alkenyl or heterocycle-alkynyl optionally include one or more heteroatoms, said heteroatom selected from O, S, P and N, and wherein said aryl, heterocycle, arylalkyl, arylalkenyl, arylalkynyl, heterocycle-alkyl, heterocycle-alkenyl or heterocycle-alkynyl can be unsubstituted or substituted with one or more Z¹,

L² is selected from —CH₂NZ⁶—, CH₂NH—, —CO—NZ⁶—, —CH₂O— or —CONH—,

each R⁴ is independently selected from hydrogen, halogen, —OH, —OR⁵, —SH, —SR⁵, —S(O)R⁶, —S(O)₂R⁶, —SO₂NR⁷R⁸, trifluoromethyl, nitro, —NR⁵C(O)R⁵, —NR⁵S(O)₂R⁵, —NR⁵C(O)NR⁷R⁸, —NR⁷R⁸, -cyano, —COOH, —COOR⁵, —C(O)NR⁷R⁸, —C(O)R⁶, alkyl, alkenyl, alkynyl, aryl, heterocycle, arylalkyl, arylalkenyl, arylalkynyl, heterocycle-alkyl, heterocycle-alkenyl, heterocycle-alkynyl,
and wherein said alkyl, alkenyl, alkynyl, aryl, heterocycle, arylalkyl, arylalkenyl, arylalkynyl, heterocycle-alkyl, heterocycle-alkenyl or heterocycle-alkynyl, optionally includes one or more heteroatoms, said heteroatoms being selected from the atoms O, S, P and N,
and wherein said alkyl, alkenyl, alkynyl, aryl, heterocycle, arylalkyl, arylalkenyl, arylalkynyl, heterocycle-alkyl, heterocycle-alkenyl or heterocycle-alkynyl can be unsubstituted or substituted with halogen, —OH, —OR⁵, —SH, —SR⁵, —S(O)R⁶, —S(O)₂R⁶, —SO₂NR⁷R⁸, trifluoromethyl, nitro, —NR⁵C(O)R⁵, —NR⁵S(O)₂R⁵, —NR⁵C(O)NR⁷R⁸, —NR⁷R⁸, -cyano, —COOH, —COOR⁵, —C(O)NR⁷R⁸, —C(O)R⁶,

n is selected from 0, 1, 2, 3 or 4,

each Z⁶ is independently selected from alkyl, alkenyl, alkynyl, aryl, heterocycle, arylalkyl, arylalkenyl, arylalkynyl, heterocycle-alkyl, heterocycle-alkenyl or heterocycle-alkynyl, alkyl, alkenyl, alkynyl, aryl, heterocycle, arylalkyl, arylalkenyl, arylalkynyl, heterocycle-alkyl, heterocycle-alkenyl or heterocycle-alkynyl optionally includes one or more heteroatoms, said heteroatom selected from O, S, P and N, and wherein said alkyl, alkenyl, alkynyl, aryl, heterocycle, arylalkyl, arylalkenyl, arylalkynyl, heterocycle-alkyl, heterocycle-alkenyl or heterocycle-alkynyl can be unsubstituted or substituted with hydroxyl, halogen, —SH, trifluoromethyl, —OCH₃, —OCF₃, cyano, nitro, —COOH or NH₂, and wherein Z⁶ can be taken together with R³ in order to form a (5-, 6-, or 7-membered) heterocycle which can be unsubstituted or substituted with one or more Z¹,

each R⁵ is independently selected from alkyl, alkenyl, alkynyl, aryl, heterocycle, arylalkyl, arylalkenyl, arylalkynyl, heterocycle-alkyl, heterocycle-alkenyl or heterocycle-alkynyl, and wherein said alkyl, alkenyl, alkynyl, aryl, heterocycle, arylalkyl, arylalkenyl, arylalkynyl, heterocycle-alkyl, heterocycle-alkenyl or heterocycle-alkynyl optionally include one or more heteroatoms, said heteroatom selected from O, S, P and N, and wherein said alkyl, alkenyl, alkynyl, aryl, heterocycle, arylalkyl, arylalkenyl, arylalkynyl, heterocycle-alkyl, heterocycle-alkenyl or heterocycle-alkynyl can be unsubstituted or substituted,

each R⁶ is independently selected from hydroxyl, alkyl, alkenyl, alkynyl, aryl, heterocycle, arylalkyl, arylalkenyl, arylalkynyl, heterocycle-alkyl, heterocycle-alkenyl or heterocycle-alkynyl, and wherein said alkyl, alkenyl, alkynyl, aryl, heterocycle, arylalkyl, arylalkenyl, arylalkynyl, heterocycle-alkyl, heterocycle-alkenyl or heterocycle-alkynyl optionally includes one or more heteroatoms, said heteroatom selected from O, S, P and N, and wherein said alkyl, alkenyl, alkynyl, aryl, heterocycle, arylalkyl, arylalkenyl, arylalkynyl, heterocycle-alkyl, heterocycle-alkenyl or heterocycle-alkynyl can be unsubstituted or substituted,

each R⁷ and R⁸ is independently selected from hydrogen, alkyl, alkenyl, alkynyl, aryl, heterocycle, arylalkyl, arylalkenyl, arylalkynyl, heterocycle-alkyl, heterocycle-alkenyl or heterocycle-alkynyl, alkyl, alkenyl, alkynyl, aryl, heterocycle, arylalkyl, arylalkenyl, arylalkynyl, heterocycle-alkyl, heterocycle-alkenyl or heterocycle-alkynyl optionally includes one or more heteroatoms, said heteroatom selected from O, S, P and N, and wherein said alkyl, alkenyl, alkynyl, aryl, heterocycle, arylalkyl, arylalkenyl, arylalkynyl, heterocycle-alkyl, heterocycle-alkenyl or heterocycle-alkynyl can be unsubstituted or substituted with hydroxyl, halogen, —SH, trifluoromethyl, —OCH₃, —OCF₃, cyano, nitro, —COOH or NH₂, and wherein R⁷ and R⁸ can be taken together in order to form a (5-, 6-, or 7-membered) heterocycle which can be unsubstituted or substituted,

Z¹ is independently selected from the group consisting of hydrogen, halogen, hydroxyl, —SZ², —S(O)Z³, —S(O)₂Z³, —SO₂NZ⁴Z⁵, trifluoromethyl, nitro, —NZ⁴Z⁵, ⁻cyano, —COOZ², —C(O)NZ⁴Z⁵, —C(O)Z³, alkyl, alkenyl, alkynyl, aryl, heterocycle, arylalkyl, arylalkenyl, arylalkynyl, heterocycle-alkyl, heterocycle-alkenyl, heterocycle-alkynyl,
and wherein said alkyl, alkenyl, alkynyl, aryl, heterocycle, arylalkyl, arylalkenyl, arylalkynyl, heterocycle-alkyl, heterocycle-alkenyl or heterocycle-alkynyl, optionally includes one or more heteroatoms, said heteroatoms being selected from the atoms O, S, P and N,
and wherein said alkyl, alkenyl, alkynyl, aryl, heterocycle, arylalkyl, arylalkenyl, arylalkynyl, heterocycle-alkyl, heterocycle-alkenyl or heterocycle-alkynyl can be unsubstituted or substituted with hydroxyl, halogen, —SH, trifluoromethyl, —OCH₃, —OCF₃, cyano, nitro, —COOH or NH₂,

each of Z² and Z³ is independently selected from hydrogen, alkyl, alkenyl, alkynyl, aryl, heterocycle, arylalkyl, arylalkenyl, arylalkynyl, heterocycle-alkyl, heterocycle-alkenyl or heterocycle-alkynyl, and wherein said alkyl, alkenyl, alkynyl, aryl, heterocycle, arylalkyl, arylalkenyl, arylalkynyl, heterocycle-alkyl, heterocycle-alkenyl or heterocycle-alkynyl optionally include one or more heteroatoms, said heteroatom selected from O, S, P and N, and wherein said alkyl, alkenyl, alkynyl, aryl, heterocycle, arylalkyl, arylalkenyl, arylalkynyl, heterocycle-alkyl, heterocycle-alkenyl or heterocycle-alkynyl can be unsubstituted or substituted with hydroxyl, halogen, —SH, trifluoromethyl, —OCH₃, —OCF₃, cyano, nitro, —COOH or NH₂,

each Z⁴ and Z⁵ is independently selected from hydrogen, alkyl, alkenyl, alkynyl, aryl, heterocycle, arylalkyl, arylalkenyl, arylalkynyl, heterocycle-alkyl, heterocycle-alkenyl or heterocycle-alkynyl, alkyl, alkenyl, alkynyl, aryl, heterocycle, arylalkyl, arylalkenyl, arylalkynyl, heterocycle-alkyl, heterocycle-alkenyl or heterocycle-alkynyl optionally includes one or more heteroatoms, said heteroatom selected from O, S and N, and wherein said alkyl, alkenyl, alkynyl, aryl, heterocycle, arylalkyl, arylalkenyl, arylalkynyl, heterocycle-alkyl, heterocycle-alkenyl or heterocycle-alkynyl can be unsubstituted or substituted with hydroxyl, halogen, —SH, trifluoromethyl, —OCH₃, —OCF₃, cyano, nitro, —COOH or NH₂, and wherein Z⁴ and Z⁵ can be taken together in order to form a (5-, 6-, or 7-membered) heterocycle which can be unsubstituted or substituted with hydroxyl, halogen, —SH, trifluoromethyl, —OCH₃, —OCF₃, cyano, nitro, —COOH or —NH₂, and isomers (in particular stereo-isomers or tautomers), solvates, hydrates, salts (in particular pharmaceutically acceptable salts) or prodrugs thereof.

In yet another particular embodiment, the compounds of the invention have a structure according to formula (III) or (IIIa),

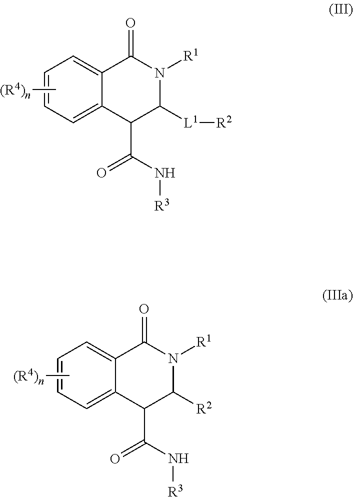

wherein,
each of R¹, R², R³, R⁴, L¹ and n are as described for the formulas herein and their embodiments.

In still another particular embodiment, the compounds of the invention have a structure according to formula (IV) or (IVb)),

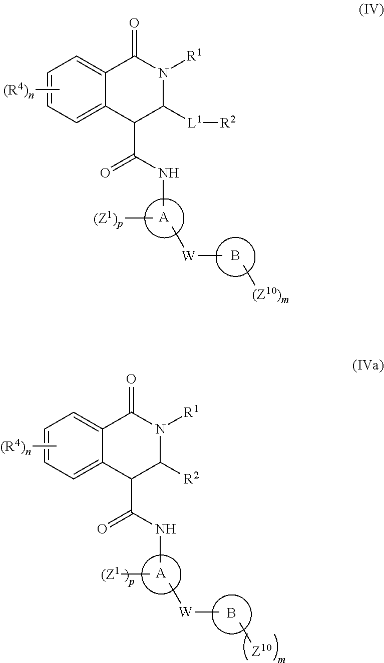

wherein,
each of R¹, R², R⁴, L¹, n, cycle A, cycle B, Z¹, Z¹⁰, W, p and m are as described for the formulas herein and their embodiments.

In still another particular embodiment, the compounds of the invention have a structure according to formula (V), (Va), (Vb) or (Vc),

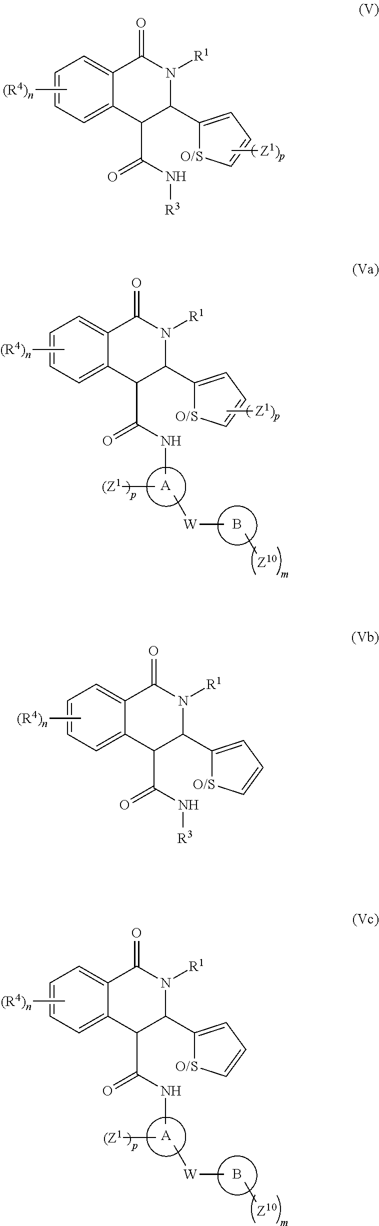

wherein,
each of R¹, R⁴, n, cycle A, cycle B, Z¹, Z¹⁰, W, p and m are as described for the formulas herein and their embodiments.

R¹ is selected from the group consisting of 2-methoxyethyl, 3-methoxypropyl, 2-ethoxyethyl, ethyl, n-butyl, methoxycarbonylmethyl, cyclohexylmethyl, 3-furylmethyl, dimethylaminoethyl, 2-hydroxyethyl, cyclohexyl, benzyl, trifluoromethyl, 2-methylthioethyl, 3-(N-morpholino)propyl, 2-isopropoxyethyl and (N-ethylpyrrolidin-2-yl)methyl.

Q=L¹R² is selected from the group consisting of thien-2-yl, 2-furyl, o-fluorophenyl, m-fluorophenyl, p-fluorophenyl, o,o-difluorophenyl, 3-methylthien-2-yl, 5-chlorothien-2-yl, 3-chlorothien-2-yl, pyrid-2-yl, pyrid-4-yl, pyrid-3-yl, benzothienyl, phenyl, pyrrol-2-yl, diazol-2,5-yl, 5-methyldiazol-2,4-yl, 2,4-dimethyldiazol-2,3-yl, 3,4-dimethyldiazol-2,3-yl, 2-methyldiazol-2,3-yl, ethyl, tert-butyl, methyl, benzyl, 2,2,2-trifluoroethyl, methylthiomethyl, trimethylsilylethynyl, ethynyl, 3,5-dimethylthiazol-2,4-yl, 4-methylthiazol-3,5-yl, thiazol-2,5-yl, 2,5-dimethyloxazol-3,4-yl, 3-methyloxazol-4,5-yl, 4-methyloxazol-2,3-yl, (tetrahydropyran-4-yl)methyl, 1-methylpyrazol-5-yl, 1-methylpyrrol-2-yl, cyclopentyl, phenylethynyl, 2,2-dimethylethenyl, o-methoxyphenyl, m-methoxyphenyl, benzyloxymethyl, hydroxymethyl, cyano, carboxamido and sulfonyldimethyl.

R³ is selected from the group consisting of m-methoxyphenyl, phenyl, m-trifluoromethylphenyl, p-trifluoromethylphenyl, p-cyanophenyl, m-biphenyl, o-biphenyl, benzyl, 2-phenoxyethyl, 2-(1-methylindol-3-yl)ethyl, p-methoxycarbonylphenyl, m-ethoxycarbonylphenyl, m-fluorophenyl, p-fluorophenyl, m-chlorophenyl, p-chorophenyl, m,m-dimethoxyphenyl, m,p-dimethoxyphenyl, m-ethoxyphenyl, p-methoxyphenyl, naphth-1-yl, m-toluoyl, p-toluoyl, pyrid-4-ylmethyl, benzothiazolyl, m,p-difluorophenyl, m,p-dichlorophenyl, m-benzylphenyl, p-benzylphenyl, m-chloro-p-fluorophenyl, p-(piperid-1-yl)phenyl, p-(pyrrol-1-yl)phenyl, m-(pyrrol-1-yl)phenyl, benzodioxanyl, m-acetamidophenyl, p-acetamidophenyl, p-chloro-m-methoxyphenyl, m-trifluoromethoxyphenyl, p-trifluoromethoxyphenyl, m-methoxy-p-methoxycarbonylphenyl, p-(N-morpholinyl)phenyl, 1-methylindol-5-yl, 4-methylcyclohexyl, 3-ethyl-oxadiazol-2,4,5-yl, 4-methyl-oxazol-2,3-yl, 4-methyl-oxazol-3,2-yl, 4-methyl-oxazol-2,5-yl, 4-methyl-5-cyano-oxazol-2,3-yl, p-(dimethylaminocarbonyl)phenyl, p-(benzylaminocarbonyl)phenyl, p-(methylaminocarbonyl)phenyl, 4-tert-butyl-oxazol-3,2-yl, o-amino-p-chorophenyl, o-amino-m-chorophenyl, 3,4-dimethylthiazol-2,5-yl, p-(4-methylthiazol-3,5yl)phenyl, 4-phenoxypyrid-3-yl, p-(fur-2-yl)phenyl, 4-(4-fluorophenyl)-oxazol-3,2-yl, pyrimidin-4-yl, pyrazin-2-yl, benzothiazolyl, p-isopropylphenyl, m-hydroxymethylphenyl, p-hydroxymethylphenyl, p-triazol-1,2,4-yl)phenyl, 2-methylquinolin-6-yl, quinoxalin-6-yl, m-(pyrazol-1-yl)phenyl, p-(pyrid-3-yl)phenyl, p-(pyrazol-1-ylmethyl)phenyl, p-(pyrimidin-2-yl)phenyl, 1-methylpyrazol-3-yl, 5-(fur-2-yl)pyrazol-3-yl, pyrazol-3-yl, 5-(tert-butyl)pyrazol-3-yl, 4-phenylpyrid-3-yl, p-phenoxyphenyl, 3-ethoxycarbonyl-oxadiazol-2,4,5-yl, p-(6-methylpyrazin-2-oxy)phenyl, tetrahydrobenzofuryl, p-(N-morpholinomethyl)phenyl, 1-methyl-3-cyclopropyl-pyrazol-5-yl, m-(triazol-1,2,4-yl)phenyl, 1-methyl-5-(methoxycarbonyl)-pyrrol-3-yl, p-(piperid-1-ylmethyl)phenyl, 5-chloropyrid-2-yl, p-cyano-m-trifluoromethylphenyl and m-cyano-p-(pyrrol-1-yl)-phenyl.

One aspect of the present invention is the provision of isoquinolin-1-one derivatives, namely compounds of formula (I)

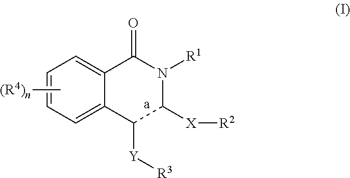

wherein,

the dotted line “a” is selected from a single bond or a double bond,

each of R¹, R² and R³ is independently selected from hydrogen or a hydrocarbyl group which optionally includes one or more heteroatoms, said heteroatoms being selected from the heteroatoms consisting of O, S, P, and N, wherein said hydrocarbyl group can be unsubstituted or substituted,

each R⁴ is independently selected from hydrogen, halogen, —OH, —OR⁵, —SH, —SR⁵, —S(O)R⁶, —S(O)₂R⁶, —SO₂NR⁷R⁸, trifluoromethyl, nitro, —NR⁵C(O)R⁵, —NR⁵S(O)₂R⁵, —NR⁵C(O)NR⁷R⁸, —NR⁷R⁸, -cyano, —COOH, —COOR⁵, —C(O)NR⁷R⁸, —C(O)R⁶, alkyl, alkenyl, alkynyl, aryl, heterocycle, arylalkyl, arylalkenyl, arylalkynyl, heterocycle-alkyl, heterocycle-alkenyl, heterocycle-alkynyl,
and wherein said alkyl, alkenyl, alkynyl, aryl, heterocycle, arylalkyl, arylalkenyl, arylalkynyl, heterocycle-alkyl, heterocycle-alkenyl or heterocycle-alkynyl, optionally includes one or more heteroatoms, said heteroatoms being selected from the atoms O, S, P and N,
and wherein said alkyl, alkenyl, alkynyl, aryl, heterocycle, arylalkyl, arylalkenyl, arylalkynyl, heterocycle-alkyl, heterocycle-alkenyl or heterocycle-alkynyl can be unsubstituted or substituted with halogen, —OH, —OR⁵, —SH, —SR⁵, —S(O)R⁶, —S(O)₂R⁶, —SO₂NR⁷R⁸, trifluoromethyl, nitro, —NR⁵C(O)R⁵, —NR⁵S(O)₂R⁵, —NR⁵C(O)NR⁷R⁸, —NR⁷R⁸, -cyano, —COOH, —COOR⁵, —C(O)NR⁷R⁸, —C(O)R⁶,

n is selected from 0, 1, 2, 3 or 4,

X is not present or is selected from C₁₋₆alkyl, C₁₋₆alkenyl or C₁₋₆alkynyl, wherein each of said C₁₋₆alkyl, C₁₋₆alkenyl or C₁₋₆alkynyl optionally includes one or more heteroatoms, said heteroatoms being selected from the heteroatoms consisting of O, S, P, and N, and wherein each of said C₁₋₆alkyl, C₁₋₆alkenyl or C₁₋₆alkynyl can be unsubstituted or substituted,

Y is not present or is selected from C₁₋₆alkyl, C₁₋₆alkenyl or C₁₋₆alkynyl, wherein each of said C₁₋₆alkyl, C₁₋₆alkenyl or C₁₋₆alkynyl optionally includes one or more heteroatoms, said heteroatoms being selected from the heteroatoms consisting of O, S, P, and N, and wherein each of said C₁₋₆alkyl, C₁₋₆alkenyl or C₁₋₆alkynyl can be unsubstituted or substituted,

each R⁵ is independently selected from alkyl, alkenyl, alkynyl, aryl, heterocycle, arylalkyl, arylalkenyl, arylalkynyl, heterocycle-alkyl, heterocycle-alkenyl or heterocycle-alkynyl, and wherein said alkyl, alkenyl, alkynyl, aryl, heterocycle, arylalkyl, arylalkenyl, arylalkynyl, heterocycle-alkyl, heterocycle-alkenyl or heterocycle-alkynyl optionally include one or more heteroatoms, said heteroatom selected from O, S, P and N, and wherein said alkyl, alkenyl, alkynyl, aryl, heterocycle, arylalkyl, arylalkenyl, arylalkynyl, heterocycle-alkyl, heterocycle-alkenyl or heterocycle-alkynyl can be unsubstituted or substituted,

each R⁶ is independently selected from hydroxyl, alkyl, alkenyl, alkynyl, aryl, heterocycle, arylalkyl, arylalkenyl, arylalkynyl, heterocycle-alkyl, heterocycle-alkenyl or heterocycle-alkynyl, and wherein said alkyl, alkenyl, alkynyl, aryl, heterocycle, arylalkyl, arylalkenyl, arylalkynyl, heterocycle-alkyl, heterocycle-alkenyl or heterocycle-alkynyl optionally includes one or more heteroatoms, said heteroatom selected from O, S, P and N, and wherein said alkyl, alkenyl, alkynyl, aryl, heterocycle, arylalkyl, arylalkenyl, arylalkynyl, heterocycle-alkyl, heterocycle-alkenyl or heterocycle-alkynyl can be unsubstituted or substituted,

each R⁷ and R⁸ is independently selected from hydrogen, alkyl, alkenyl, alkynyl, aryl, heterocycle, arylalkyl, arylalkenyl, arylalkynyl, heterocycle-alkyl, heterocycle-alkenyl or heterocycle-alkynyl, alkyl, alkenyl, alkynyl, aryl, heterocycle, arylalkyl, arylalkenyl, arylalkynyl, heterocycle-alkyl, heterocycle-alkenyl or heterocycle-alkynyl optionally includes one or more heteroatoms, said heteroatom selected from O, S, P and N, and wherein said alkyl, alkenyl, alkynyl, aryl, heterocycle, arylalkyl, arylalkenyl, arylalkynyl, heterocycle-alkyl, heterocycle-alkenyl or heterocycle-alkynyl can be unsubstituted or substituted with hydroxyl, halogen, —SH, trifluoromethyl, —OCH₃, —OCF₃, cyano, nitro, —COOH or NH₂, and wherein R⁷ and R⁸ can be taken together in order to form a (5-, 6-, or 7-membered) heterocycle which can be unsubstituted or substituted,

and isomers (in particular stereo-isomers or tautomers), solvates, hydrates, salts (in particular pharmaceutically acceptable salts) or prodrugs thereof,
for use as a medicament for the prevention or treatment of a RNA virus infection in an animal, a mammal (including a human).

In a particular embodiment, the compounds of the invention have a formula according to the formula (Ia), (Ib), (Ic) or (Id):

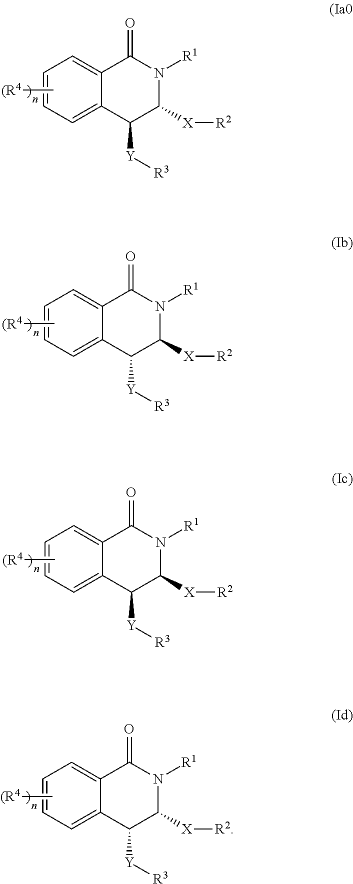

In yet another particular embodiment, the compounds of the invention are according to formula (I), (Ia), (Ib), (Ic), or (Id), wherein,

each of R¹, R² and R³ is independently selected from alkyl, alkenyl, alkynyl, aryl, heterocycle, arylalkyl, arylalkenyl, arylalkynyl, heterocycle-alkyl, heterocycle-alkenyl or heterocycle-alkynyl, and wherein said alkyl, alkenyl, alkynyl, aryl, heterocycle, arylalkyl, arylalkenyl, arylalkynyl, heterocycle-alkyl, heterocycle-alkenyl or heterocycle-alkynyl optionally include one or more heteroatoms, said heteroatom selected from O, S, P and N, and wherein said alkyl, alkenyl, alkynyl, aryl, heterocycle, arylalkyl, arylalkenyl, arylalkynyl, heterocycle-alkyl, heterocycle-alkenyl or heterocycle-alkynyl can be unsubstituted or substituted with Z¹,

X is not present or is selected from C₁₋₆alkyl, C₁₋₆alkenyl or C₁₋₆alkynyl, wherein each of said C₁₋₆alkyl, C₁₋₆alkenyl or C₁₋₆alkynyl optionally includes one or more heteroatoms, said heteroatoms being selected from the heteroatoms consisting of O, S, P, and N, and wherein each of said C₁₋₆alkyl, C₁₋₆alkenyl or C₁₋₆alkynyl can be unsubstituted or substituted with Z¹,

Y is not present or is selected from C₁₋₆alkyl, C₁₋₆alkenyl or C₁₋₆alkynyl, wherein each of said C₁₋₆alkyl, C₁₋₆alkenyl or C₁₋₆alkynyl optionally includes one or more heteroatoms, said heteroatoms being selected from the heteroatoms consisting of O, S, P, and N, and wherein each of said C₁₋₆alkyl, C₁₋₆alkenyl or C₁₋₆alkynyl can be unsubstituted or substituted with Z¹,

each R⁵ is independently selected from alkyl, alkenyl, alkynyl, aryl, heterocycle, arylalkyl, arylalkenyl, arylalkynyl, heterocycle-alkyl, heterocycle-alkenyl or heterocycle-alkynyl, and wherein said alkyl, alkenyl, alkynyl, aryl, heterocycle, arylalkyl, arylalkenyl, arylalkynyl, heterocycle-alkyl, heterocycle-alkenyl or heterocycle-alkynyl optionally include one or more heteroatoms, said heteroatom selected from O, S, P and N, and wherein said alkyl, alkenyl, alkynyl, aryl, heterocycle, arylalkyl, arylalkenyl, arylalkynyl, heterocycle-alkyl, heterocycle-alkenyl or heterocycle-alkynyl can be unsubstituted or substituted with Z¹,

each R⁶ is independently selected from hydroxyl, alkyl, alkenyl, alkynyl, aryl, heterocycle, arylalkyl, arylalkenyl, arylalkynyl, heterocycle-alkyl, heterocycle-alkenyl or heterocycle-alkynyl, and wherein said alkyl, alkenyl, alkynyl, aryl, heterocycle, arylalkyl, arylalkenyl, arylalkynyl, heterocycle-alkyl, heterocycle-alkenyl or heterocycle-alkynyl optionally includes one or more heteroatoms, said heteroatom selected from O, S, P and N, and wherein said alkyl, alkenyl, alkynyl, aryl, heterocycle, arylalkyl, arylalkenyl, arylalkynyl, heterocycle-alkyl, heterocycle-alkenyl or heterocycle-alkynyl can be unsubstituted or substituted with Z¹,

each R⁷ and R⁸ is independently selected from hydrogen, alkyl, alkenyl, alkynyl, aryl, heterocycle, arylalkyl, arylalkenyl, arylalkynyl, heterocycle-alkyl, heterocycle-alkenyl or heterocycle-alkynyl, alkyl, alkenyl, alkynyl, aryl, heterocycle, arylalkyl, arylalkenyl, arylalkynyl, heterocycle-alkyl, heterocycle-alkenyl or heterocycle-alkynyl optionally includes one or more heteroatoms, said heteroatom selected from O, S, P and N, and wherein said alkyl, alkenyl, alkynyl, aryl, heterocycle, arylalkyl, arylalkenyl, arylalkynyl, heterocycle-alkyl, heterocycle-alkenyl or heterocycle-alkynyl can be unsubstituted or substituted with hydroxyl, halogen, —SH, trifluoromethyl, —OCH₃, —OCF₃, cyano, nitro, —COOH or NH₂, and wherein R⁷ and R⁸ can be taken together in order to form a (5-, 6-, or 7-membered) heterocycle which can be unsubstituted or substituted with Z¹,

Z¹ is independently selected from the group consisting of hydrogen, halogen, hydroxyl, —SZ², —S(O)Z³, —S(O)₂Z³, —SO₂NZ⁴Z⁵, trifluoromethyl, nitro, —NZ⁴Z⁵, ⁻cyano, —COOZ², —C(O)NZ⁴Z⁵, —C(O)Z³, alkyl, alkenyl, alkynyl, aryl, heterocycle, arylalkyl, arylalkenyl, arylalkynyl, heterocycle-alkyl, heterocycle-alkenyl, heterocycle-alkynyl,
and wherein said alkyl, alkenyl, alkynyl, aryl, heterocycle, arylalkyl, arylalkenyl, arylalkynyl, heterocycle-alkyl, heterocycle-alkenyl or heterocycle-alkynyl, optionally includes one or more heteroatoms, said heteroatoms being selected from the atoms O, S, P and N,
and wherein said alkyl, alkenyl, alkynyl, aryl, heterocycle, arylalkyl, arylalkenyl, arylalkynyl, heterocycle-alkyl, heterocycle-alkenyl or heterocycle-alkynyl can be unsubstituted or substituted with hydroxyl, halogen, —SH, trifluoromethyl, —OCH₃, —OCF₃, cyano, nitro, —COOH or NH₂,

each Z² is independently selected from hydrogen, alkyl, alkenyl, alkynyl, aryl, heterocycle, arylalkyl, arylalkenyl, arylalkynyl, heterocycle-alkyl, heterocycle-alkenyl or heterocycle-alkynyl, and wherein said alkyl, alkenyl, alkynyl, aryl, heterocycle, arylalkyl, arylalkenyl, arylalkynyl, heterocycle-alkyl, heterocycle-alkenyl or heterocycle-alkynyl optionally include one or more heteroatoms, said heteroatom selected from O, S, P and N, and wherein said alkyl, alkenyl, alkynyl, aryl, heterocycle, arylalkyl, arylalkenyl, arylalkynyl, heterocycle-alkyl, heterocycle-alkenyl or heterocycle-alkynyl can be unsubstituted or substituted with hydroxyl, halogen, —SH, trifluoromethyl, —OCH₃, —OCF₃, cyano, nitro, —COOH or NH₂,

each Z³ is independently selected from hydroxyl, alkyl, alkenyl, alkynyl, aryl, heterocycle, arylalkyl, arylalkenyl, arylalkynyl, heterocycle-alkyl, heterocycle-alkenyl or heterocycle-alkynyl, and wherein said alkyl, alkenyl, alkynyl, aryl, heterocycle, arylalkyl, arylalkenyl, arylalkynyl, heterocycle-alkyl, heterocycle-alkenyl or heterocycle-alkynyl optionally includes one or more heteroatoms, said heteroatom selected from O, S, P and N, and wherein said alkyl, alkenyl, alkynyl, aryl, heterocycle, arylalkyl, arylalkenyl, arylalkynyl, heterocycle-alkyl, heterocycle-alkenyl or heterocycle-alkynyl can be unsubstituted or substituted with hydroxyl, halogen, —SH, trifluoromethyl, —OCH₃, —OCF₃, cyano, nitro, —COOH or NH₂,

each Z⁴ and Z⁵ is independently selected from hydrogen, alkyl, alkenyl, alkynyl, aryl, heterocycle, arylalkyl, arylalkenyl, arylalkynyl, heterocycle-alkyl, heterocycle-alkenyl or heterocycle-alkynyl, alkyl, alkenyl, alkynyl, aryl, heterocycle, arylalkyl, arylalkenyl, arylalkynyl, heterocycle-alkyl, heterocycle-alkenyl or heterocycle-alkynyl optionally includes one or more heteroatoms, said heteroatom selected from O, S, P and N, and wherein said alkyl, alkenyl, alkynyl, aryl, heterocycle, arylalkyl, arylalkenyl, arylalkynyl, heterocycle-alkyl, heterocycle-alkenyl or heterocycle-alkynyl can be unsubstituted or substituted with hydroxyl, halogen, —SH, trifluoromethyl, —OCH₃, —OCF₃, cyano, nitro, —COOH or NH₂, and wherein Z⁴ and Z⁵ can be taken together in order to form a (5-, 6-, or 7-membered) heterocycle which can be unsubstituted or substituted with hydroxyl, halogen, —SH, trifluoromethyl, —OCH₃, —OCF₃, cyano, nitro, —COOH or —NH₂.

In yet another particular embodiment, wherein n is selected from 2, 3 or 4. In another embodiment, the dotted line “a” is a single bond. In still a more particular embodiment, R¹ is selected from alkyl, alkenyl, or alkynyl, wherein said alkyl, alkenyl or alkynyl optionally include one or more heteroatoms, said heteroatom selected from O, S, P and N, and wherein said alkyl, alkenyl or alkynyl can be unsubstituted or substituted. In a more preferred embodiment, R¹ is selected from a linear, straight or branched, alkyl, alkenyl, or alkynyl, wherein said alkyl, alkenyl or alkynyl optionally include one or more heteroatoms, said heteroatom selected from O, S, P and N, and wherein said alkyl, alkenyl or alkynyl can be unsubstituted or substituted,
In another more particular embodiment, X is not present and R² is selected from aryl, heterocycle, arylalkyl, arylalkenyl, arylalkynyl, heterocycle-alkyl, heterocycle-alkenyl or heterocycle-alkynyl, wherein said aryl, heterocycle, arylalkyl, arylalkenyl, arylalkynyl, heterocycle-alkyl, heterocycle-alkenyl or heterocycle-alkynyl optionally include one or more heteroatoms, said heteroatom selected from O, S, P and N, and wherein said aryl, heterocycle, arylalkyl, arylalkenyl, arylalkynyl, heterocycle-alkyl, heterocycle-alkenyl or heterocycle-alkynyl can be unsubstituted or substituted. In yet another embodiment, R² is selected from unsubstituted or substituted thienyl or furanyl.
In another particular embodiment, Y is selected from —CH₂NZ⁶—, CH₂NH—, —CO—NZ⁶—, —CONH—, or —CH₂O—, and each Z⁶ is independently selected from alkyl, alkenyl, alkynyl, aryl, heterocycle, arylalkyl, arylalkenyl, arylalkynyl, heterocycle-alkyl, heterocycle-alkenyl or heterocycle-alkynyl, alkyl, alkenyl, alkynyl, aryl, heterocycle, arylalkyl, arylalkenyl, arylalkynyl, heterocycle-alkyl, heterocycle-alkenyl or heterocycle-alkynyl optionally includes one or more heteroatoms, said heteroatom selected from O, S, P and N, and wherein said alkyl, alkenyl, alkynyl, aryl, heterocycle, arylalkyl, arylalkenyl, arylalkynyl, heterocycle-alkyl, heterocycle-alkenyl or heterocycle-alkynyl can be unsubstituted or substituted with hydroxyl, halogen, —SH, trifluoromethyl, —OCH₃, —OCF₃, cyano, nitro, —COOH or NH₂, and wherein Z⁶ can be taken together with R³ in order to form a (5-, 6-, or 7-membered) heterocycle which can be unsubstituted or substituted with Z¹.
In a more particular embodiment, R³ is selected from aryl, heterocycle, arylalkyl, arylalkenyl, arylalkynyl, heterocycle-alkyl, heterocycle-alkenyl or heterocycle-alkynyl, and wherein said alkyl, alkenyl, alkynyl, aryl, heterocycle, arylalkyl, arylalkenyl, arylalkynyl, heterocycle-alkyl, heterocycle-alkenyl or heterocycle-alkynyl optionally include one or more heteroatoms, said heteroatom selected from O, S, P and N, and wherein said alkyl, alkenyl, alkynyl, aryl, heterocycle, arylalkyl, arylalkenyl, arylalkynyl, heterocycle-alkyl, heterocycle-alkenyl or heterocycle-alkynyl can be unsubstituted or substituted.

In a preferred embodiment, the compounds of the invention are according to the formulas (I), (Ia), (Ib), (Ic), or (Id), wherein

R¹ is independently selected from alkyl, alkenyl, or alkynyl, wherein said alkyl, alkenyl, or alkynyl, optionally include one or more heteroatoms, said heteroatom selected from O, S, P and N, and wherein said alkyl, alkenyl, or alkynyl, can be unsubstituted or substituted with Z¹,

R² is independently selected from aryl, heterocycle, arylalkyl, arylalkenyl, arylalkynyl, heterocycle-alkyl, heterocycle-alkenyl or heterocycle-alkynyl, wherein said aryl, heterocycle, arylalkyl, arylalkenyl, arylalkynyl, heterocycle-alkyl, heterocycle-alkenyl or heterocycle-alkynyl optionally include one or more heteroatoms, said heteroatom selected from O, S, P and N, and wherein said aryl, heterocycle, arylalkyl, arylalkenyl, arylalkynyl, heterocycle-alkyl, heterocycle-alkenyl or heterocycle-alkynyl can be unsubstituted or substituted with Z¹,

R³ is independently selected from aryl, heterocycle, arylalkyl, arylalkenyl, arylalkynyl, heterocycle-alkyl, heterocycle-alkenyl or heterocycle-alkynyl, wherein said aryl, heterocycle, arylalkyl, arylalkenyl, arylalkynyl, heterocycle-alkyl, heterocycle-alkenyl or heterocycle-alkynyl optionally include one or more heteroatoms, said heteroatom selected from O, S, P and N, and wherein said aryl, heterocycle, arylalkyl, arylalkenyl, arylalkynyl, heterocycle-alkyl, heterocycle-alkenyl or heterocycle-alkynyl can be unsubstituted or substituted with Z¹,

X is not present,

Y is selected from —CH₂NZ⁶—, CH₂NH—, —CO—NZ⁶—, or —CONH—,

each R⁴ is independently selected from hydrogen, halogen, —OH, —OW, —SH, —SR⁵, —S(O)R⁶, —S(O)₂R⁶, —SO₂NR⁷R⁸, trifluoromethyl, nitro, —NR⁵C(O)R⁵, —NR⁵S(O)₂R⁵, —NR⁵C(O)NR⁷R⁸, —NR⁷R⁸, -cyano, —COOH, —COOR⁵, —C(O)NR⁷R⁸, —C(O)R⁶, alkyl, alkenyl, alkynyl, aryl, heterocycle, arylalkyl, arylalkenyl, arylalkynyl, heterocycle-alkyl, heterocycle-alkenyl, heterocycle-alkynyl,
and wherein said alkyl, alkenyl, alkynyl, aryl, heterocycle, arylalkyl, arylalkenyl, arylalkynyl, heterocycle-alkyl, heterocycle-alkenyl or heterocycle-alkynyl, optionally includes one or more heteroatoms, said heteroatoms being selected from the atoms O, S, P and N,
and wherein said alkyl, alkenyl, alkynyl, aryl, heterocycle, arylalkyl, arylalkenyl, arylalkynyl, heterocycle-alkyl, heterocycle-alkenyl or heterocycle-alkynyl can be unsubstituted or substituted with halogen, —OH, —OR⁵, —SH, —SR⁵, —S(O)R⁶, —S(O)₂R⁶, —SO₂NR⁷R⁸, trifluoromethyl, nitro, —NR⁵C(O)R⁵, —NR⁵S(O)₂R⁵, —NR⁵C(O)NR⁷R⁸, —NR⁷R⁸, -cyano, —COOH, —COOR⁵, —C(O)NR⁷R⁸, —C(O)R⁶,

n is selected from 0, 1, 2, 3 or 4,

each Z⁶ is independently selected from alkyl, alkenyl, alkynyl, aryl, heterocycle, arylalkyl, arylalkenyl, arylalkynyl, heterocycle-alkyl, heterocycle-alkenyl or heterocycle-alkynyl, alkyl, alkenyl, alkynyl, aryl, heterocycle, arylalkyl, arylalkenyl, arylalkynyl, heterocycle-alkyl, heterocycle-alkenyl or heterocycle-alkynyl optionally includes one or more heteroatoms, said heteroatom selected from O, S, P and N, and wherein said alkyl, alkenyl, alkynyl, aryl, heterocycle, arylalkyl, arylalkenyl, arylalkynyl, heterocycle-alkyl, heterocycle-alkenyl or heterocycle-alkynyl can be unsubstituted or substituted with hydroxyl, halogen, —SH, trifluoromethyl, —OCH₃, —OCF₃, cyano, nitro, —COOH or NH₂, and wherein Z⁶ can be taken together with R³ in order to form a (5-, 6-, or 7-membered) heterocycle which can be unsubstituted or substituted with Z¹.

The compounds of the invention optionally are bound covalently to an insoluble matrix and used for affinity chromatography (separations, depending on the nature of the groups of the compounds, for example compounds with pendant aryl are useful in hydrophobic affinity separations.

The compounds of the invention are employed for the treatment or prophylaxis of viral infections, more particularly flaviviral infections, in particular HCV infections. When using one or more derivatives of the formulae as defined herein:
the active ingredients of the compound(s) may be administered to the animal or mammal (including a human) to be treated by any means well known in the art, i.e. orally, intranasally, subcutaneously, intramuscularly, intradermally, intravenously, intra-arterially, parenterally or by catheterization.
the therapeutically effective amount of the preparation of the compound(s), especially for the treatment of viral infections in humans and other mammals, preferably is a flaviviral replication inhibiting amount of the formulae as defined herein corresponds to an amount which ensures a plasma level of between 1 μg/ml and 100 mg/ml, optionally of 10 mg/ml.

The present invention further relates to a method for preventing or treating a viral infections in a subject or patient by administering to the patient in need thereof a therapeutically effective amount isoquinolinone derivatives of the present invention. The therapeutically effective amount of the preparation of the compound(s), especially for the treatment of viral infections in humans and other mammals, preferably is a flaviviral replication inhibiting amount. Suitable dosage is usually in the range of 0.001 mg to 60 mg, optionally 0.01 mg to 10 mg, optionally 0.1 mg to 1 mg per day per kg bodyweight for humans. Depending upon the pathologic condition to be treated and the patient's condition, the said effective amount may be divided into several sub-units per day or may be administered at more than one day intervals.

As is conventional in the art, the evaluation of a synergistic effect in a drug combination may be made by analyzing the quantification of the interactions between individual drugs, using the median effect principle described by Chou et al. in Adv. Enzyme Reg. (1984) 22:27. Briefly, this principle states that interactions (synergism, additivity, antagonism) between two drugs can be quantified using the combination index (hereinafter referred as CI) defined by the following equation:

$${CI}_{x} = \frac{{ED}_{x}^{1 c}}{{ED}_{x}^{1 a}} + \frac{{ED}_{x}^{2 c}}{{ED}_{x}^{2 a}}$$

wherein EDₓ is the dose of the first or respectively second drug used alone (1a, 2a), or in combination with the second or respectively first drug (1c, 2c), which is needed to produce a given effect. The said first and second drug have synergistic or additive or antagonistic effects depending upon CI<1, CI=1, or CI>1, respectively.

Synergistic activity of the pharmaceutical compositions or combined preparations of this invention against viral infection may also be readily determined by means of one or more tests such as, but not limited to, the isobologram method, as previously described by Elion et al. in J. Biol. Chem. (1954) 208:477-488 and by Baba et al. in Antimicrob. Agents Chemother. (1984) 25:515-517, using EC₅₀ for calculating the fractional inhibitory concentration (hereinafter referred as FIC). When the minimum FIC index corresponding to the FIC of combined compounds (e.g., FICₓ+FIC_{y}) is equal to 1.0, the combination is said to be additive, when it is between 1.0 and 0.5, the combination is defined as subsynergistic, and when it is lower than 0.5, the combination is by defined as synergistic. When the minimum FIC index is between 1.0 and 2.0, the combination is defined as subantagonistic and, when it is higher than 2.0, the combination is defined as antagonistic.

This principle may be applied to a combination of different antiviral drugs of the invention or to a combination of the antiviral drugs of the invention with other drugs that exhibit anti-HCV activity.

The invention thus relates to a pharmaceutical composition or combined preparation having synergistic effects against a viral infection and containing:

### Either:

### A)

(a) a combination of two or more of the isoquinolinone derivatives of the present invention, and
(b) optionally one or more pharmaceutical excipients or pharmaceutically acceptable carriers,
   for simultaneous, separate or sequential use in the treatment or prevention of a flaviviridae infection
   or

### B)

(c) one or more anti-viral agents, and
(d) at least one of the isoquinolinone derivatives of the present invention, and
(e) optionally one or more pharmaceutical excipients or pharmaceutically acceptable carriers,
   for simultaneous, separate or sequential use in the treatment or prevention of a flaviviridae infection.

Suitable anti-viral agents for inclusion into the synergistic antiviral compositions or combined preparations of this invention include, for instance, interferon-alfa (either pegylated or not), ribavirin and other selective inhibitors of the replication of HCV.

The pharmaceutical composition or combined preparation with synergistic activity against viral infection according to this invention may contain the isoquinolinone derivatives of the present invention over a broad content range depending on the contemplated use and the expected effect of the preparation. Generally, the content of the isoquinolinone derivatives of the present invention of the combined preparation is within the range of 0.1 to 99.9% by weight, preferably from 1 to 99% by weight, more preferably from 5 to 95% by weight.

According to a particular embodiment of the invention, the compounds of the invention may be employed in combination with other therapeutic agents for the treatment or prophylaxis of flaviviral infections, more preferably HCV. The invention therefore relates to the use of a composition comprising:
(a) one or more compounds of the formulae herein, and
(b) one or more flaviviral enzyme inhibitors as biologically active agents in respective proportions such as to provide a synergistic effect against a viral infection, particularly a flaviviral infection in a mammal, for instance in the form of a combined preparation for simultaneous, separate or sequential use in viral infection therapy, such as of HCV. Examples of such further therapeutic agents for use in combinations include agents that are effective for the treatment or prophylaxis of these infections, including interferon alpha, ribavirin, a compound falling within the scope of disclosure EP1162196, WO 03/010141, WO 03/007945 and WO 03/010140, a compound falling within the scope of disclosure WO 00/204425, and other patents or patent applications within their patent families or all the foregoing filings and/or an inhibitor of flaviviral protease and/or one or more additional flavivirus polymerase inhibitors.

More generally, the invention relates to the compounds of the formulas herein described being useful as agents having biological activity (particularly antiviral activity) or as diagnostic agents. Any of the uses mentioned with respect to the present invention may be restricted to a non-medical use, a non-therapeutic use, a non-diagnostic use, or exclusively an in vitro use, or a use related to cells remote from an animal.

Those of skill in the art will also recognize that the compounds of the invention may exist in many different protonation states, depending on, among other things, the pH of their environment. While the structural formulae provided herein depict the compounds in only one of several possible protonation states, it will be understood that these structures are illustrative only, and that the invention is not limited to any particular protonation state—any and all protonated forms of the compounds are intended to fall within the scope of the invention.

The term “pharmaceutically acceptable salts” as used herein means the therapeutically active non-toxic salt forms which the compounds of formulae herein are able to form. Therefore, the compounds of this invention optionally comprise salts of the compounds herein, especially pharmaceutically acceptable non-toxic salts containing, for example, Na+, Li+, K+, Ca+2 and Mg+2. Such salts may include those derived by combination of appropriate cations such as alkali and alkaline earth metal ions or ammonium and quaternary amino ions with an acid anion moiety, typically a carboxylic acid. The compounds of the invention may bear multiple positive or negative charges. The net charge of the compounds of the invention may be either positive or negative. Any associated counter ions are typically dictated by the synthesis and/or isolation methods by which the compounds are obtained. Typical counter ions include, but are not limited to ammonium, sodium, potassium, lithium, halides, acetate, trifluoroacetate, etc., and mixtures thereof. It will be understood that the identity of any associated counter ion is not a critical feature of the invention, and that the invention encompasses the compounds in association with any type of counter ion. Moreover, as the compounds can exist in a variety of different forms, the invention is intended to encompass not only forms of the compounds that are in association with counter ions (e.g., dry salts), but also forms that are not in association with counter ions (e.g., aqueous or organic solutions). Metal salts typically are prepared by reacting the metal hydroxide with a compound of this invention. Examples of metal salts which are prepared in this way are salts containing Li+, Na+, and K+. A less soluble metal salt can be precipitated from the solution of a more soluble salt by addition of the suitable metal compound. In addition, salts may be formed from acid addition of certain organic and inorganic acids to basic centers, typically amines, or to acidic groups. Examples of such appropriate acids include, for instance, inorganic acids such as hydrohalogen acids, e.g. hydrochloric or hydrobromic acid, sulfuric acid, nitric acid, phosphoric acid and the like, or organic acids such as, for example, acetic, propanoic, hydroxyacetic, 2-hydroxypropanoic, 2-oxopropanoic, lactic, pyruvic, oxalic (i.e. ethanedioic), malonic, succinic (i.e. butanedioic acid), maleic, fumaric, malic, tartaric, citric, methanesulfonic, ethanesulfonic, benzenesulfonic, p-toluenesulfonic, cyclohexanesulfamic, salicylic (i.e. 2-hydroxybenzoic), p-aminosalicylic and the like. Furthermore, this term also includes the solvates which the compounds of formulae herein as well as their salts are able to form, such as for example hydrates, alcoholates and the like. Finally, it is to be understood that the compositions herein comprise compounds of the invention in their unionized, as well as zwitterionic form, and combinations with stoichiometric amounts of water as in hydrates.

Also included within the scope of this invention are the salts of the parental compounds with one or more amino acids, especially the naturally-occurring amino acids found as protein components. The amino acid typically is one bearing a side chain with a basic or acidic group, e.g., lysine, arginine or glutamic acid, or a neutral group such as glycine, serine, threonine, alanine, isoleucine, or leucine.

The compounds of the invention also include physiologically acceptable salts thereof. Examples of physiologically acceptable salts of the compounds of the invention include salts derived from an appropriate base, such as an alkali metal (for example, sodium), an alkaline earth (for example, magnesium), ammonium and NX₄⁺ (wherein X is C₁-C₄ alkyl). Physiologically acceptable salts of an hydrogen atom or an amino group include salts of organic carboxylic acids such as acetic, benzoic, lactic, fumaric, tartaric, maleic, malonic, malic, isethionic, lactobionic and succinic acids, organic sulfonic acids, such as methanesulfonic, ethanesulfonic, benzenesulfonic and p-toluenesulfonic acids, and inorganic acids, such as hydrochloric, sulfuric, phosphoric and sulfamic acids. Physiologically acceptable salts of a compound containing a hydroxy group include the anion of said compound in combination with a suitable cation such as Na+ and NX₄⁺ (wherein X typically is independently selected from H or a C₁-C₄ alkyl group). However, salts of acids or bases which are not physiologically acceptable may also find use, for example, in the preparation or purification of a physiologically acceptable compound. All salts, whether or not derived form a physiologically acceptable acid or base, are within the scope of the present invention.

As used herein and unless otherwise stated, the term “enantiomer” means each individual optically active form of a compound of the invention, having an optical purity or enantiomeric excess (as determined by methods standard in the art) of at least 80% (i.e. at least 90% of one enantiomer and at most 10% of the other enantiomer), preferably at least 90% and more preferably at least 98%.

The term “isomers” as used herein means all possible isomeric forms, including tautomeric and stereochemical forms, which the compounds of formulae herein may possess, but not including position isomers. Typically, the structures shown herein exemplify only one tautomeric or resonance form of the compounds, but the corresponding alternative configurations are contemplated as well. Unless otherwise stated, the chemical designation of compounds denotes the mixture of all possible stereochemically isomeric forms, said mixtures containing all diastereomers and enantiomers (since the compounds of formulae herein may have at least one chiral center) of the basic molecular structure, as well as the stereochemically pure or enriched compounds. More particularly, stereogenic centers may have either the R- or S-configuration, and multiple bonds may have either cis- or trans-configuration.

Pure isomeric forms of the said compounds are defined as isomers substantially free of other enantiomeric or diastereomeric forms of the same basic molecular structure. In particular, the term “stereoisomerically pure” or “chirally pure” relates to compounds having a stereoisomeric excess of at least about 80% (i.e. at least 90% of one isomer and at most 10% of the other possible isomers), preferably at least 90%, more preferably at least 94% and most preferably at least 97%. The terms “enantiomerically pure” and “diastereomerically pure” should be understood in a similar way, having regard to the enantiomeric excess, respectively the diastereomeric excess, of the mixture in question.

Separation of stereoisomers is accomplished by standard methods known to those in the art. One enantiomer of a compound of the invention can be separated substantially free of its opposing enantiomer by a method such as formation of diastereomers using optically active resolving agents (“Stereochemistry of Carbon Compounds,” (1962) by E. L. Eliel, McGraw Hill, Lochmuller, C. H., (1975) J. Chromatogr., 113:(3) 283-302). Separation of isomers in a mixture can be accomplished by any suitable method, including: (1) formation of ionic, diastereomeric salts with chiral compounds and separation by fractional crystallization or other methods, (2) formation of diastereomeric compounds with chiral derivatizing reagents, separation of the diastereomers, and conversion to the pure enantiomers, or (3) enantiomers can be separated directly under chiral conditions. Under method (1), diastereomeric salts can be formed by reaction of enantiomerically pure chiral bases such as brucine, quinine, ephedrine, strychnine, a-methyl-b-phenylethylamine (amphetamine), and the like with asymmetric compounds bearing acidic functionality, such as carboxylic acid and sulfonic acid. The diastereomeric salts may be induced to separate by fractional crystallization or ionic chromatography. For separation of the optical isomers of amino compounds, addition of chiral carboxylic or sulfonic acids, such as camphorsulfonic acid, tartaric acid, mandelic acid, or lactic acid can result in formation of the diastereomeric salts. Alternatively, by method (2), the substrate to be resolved may be reacted with one enantiomer of a chiral compound to form a diastereomeric pair (Eliel, E. and Wilen, S. (1994) Stereochemistry of Organic Compounds, John Wiley & Sons, Inc., p. 322). Diastereomeric compounds can be formed by reacting asymmetric compounds with enantiomerically pure chiral derivatizing reagents, such as menthyl derivatives, followed by separation of the diastereomers and hydrolysis to yield the free, enantiomerically enriched xanthene. A method of determining optical purity involves making chiral esters, such as a menthyl ester or Mosher ester, a-methoxy-a-(trifluoromethyl)phenyl acetate (Jacob III. (1982) J. Org. Chem. 47:4165), of the racemic mixture, and analyzing the NMR spectrum for the presence of the two atropisomeric diastereomers. Stable diastereomers can be separated and isolated by normal- and reverse-phase chromatography following methods for separation of atropisomeric naphthyl-isoquinolines (Hoye, T., WO 96/15111). Under method (3), a racemic mixture of two asymmetric enantiomers is separated by chromatography using a chiral stationary phase. Suitable chiral stationary phases are, for example, polysaccharides, in particular cellulose or amylose derivatives. Commercially available polysaccharide based chiral stationary phases are ChiralCel™ CA, OA, OB5, OC5, OD, OF, OG, OJ and OK, and Chiralpak™ AD, AS, OP(+) and OT(+). Appropriate eluents or mobile phases for use in combination with said polysaccharide chiral stationary phases are hexane and the like, modified with an alcohol such as ethanol, isopropanol and the like. (“Chiral Liquid Chromatography” (1989) W. J. Lough, Ed. Chapman and Hall, New York, Okamoto, (1990) “Optical resolution of dihydropyridine enantiomers by High-performance liquid chromatography using phenylcarbamates of polysaccharides as a chiral stationary phase”, J. of Chromatogr. 513:375-378).

The terms cis and trans are used herein in accordance with Chemical Abstracts nomenclature and include reference to the position of the substituents on a ring moiety. The absolute stereochemical configuration of the compounds of formula (I) may easily be determined by those skilled in the art while using well-known methods such as, for example, X-ray diffraction.

The compounds of the invention may be formulated with conventional carriers and excipients, which will be selected in accord with ordinary practice. Tablets will contain excipients, glidants, fillers, binders and the like. Aqueous formulations are prepared in sterile form, and when intended for delivery by other than oral administration generally will be isotonic. Formulations optionally contain excipients such as those set forth in the “Handbook of Pharmaceutical Excipients” (1986) and include ascorbic acid and other antioxidants, chelating agents such as EDTA, carbohydrates such as dextrin, hydroxyalkylcellulose, hydroxyalkylmethylcellulose, stearic acid and the like.

Subsequently, the term “pharmaceutically acceptable carrier” as used herein means any material or substance with which the active ingredient is formulated in order to facilitate its application or dissemination to the locus to be treated, for instance by dissolving, dispersing or diffusing the said composition, and/or to facilitate its storage, transport or handling without impairing its effectiveness. The pharmaceutically acceptable carrier may be a solid or a liquid or a gas which has been compressed to form a liquid, i.e. the compositions of this invention can suitably be used as concentrates, emulsions, solutions, granulates, dusts, sprays, aerosols, suspensions, ointments, creams, tablets, pellets or powders.

Suitable pharmaceutical carriers for use in the said pharmaceutical compositions and their formulation are well known to those skilled in the art, and there is no particular restriction to their selection within the present invention. They may also include additives such as wetting agents, dispersing agents, stickers, adhesives, emulsifying agents, solvents, coatings, antibacterial and antifungal agents (for example phenol, sorbic acid, chlorobutanol), isotonic agents (such as sugars or sodium chloride) and the like, provided the same are consistent with pharmaceutical practice, i.e. carriers and additives which do not create permanent damage to mammals. The pharmaceutical compositions of the present invention may be prepared in any known manner, for instance by homogeneously mixing, coating and/or grinding the active ingredients, in a one-step or multi-steps procedure, with the selected carrier material and, where appropriate, the other additives such as surface-active agents. may also be prepared by micronisation, for instance in view to obtain them in the form of microspheres usually having a diameter of about 1 to 10 gm, namely for the manufacture of microcapsules for controlled or sustained release of the active ingredients.

Suitable surface-active agents, also known as emulgent or emulsifier, to be used in the pharmaceutical compositions of the present invention are non-ionic, cationic and/or anionic materials having good emulsifying, dispersing and/or wetting properties. Suitable anionic surfactants include both water-soluble soaps and water-soluble synthetic surface-active agents. Suitable soaps are alkaline or alkaline-earth metal salts, unsubstituted or substituted ammonium salts of higher fatty acids (C₁₀-C₂₂), e.g. the sodium or potassium salts of oleic or stearic acid, or of natural fatty acid mixtures obtainable from coconut oil or tallow oil. Synthetic surfactants include sodium or calcium salts of polyacrylic acids, fatty sulphonates and sulphates, sulphonated benzimidazole derivatives and alkylarylsulphonates. Fatty sulphonates or sulphates are usually in the form of alkaline or alkaline-earth metal salts, unsubstituted ammonium salts or ammonium salts substituted with an alkyl or acyl radical having from 8 to 22 carbon atoms, e.g. the sodium or calcium salt of lignosulphonic acid or dodecylsulphonic acid or a mixture of fatty alcohol sulphates obtained from natural fatty acids, alkaline or alkaline-earth metal salts of sulphuric or sulphonic acid esters (such as sodium lauryl sulphate) and sulphonic acids of fatty alcohol/ethylene oxide adducts. Suitable sulphonated benzimidazole derivatives preferably contain 8 to 22 carbon atoms. Examples of alkylarylsulphonates are the sodium, calcium or alcoholamine salts of dodecylbenzene sulphonic acid or dibutyl-naphthalenesulphonic acid or a naphthalene-sulphonic acid/formaldehyde condensation product. Also suitable are the corresponding phosphates, e.g. salts of phosphoric acid ester and an adduct of p-nonylphenol with ethylene and/or propylene oxide, or phospholipids. Suitable phospholipids for this purpose are the natural (originating from animal or plant cells) or synthetic phospholipids of the cephalin or lecithin type such as e.g. phosphatidylethanolamine, phosphatidylserine, phosphatidylglycerine, lysolecithin, cardiolipin, dioctanylphosphatidyl-choline, dipalmitoylphoshatidyl-choline and their mixtures.

Suitable non-ionic surfactants include polyethoxylated and polypropoxylated derivatives of alkylphenols, fatty alcohols, fatty acids, aliphatic amines or amides containing at least 12 carbon atoms in the molecule, alkylarenesulphonates and dialkylsulphosuccinates, such as polyglycol ether derivatives of aliphatic and cycloaliphatic alcohols, saturated and unsaturated fatty acids and alkylphenols, said derivatives preferably containing 3 to 10 glycol ether groups and 8 to 20 carbon atoms in the (aliphatic) hydrocarbon moiety and 6 to 18 carbon atoms in the alkyl moiety of the alkylphenol. Further suitable non-ionic surfactants are water-soluble adducts of polyethylene oxide with poylypropylene glycol, ethylenediaminopolypropylene glycol containing 1 to 10 carbon atoms in the alkyl chain, which adducts contain 20 to 250 ethyleneglycol ether groups and/or 10 to 100 propyleneglycol ether groups. Such compounds usually contain from 1 to 5 ethyleneglycol units per propyleneglycol unit. Representative examples of non-ionic surfactants are nonylphenol-polyethoxyethanol, castor oil polyglycolic ethers, polypropylene/polyethylene oxide adducts, tributylphenoxypolyethoxyethanol, polyethyleneglycol and octylphenoxypolyethoxyethanol. Fatty acid esters of polyethylene sorbitan (such as polyoxyethylene sorbitan trioleate), glycerol, sorbitan, sucrose and pentaerythritol are also suitable non-ionic surfactants.

Suitable cationic surfactants include quaternary ammonium salts, particularly halides, having 4 hydrocarbon radicals optionally substituted with halo, phenyl, substituted phenyl or hydroxy, for instance quaternary ammonium salts containing as N-substituent at least one C8C22 alkyl radical (e.g. cetyl, lauryl, palmityl, myristyl, oleyl and the like) and, as further substituents, unsubstituted or halogenated lower alkyl, benzyl and/or hydroxy-lower alkyl radicals.

A more detailed description of surface-active agents suitable for this purpose may be found for instance in “McCutcheon's Detergents and Emulsifiers Annual” (MC Publishing Crop., Ridgewood, N.J., 1981), “Tensid-Taschenbucw’, 2 d ed. (Hanser Verlag, Vienna, 1981) and “Encyclopaedia of Surfactants, (Chemical Publishing Co., New York, 1981).

Compounds of the invention and their physiologically acceptable salts (hereafter collectively referred to as the active ingredients) may be administered by any route appropriate to the condition to be treated, suitable routes including oral, rectal, nasal, topical (including ocular, buccal and sublingual), vaginal and parenteral (including subcutaneous, intramuscular, intravenous, intradermal, intrathecal and epidural). The preferred route of administration may vary with for example the condition of the recipient.

While it is possible for the active ingredients to be administered alone it is preferable to present them as pharmaceutical formulations. The formulations, both for veterinary and for human use, of the present invention comprise at least one active ingredient, as above described, together with one or more pharmaceutically acceptable carriers therefore and optionally other. therapeutic ingredients. The carrier(s) optimally are “acceptable” in the sense of being compatible with the other ingredients of the formulation and not deleterious to the recipient thereof. The formulations include those suitable for oral, rectal, nasal, topical (including buccal and sublingual), vaginal or parenteral (including subcutaneous, intramuscular, intravenous, intradermal, intrathecal and epidural) administration. The formulations may conveniently be presented in unit dosage form and may be prepared by any of the methods well known in the art of pharmacy. Such methods include the step of bringing into association the active ingredient with the carrier which constitutes one or more accessory ingredients. In general the formulations are prepared by uniformly and intimately bringing into association the active ingredient with liquid carriers or finely divided solid carriers or both, and then, if necessary, shaping the product.

Formulations of the present invention suitable for oral administration may be presented as discrete units such as capsules, cachets or tablets each containing a predetermined amount of the active ingredient, as a powder or granules, as solution or a suspension in an aqueous liquid or a non-aqueous liquid, or as an oil-in-water liquid emulsion or a water-in-oil liquid emulsion. The active ingredient may also be presented as a bolus, electuary or paste.

A tablet may be made by compression or molding, optionally with one or more accessory ingredients. Compressed tablets may be prepared by compressing in a suitable machine the active ingredient in a free-flowing form such as a powder or granules, optionally mixed with a binder, lubricant, inert diluent, preservative, surface active or dispersing agent. Molded tablets may be made by molding in a suitable machine a mixture of the powdered compound moistened with an inert liquid diluent. The tablets may optionally be coated or scored and may be formulated so as to provide slow or controlled release of the active ingredient therein. For infections of the eye or other external tissues e.g. mouth and skin, the formulations are optionally applied as a topical ointment or cream containing the active ingredient(s) in an amount of, for example, 0.075 to 20% w/w (including active ingredient(s) in a range between 0.1% and 20% in increments of 0.1% w/w such as 0.6% w/w, 0.7% w/w, etc), preferably 0.2 to 15% w/w and most preferably 0.5 to 10% w/w. When formulated in an ointment, the active ingredients may be employed with either a paraffinic or a water-miscible ointment base. Alternatively, the active ingredients may be formulated in a cream with an oil-in-water cream base. If desired, the aqueous phase of the cream base may include, for example, at least 30% w/w of a polyhydric alcohol, i.e. an alcohol having two or more hydroxyl groups such as propylene glycol, butane 1,3-diol, mannitol, sorbitol, glycerol and polyethylene glycol (including PEG400) and mixtures thereof. The topical formulations may desirably include a compound which enhances absorption or penetration of the active ingredient through the skin or other affected areas. Examples of such dermal penetration enhancers include dimethylsulfoxide and related analogs.

The oily phase of the emulsions of this invention may be constituted from known ingredients in a known manner. While the phase may comprise merely an emulsifier (otherwise known as an emulgent), it desirably comprises a mixture of at least one emulsifier with a fat or an oil or with both a fat and an oil. Optionally, a hydrophilic emulsifier is included together with a lipophilic emulsifier which acts as a stabilizer. It is also preferred to include both an oil and a fat. Together, the emulsifier(s) with or without stabilizer(s) make up the so-called emulsifying wax, and the wax together with the oil and fat make up the so-called emulsifying ointment base which forms the oily dispersed phase of the cream formulations.

The choice of suitable oils or fats for the formulation is based on achieving the desired cosmetic properties, since the solubility of the active compound in most oils likely to be used in pharmaceutical emulsion formulations is very low. Thus the cream should optionally be a non-greasy, non-staining and washable product with suitable consistency to avoid leakage from tubes or other containers. Straight or branched chain, mono- or dibasic alkyl esters such as di-isoadipate, isocetyl stearate, propylene glycol diester of coconut fatty acids, isopropyl myristate, decyl oleate, isopropyl palmitate, butyl stearate, 2-ethylhexyl palmitate or a blend of branched chain esters known as Crodamol CAP may be used, the last three being preferred esters. These may be used alone or in combination depending on the properties required. Alternatively, high melting point lipids such as white soft paraffin and/or liquid paraffin or other mineral oils can be used.

Formulations suitable for topical administration to the eye also include eye drops wherein the active ingredient is dissolved or suspended in a suitable carrier, especially an aqueous solvent for the active ingredient. The active ingredient is optionally present in such formulations in a concentration of 0.5 to 20%, advantageously 0.5 to 10% particularly about 1.5% w/w. Formulations suitable for topical administration in the mouth include lozenges comprising the active ingredient in a flavored basis, usually sucrose and acacia or tragacanth, pastilles comprising the active ingredient in an inert basis such as gelatin and glycerin, or sucrose and acacia, and mouthwashes comprising the active ingredient in a suitable liquid carrier.

Formulations for rectal administration may be presented as a suppository with a suitable base comprising for example cocoa butter or a salicylate. Formulations suitable for nasal administration wherein the carrier is a solid include a coarse powder having a particle size for example in the range 20 to 500 microns (including particle sizes in a range between 20 and 500 microns in increments of 5 microns such as 30 microns, 35 microns, etc), which is administered in the manner in which snuff is taken, i.e. by rapid inhalation through the nasal passage from a container of the powder held close up to the nose. Suitable formulations wherein the carrier is a liquid, for administration as for example a nasal spray or as nasal drops, include aqueous or oily solutions of the active ingredient. Formulations suitable for aerosol administration may be prepared according to conventional methods and may be delivered with other therapeutic agents.

Formulations suitable for vaginal administration may be presented as pessaries, tampons, creams, gels, pastes, foams or spray formulations containing in addition to the active ingredient such carriers as are known in the art to be appropriate.

Formulations suitable for parenteral administration include aqueous and non-aqueous sterile injection solutions which may contain anti-oxidants, buffers, bacteriostats and solutes which render the formulation isotonic with the blood of the intended recipient, and aqueous and non-aqueous sterile suspensions which may include suspending agents and thickening agents. The formulations may be presented in unit-dose or multi-dose containers, for example sealed ampoules and vials, and may be stored in a freeze-dried (lyophilized) condition requiring only the addition of the sterile liquid carrier, for example water for injections, immediately prior to use. Extemporaneous injection solutions and suspensions may be prepared from sterile powders, granules and tablets of the kind previously described.

Preferred unit dosage formulations are those containing a daily dose or unit daily sub-dose, as herein above recited, or an appropriate fraction thereof, of an active ingredient.

It should be understood that in addition to the ingredients particularly mentioned above the formulations of this invention may include other agents conventional in the art having regard to the type of formulation in question, for example those suitable for oral administration may include flavoring agents.

Compounds of the invention can be used to provide controlled release pharmaceutical formulations containing as active ingredient one or more compounds of the invention (“controlled release formulations”) in which the release of the active ingredient can be controlled and regulated to allow less frequency dosing or to improve the pharmacokinetic or toxicity profile of a given invention compound. Controlled release formulations adapted for oral administration in which discrete units comprising one or more compounds of the invention can be prepared according to conventional methods.

Additional ingredients may be included in order to control the duration of action of the active ingredient in the composition. Control release compositions may thus be achieved by selecting appropriate polymer carriers such as for example polyesters, polyamino acids, polyvinyl pyrrolidone, ethylene-vinyl acetate copolymers, methylcellulose, carboxymethylcellulose, protamine sulfate and the like. The rate of drug release and duration of action may also be controlled by incorporating the active ingredient into particles, e.g. microcapsules, of a polymeric substance such as hydrogels, polylactic acid, hydroxymethylcellulose, polymethyl methacrylate and the other above-described polymers. Such methods include colloid drug delivery systems like liposomes, microspheres, microemulsions, nanoparticles, nanocapsules and so on. Depending on the route of administration, the pharmaceutical composition may require protective coatings. Pharmaceutical forms suitable for injectionable use include sterile aqueous solutions or dispersions and sterile powders for the extemporaneous preparation thereof. Typical carriers for this purpose therefore include biocompatible aqueous buffers, ethanol, glycerol, propylene glycol, polyethylene glycol and the like and mixtures thereof.

In view of the fact that, when several active ingredients are used in combination, they do not necessarily bring out their joint therapeutic effect directly at the same time in the mammal to be treated, the corresponding composition may also be in the form of a medical kit or package containing the two ingredients in separate but adjacent repositories or compartments. In the latter context, each active ingredient may therefore be formulated in a way suitable for an administration route different from that of the other ingredient, e.g. one of them may be in the form of an oral or parenteral formulation whereas the other is in the form of an ampoule for intravenous injection or an aerosol.

When several active ingredients are used in combination, they do not necessarily bring out their joint therapeutic effect directly at the same time into the animal or mammal to be treated, the corresponding composition may also be in the form of a medical kit or package containing the two ingredients in separate but adjacent repositories or compartments. In the latter context, each active ingredient may therefore be formulated in a way suitable for an administration route different from that of the other ingredient, e.g. one of them may be in the form of an oral or parenteral formulation whereas the other is in the form of an ampoule for intravenous injection.

Another embodiment of this invention relates to various precursor or “pro-drug” forms of the compounds of the present invention. It may be desirable to formulate the compounds of the present invention in the form of a chemical species which itself is not significantly biologically-active, but which when delivered to the animal or mammal will undergo a chemical reaction catalyzed by the normal function of the body of the animal or mammal, inter alia, enzymes present in the stomach or in blood serum, said chemical reaction having the effect of releasing a compound as defined herein. The term “pro-drug” thus relates to these species which are converted in vivo into the active pharmaceutical ingredient.

The pro-drugs of the present invention can have any form suitable to the formulator, for example, esters are non-limiting common pro-drug forms. In the present case, however, the pro-drug may necessarily exist in a form wherein a covalent bond is cleaved by the action of an enzyme present at the target locus. For example, a C—C covalent bond may be selectively cleaved by one or more enzymes at said target locus and, therefore, a pro-drug in a form other than an easily hydrolysable precursor, inter alia an ester, an amide, and the like, may be used. The counterpart of the active pharmaceutical ingredient in the pro-drug can have different structures such as an amino acid or peptide structure, alkyl chains, sugar moieties and others as known in the art.

Scheme 1 shows schematically a method for preparing 1-oxo-1,2,3,4-tetrahydro-isoquinoline-4-carboxylic acids, key intermediates in the preparation of the compounds of the present invention in which the dotted line “a” in formula (I) represents a single bond or a double bond, and the derivitisation of 1-oxo-1,2,3,4-tetrahydroisoquinoline-4-carboxylic acids to provide compounds according to the present invention according to formula (I) in which the dotted line “a” in formula (I) represents a single bond. The method of preparing 1-oxo-1,2,3,4-tetrahydroisoquinoline-4-carboxylic acids comprises the steps of:

reacting an aromatic or heteroaromatic aldehyde (represented in scheme 1 by R^{k}CHO) with a primary amine (represented in scheme 1 by H₂NR^{c}) to obtain an azomethine (represented in scheme 1 by R^{k}CH═NR^{c},

reacting a homophthalic acid optionally substituted in the benzene ring with an anhydride to obtain the corresponding homophthalic anhydride (shown in scheme 1), and

reacting the homophthalic anhydride with the azomethine in a polar or apolar solvent to obtain a 1-oxo-1,2,3,4-tetrahydroisoquinoline-4-carboxylic acid (shown in scheme 1).

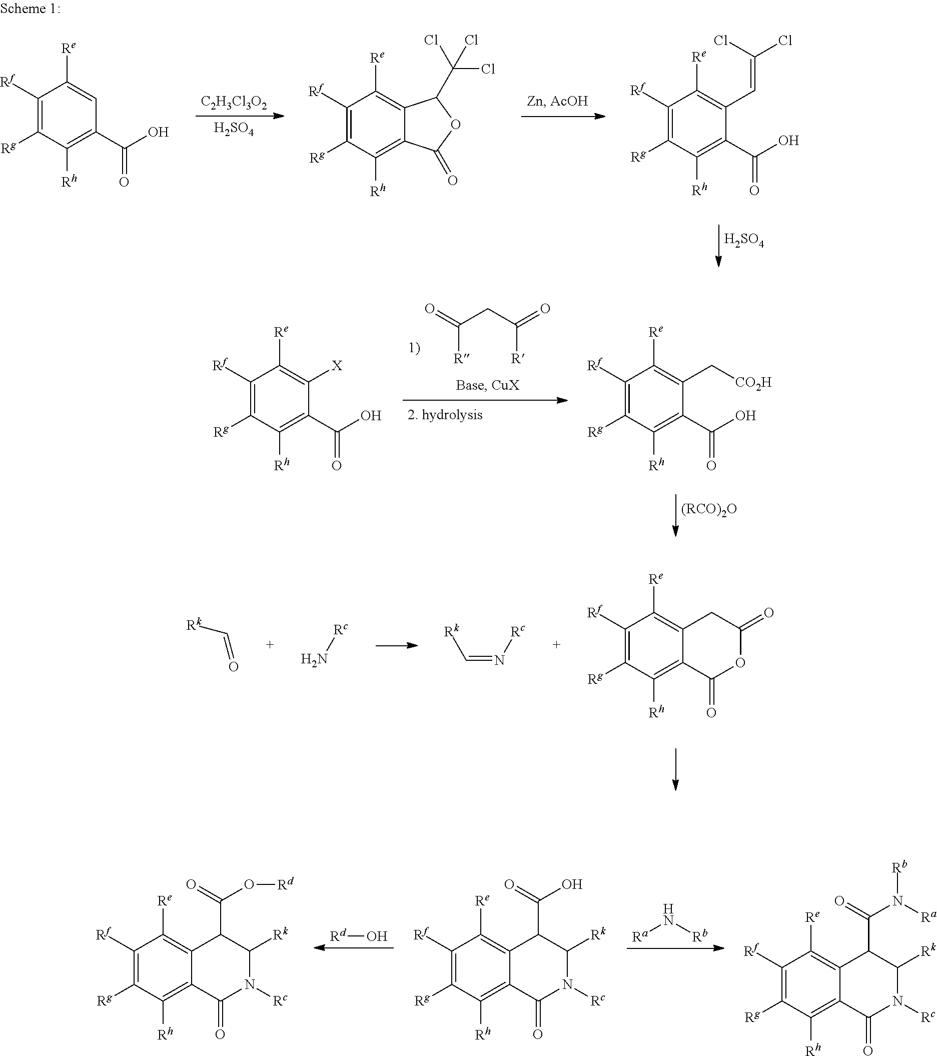

The synthesis is based on a key diacid intermediate (unsubstituted or substituted homophtalic acid), whose preparation has been reported in the literature and which is known to the skilled in the art. More detailed information can be found in the following references (e.g., Journal of Indian Chemical Society, 1965, 42, 439, JOC, 1988, 53, 1114, JOC, 1998, 63, 4116, JOC, 2003, 68, 5967). Many of such unsubstituted or substituted homophthalic acids are furthermore commercially available.

This unsubstituted or substituted with R^{e}, R^{f}, R^{g} and/or R^{h} homophthalic acid (hereinafter sometimes referred to a diacid), is then converted to the corresponding homophthalic anhydride by treatment with an anhydride (e.g., acetic anhydride, trifluoroacetic anhydride) or an acyl chloride (e.g., acetyl chloride). More detailed information can be found in the following articles (cf. e.g., JOC, 2003, 68, 5967, Angew. Chem.Int.Ed. 2007, 46, 5352).

The novel key step in this synthesis route is the reaction of homophthalic anhydride with an azomethine (Schiff's base) in various apolar aprotic solvents (e.g., benzene, toluene, . . . ) or polar aprotic solvents (e.g., dichloromethane, DMF . . . ) to deliver the desired carboxylic acid. The azomethine itself is obtained via a reaction between an aldehyde and a primary amine in a polar aprotic solvent (e.g., dichloromethane, THF, chloroform, . . . ) or without solvent at a temperature increasing from 20 to 65° C.

The compounds of the present invention are then prepared by esterification, amidation and reduction reactions using 1-oxo-1,2,3,4-tetrahydroisoquinoline-4-carboxylic acids. For example the 1-oxo-1,2,3,4-terahydro-isoquinoline-4-carboxylic acid derivative is tconverted to carboxamides by using standard peptide coupling conditions (e.g., EDCI, HATU, . . . ) or to esters by using similar conditions or preferably treatment with SOCl₂ and reaction with an alcohol.

Compounds of the present invention can be synthesized from 1-oxo-1,2,3,4-tetrahydroisoquinoline-4-carboxylic acids in which the dotted line “a” in formula (I) represents a single bond using different processes including the processes shown in Scheme 2 below in which the acid is first esterified (preferably methyl or ethyl esters) and the resulting ester reduced to a —CH₂OH group by using conventional reduction methods (e.g., LiAlH₄, NaBH₄, etc). which can then be derivatised using the following processes:

i) converting the —CH₂OH group to A —CH₂OR^{I} group with an appropriate halide R^{I}X (such as a hydrocarbylhalide, for example a alkylhalide, arylhalide, or the like, in which the halogen can be chloro, bromo or iodo) in a polar aprotic solvent (e.g., acetonitrile, THF, DMF, . . . ) in the presence of a base (e.g., NaH, K₂CO₃, Et₃N, . . . ) at a temperature increasing from 0° C. to 90° C. to provide the desired ethers,
(ii) oxidising the —CH₂OH group to an aldehyde group —CHO (e.g., DESS MARTIN reagent, IBX, . . . ) that is subsequently submitted to reductive amination conditions with primary or secondary amines R^{a}R^{b}NH in order to obtain the —CH₂—NR^{a}R^{b} group,
(iii) convert the —CH₂OH group with Cl—SO₂R to a mesylate or tosylate in the presence of a base and then reacting the resulting sulfonates with primary or secondary amines R^{a}R^{b}NH forming the —CH₂—NR^{a}R^{b} group,
as summarised in Scheme 2 below:

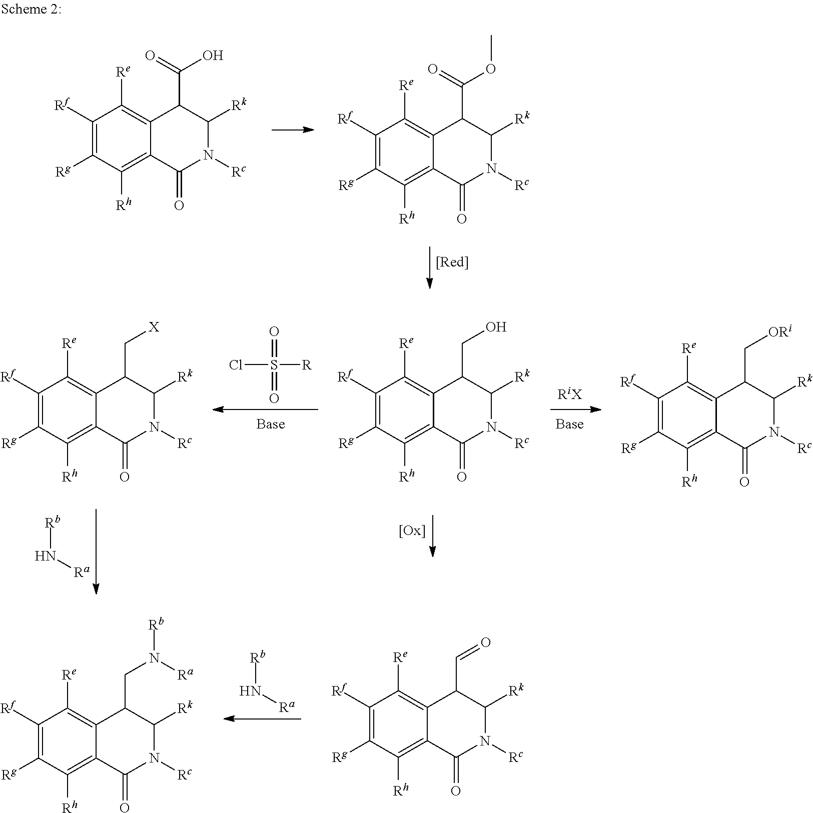

Methods according to process (ii) are known to those skilled in the art as are those of process (iii) (cf. e.g., Molecules, 2006, 11, 403, journal of heterocyclic chemistry, 2003, 40, 795).

The compounds of the present invention in which the dotted line “a” in formula (I) represents a double bond can be prepared by treating an ester of the 1-oxo-1,2,3,4-tetrahydroisoquinoline-4-carboxylic acid with a strong base (e.g., n-BuLi, NaHMDS . . . ) at low temperature, (preferably −80° C.) in a polar aprotic solvent (e.g., THF, DMF . . . ) and then trating the resulting reaction mixture with a hypochloroselenoite or a sulfinic chloride to provide the desired 1,2-dihydroisoquinoline ester. The latter is then converted to amides by classical methods. More detailed descriptions can be found in the following references (JOC, 2005, 70, 6496, Tetrahedron, 2006, 62, 9705). Another approach using isoquinolin-1(2H)-one as starting material is also available from literature (WO 98/00423). This process is summarised in Scheme 3 below:

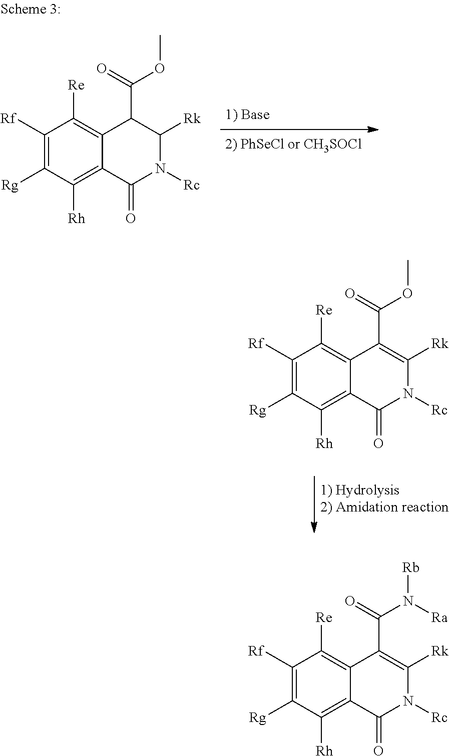

### EXAMPLES

The following examples are provided for the purpose of illustrating the present invention and by no means should be interpreted to limit the scope of the present invention.
Part A represents the preparation of the compounds, whereas Part B represents the pharmacological examples.

TABLE 1
Structures of example compounds of the invention and their respective codes.
(I)

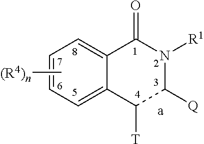

| | R¹ | Q | L²* | R³ | R⁴ | a |
|---|---|---|---|---|---|---|
| C1 | 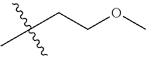 | 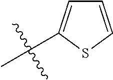 | 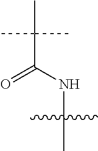 | 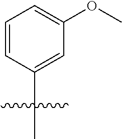 | — | single |
| C2 | 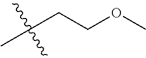 | 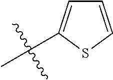 | 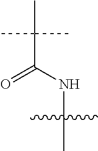 | 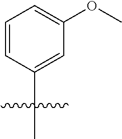 | — | single |
| C3 | 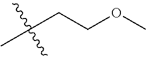 | 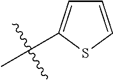 | 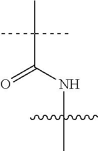 | 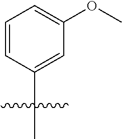 | 7-NO₂ | single |
| C4 | 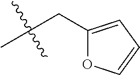 | 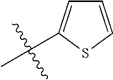 | 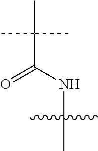 | 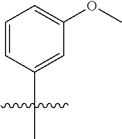 | — | single |
| C5 | Et | 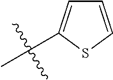 | 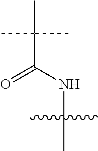 | 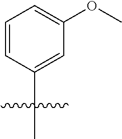 | — | single |
| C6 | 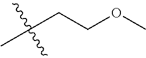 | 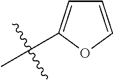 | 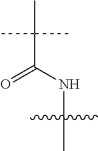 | 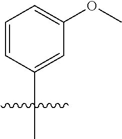 | — | single |
| C7 | 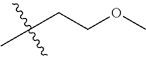 | 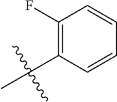 | 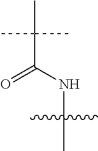 | 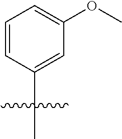 | — | single |
| C8 | 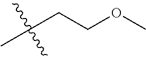 | 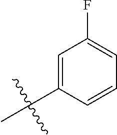 | 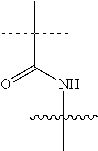 | 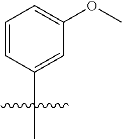 | — | single |
| C9 | 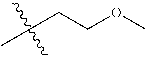 | 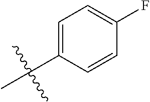 | 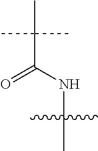 | 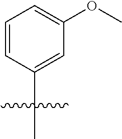 | — | single |
| C10 | 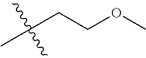 | 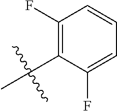 | 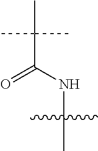 | 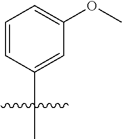 | — | single |
| C11 | 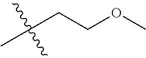 | 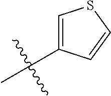 | 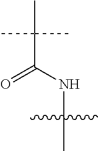 | 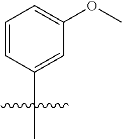 | — | single |
| C12 | 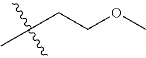 | 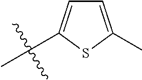 | 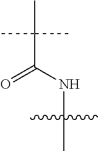 | 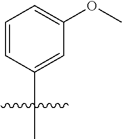 | — | single |
| C13 | 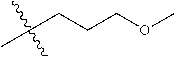 | 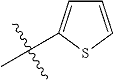 | 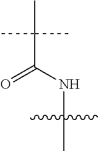 | 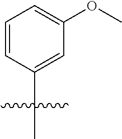 | — | single |
| | 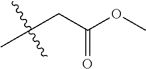 | 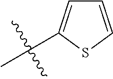 | 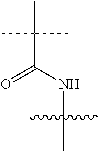 | 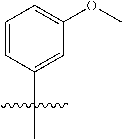 | | |
| C1 | 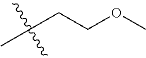 | 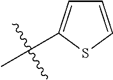 | 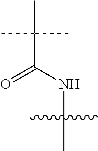 | 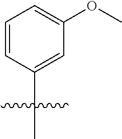 | — | single |
| C2 | 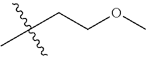 | 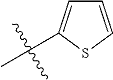 | 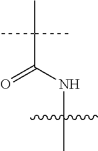 | 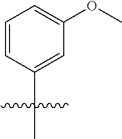 | — | single |
| C3 | 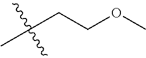 | 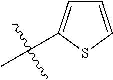 | 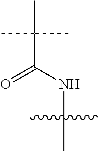 | 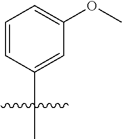 | 7-NO₂ | single |
| C4 | 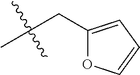 | 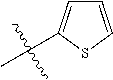 | 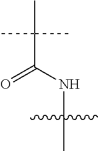 | 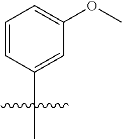 | — | single |
| C5 | Et | 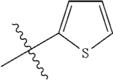 | 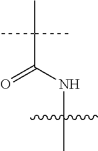 | 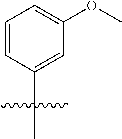 | — | single |
| C6 | 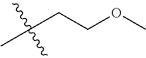 | 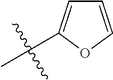 | 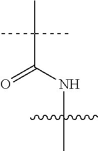 | 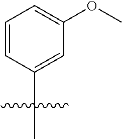 | — | single |
| C7 | 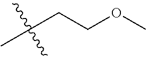 | 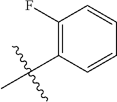 | 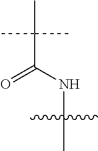 | 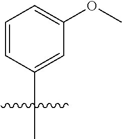 | — | single |
| C8 | 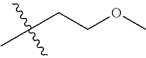 | 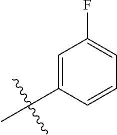 | 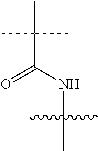 | 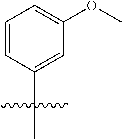 | — | single |
| C9 | 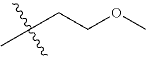 | 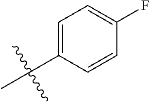 | 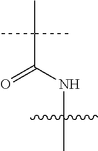 | 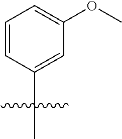 | — | single |
| C10 | 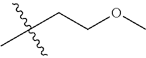 | 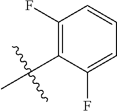 | 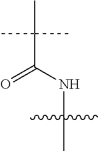 | 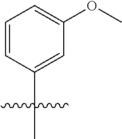 | — | single |
| C11 | 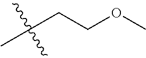 | 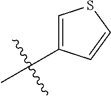 | 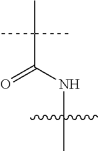 | 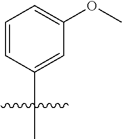 | — | single |
| C12 | 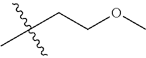 | 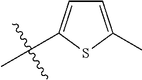 | 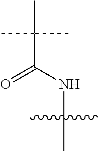 | 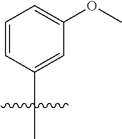 | — | single |
| C13 | 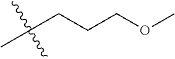 | 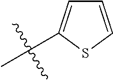 | 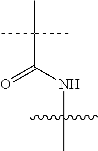 | 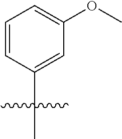 | — | single |
| | 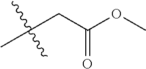 | 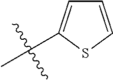 | 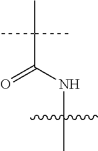 | 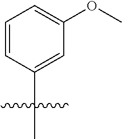 | | |
| C14 | 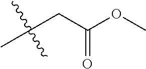 | 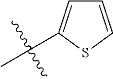 | 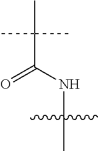 | 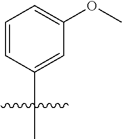 | — | single |
| C15 | 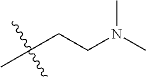 | 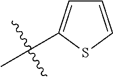 | 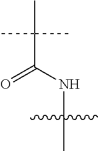 | 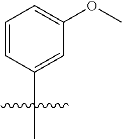 | — | single |
| C16 | 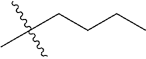 | 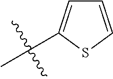 | 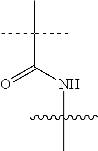 | 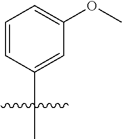 | — | single |
| C17 | 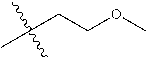 | 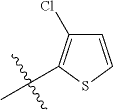 | 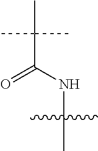 | 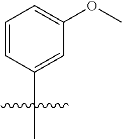 | — | single |
| C18 | 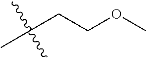 | 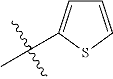 | 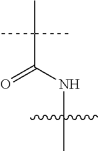 | 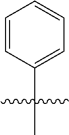 | — | single |
| C19 | 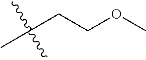 | 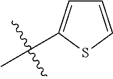 | 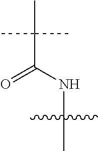 | 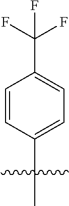 | — | single |
| C20 | 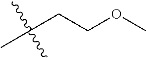 | 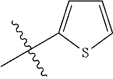 | 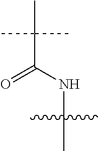 | 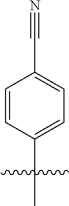 | — | single |
| C21 | 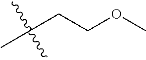 | 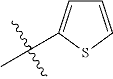 | 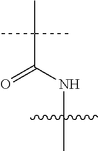 | 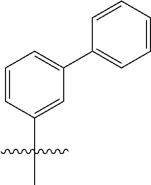 | — | single |
| C22 | 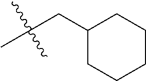 | 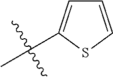 | 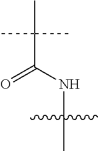 | 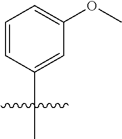 | — | single |
| C23 | 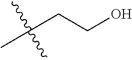 | 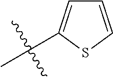 | 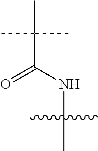 | 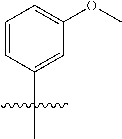 | — | single |
| C24 | 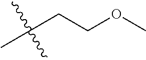 | 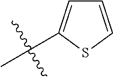 | 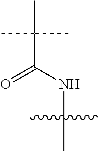 | 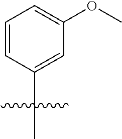 | 8-Cl | single |
| C25 | 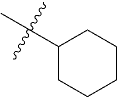 | 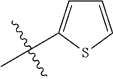 | 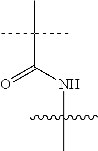 | 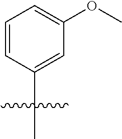 | — | single |
| C26 | 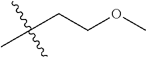 | 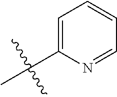 | 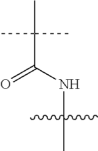 | 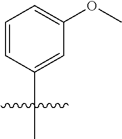 | — | single |
| C27 | 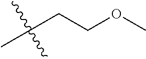 | 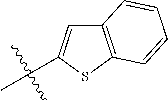 | 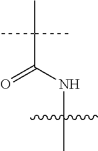 | 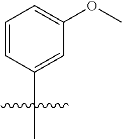 | — | single |
| C28 | 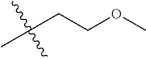 | 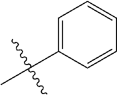 | 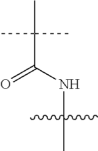 | 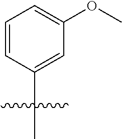 | — | single |
| C29 | 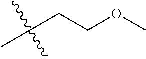 | 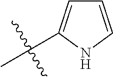 | 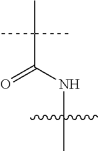 | 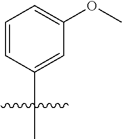 | — | single |
| C30 | 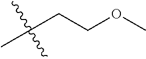 | 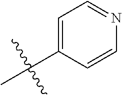 | 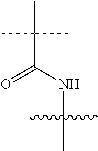 | 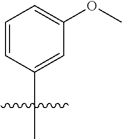 | — | single |
| C31 | 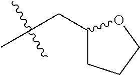 | 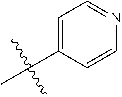 | 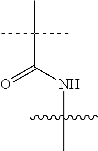 | 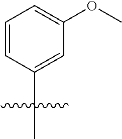 | — | single |
| C32 | 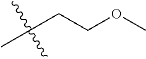 | 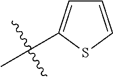 | 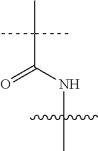 | 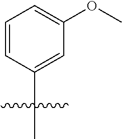 | — | single |
| C33 | 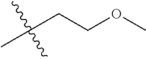 | 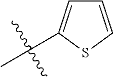 | 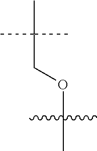 | H | — | single |
| C34 | 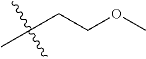 | 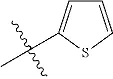 | 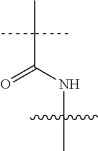 | 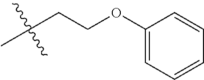 | — | single |
| C35 | 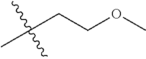 | 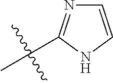 | 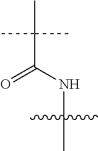 | 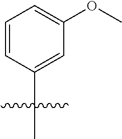 | — | single |
| C36 | 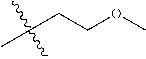 | 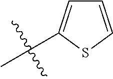 | 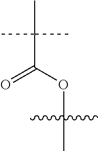 | H | — | single |
| C37 | 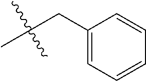 | 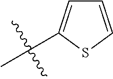 | 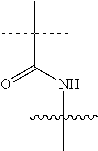 | 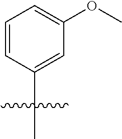 | — | single |
| C38 | 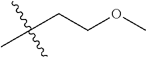 | 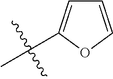 | 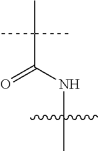 | 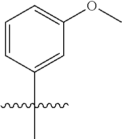 | — | single |
| C39 | 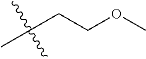 | 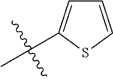 | 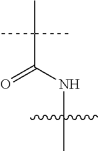 | 5-Me | — | single |
| C40 | 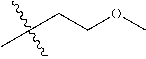 | 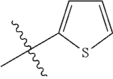 | 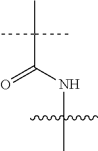 | 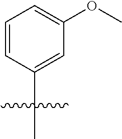 | 6-Me | single |
| C41 | 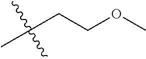 | 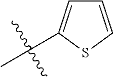 | 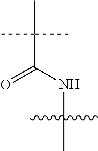 | 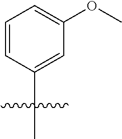 | 7-Me | single |
| C42 | 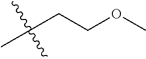 | 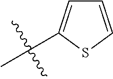 | 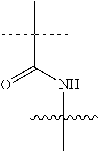 | 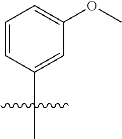 | 8-Me | single |
| C43 | 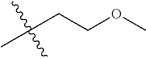 | 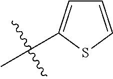 | 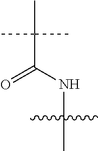 | 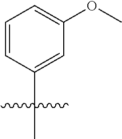 | 7-Cl | single |
| C44 | 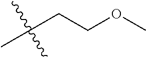 | 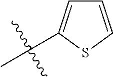 | 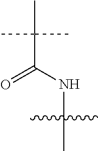 | 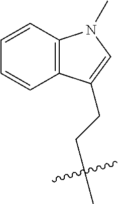 | — | single |
| C45 | 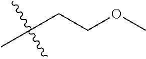 | 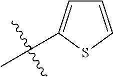 | 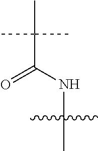 | 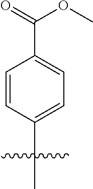 | — | single |
| C46 | 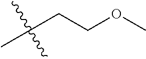 | 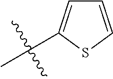 | 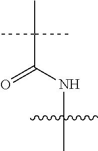 | 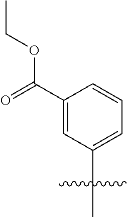 | — | single |
| C47 | 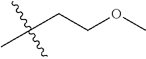 | 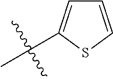 | 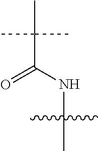 | 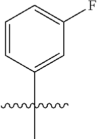 | — | single |
| C48 | 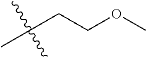 | 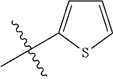 | 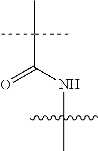 | 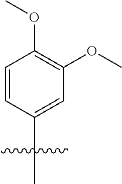 | — | single |
| C49 | 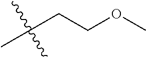 | 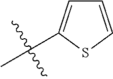 | 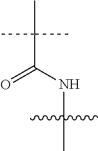 | 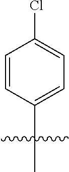 | — | single |
| C50 | 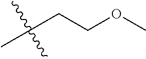 | 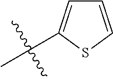 | 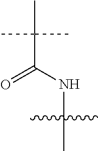 | 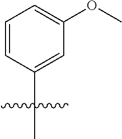 | 7- OMe | single |
| C51 | 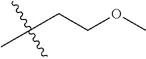 | 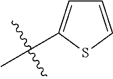 | 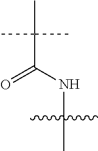 | 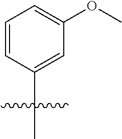 | 8-F | single |
| C52 | 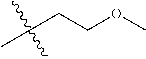 | 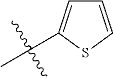 | 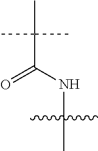 | 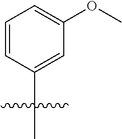 | 7-F | single |
| C53 | 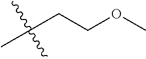 | 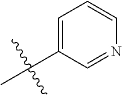 | 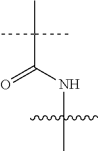 | 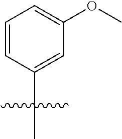 | — | single |
| C54 | 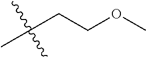 | 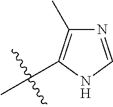 | 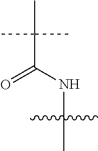 | 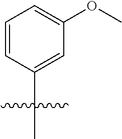 | — | single |
| C55 | 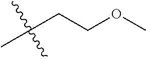 | 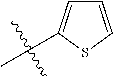 | 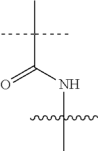 | 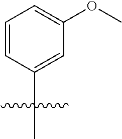 | 6-Cl | single |
| C56 | 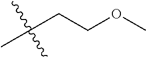 | 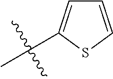 | 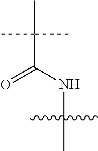 | 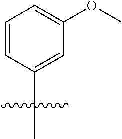 | 7-I | single |
| C57 | 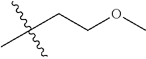 | t-Bu | 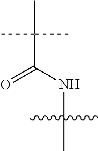 | 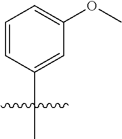 | — | single |
| C58 | 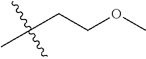 | Et | 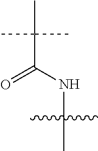 | 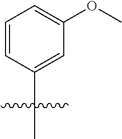 | — | single |
| C59 | 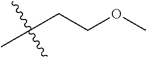 | 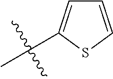 | 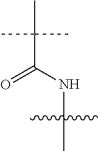 | 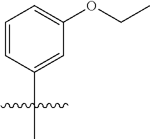 | — | single |
| C60 | 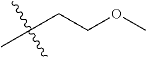 | 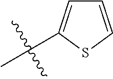 | 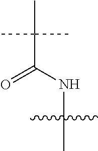 | 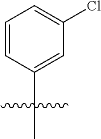 | — | single |
| C61 | 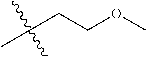 | 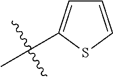 | 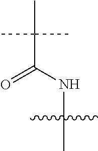 | 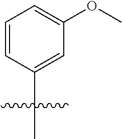 | 5-F | single |
| C62 | 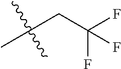 | 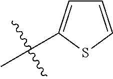 | 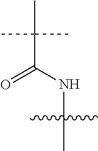 | 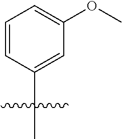 | — | single |
| C63 | 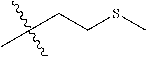 | 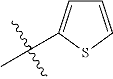 | 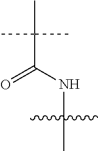 | 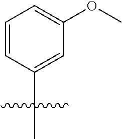 | — | single |
| C64 | 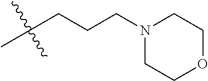 | 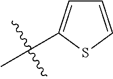 | 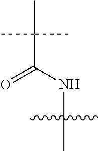 | 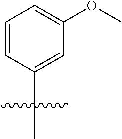 | — | single |
| C65 | 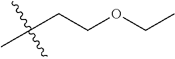 | 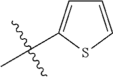 | 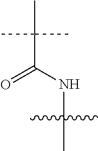 | 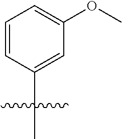 | — | single |
| C66 | 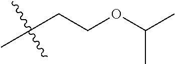 | 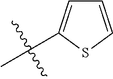 | 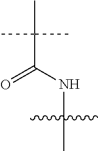 | 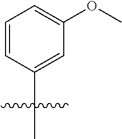 | — | single |
| C67 | 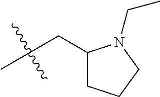 | 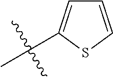 | 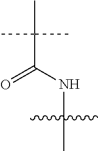 | 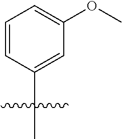 | — | single |
| C68 | 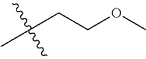 | 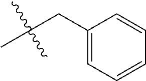 | 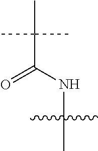 | 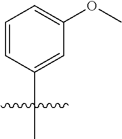 | — | single |
| C69 | 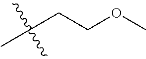 | Me | 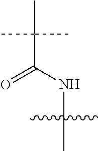 | 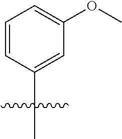 | — | single |
| C70 | 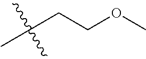 | 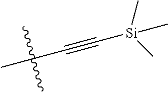 | 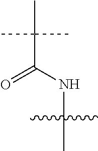 | 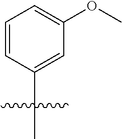 | — | single |
| C71 | 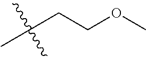 | 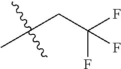 | 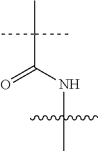 | 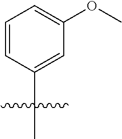 | — | single |
| C72 | 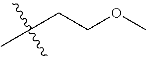 | 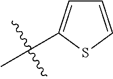 | 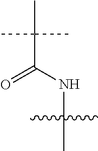 | 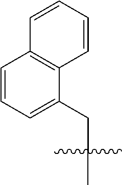 | — | single |
| C73 | 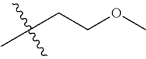 | 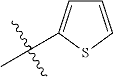 | 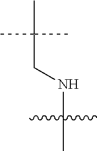 | 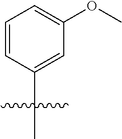 | — | single |
| C74 | 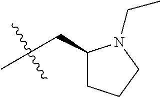 | 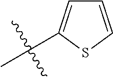 | 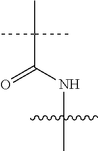 | 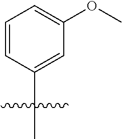 | — | single |
| C75 | 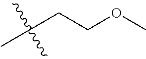 | 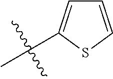 | 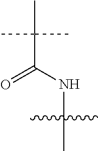 | 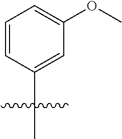 | 6,7- OMe | single |
| C76 | 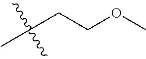 | 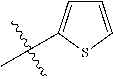 | 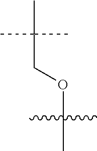 | 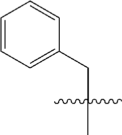 | — | single |
| C77 | 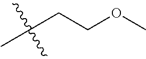 | 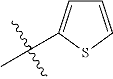 | 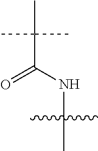 | 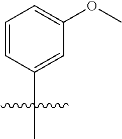 | 6-F | single |
| C78 | 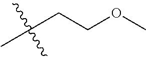 | 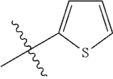 | 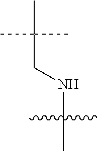 | 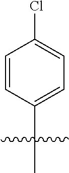 | — | single |
| C79 | 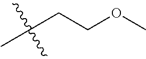 | 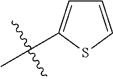 | 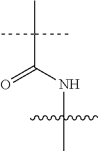 | 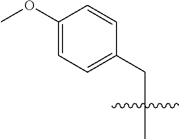 | — | single |
| C80 | 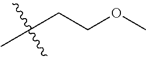 | 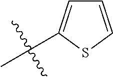 | 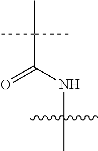 | 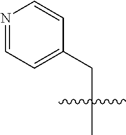 | — | single |
| C81 | 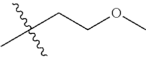 | 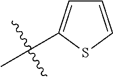 | 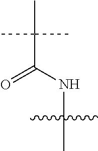 | 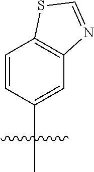 | — | single |
| C82 | 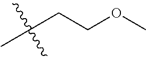 | 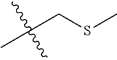 | 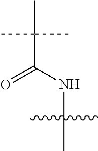 | 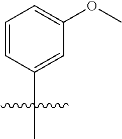 | — | single |
| C83 | 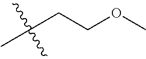 | 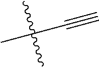 | 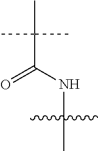 | 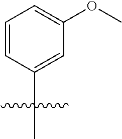 | — | single |
| C84 | 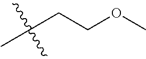 | 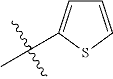 | 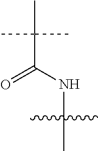 | 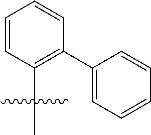 | — | single |
| C85 | 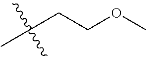 | 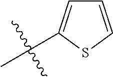 | 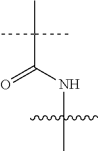 | 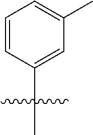 | — | single |
| C86 | 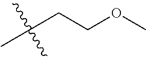 | 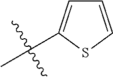 | 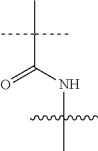 | 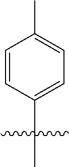 | — | single |
| C87 | 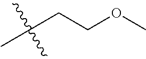 | 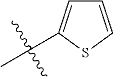 | 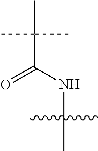 | 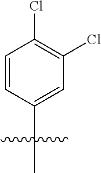 | — | single |
| C88 | 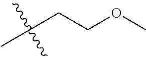 | 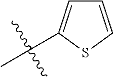 | 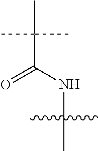 | 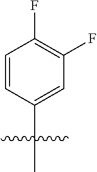 | — | single |
| C89 | 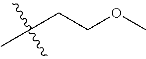 | 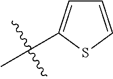 | 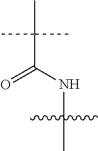 | 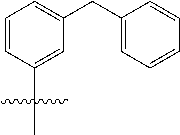 | — | single |
| C90 | 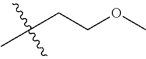 | 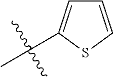 | 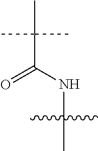 | 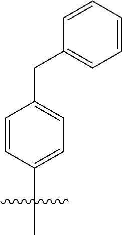 | — | single |
| C91 | 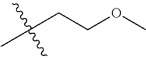 | 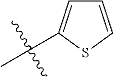 | 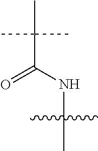 | 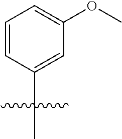 | 5,8-F | single |
| C92 | 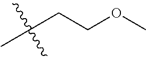 | 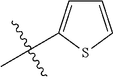 | 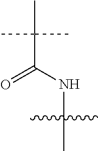 | 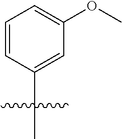 | 7-CF₃ | single |
| C93 | 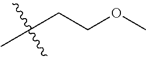 | 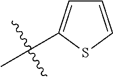 | 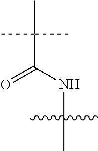 | 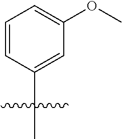 | 8-F, 7-Me | single |
| C94 | 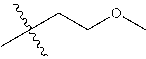 | 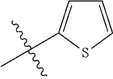 | 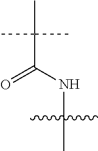 | 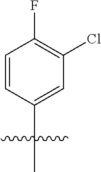 | — | single |
| C95 | 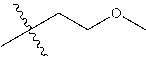 | 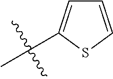 | 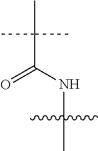 | 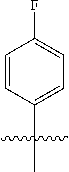 | — | single |
| C96 | 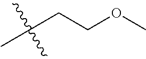 | 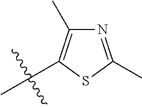 | 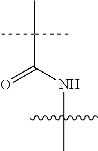 | 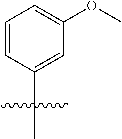 | — | single |
| C97 | 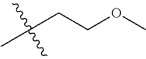 | 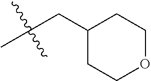 | 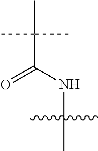 | 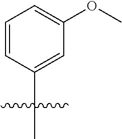 | — | single |
| C98 | 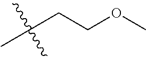 | 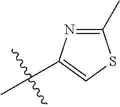 | 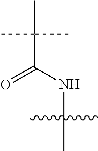 | 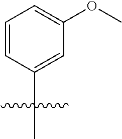 | — | single |
| C99 | 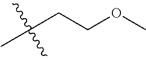 | 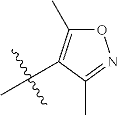 | 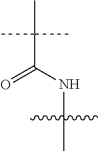 | 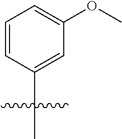 | — | single |
| C100 | 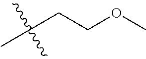 | 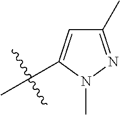 | 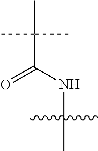 | 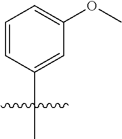 | — | single |
| C101 | 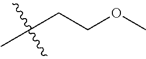 | 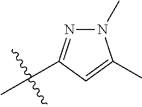 | 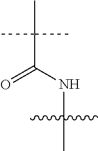 | 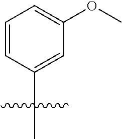 | — | single |
| C102 | 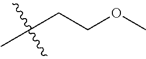 | 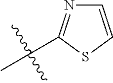 | 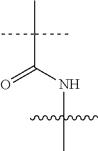 | 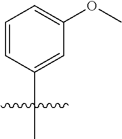 | — | single |
| C103 | 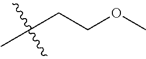 | 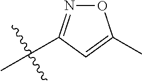 | 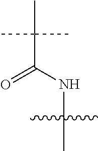 | 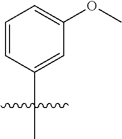 | — | single |
| C104 | 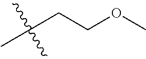 | 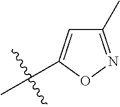 | 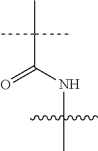 | 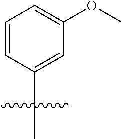 | — | single |
| C105 | 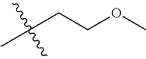 | 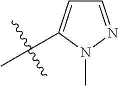 | 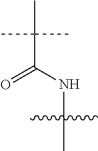 | 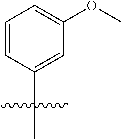 | — | single |
| C106 | 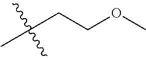 | 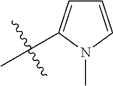 | 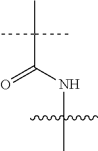 | 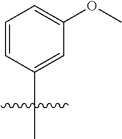 | — | single |
| C107 | 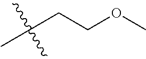 | 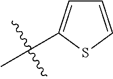 | 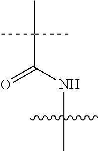 | 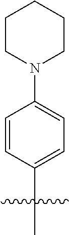 | — | single |
| C108 | 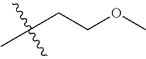 | 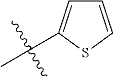 | 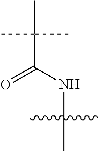 | 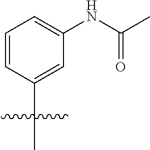 | — | single |
| C109 | 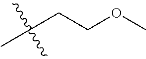 | 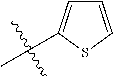 | 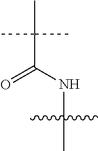 | 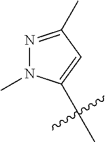 | — | single |
| C110 | 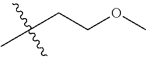 | 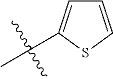 | 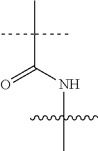 | 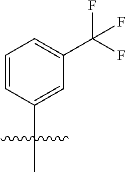 | — | single |
| C111 | 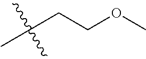 | 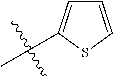 | 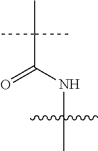 | 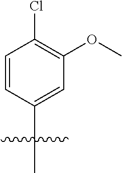 | — | single |
| C112 | 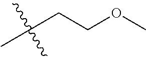 | 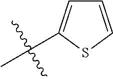 | 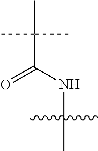 | 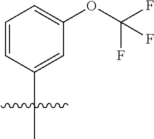 | — | single |
| C113 | 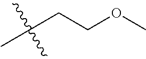 | 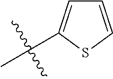 | 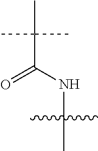 | 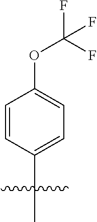 | — | single |
| C114 | 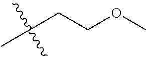 | 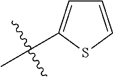 | 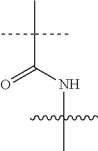 | 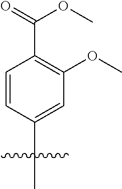 | — | single |
| C115 | 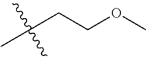 | 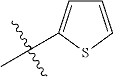 | 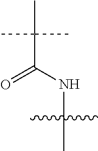 | 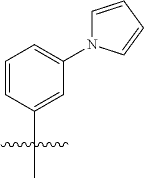 | — | single |
| C116 | 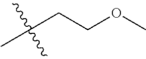 | 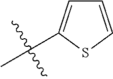 | 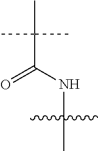 | 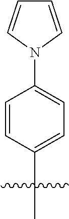 | — | single |
| C117 | 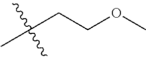 | 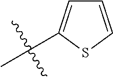 | 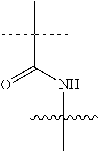 | 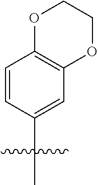 | — | single |
| C118 | 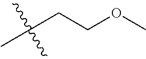 | 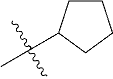 | 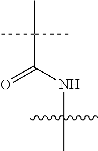 | 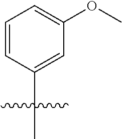 | — | single |
| C119 | 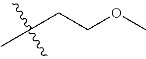 | 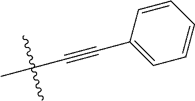 | 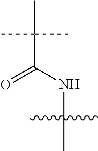 | 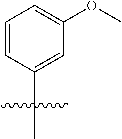 | — | single |
| C120 | 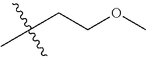 | 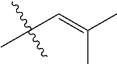 | 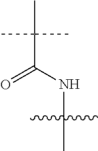 | 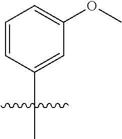 | — | single |
| C121 | 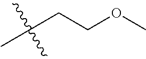 | 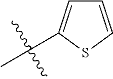 | 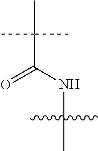 | 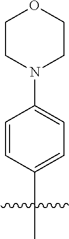 | — | single |
| C122 | 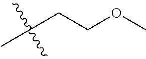 | 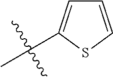 | 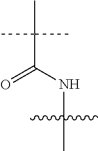 | 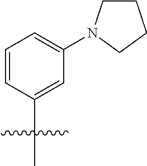 | — | single |
| C123 | 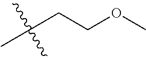 | 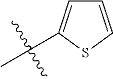 | 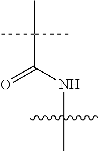 | 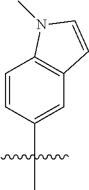 | — | single |
| C124 | 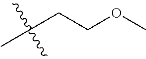 | 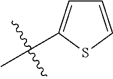 | 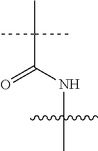 | 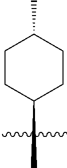 | — | single |
| C125 | 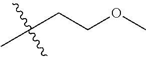 | 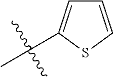 | 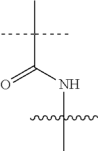 | 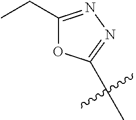 | — | single |
| C126 | 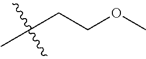 | 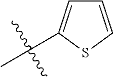 | 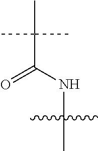 | 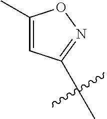 | — | single |
| C127 | 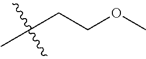 | 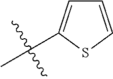 | 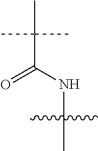 | 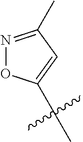 | — | single |
| C128 | 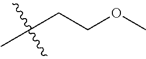 | 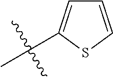 | 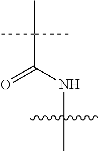 | 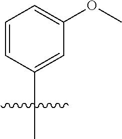 | 8-F, 7-OMe | single |
| C129 | 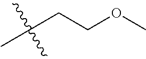 | 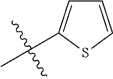 | 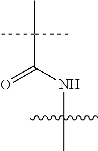 | 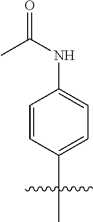 | — | single |
| C130 | 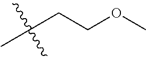 | 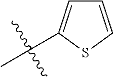 | 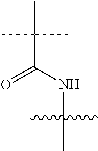 | 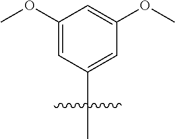 | — | single |
| C131 | 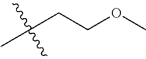 | 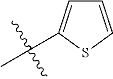 | 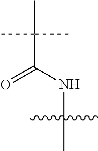 | 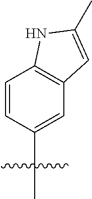 | — | single |
| C132 | 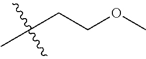 | 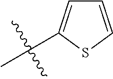 | 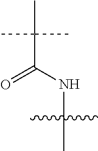 | 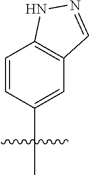 | — | single |
| C133 | 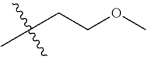 | 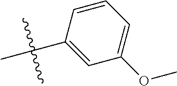 | 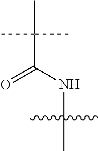 | 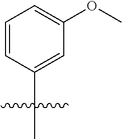 | — | single |
| C134 | 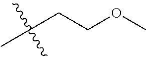 | 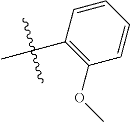 | 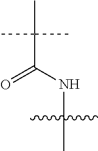 | 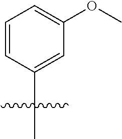 | — | single |
| C135 | 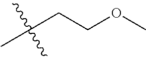 | 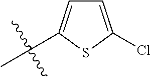 | 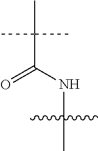 | 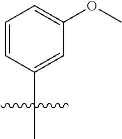 | — | single |
| C136 | 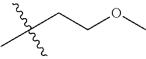 | 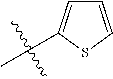 | 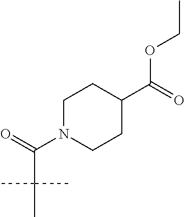 | | — | single |
| C137 | 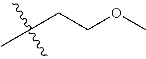 | 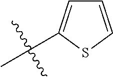 | 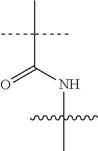 | 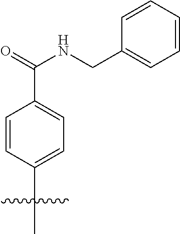 | — | single |
| C138 | 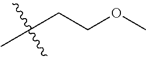 | 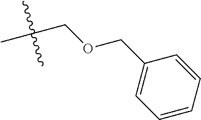 | 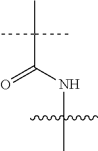 | 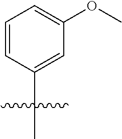 | — | single |
| C139 | 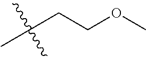 | 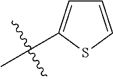 | 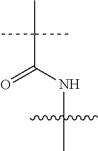 | 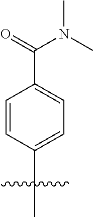 | — | single |
| C140 | 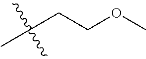 | 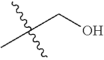 | 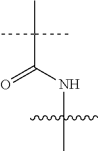 | 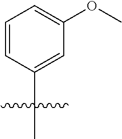 | — | single |
| C141 | 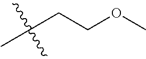 | 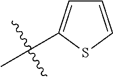 | 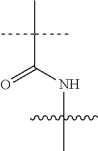 | 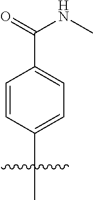 | — | single |
| C142 | 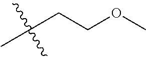 | 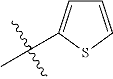 | 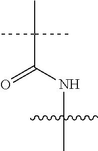 | 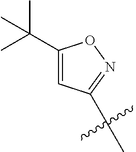 | — | single |
| C143 | 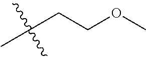 | CN | 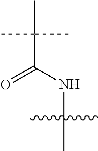 | 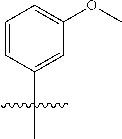 | — | single |
| C144 | 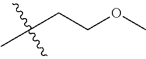 | 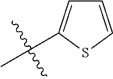 | 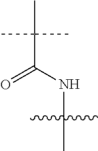 | 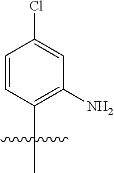 | — | single |
| C145 | 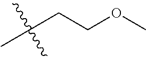 | 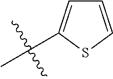 | 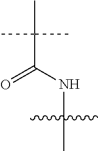 | 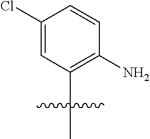 | — | single |
| C146 | 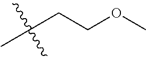 | 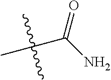 | 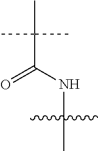 | 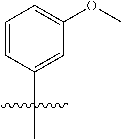 | — | single |
| C147 | 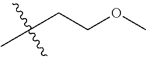 | 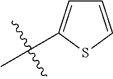 | 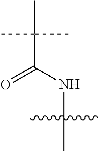 | 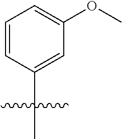 | 7-NH2 | single |
| C148 | 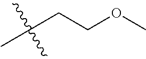 | 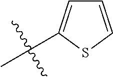 | 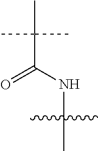 | 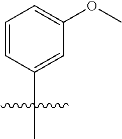 | 5-OMe | single |
| C149 | 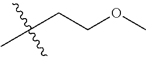 | 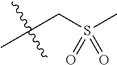 | 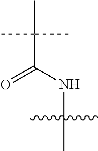 | 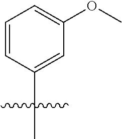 | — | single |
| C150 | 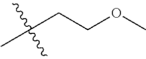 | 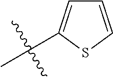 | 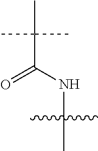 | 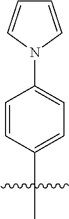 | 8-F, 7-OMe | single |
| C151 | 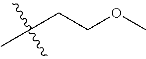 | 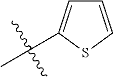 | 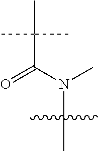 | 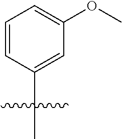 | — | single |
| C152 | 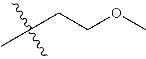 | 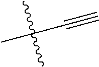 | 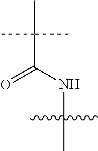 | 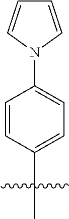 | — | single |
| C153 | 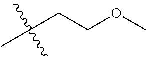 | 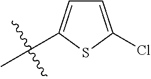 | 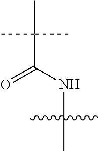 | 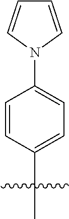 | — | single |
| C154 | 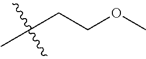 | 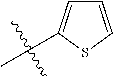 | 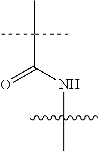 | 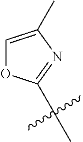 | — | single |
| C155 | 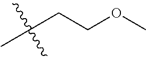 | 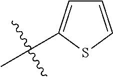 | 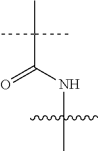 | 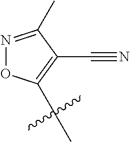 | — | single |
| C156 | 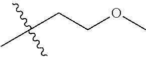 | 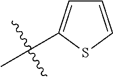 | 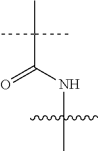 | 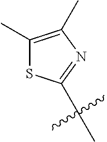 | — | single |
| C157 | 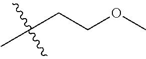 | 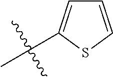 | 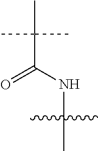 | 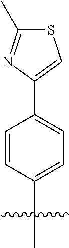 | — | single |
| C158 | 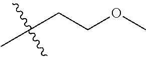 | 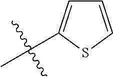 | 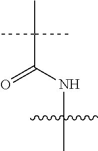 | 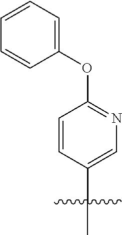 | — | single |
| C159 | 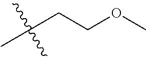 | 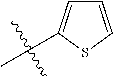 | 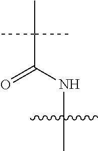 | 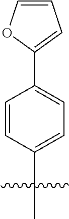 | — | single |
| C160 | 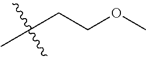 | 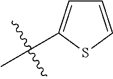 | 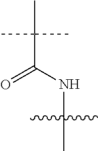 | 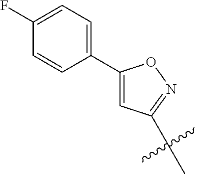 | — | single |
| C161 | 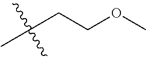 | 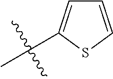 | 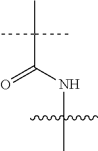 | 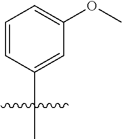 | — | single |
| C162 | 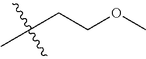 | 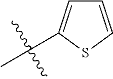 | 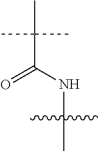 | 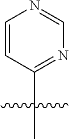 | — | single |
| C163 | 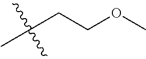 | 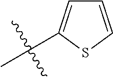 | 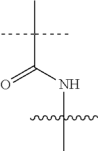 | 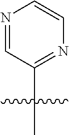 | — | single |
| C164 | 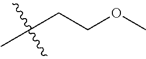 | 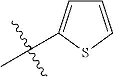 | 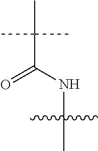 | 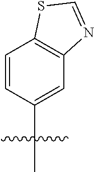 | — | single |
| C165 | 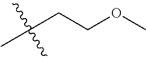 | 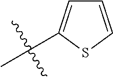 | 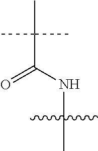 | 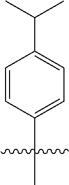 | — | single |
| C166 | 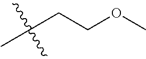 | 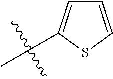 | 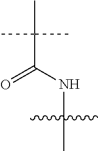 | 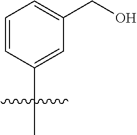 | — | single |
| C167 | 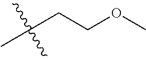 | 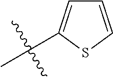 | 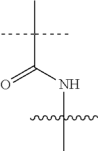 | 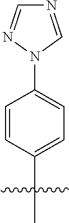 | — | single |
| C168 | 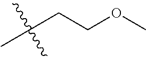 | 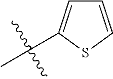 | 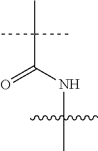 | 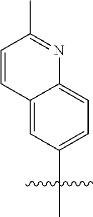 | — | single |
| C169 | 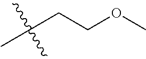 | 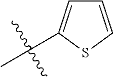 | 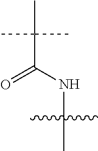 | 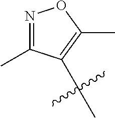 | — | single |
| C170 | 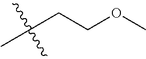 | 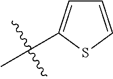 | 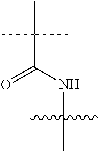 | 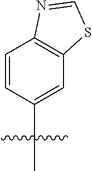 | — | single |
| C171 | 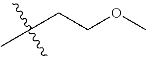 | 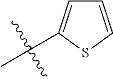 | 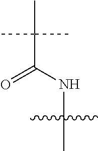 | 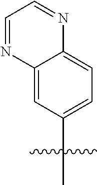 | — | single |
| C172 | 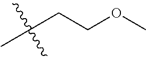 | 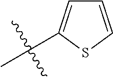 | 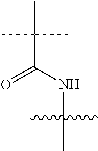 | 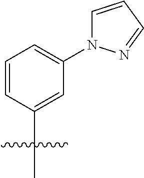 | — | single |
| C173 | 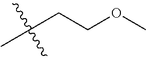 | 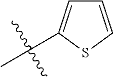 | 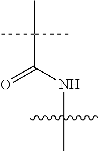 | 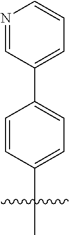 | — | single |
| C174 | 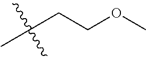 | 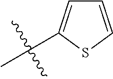 | 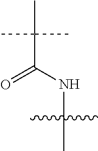 | 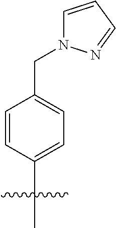 | — | single |
| C175 | 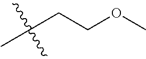 | 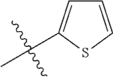 | 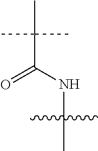 | 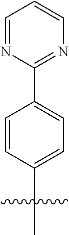 | — | single |
| C176 | 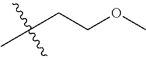 | 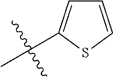 | 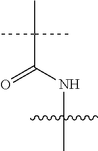 | 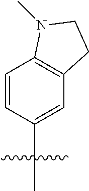 | — | single |
| C177 | 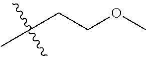 | 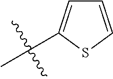 | 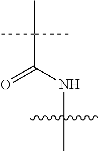 | 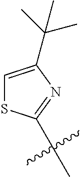 | — | single |
| C178 | 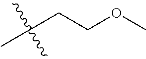 | 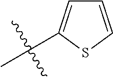 | 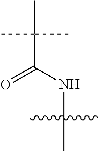 | 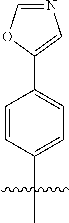 | — | single |
| C179 | 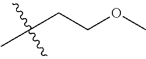 | 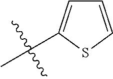 | 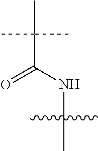 | 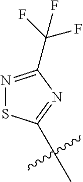 | — | single |
| C180 | 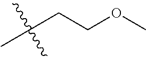 | 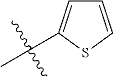 | 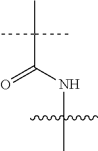 | 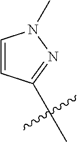 | — | single |
| C181 | 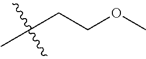 | 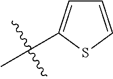 | 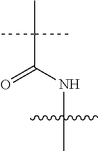 | 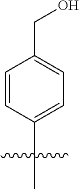 | — | single |
| C182 | 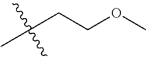 | 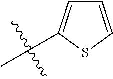 | 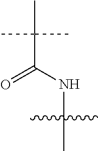 | 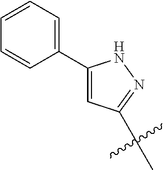 | — | single |
| C183 | 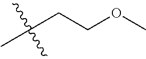 | 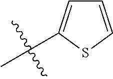 | 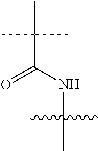 | 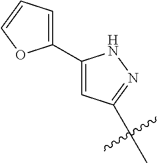 | — | single |
| C184 | 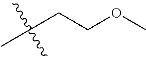 | 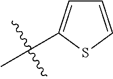 | 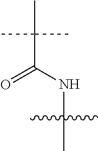 | 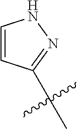 | — | single |
| C185 | 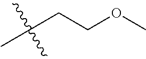 | 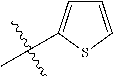 | 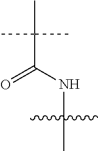 | 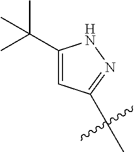 | — | single |
| C186 | 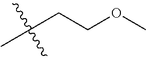 | 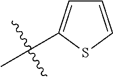 | 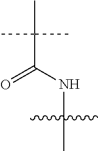 | 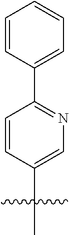 | — | single |
| C187 | 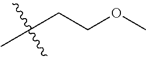 | 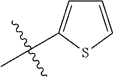 | 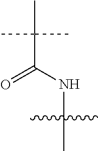 | 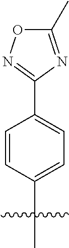 | — | single |
| C188 | 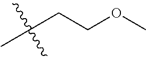 | 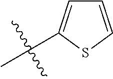 | 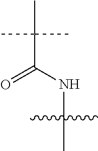 | 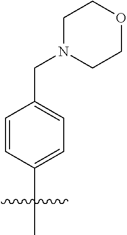 | — | single |
| C189 | 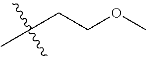 | 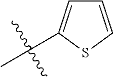 | 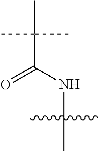 | 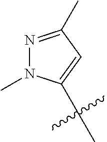 | — | single |
| C190 | 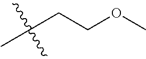 | 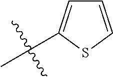 | 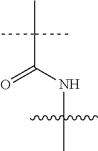 | 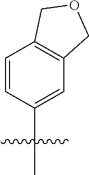 | — | single |
| C191 | 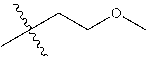 | 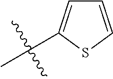 | 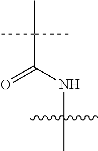 | 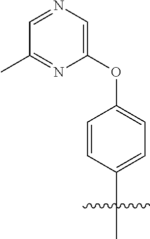 | — | single |
| C192 | 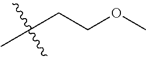 | 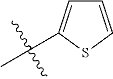 | 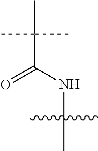 | 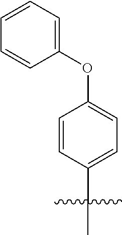 | — | single |
| C193 | 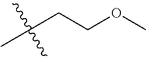 | 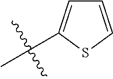 | 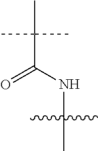 | 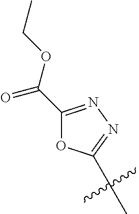 | — | single |
| C194 | 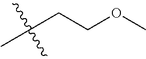 | 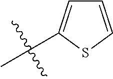 | 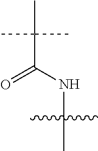 | 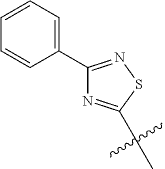 | — | single |
| C195 | 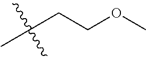 | 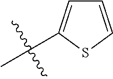 | 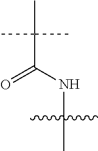 | 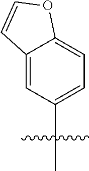 | — | single |
| C196 | 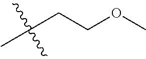 | 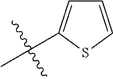 | 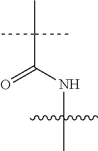 | 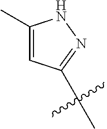 | — | single |
| C197 | 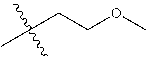 | 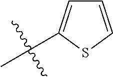 | 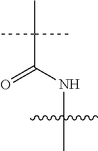 | 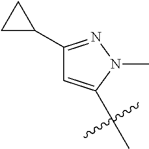 | — | single |
| C198 | 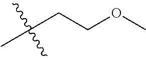 | 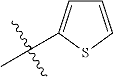 | 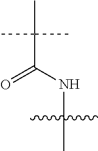 | 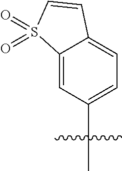 | — | single |
| C199 | 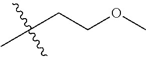 | 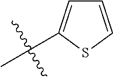 | 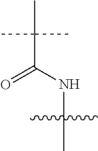 | 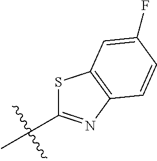 | — | single |
| C200 | 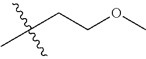 | 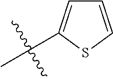 | 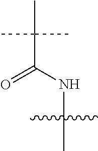 | 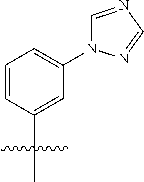 | — | single |
| C201 | 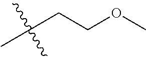 | 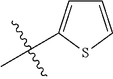 | 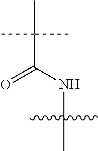 | 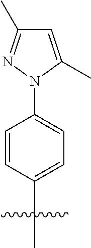 | — | single |
| C202 | 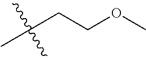 | 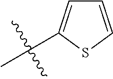 | 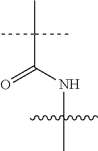 | 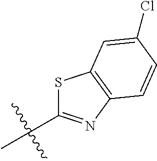 | — | single |
| C203 | 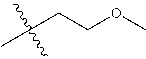 | 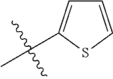 | 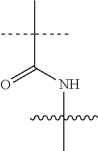 | 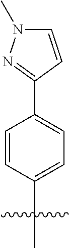 | — | single |
| C204 | 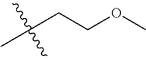 | 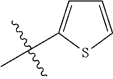 | 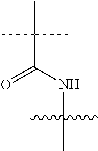 | 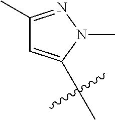 | — | single |
| C205 | 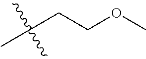 | 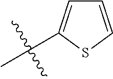 | 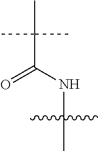 | 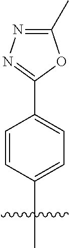 | — | single |
| C206 | 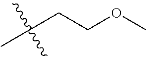 | 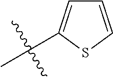 | 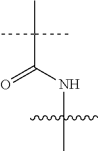 | 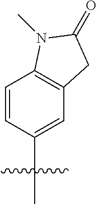 | — | single |
| C207 | 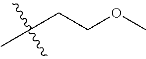 | 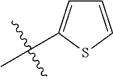 | 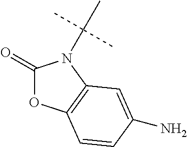 | | — | single |
| C208 | 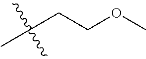 | 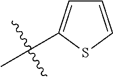 | 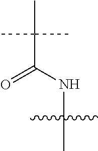 | 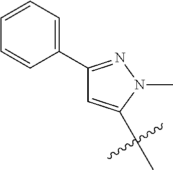 | — | single |
| C209 | 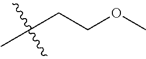 | 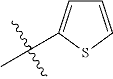 | 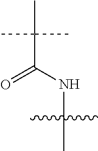 | 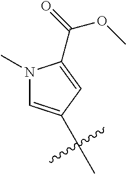 | — | single |
| C210 | 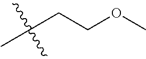 | 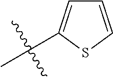 | 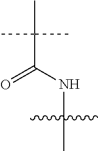 | 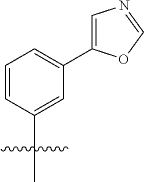 | — | single |
| C211 | 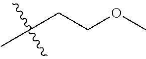 | 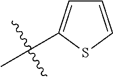 | 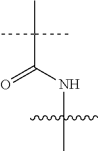 | 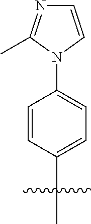 | — | single |
| C212 | 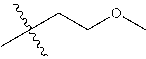 | 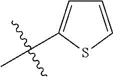 | 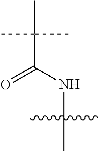 | 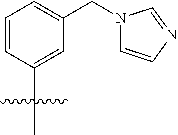 | — | single |
| C213 | 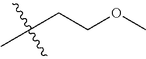 | 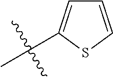 | 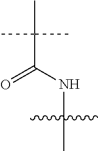 | 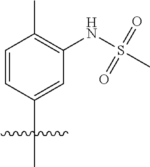 | — | single |
| C214 | 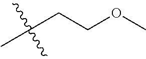 | 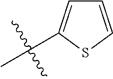 | 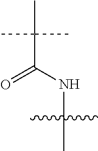 | 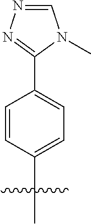 | — | single |
| C215 | 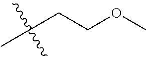 | 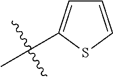 | 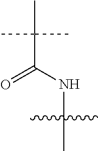 | 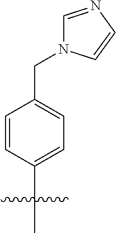 | — | single |
| C216 | 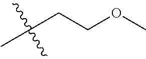 | 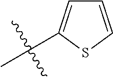 | 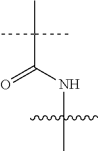 | 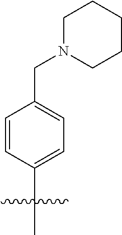 | — | single |
| C217 | 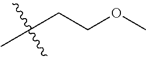 | 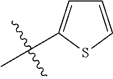 | 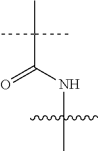 | 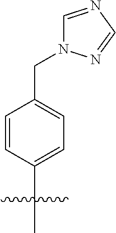 | — | single |
| C218 | 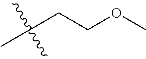 | 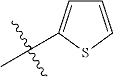 | 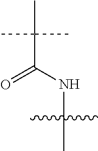 | 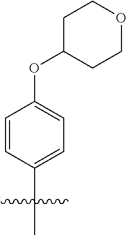 | — | single |
| C219 | 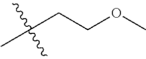 | 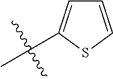 | 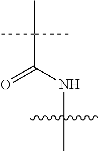 | 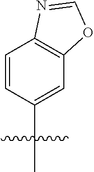 | — | single |
| C220 | 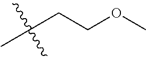 | 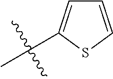 | 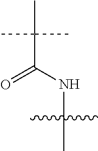 | 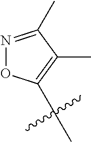 | — | single |
| C221 | 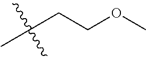 | 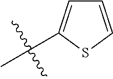 | 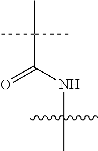 | 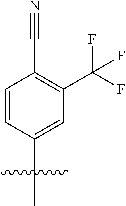 | — | single |
| C222 | 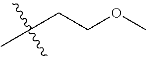 | 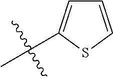 | 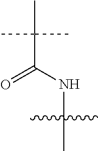 | 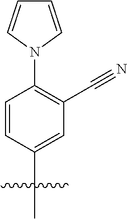 | — | single |
| C223 | 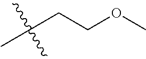 | 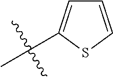 | 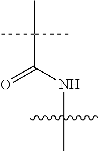 | 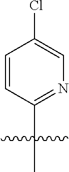 | — | single |
| C224 | 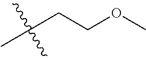 | 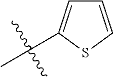 | 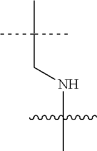 | 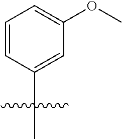 | — | single |
| C225 | 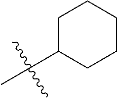 | 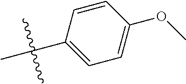 | 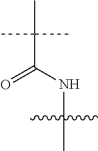 | 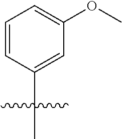 | — | single |
| C226 | 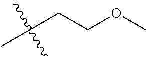 | 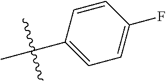 | 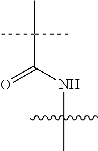 | 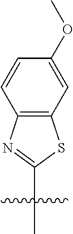 | — | single |
| C227 | 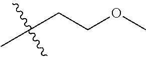 | 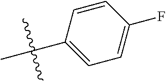 | 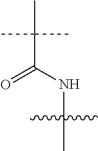 | 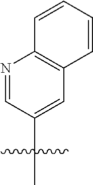 | — | single |
| C228 | 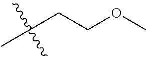 | 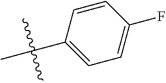 | 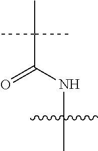 | 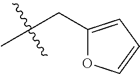 | — | single |
| C229 | 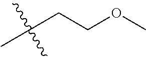 | 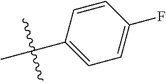 | 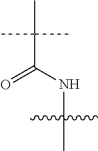 | 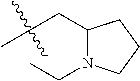 | — | single |
| C230 | 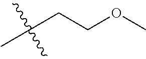 | 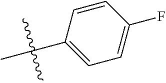 | 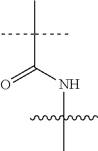 | 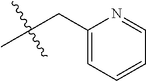 | — | single |
| C231 | Me | 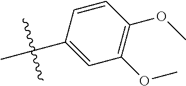 | 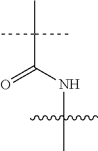 | 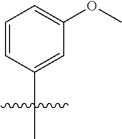 | — | single |
| C232 | 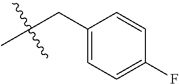 | 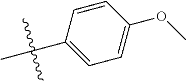 | 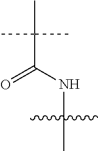 | 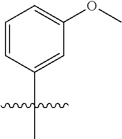 | — | single |
| C233 | Me | 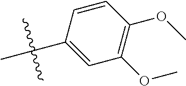 | 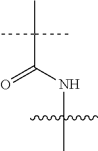 | 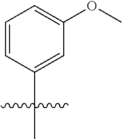 | — | single |
| C234 | Me | 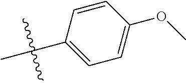 | 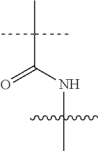 | 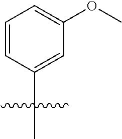 | — | single |
| C235 | Me | 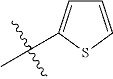 | 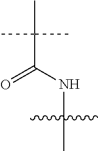 | 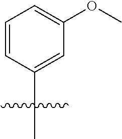 | — | single |
| C236 | Me | 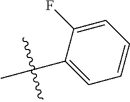 | 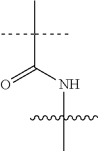 | 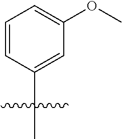 | — | single |
| C237 | Me | 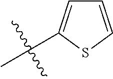 | 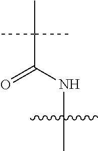 | 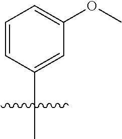 | — | single |
| C238 | 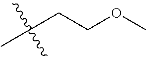 | 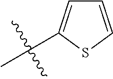 | 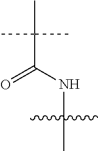 | 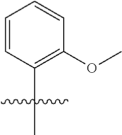 | — | single |
| C239 | 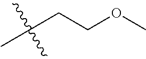 | 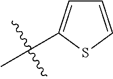 | 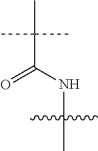 | 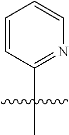 | — | single |
| C240 | 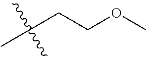 | 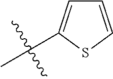 | 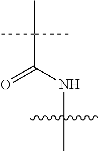 | 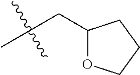 | — | single |
| C241 | 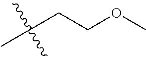 | 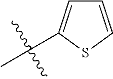 | 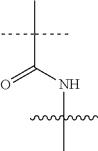 | 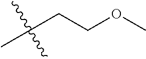 | — | single |
| C242 | 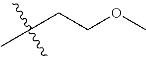 | 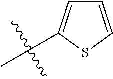 | 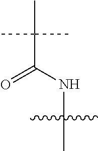 | 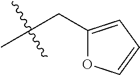 | — | single |
| C243 | 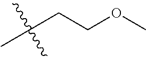 | 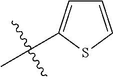 | 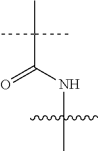 | 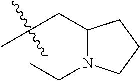 | — | single |
| C244 | 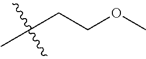 | 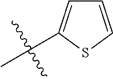 | 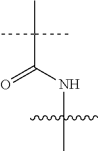 | 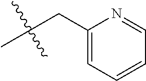 | — | single |
| C245 | 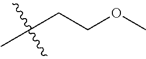 | 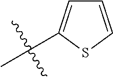 | 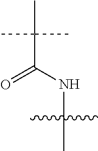 | 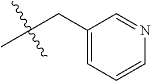 | — | single |
| C246 | 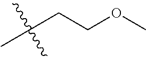 | 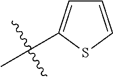 | 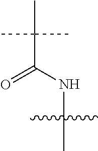 | 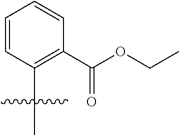 | — | single |
| C247 | 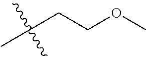 | 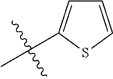 | 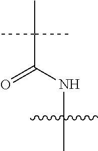 | 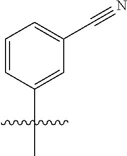 | — | single |
| C248 | 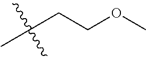 | 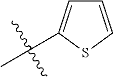 | 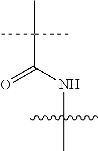 | 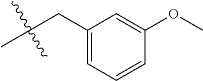 | — | single |
| C249 | 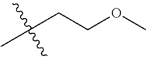 | 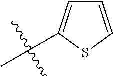 | 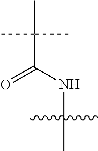 | 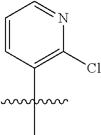 | — | single |
| C250 | 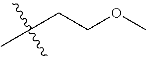 | 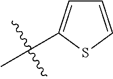 | 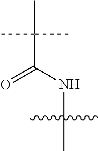 | 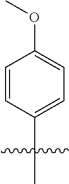 | — | single |
| C251 | 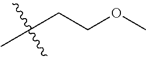 | 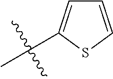 | 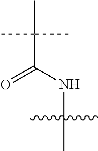 | 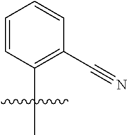 | — | single |
| C252 | 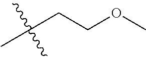 | 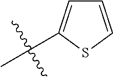 | 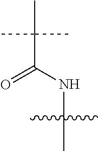 | 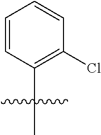 | — | single |
| C253 | 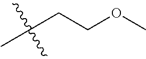 | 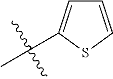 | 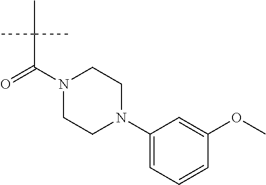 | | — | single |
| C254 | 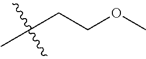 | 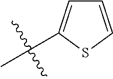 | 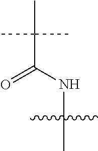 | 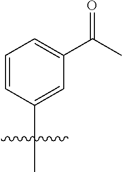 | — | single |
| C255 | 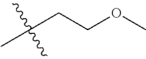 | 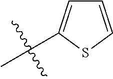 | 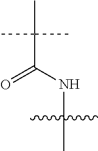 | 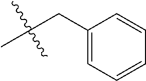 | — | single |
| C256 | 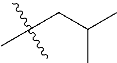 | 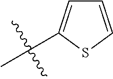 | 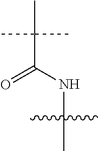 | 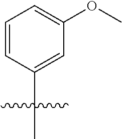 | — | single |
| C257 | 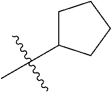 | 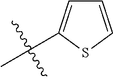 | 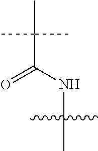 | 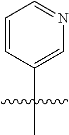 | — | single |
| C258 | 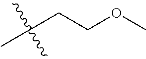 | 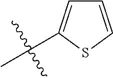 | 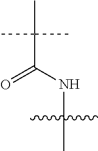 | 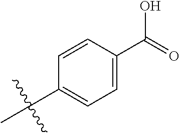 | — | single |
| C259 | 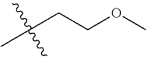 | 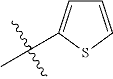 | 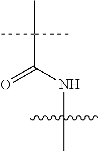 | 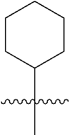 | — | single |
| C260 | 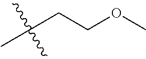 | 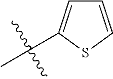 | 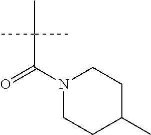 | | — | single |
| C261 | 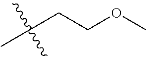 | 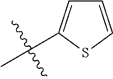 | 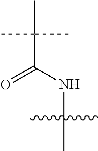 | 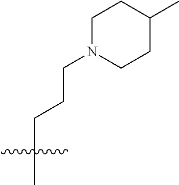 | — | single |
| C262 | 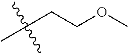 | 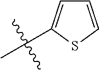 | 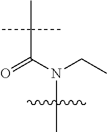 | 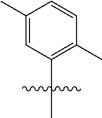 | — | single |
| C263 | 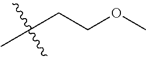 | 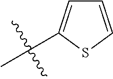 | 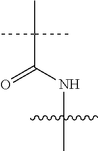 | 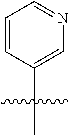 | — | single |
| C264 | 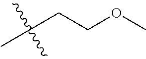 | 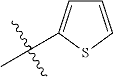 | 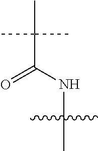 | 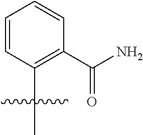 | — | single |
| C265 | 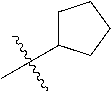 | 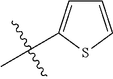 | 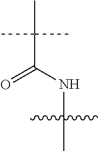 | 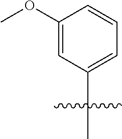 | — | single |
| C266 | 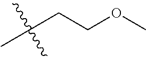 | 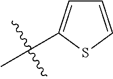 | 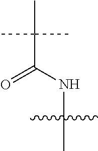 | 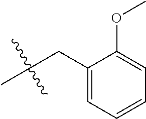 | — | single |
| C267 | 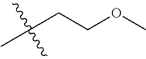 | 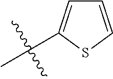 | 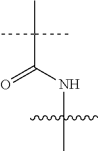 | 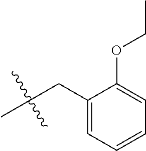 | — | single |
| C268 | 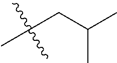 | 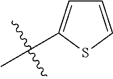 | 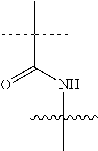 | 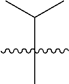 | — | single |
| C269 | 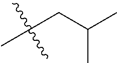 | 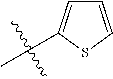 | 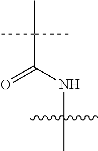 | 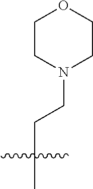 | — | single |
| C270 | 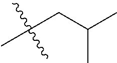 | 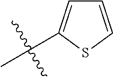 | 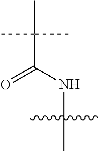 | 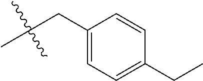 | — | single |
| C271 | 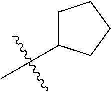 | 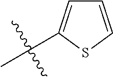 | 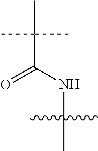 | 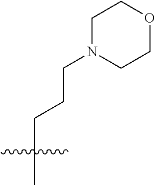 | — | single |
| C272 | 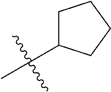 | 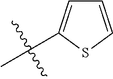 | 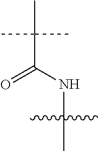 | 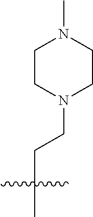 | — | single |

*L² is attached to the isoquinolinone core via the dotted line - - - -

N.B. Q=R² except for C70, C71, C82, C97, C119, C138, C140, C146 and C149.

### Part A

### Example 1

### Preparation of Unsubstituted or Substituted Homophthalic Acids

### Method 1, Exemplified Via the Preparation of 2-(carboxymethyl)-4,5-dimethoxybenzoic acid

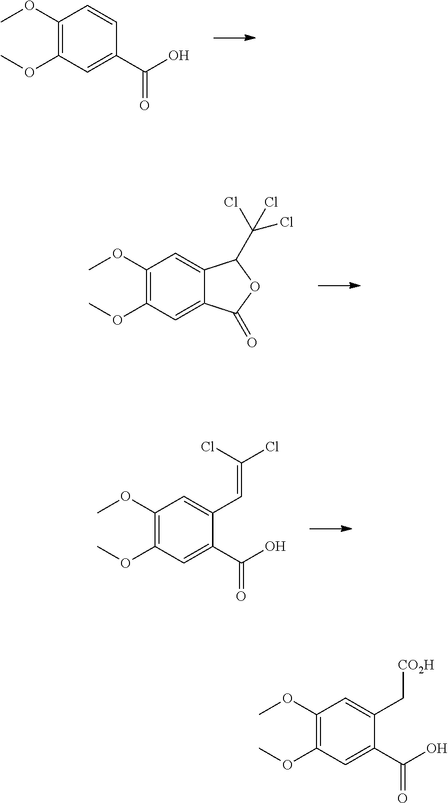

Veratric acid (3.641 g, 20 mmol), chloral hydrate (3.9 g) and sulfuric acid (10 mL) were stirred at RT for 48 h. The mixture was poured onto ice. The precipitate was collected and added to a saturated sodium hydrogen carbonate solution and sonicated (pH was just above 7). The solid was filtered, rinsed with water and dried under vacuum to give 1.60 g (25%) of 5,6-dimethoxy-3-(trichloromethyl)isobenzofuran-1(3H)-one.

5,6-Dimethoxy-3-(trichloromethyl)isobenzofuran-1(3H)-one (1.40 g, 4.53 mmol) was introduced in a 100 mL flask with acetic acid (10 mL). To this mixture was added zinc powder (1.06 g), per portion over 1 h. The reaction mixture was stirred for 2 more hours before being heated to 90° C., the zinc residue and zinc acetate were filtered off while hot (on a filter paper). The filtrate was cooled, and water was added. The resulting precipitate was filtered, rinsed with water and dried to give 434 mg (35%) of 2-(2,2-dichlorovinyl)-4,5-dimethoxybenzoic acid. ESI/APCI (+): 276.8 (M+H).

2-(2,2-Dichlorovinyl)-4,5-dimethoxybenzoic acid (414 mg, 1.5 mmol) was added per portion to 1 mL sulfuric acid. After total dissolution, 1 mL sulfuric acid was added and the mixture heated to 90° C. for 1 h, then poured onto ice. A sticky brown solid was formed, which was filtered on a filter paper. The filtrate was concentrated and left at 4° C. overnight. The new precipitate was combined with the brown solid and crystallized from a mixture water/ethanol to give 35 mg (10%) of 2-(carboxymethyl)-4,5-dimethoxybenzoic acid. ESI/APCI (−): 239 (M−H).

### Method 2, Exemplified Via the Preparation of 2-(carboxymethyl)-4,5-dimethoxybenzoic acid

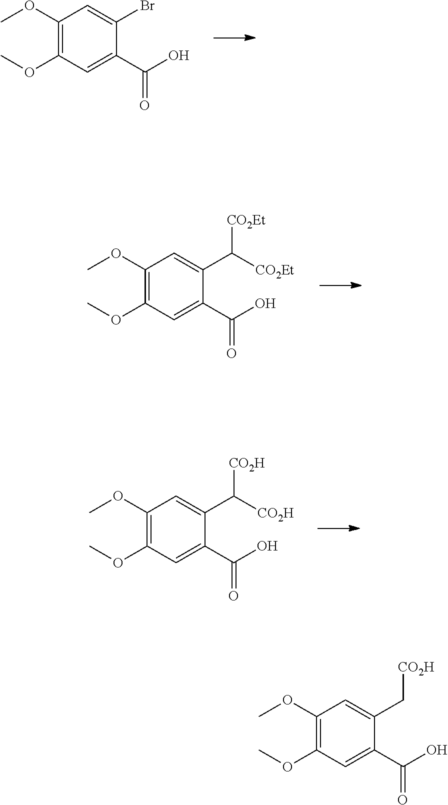

In a 2-necks 250 mL flask, equipped with a strong stirring bar, were introduced 2-bromoveratric acid (6.0 g, 23.1 mmol), diethylmalonate (60 mL) and copper bromide (331 mg, 2.31 mmol). The suspension was degassed and placed under nitrogen at 0° C. Sodium hydride (60% in mineral oil, 2.21 g) was added per portions over 20-25 min. After complete addition of sodium hydride, the mixture was heated to 90° C., and stirring was helped to homogenise the solution. The heating was maintained for 2 h. After cooling to RT, 300 mL water were added and the mixture was extracted twice with 150 mL ether.

The brown solid present in the mixture was removed by filtration to facilitate work-up. The aqueous layer was acidified with 6N HCl and extracted 4 times with ethyl acetate. Combined ethyl acetate layers were concentrated under vacuum to give 2-(1,3-diethoxy-1,3-dioxopropan-2-yl)-4,5-dimethoxybenzoic acid.

The isolated solid was dissolved in 60 mL THF and 60 mL water containing 4.6 g of sodium hydroxide was added dropwise. The mixture was stirred at RT overnight. THF was evaporated, the resulting aqueous layer was acidified with HCl and extracted 4 times with 100 mL ethyl acetate.

Combined organic layers were dried over magnesium sulfate, filtered and concentrated to give 7 g of a white oily solid (probably containing traces of malonate.

The residue was dissolved in about 60 mL of ethyl acetate and heated to reflux overnight. Ethyl acetate was partially evaporated and the mixture was left at 4° C. to crystallize. At this stage the di-acid contained some ester. The mixture was then further hydrolysed in a mixture of THF and sodium hydroxide solution. After 24 h, THF was evaporated and concentrated HCl was added until pH=1. The precipitate was filtered, rinsed with water and dried in a desiccator to give 6.2 g (56%) of 2-(carboxymethyl)-4,5-dimethoxybenzoic acid. ESI/APCI (−): 239 (M−H).

### Method 3, Exemplified Via the Preparation of 2-(carboxymethyl)-5-chlorobenzoic acid

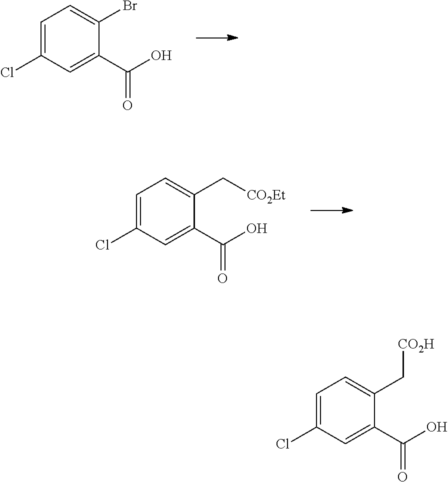

Sodium metal (138 mg, 6 mmol) was dissolved in 15 mL of absolute ethanol. To this solution were added ethyl acetoacetate (555 μL, 4 mmol), copper (I) bromide (286 mg, 2 mmol) and 2-bromo-5-chlorobenzoic acid (468 mg, 2 mmol). This mixture was heated at reflux for 3 h, cooled at RT, and filtered through a celite pad. The solvent was removed in vacuum and the residue partitioned between 2N HCl and dichloromethane. The milky dichloromethane layer was separated and concentrated. The residue was adsorbed onto silica gel and purified by flash chromatography, eluting with dichloromethane-1 to 10% methanol (containing 10% acetic acid) to furnish 455 mg (94%) of 5-chloro-2-(2-ethoxy-2-oxoethyl)benzoic acid. ESI/APCI(−): 241 (M−H).

The 5-chloro-2-(2-ethoxy-2-oxoethyl)benzoic acid was dissolved in 5 mL THF and 3 mL of 6N NaOH solution was added. The mixture was stirred at RT for 2 h. THF was evaporated, the residue was diluted with water and the mixture was acidified with concentrated HCl. The mixture was cooled down a few minutes in the fridge. The precipitate was filtered, rinsed with water. The filtrate was further extracted with ethyl acetate. Residue was combined with solid and dried in a desiccator to give 2-(carboxymethyl)-5-chlorobenzoic acid (365 mg, 85%). ESI/APCI(−): 169 (M-CO₂H).
2-(carboxymethyl)-6-chlorobenzoic acid was obtained following method 3, from 2-bromo-6-chlorobenzoic acid, with 79% yield. ESI/APCI(+): 237 (M+Na)
2-(carboxymethyl)-4-chlorobenzoic acid was obtained following method 3, from 2-bromo-4-chlorobenzoic acid, with 90% yield. ESI/APCI(−): 169 (M-CO₂H)
2-(carboxymethyl)-5-iodobenzoic acid was obtained following method 3, from 2-bromo-5-iodobenzoic acid, with 71% yield. ESI/APCI(−): 261 (M-CO₂H)
2-(carboxymethyl)-3-methylbenzoic acid was obtained following method 3, from 2-bromo-3-methylbenzoic acid, with 97% yield. ESI/APCI(−): 149 (M-CO₂H).
2-(carboxymethyl)-4-methylbenzoic acid was obtained following method 3, from 2-bromo-4-methylbenzoic acid, with 67% yield. ESI/APCI(−): 149 (M-CO₂H).
2-(carboxymethyl)-5-methylbenzoic acid was obtained following method 3, from 2-bromo-5-methylbenzoic acid, with 80% yield. ESI/APCI(−): 149 (M-CO₂H).
2-(carboxymethyl)-6-methylbenzoic acid was obtained following method 3, from 2-bromo-6-methylbenzoic acid, with 88% yield. ESI/APCI(−): 149 (M-CO₂H).
2-(carboxymethyl)-5-methoxyl benzoic acid was obtained following method 1 from 3-methoxybenzoic acid with 33% yield. ESI/APCI(−): 209 (M−H).
2-(carboxymethyl)-3,6-difluorolbenzoic acid was obtained following method 3 from 2-chloro-3,6-difluorobenzoic acid with 63% yield. ESI/APCI(−): 215 (M−H).
2-(carboxymethyl)-5-trifluoromethylbenzoic acid was obtained following method 3 from 2-chloro-5-trifluoromethylbenzoic acid with 76% yield. ESI/APCI(−): 517 (2(M−H)+Na).
2-(carboxymethyl)-6-fluoro-5-methylbenzoic acid was obtained following method 3 from 2-chloro-6-fluoro-5-methyl acid with 72% yield. ESI/APCI(+): 235 (M+Na).
6-(carboxymethyl)-2-fluoro-3-methylbenzoic acid was obtained following method 3 from 6-chloro-2-fluoro-3-methoxybenzoic acid with 98% yield. ESI/APCI(−): 227 (M−H).
2-(carboxymethyl)-5-nitrobenzoic acid
   Fuming nitric acid (20 mL) was added dropwise to homophthalic acid at 0° C. The mixture was then stirred at RT for 5 h before being poured onto ice. The solid was filtered, rinsed with cold water and dried in a desiccator to give 2-(carboxymethyl)-5-nitrobenzoic acid (2.01 g, 32%). ESI/APCI(+): 207.8 (M+H).
   Alternatively, as stated above, multiple substituted homophthalic acids and/or esters are commercially available.

### Example 2

### Preparation of Unsubstituted or Substituted Homophthalic Anhydrides

7-Nitrohomophthalic anhydride
   2-(Carboxymethyl)-5-nitrobenzoic acid (1.125 g, 5 mmol) was suspended in 10 mL toluene and acetic anhydride (567 μL, 6 mmol) was added. The mixture was stirred at 115° C. for 1 h. Heptane was added and the reaction mixture was cooled down to RT to allow precipitation of product. The solid was filtered, and rinsed with heptane to give title compound (900 mg, 87%). ESI/APCI(−): 206.
5-Methylhomophthalic anhydride was obtained from 2-(carboxymethyl)-3-methylbenzoic acid in 34% yield.
6-Methylhomophthalic anhydride was obtained from 2-(carboxymethyl)-4-methylbenzoic acid in 69% yield. ¹H NMR (CDCl₃) δ 2.46 (s, 3H), 4.09 (s, 2H), 7.14 (s, 1H), 7.31 (d, 1H), 8.10 (d, 1H).
7-Methylhomophthalic anhydride was obtained from 2-(carboxymethyl)-5-methylbenzoic acid in 73% yield. ¹H NMR (CDCl₃) δ 2.44 (s, 3H), 4.09 (s, 2H), 7.22 (d, 1H), 7.50 (d, 1H), 8.01 (s, 1H).
8-Methylhomophthalic anhydride was obtained from 2-(carboxymethyl)-6-methylbenzoic acid in 56% yield. ¹H NMR (CDCl₃) δ 2.73 (s, 3H), 4.08 (s, 2H), 7.16 (d, 1H), 7.32 (d, 1H), 7.52 (t, 1H).
8-Chlorohomophthalic anhydride was obtained from 2-(carboxymethyl)-6-chlorobenzoic acid in 25% yield.
7-Chlorohomophthalic anhydride was obtained from 2-(carboxymethyl)-5-chlorobenzoic acid in 92% yield. ¹H NMR (CDCl₃) δ 4.12 (s, 2H), 7.30 (d, 1H), 7.66 (d, 1H), 8.20 (s, 1H).
6-Chlorohomophthalic anhydride was obtained from 2-(carboxymethyl)-4-chlorobenzoic acid in 55% yield. ¹H NMR (CDCl₃) δ 4.11 (s, 2H), 7.35 (s, 1H), 7.49 (d, 1H), 8.15 (d, 1H).
7-Iodohomophthalic anhydride was obtained from 2-(carboxymethyl)-5-iodobenzoic acid in 85% yield. ¹H NMR (CDCl₃) δ 4.08 (s, 2H), 7.09 (d, 1H), 8.01 (d, 1H), 8.54 (s, 1H).
6,7-dimethoxyhomophthalic anhydride
   2-(Carboxymethyl)-4,5-dimethoxybenzoic acid (300 mg, 1.25 mmol) was suspended in 4 mL of trifluoroacetic anhydride and the reaction mixture was stirred at RT for a period of 4 hours. The precipitate was filtered off and washed with cold heptane to give title compound (210 mg, 72%). ¹H NMR (DMSO-d₆) δ 3.84 (s, 3H), 3.87 (s, 3H), 4.18 (s, 2H), 7.03 (s, 1H), 7.43 (s, 1H).
6-dimethoxyhomophthalic anhydride was obtained following the same method from 2-(carboxymethyl)-5-fluorobenzoic acid in 88% yield. ¹H NMR (CDCl₃) ¹H NMR δ 4.15 (s, 2H), 7.04 (dd, 2H), 7.22 (s, 1H), 8.23 (dd, 1H).
5,8-difluorohomophthalic anhydride was obtained following the same method from 2-(carboxymethyl)-3,6-difluorobenzoic in quantitative yield, and was used in the next step without further purification.
7-trifluoromethylhomophthalic anhydride was obtained following the same method from 2-(carboxymethyl)-5-trifluoromethylbenzoic acid in quantitative yield, and was used in the next step without further purification.
8-fluoro-7-methylhomophthalic anhydride was obtained following the same method from 2-(carboxymethyl)-6-fluoro-5-methylbenzoic acid in quantitative yield, and was used in the next step without further purification.
8-fluoro-7-methoxylhomophthalic anhydride was obtained following the same method from 6-(carboxymethyl)-2-fluoro-3-methoxybenzoic acid in quantitative yield, and was used in the next step without further purification.

### Example 3

### Preparation of 2-(2-methoxyethyl)-N-(3-methoxyphenyl)-7-nitro-1-oxo-3-(thiophen-2-yl)-1,2,3,4-tetrahydroisoquinoline-4-carboxamide (C3) (Method A)

Thiophene-2-carboxaldehyde (936 μL, 10 mmol) and methoxyethylamine (873 μL, 10 mmol) were introduced in a sealed tube and the reaction mixture was heated at 60° C. for 6 h. The crude material was dried in vacuum overnight to give the Schiff's base quantitatively.

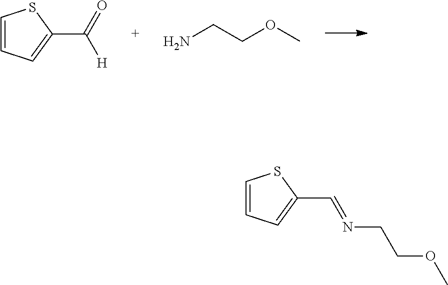

7-Nitrohomophthalic anhydride (414 mg, 2 mmol) was suspended in 15 mL chloroform and 2-methoxy-N-(thiophen-2-ylmethylene)ethanamine (316 μL, 2 mmol) was added. The reaction was stirred at RT overnight. The mixture was rinsed with brine and water and evaporated to give a yellow foam. The resulting compound was dissolved in a sodium carbonate solution and the aqueous layer was washed with dichloromethane twice. The aqueous layer was acidified with concentrated HCl, a solid precipitates. The aqueous layer (containing the precipitate) was extracted once with dichloromethane and once with ethyl acetate. The organic layers were combined, dried over magnesium sulfate, filtered and concentrated to give 2-(2-methoxyethyl)-7-nitro-1-oxo-3-(thiophen-2-yl)-1,2,3,4-tetrahydroisoquinoline-4-carboxylic acid. ESI/APCI(−): 331 (M−H).
2-(2-Methoxyethyl)-7-nitro-1-oxo-3-(thiophen-2-yl)-1,2,3,4-tetrahydroisoquinoline-4-carboxylic acid (112 mg, 0.3 mmol) and 3-methoxyaniline (22.4 mg, 0.2 mmol) were dissolved in dichloromethane (3.2 mL). The mixture was stirred 10 min before adding PS-carbodiimide (Biotage reagent, 0.32 g, 0.4 mmol). The mixture was stirred overnight at RT. The resin was filtered off and rinsed 3 times with dichloromethane. The filtrate was adsorbed onto silica and purified by flash chromatography on silica gel, eluting with dichloromethane, 0 to 100% ethyl acetate to give 2-(2-methoxyethyl)-N-(3-methoxyphenyl)-7-nitro-1-oxo-3-(thiophen-2-yl)-1,2,3,4-tetrahydroisoquinoline-4-carboxamide (17 mg, 17%). ESI/APCI(+): 482 (M+H). ¹H NMR (DMSO-d₆) δ 3.16 (m, 2H), 3.24 (s, 3H, OMe), 3.55 (t, 2H, J=6.0 Hz), 3.73 (s, 3H, OMe), 4.88 (d, 1H, J=5.5 Hz), 5.61 (d, 1H, J=5.5 Hz), 6.69 (dd, 1H, J=1.7, 8.3 Hz), 6.85 (m, 2H), 7.09 (d, 1H, J=8.3 Hz), 7.26 (m, 3H), 7.72 (d, 1H, J=8.7 Hz), 8.41 (dd, 1H, J=2.3, 8.7 Hz), 8.70 (d, 1H, J=2.3 Hz), 10.5 (s, 1H, NH).

### Example 4

### Preparation of N-(3-methoxyphenyl)-2-(3-methoxypropyl)-1-oxo-3-(thiophen-2-yl)-1,2,3,4-tetrahydroisoquinoline-4-carboxamide (C13) (Method B)

Thiophene-2-carboxaldehyde (500 μL, 5.35 mmol) and 3-methoxypropylamine (547 μL, 5.35 mmol) were introduced in a sealed tube and the reaction mixture was heated at 60° C. for 6 h. The crude material was dried in vacuum overnight to give the Schiff's base quantitatively.

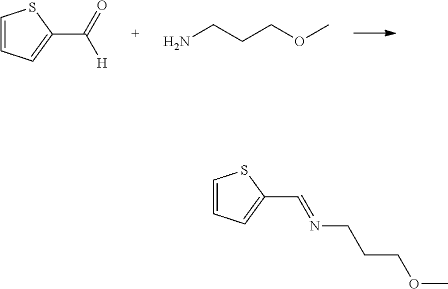

Homophthalic acid (100 mg, 0.62 mmol) was dissolved in chloroform (5 mL) and 3-methoxy-N-(thiophen-2-ylmethylene)propanamine (113 mg, 0.62 mmol) was added. The mixture was stirred at RT overnight. 5 mL of sodium carbonate aqueous solution were added, and the organic layer was removed. The aqueous layer was acidified and extracted twice with ethyl acetate. Combined ethyl acetate layers were dried on magnesium sulfate, filtered and concentrated to give 2-(3-methoxypropyl)-1-oxo-3-(thiophen-2-yl)-1,2,3,4-tetrahydroisoquinoline-4-carboxylic acid as a foam (178 mg, 83%). ESI/APCI(+): 346 (M+H).

To a solution of 2-(3-methoxypropyl)-1-oxo-3-(thiophen-2-yl)-1,2,3,4-tetrahydroisoquinoline-4-carboxylic acid (69 mg, 0.15 mmol) in dry dichloromethane (3 mL) was added 3-methoxyaniline (20 μL, 0.18 mmol), diisopropylethylamine (31 μL) and HATU (68 mg, 0.18 mmol) and the mixture was stirred at RT for 5 h. Dichloromethane was added. The solution was washed once with water and the residue was purified by flash chromatography eluting with dichloromethane, 0 to 50% ethyl acetate to give title compound which was crystallized from ethanol (11 mg, 16%). ESI/APCI(+): 451 (M+H). ¹H NMR (DMSO-d₆) δ 1.72 (m, 2H), 2.90 (m, 1H), 3.12 (s, 3H, OMe), 3.39 (m, 2H), 3.71 (s, 3 H, OMe), 3.94 (m, 1H), 4.28 (s, 1H, H-3), 5.43 (s, 1H, H-2), 6.64 (d, 1H, J=7.8 Hz), 6.91 (t, 1H, J=4.0 Hz), 7.01 (s, 1H), 7.10 (d, 1H, J=7.3 Hz), 7.28 (m, 4H), 7.45 (m, 2H), 7.92 (d, 1H, J=7.2 Hz), 10.41 (s, 1H, NH).

For the examples 5 to 223, all compounds were isolated as pure stereoisomers (cis or trans—which isomer was purified can be determined by using the NMR spectra).

### Example 5

### Preparation of 2-(2-methoxyethyl)-N-(3-methoxyphenyl)-1-oxo-3-(thiophen-2-yl)-1,2,3,4-tetrahydroisoquinoline-4-carboxamide (C1, C2)

This compound was obtained following method B, from thiophene-2-carboxaldehyde, 2-methoxyethylamine, homophthalic anhydride and 3-methoxyaniline.
Two isomers were isolated from flash chromatography on silica gel eluting with dichloromethane, 2 to 20% ethyl acetate:

(±)-trans 2-(2-methoxyethyl)-N-(3-methoxyphenyl)-1-oxo-3-(thiophen-2-yl)-1,2,3,4-tetrahydroisoquinoline-4-carboxamide (C1). ESI/APCI(+): 437 (M+H). ¹H NMR (DMSO-d₆) δ 3.01 (s, 3H), 3.06 (dt, 1H), 3.34-3.44 (m, 2H), 3.71 (s, 3H), 4.08 (dt, 1H), 4.27 (s, 1H), 5.58 (s, 1H), 6.63 (dd, 1H), 6.90 (dd, 1H), 7.01 (d, 1H), 7.13 (d, 1H), 7.22 (t, 1H), 7.29-7.35 (m, 3H), 7.39-7.52 (m, 2H), 7.91 (dd, 1H), 10.39 (s, 1H, NH).

(±)-cis 2-(2-methoxyethyl)-N-(3-methoxyphenyl)-1-oxo-3-(thiophen-2-yl)-1,2,3,4-tetrahydroisoquinoline-4-carboxamide (C2). ESI/APCI(+): 437 (M+H). ¹H NMR (DMSO-d₆) δ 3.06 (dt, 1H), 3.25 (s, 3H), 3.49-3.55 (m, 2H), 3.72 (s, 3H), 4.01 (dt, 1H), 4.72 (d, J=5.4 Hz, 1H), 5.48 (d, J=5.4 Hz, 1H), 5.58 (s, 1H), 6.66 (dd, 1H), 6.82-6.86 (m, 2H), 7.09 (d, 1H), 7.20-7.23 (m, 2H), 7.27 (t, 1H), 7.35 (d, 1H), 7.49 (t, 1H), 7.54 (td, 1H), 7.99 (dd, 1H), 10.45 (s, 1H, NH).

### Example 6

### Preparation of Methyl 2-4-(3-methoxyphenylcarbamoyl)-1-oxo-3-(thiophen-2-yl)-3,4-dihydroisoquinolin-2(1H)-yl)acetate (C14)

This compound was obtained following method B, from thiophene-2-carboxaldehyde, glycine methyl ester, homophthalic anhydride and 3-methoxyaniline in 5% overall yield. ESI/APCI(+): 451 (M+H). ¹H NMR (DMSO-d₆) δ 3.62 (s, 3H, OMe), 3.67 (d, 1H, J=17.3 Hz), 3.70 (s, 3H, OMe), 4.58 (d, 1H, J=17.3 Hz), 4.67 (d, 1H, J=4.6 Hz), 5.61 (d, 1H, J=4.6 Hz), 6.64 (dd, 1H, J=1.8, 8.1 Hz), 6.89 (t, 1H, J=4.2 Hz), 6.98 (d, 1H, J=2.8 Hz), 7.07 (d, 1H, J=8.1 Hz), 7.19 (d, 1H, J=8.1 Hz), 7.23 (s, 1H), 7.39 (d, 2H, J=6.3 Hz), 7.49 (t, 1H, J=7.5 Hz), 7.60 (t, 1H, J=6.9 Hz), 7.98 (d, 1H, J=7.5 Hz), 10.44 (s, 1H, NH).

### Example 7

### Preparation of 2-(2-(Dimethylamino)ethyl)-N-(3-methoxyphenyl)-1-oxo-3-(thiophen-2-yl)-1,2,3,4-tetrahydroisoquinoline-4-carboxamide (C15)

This compound was obtained following method B, from thiophene-2-carboxaldehyde, N,N-dimethylethylenediamine, homophthalic anhydride and 3-methoxyaniline in 37% overall yield. The intermediate acid was not purified by acido-basic extractions but triturated with diethyl ether. Final purification was done by flash chromatography eluting with dichloromethane containing 4 to 7% methanol. ESI/APCI(+): 450 (M+H). ¹H NMR (DMSO-d₆) δ 2.72 (s, 6H, NMe₂), 3.02 (m, 2H), 3.34 (m, 1H), 3.72 (s, 3H, OMe), 4.18 (m, 1H), 5.95 (d, 1H, J=5.5 Hz, H-3), 5.58 (d, 1H, J=5.5 Hz, H-2), 6.66 (d, 1H, J=8.6 Hz), 6.85 (m, 2H), 7.19 (m, 2H), 7.27 (m, 2H), 7.45 (m, 2H), 7.58 (t, 1H, J=7.1 Hz), 8.01 (d, 1H, J=7.7 Hz), 10.87 (s, 1H, NH).

### Example 8

### Preparation of 2-Butyl-N-(3-methoxyphenyl)-1-oxo-3-(thiophen-2-yl)-1,2,3,4-tetrahydroisoquinoline-4-carboxamide (C16)

This compound was obtained following method B, from thiophene-2-carboxaldehyde, butylamine, homophthalic anhydride and 3-methoxyaniline in 6% overall yield. The title compound was purified by flash chromatography eluting with dichloromethane, 5 to 30% ethyl acetate and was crystallized from ethanol. ESI/APCI(+): 435 (M+H). ¹H NMR (DMSO-d₆) δ 0.73 (t, 3H, J=2.7 Hz), 1.19 (m, 2H), 1.44 (m, 2H), 2.85 (m, 1H), 3.71 (s, 3H, OMe), 3.94 (m, 1H), 4.28 (s, 1H, H-3), 5.45 (s, 1H, H-2), 6.64 (dd, 1H, J=2.0, 8.1 Hz), 6.90 (dd, 1H, J=3.7, 5.2 Hz), 7.02 (d, 1H, J=3.1 Hz), 7.10 (d, 1H, J=8.2 Hz), 7.22 (t, 1H, J=8.2 Hz), 7.29 (m, 2H), 7.34 (d, J=7.0 Hz, 1H), 7.45 (m, 2H), 7.91 (d, 1H, J=7.1 Hz), 10.40 (s, 1H, NH).

### Example 9

### Preparation of 2-(2-Methoxyethyl)-N-(3-methoxyphenyl)-5-methyl-1-oxo-3-(thiophen-2-yl)-1,2,3,4-tetrahydroisoquinoline-4-carboxamide (C39)

This compound was obtained following method B, from thiophene-2-carboxaldehyde, 2-methoxyethylamine, 5-methylhomophthalic anhydride and 3-methoxyaniline in 9% overall yield. The title compound was purified by flash chromatography eluting with dichloromethane, 5 to 30% ethyl acetate and was crystallized from ethanol. ESI/APCI(+): 451 (M+H). ¹H NMR (DMSO-d₆) δ 2.19 (s, 3H), 2.95 (s, 3H), 3.02 (m, 1H), 3.42 (m, 2H), 3.71 (s, 3H), 4.13 (m, 1H), 4.42 (s, 1H), 5.56 (s, 1H), 6.64 (dd, 1H), 6.90 (d, 1H), 7.02 (s, 1H), 7.14 (d, 2H), 7.20-7.75 (m, 4H), 7.76 (d, 1H), 10.55 (s, 1H, NH).

### Example 10

### Preparation of 2-(2-Methoxyethyl)-N-(3-methoxyphenyl)-6-methyl-1-oxo-3-(thiophen-2-yl)-1,2,3,4-tetrahydroisoquinoline-4-carboxamide (C40)

This compound was obtained following method B, from thiophene-2-carboxaldehyde, 2-methoxyethylamine, 6-methylhomophthalic anhydride and 3-methoxyaniline in 27% overall yield. The title compound was purified by flash chromatography eluting with dichloromethane, 5 to 30% ethyl acetate and was crystallized from ethanol. ESI/APCI(+): 451 (M+H). ¹H NMR (DMSO-d₆) δ 2.31 (s, 3H), 3.00 (s, 3H), 3.05 (m, 1H), 3.41 (m, 2H), 3.71 (s, 3H), 4.07 (m, 1H), 4.20 (s, 1H), 5.55 (s, 1H), 6.63 (d, 1H), 6.91 (t, 1H), 7.00 (d, 1H), 7.12 (m, 2H), 7.21 (m, 2H), 7.30 (m, 2H), 7.80 (d, 1H), 10.37 (s, 1H, NH).

### Example 11

### Preparation of 2-(2-Methoxyethyl)-N-(3-methoxyphenyl)-7-methyl-1-oxo-3-(thiophen-2-yl)-1,2,3,4-tetrahydroisoquinoline-4-carboxamide (C41)

This compound was obtained following method B, from thiophene-2-carboxaldehyde, 2-methoxyethylamine, 7-methylhomophthalic anhydride and 3-methoxyaniline in 27% overall yield. The title compound was purified by flash chromatography eluting with dichloromethane, 5 to 30% ethyl acetate and was crystallized from ethanol. ESI/APCI(+): 451 (M+H). ¹H NMR (DMSO-d₆) δ 2.34 (s, 3H), 3.00 (s, 3H), 3.06 (m, 1H), 3.42 (m, 2H), 3.71 (s, 3H), 4.06 (m, 1H), 4.21 (s, 1H), 5.55 (s, 1H), 6.63 (dd, 1H), 6.90 (t, 1H), 7.00 (s, 1H), 7.10-7.30 (m, 6H), 7.73 (s, 1H), 10.34 (s, 1H, NH).

### Example 12

### Preparation of 2-(2-Methoxyethyl)-N-(3-methoxyphenyl)-8-methyl-1-oxo-3-(thiophen-2-yl)-1,2,3,4-tetrahydroisoquinoline-4-carboxamide (C42)

This compound was obtained following method B, from thiophene-2-carboxaldehyde, 2-methoxyethylamine, 8-methylhomophthalic anhydride and 3-methoxyaniline in 39% overall yield. The title compound was purified by flash chromatography eluting with dichloromethane, 5 to 30% ethyl acetate and was crystallized from ethanol. ESI/APCI(+): 451 (M+H). ¹H NMR (DMSO-d₆) δ 2.60 (s, 3H), 3.07 (s, 3H), 3.10 (m, 1H), 3.40 (m, 2H), 3.71 (s, 3H), 3.99 (m, 1H), 4.21 (s, 1H), 5.51 (s, 1H), 6.63 (dd, 1H), 6.89 (t, 1H), 6.99 (s, 1H), 7.11-7.33 (m, 7H), 10.38 (s, 1H, NH).

### Example 13

### Preparation of 7-Chloro-2-(2-methoxyethyl)-N-(3-methoxyphenyl)-1-oxo-3-(thiophen-2-yl)-1,2,3,4-tetrahydroisoquinoline-4-carboxamide (C43)

This compound was obtained following method B, from thiophene-2-carboxaldehyde, 2-methoxyethylamine, 7-chlorohomophthalic anhydride and 3-methoxyaniline in 13% overall yield. The title compound was purified by flash chromatography eluting with dichloromethane, 5 to 30% ethyl acetate and was crystallized from ethanol. ESI/APCI(+): 471 (M+H). ¹H NMR (DMSO-d₆) δ 2.97 (s, 3H), 3.07 (m, 1H), 3.41 (m, 2H), 3.71 (s, 3H), 4.08 (m, 1H), 4.32 (s, 1H), 5.61 (s, 1H), 6.65 (d, 1H), 6.92 (t, 1H), 7.02 (s, 1H), 7.12 (d, 2H), 7.22 (t, 1H), 7.31 (m, 2H), 7.41 (d, 1H), 7.57 (d, 1H), 7.86 (s, 1H), 10.43 (s, 1H, NH).

### Example 14

### Preparation of 8-Chloro-2-(2-methoxyethyl)-N-(3-methoxyphenyl)-1-oxo-3-(thiophen-2-yl)-1,2,3,4-tetrahydroisoquinoline-4-carboxamide (C24)

This compound was obtained following method B, from thiophene-2-carboxaldehyde, 2-methoxyethylamine, 8-chlorohomophthalic anhydride and 3-methoxyaniline in 4% overall yield. The title compound was purified by flash chromatography eluting with dichloromethane, 5 to 30% ethyl acetate and was crystallized from ethanol. ESI/APCI(+): 471 (M+H). ¹H NMR (DMSO-d₆) δ 2.97 (s, 3H), 3.07 (m, 1H), 3.41 (m, 2H), 3.71 (s, 3H), 4.08 (m, 1H), 4.32 (s, 1H), 5.61 (s, 1H), 6.65 (d, 1H), 6.92 (t, 1H), 7.02 (s, 1H), 7.12 (d, 2H), 7.22 (t, 1H), 7.31 (m, 2H), 7.41 (d, 1H), 7.57 (d, 1H), 7.86 (s, 1H), 10.43 (s, 1H, NH).

### Example 15

### Preparation of 7-Iodo-2-(2-methoxyethyl)-N-(3-methoxyphenyl)-1-oxo-3-(thiophen-2-yl)-1,2,3,4-tetrahydroisoquinoline-4-carboxamide (C56)

This compound was obtained following method B, from thiophene-2-carboxaldehyde, 2-methoxyethylamine, 7-iodohomophthalic anhydride and 3-methoxyaniline in 6% overall yield. The title compound was purified by flash chromatography eluting with dichloromethane, 5 to 30% ethyl acetate and was crystallized from ethanol. ESI/APCI(+): 563 (M+H). ¹H NMR (DMSO-d₆) δ 2.97 (s, 3H), 3.05 (m, 1H), 3.42 (m, 2H), 3.71 (s, 3H), 4.07 (m, 1H), 4.27 (s, 1H), 5.60 (s, 1H), 6.64 (d, 1H), 6.91 (m, 1H), 7.01 (s, 1H), 7.10 (m, 5H), 7.85 (d, 1H), 8.18 (s, 1H), 10.42 (s, 1H, NH).

### Example 16

### Preparation of 2-(2-Methoxyethyl)-1-oxo-N-phenyl-3-(thiophen-2-yl)-1,2,3,4-tetrahydroisoquinoline-4-carboxamide (C18)

This compound was obtained following method B, from thiophene-2-carboxaldehyde, 2-methoxyethylamine, homophthalic anhydride and aniline in 9% overall yield. The title compound was purified by flash chromatography eluting with heptane, 10 to 80% ethyl acetate. ESI/APCI(+): 407 (M+H). ¹H NMR (DMSO-d₆) δ 3.00 (s, 3H), 3.070 (t, 2H), 3.427 (t, 2H), 4.28 (s, 1H), 5.59 (s, 1H), 6.91 (t, 1H), 7.01-7.07 (m, 2H), 7.27-7.36 (m, 4H), 7.40-7.49 (m, 2H), 7.59 (d, 2H), 7.91 (d, 1H), 10.38 (s, NH).

### Example 17

### Preparation of 2-(2-Methoxyethyl)-1-oxo-3-(thiophen-2-yl)-N-(4-(trifluoromethyl)phenyl)-1,2,3,4-tetrahydroisoquinoline-4-carboxamide (C19)

This compound was obtained following method B, from thiophene-2-carboxaldehyde, 2-methoxyethylamine, homophthalic anhydride and 4-trifluoromethylaniline in 18% overall yield. The title compound was purified by flash chromatography eluting with heptane, 10 to 80% ethyl acetate. ESI/APCI(+): 475 (M+H). ¹H NMR (DMSO-d₆) δ 2.97 (s, 3H), 3.01-3.10 (m, 2H), 3.38-3.44 (m, 2H), 4.32 (s, 1H), 5.61 (s, 1H), 6.91 (t, 1H), 7.01 (d, 1H), 7.31 (d, 1H), 7.35 (d, 1H), 7.41-7.52 (m, 2H), 7.68 (d, 2H), 7.81 (d, 2H), 7.93 (t, 1H), 10.78 (s, NH).

### Example 18

### Preparation of N-(4-cyanophenyl)-2-(2-methoxyethyl)-1-oxo-3-(thiophen-2-yl)-1,2,3,4-tetrahydroisoquinoline-4-carboxamide (C20)

This compound was obtained following method B, from thiophene-2-carboxaldehyde, 2-methoxyethylamine, homophthalic anhydride and 4-aminobenzonitrile in 8% overall yield. The title compound was purified by flash chromatography eluting with heptane, 10 to 80% ethyl acetate. ESI/APCI(+): 432 (M+H). ¹H NMR (DMSO-d₆) δ 2.95 (s, 3H), 3.02-3.09 (m, 2H), 3.88-3.43 (m, 2H), 4.32 (s, H), 5.60 (s, 1H), 6.91 (t, 1H), 7.00 (d, 1H), 7.31-7.38 (m, 2H), 7.42-7.52 (m, 2H), 7.79 (s, 4H), 7.91 (d, 1H), 10.86 (s, NH).

### Example 19

### Preparation of N-(biphenyl-3-yl)-2-(2-methoxyethyl)-1-oxo-3-(thiophen-2-yl)-1,2,3,4-tetrahydroisoquinoline-4-carboxamide (C21)

This compound was obtained following method B, from thiophene-2-carboxaldehyde, 2-methoxyethylamine, homophthalic anhydride and 4-aminobiphenyl in 14% overall yield. The title compound was purified by flash chromatography eluting with heptane, 10 to 80% ethyl acetate. ESI/APCI(+): 483 (M+H). ¹H NMR (DMSO-d₆) δ 2.99 (s, 3H), 3.06-3.13 (m, H), 3.43 (t, 2H), 4.31 (s, 1H), 5.62 (s, 1H), 6.92 (t, 1H), 7.02 (d, 1H), 7.31 (d, 1H), 7.37 (t, 3H), 7.42-7.52 (m, 5H), 7.59 (t, 3H), 7.94 (t, 2H), 10.51 (s, NH).

### Example 20

### Preparation of 2-(2-Methoxyethyl)-1-oxo-N-(2-phenoxyethyl)-3-(thiophen-2-yl)-1,2,3,4-tetrahydroisoquinoline-4-carboxamide (C34)

This compound was obtained following method B, from thiophene-2-carboxaldehyde, 2-methoxyethylamine, homophthalic anhydride and 2-phenoxyethylamine in 6% overall yield. The title compound was purified by flash chromatography eluting with heptane, 10 to 80% ethyl acetate. ESI/APCI(+): 451 (M+H). ¹H NMR (DMSO-d₆) δ 3.02-3.11 (m, 2H), 3.17 (s, 3H), 3.37-3.49 (m, 4H), 3.97-4.06 (m, 2H), 4.08 (s, 1H), 5.46 (s, 1H), 6.87 (t, 1H), 6.92 (d, 4H), 7.25-7.32 (m, 4H), 7.38-7.52 (m, 2H), 7.88 (d, 1H), 8.29 (t, NH).

### Example 21

### Preparation of 2-(2-Methoxyethyl)-N-(2-(1-methyl-1H-indol-3-yl)ethyl)-1-oxo-3-(thiophen-2-yl)-1,2,3,4-tetrahydroisoquinoline-4-carboxamide (C44)

This compound was obtained following method B, from thiophene-2-carboxaldehyde, 2-methoxyethylamine, homophthalic anhydride and 1-methyltrypamine in 8% overall yield. The title compound was purified twice by flash chromatography eluting with heptane, 10 to 80% ethyl acetate first and with dichloromethane, 0 to 5% methanol. ESI/APCI(+): 488 (M+H). ¹H NMR (DMSO-d₆) δ 2.73-2.83 (m, 2H), 3.07 (d, 2H), 3.18 (s, 3H), 3.24 (t, H), 3.43 (t, 2H), 3.72 (s, 3H), 4.00 (s, 1H), 5.48 (s, 1H), 6.88 (t, 1H), 6.94 (d, 1H), 7.01 (d, 2H), 7.13 (t, 1H), 7.23-7.29 (m, 2H), 7.36 (d, 1H), 7.45 (t, 2H), 7.53 (d, 1H), 7.90 (d, 1H), 7.96 (s, NH).

### Example 22

### Preparation of Methyl 4-(2-(2-methoxyethyl)-1-oxo-3-(thiophen-2-yl)-1,2,3,4-tetrahydroisoquinoline-4-carboxamido)benzoate (C45)

This compound was obtained following method B, from thiophene-2-carboxaldehyde, 2-methoxyethylamine, homophthalic anhydride and methyl 4-aminobenzoate in 1% overall yield. The title compound was purified twice by flash chromatography eluting with heptane, 10 to 80% ethyl acetate first and with dichloromethane, 0 to 5% methanol. ESI/APCI(+): 465 (M+H). ¹H NMR (DMSO-d₆) δ 2.95 (s, 3H), 3.01-3.1 (m, 2H), 3.43-3.51 (m, 2H), 3.82 (s, 3H), 4.32 (s, 1H), 5.61 (s, 1H), 6.91 (d, 1H), 7.00 (d, 1H), 7.30 (d, 1H), 7.35 (d, 1H), 7.44 (s, 1H), 7.47 (d, NH), 7.49 (s, 1H), 7.74 (d, 2H), 7.92 (d, H): 10.77 (s, 1H).

### Example 23

### Preparation of Ethyl 3-(2-(2-methoxyethyl)-1-oxo-3-(thiophen-2-yl)-1,2,3,4-tetrahydroisoquinoline-4-carboxamido)benzoate (C46)

This compound was obtained following method B, from thiophene-2-carboxaldehyde, 2-methoxyethylamine, homophthalic anhydride and ethyl 3-aminobenzoate in 2% overall yield. The title compound was purified twice by flash chromatography eluting with heptane, 10 to 80% ethyl acetate. ESI/APCI(+): 479 (M+H). ¹H NMR (DMSO-d₆) δ 1.31 (t, 3H), 2.95 (s, 3H), 3.03-3.10 (m, 2H), 3.39-3.46 (m, 2H), 4.27 (t, 2H), 4.32 (s, 1H), 5.61 (s, 1H), 6.92 (t, 1H), 7.00 (d, 1H), 7.31 (d, 1H), 7.34 (d, NH), 7.44-7.50 (m, 3H), 7.64 (d, 1H), 7.91 (t, 2H), 8.25 (s, 1H), 10.66 (d, 1H).

### Example 24

### Preparation of N-(3-fluorophenyl)-2-(2-methoxyethyl)-1-oxo-3-(thiophen-2-yl)-1,2,3,4-tetrahydroisoquinoline-4-carboxamide (C47)

This compound was obtained following method B, from thiophene-2-carboxaldehyde, 2-methoxyethylamine, homophthalic anhydride and ethyl 3-fluoroaniline in 8% overall yield. The title compound was purified twice by flash chromatography first eluting with dichloromethane, 0 to 5% methanol and then eluting with heptane, 10 to 80% ethyl acetate. ESI/APCI(+): 425 (M+H). ¹H NMR (DMSO-d₆) δ 2.98 (s, 3H), 3.01-3.10 (m, 2H), 3.38-3.44 (m, 2H), 4.28 (s, 1H), 5.59 (s, 1H), 6.87-6.93 (m, 2H), 7.00 (d, 1H), 7.30 (s, NH), 7.32 (d, 2H), 7.36 (d, 1H), 7.40-7.52 (m, 2H), 7.56 (d, 1H), 7.91 (d, 1H), 10.63 (s, 1H).

### Example 25

### Preparation of N-(3,4-dimethoxyphenyl)-2-(2-methoxyethyl)-1-oxo-3-(thiophen-2-yl)-1,2,3,4-tetrahydroisoquinoline-4-carboxamide (C48)

This compound was obtained following method B, from thiophene-2-carboxaldehyde, 2-methoxyethylamine, homophthalic anhydride and ethyl 3,4-dimethoxyaniline in 7% overall yield. The title compound was purified twice by flash chromatography eluting with dichloromethane, 0 to 5% methanol. ESI/APCI(+): 467 (M+H). ¹H NMR (DMSO-d₆) δ 3.02 (s, 3H), 3.05-3.10 (t, 2H), 3.41-3.44 (t, 2H), 3.71 (s, 6H), 4.24 (s, 1H), 5.58 (s, 1H), 6.88-6.92 (m, 2H), 7.00 (d, 1H), 7.07-7.11 (m, 1H), 7.29 (s, NH), 7.31 (d, 2H), 7.42-7.48 (m, 2H), 7.90 (d, 1H), 10.26 (s, 1H).

### Example 26

### Preparation of N-(4-chlorophenyl)-2-(2-methoxyethyl)-1-oxo-3-(thiophen-2-yl)-1,2,3,4-tetrahydroisoquinoline-4-carboxamide (C49)

This compound was obtained following method B, from thiophene-2-carboxaldehyde, 2-methoxyethylamine, homophthalic anhydride and ethyl 3,4-dimethoxyaniline in 15% overall yield. The title compound was purified by flash chromatography eluting with heptane, 10 to 80% ethyl acetate. ESI/APCI(+): 441 (M+H). ¹H NMR (DMSO-d₆) δ 2.99 (s, 3H), 3.02-3.10 (m, 2H), 3.37-3.44 (m, 2H), 4.27 (s, 1H), 5.58 (s, 1H), 6.91 (t, 1H), 7.00 (d, 1H), 7.30 (d, 1H), 7.34 (s, NH), 7.36 (d, 2H), 7.43-7.49 (m, 2H), 7.62 (d, 2H), 7.90 (d, 1H), 10.53 (s, 1H).

### Example 27

### Preparation of 2-(Furan-2-ylmethyl)-N-(3-methoxyphenyl)-1-oxo-3-(thiophen-2-yl)-1,2,3,4-tetrahydroisoquinoline-4-carboxamide (C4)

This compound was obtained following method B, from thiophene-2-carboxaldehyde, 2-furanmethylamine, homophthalic anhydride and 3-methoxyaniline in 20% overall yield. The title compound was purified by precipitation in dichloromethane and crystallization from ethanol. ESI/APCI(+): 459 (M+H). ¹H NMR (DMSO-d₆) δ 3.72 (s, 3H), 4.17 (d, 1H), 4.27 (s, 1H), 5.07 (d, 1H), 5.42 (s, 1H), 6.17 (s, 1H), 6.30 (d, 1H), 6.63 (d, 1H), 6.88 (t, 1H), 6.96 (d, 1H), 7.01 (d, 1H), 7.18-7.24 (m, 3H), 7.29 (d, 1H), 7.34 (d, 1H), 7.45-7.51 (m, 2H), 7.95 (d, 1H), 10.32 (s, 1H, NH).

### Example 28

### Preparation of 2-Ethyl-N-(3-methoxyphenyl)-1-oxo-3-(thiophen-2-yl)-1,2,3,4-tetrahydroisoquinoline-4-carboxamide (C5)

This compound was obtained following method B, from thiophene-2-carboxaldehyde, ethylamine, homophthalic anhydride and 3-methoxyaniline in 77% overall yield. The title compound was purified by flash chromatography eluting with heptane, 15 to 50% ethyl acetate. ESI/APCI(+): 407 (M+H). ¹H NMR (DMSO-d₆) δ 1.03 (t, 3H), 2.97 (dq, 1H), 3.71 (s, 3H), 3.91 (dq, 1H), 4.28 (s, 1H), 5.46 (s, 1H), 6.63 (dd, 1H), 6.89 (dd, 1H), 7.03 (d, 1H), 7.12 (d, 1H), 7.19 (d, 1H), 7.24-7.31 (m, 2H), 7.35-7.48 (m, 3H), 10.37 (s, 1H, NH).

### Example 29

### Preparation of 2-(Cyclohexylmethyl)-N-(3-methoxyphenyl)-1-oxo-3-(thiophen-2-yl)-1,2,3,4-tetrahydroisoquinoline-4-carboxamide (C22)

This compound was obtained following method B, from thiophene-2-carboxaldehyde, cyclohexylmethylamine, homophthalic anhydride and 3-methoxyaniline in 3% overall yield. The title compound was purified by flash chromatography eluting with heptane, 15 to 50% ethyl acetate. ESI/APCI(+): 475 (M+H). ¹H NMR (DMSO-d₆) δ 080-0.98 (m, 5H), 1.46-1.64 (m, 6H), 2.54 (dd, 1H), 3.71 (s, 3H), 3.82 (dd, 1H), 4.31 (s, 1H), 5.43 (s, 1H), 6.63 (dd, 1H), 6.88 (t, 1H), 7.01 (d, 1H), 7.09 (d, 1H), 7.19 (t, 1H), 7.28-7.33 (m, 3H), 7.42-7.48 (m, 2H), 7.91 (d, 1H), 10.44 (s, 1H, NH).

### Example 30

### Preparation of 2-(2-Hydroxyethyl)-N-(3-methoxyphenyl)-1-oxo-3-(thiophen-2-yl)-1,2,3,4-tetrahydroisoquinoline-4-carboxamide (C23)

This compound was obtained following method B, from thiophene-2-carboxaldehyde, ethanolamine, homophthalic anhydride and 3-methoxyaniline in 22% overall yield. The title compound was purified by flash chromatography eluting with heptane, 15 to 50% ethyl acetate. ESI/APCI(+): 423 (M+H). ¹H NMR (DMSO-d₆) δ 2.91 (dt, 1H), 3.49 (q, 2H), 3.72 (s, 3H), 3.99 (dt, 1H), 4.27 (s, 1H), 4.62 (t, 1H), 5.59 (s, 1H), 6.64 (dd, 1H), 6.90 (dd, 1H), 7.02 (d, 1H), 7.12 (d, 1H), 7.19-7.25 (m, 2H), 7.30 (dd, 1H), 7.39-7.52 (m, 3H), 7.91 (dd, 1H), 10.40 (s, 1H, NH).

### Example 31

### Preparation of 2-Cyclohexyl-N-(3-methoxyphenyl)-1-oxo-3-(thiophen-2-yl)-1,2,3,4-tetrahydroisoquinoline-4-carboxamide (C25)

This compound was obtained following method B, from thiophene-2-carboxaldehyde, cyclohexylamine, homophthalic anhydride and 3-methoxyaniline in 11% overall yield. The title compound was purified by flash chromatography eluting with heptane, 15 to 50% ethyl acetate and crystallization from ethanol. ESI/APCI(+): 461 (M+H). ¹H NMR (DMSO-d₆) δ 0.97-1.61 (m, 10H), 3.71 (s, 3H), 4.18 (s, 1H), 4.18 (t, 1H), 5.59 (s, 1H), 6.64 (dd, 1H), 6.84 (t, 1H), 6.99 (d, 1H), 7.01 (d, 1H), 7.20-7.24 (m, 4H), 7.40-7.45 (m, 2H), 7.91 (dd, 1H), 10.39 (s, 1H, NH).

### Example 32

### Preparation of N-(3-methoxyphenyl)-1-oxo-2-((tetrahydrofuran-2-yl)methyl)-3-(thiophen-2-yl)-1,2,3,4-tetrahydroisoquinoline-4-carboxamide (C31)

This compound was obtained following method B, from thiophene-2-carboxaldehyde, tetrahydrofuran-2-methylamine, homophthalic anhydride and 3-methoxyaniline in 15% overall yield. The title compound was purified by flash chromatography eluting with heptane, 15 to 50% ethyl acetate and crystallization from ethanol to give the 2 diastereoisomers. ESI/APCI(+): 463 (M+H).

¹H NMR (DMSO-d₆) δ 1.63-1.71 (m, 4H), 3.04 (dd, 1H), 3.34-3.44 (m, 2H), 3.71 (s, 3H), 3.89 (m, 1H), 4.07 (dd, 1H), 4.26 (s, 1H), 5.65 (s, 1H), 6.62 (dd, 1H), 6.90 (dd, 1H), 6.99 (m, 1H), 7.11-7.36 (m, 6H), 7.42-7.49 (m, 2H), 7.94 (dd, 1H), 10.47 (s, 1H, NH).

¹H NMR (DMSO-d₆) δ 1.51-1.81 (m, 6H), 2.74 (dd, 1H), 3.31-3.39 (m, 2H), 3.61 (q, 1H), 3.71 (s, 3H), 3.88 (dt, 1H), 4.13 (dd, 1H), 4.29 (d, J=1.2 Hz, 1H), 5.68 (s, 1H), 6.62 (dd, 1H), 6.89 (dd, 1H), 6.99 (d, 1H), 7.09 (t, 1H), 7.19 (t, 1H), 7.27-7.33 (m, 4H), 7.40-7.48 (m, 2H), 7.90 (dd, 1H), 10.39 (s, 1H, NH).

### Example 33

### Preparation of 2-Benzyl-N-(3-methoxyphenyl)-1-oxo-3-(thiophen-2-yl)-1,2,3,4-tetrahydroisoquinoline-4-carboxamide (C37)

This compound was obtained following method B, from thiophene-2-carboxaldehyde, benzylamine, homophthalic anhydride and 3-methoxyaniline in 11% overall yield. The title compound was purified by flash chromatography eluting with heptane, 15 to 50% ethyl acetate. ESI/APCI(+): 469 (M+H). ¹H NMR (DMSO-d₆) δ 3.71 (s, 3H), 3.92 (d, 1H), 4.27 (s, 1H), 5.29 (s, 1H), 5.30 (d, 1H), 6.63 (dd, 1H), 6.89 (dd, 1H), 6.98 (m, 2H), 7.04 (m, 3H), 7.17-7.25 (m, 5H), 7.31-7.34 (m, 2H), 7.41-7.54 (m, 2H), 7.99 (dd, 1H), 10.28 (s, 1H, NH).

### Example 34

### Preparation of 7-Methoxy-2-(2-methoxyethyl)-N-(3-methoxyphenyl)-1-oxo-3-(thiophen-2-yl)-1,2,3,4-tetrahydroisoquinoline-4-carboxamide (C50)

This compound was obtained following method B, from thiophene-2-carboxaldehyde, 2-methoxyethylamine, 7-methoxyhomophthalic anhydride and 3-methoxyaniline in 7% overall yield. The title compound was purified by flash chromatography eluting with heptane, 3 to 20% ethyl acetate. ESI/APCI(+): 467 (M+H). ¹H NMR (DMSO-d₆) δ 3.01 (s, 3H), 3.02 (dt, 1H), 3.42 (m, 2H), 3.71 (s, 3H), 3.80 (s, 3H), 4.01 (m, 1H), 4.19 (s, 1H), 5.56 (s, 1H), 6.61 (dd, 1H), 6.90 (dd, 1H), 7.02 (d, 1H), 7.04 (dd, 1H), 7.11 (d, 1H), 7.19 (d, 1H), 7.26-7.31 (m, 3H), 7.42 (d, 1H), 10.32 (s, 1H, NH).

### Example 35

### Preparation of 8-Fluoro-2-(2-methoxyethyl)-N-(3-methoxyphenyl)-1-oxo-3-(thiophen-2-yl)-1,2,3,4-tetrahydroisoquinoline-4-carboxamide (C51)

This compound was obtained following method B, from thiophene-2-carboxaldehyde, 2-methoxyethylamine, 8-fluorohomophthalic anhydride and 3-methoxyaniline in 6% overall yield. The title compound was purified by flash chromatography eluting with heptane, 3 to 20% ethyl acetate and crystallization from ethyl acetate-heptane. ESI/APCI(+): 455 (M+H). ¹H NMR (DMSO-d₆) δ 2.95 (s, 3H), 3.08 (dt, 1H), 3.38 (m, 2H), 3.72 (s, 3H), 3.97 (dt, 1H), 4.33 (s, 1H), 5.57 (s, 1H), 6.63 (dd, 1H), 6.91 (t, 1H), 7.01 (d, 1H), 7.12-7.23 (m, 4H), 7.32 (m, 2H), 7.48 (td, 1H), 10.45 (s, 1H, NH).

### Example 36

### Preparation of 7-Fluoro-2-(2-methoxyethyl)-N-(3-methoxyphenyl)-1-oxo-3-(thiophen-2-yl)-1,2,3,4-tetrahydroisoquinoline-4-carboxamide (C52)

This compound was obtained following method B, from thiophene-2-carboxaldehyde, 2-methoxyethylamine, 7-fluorohomophthalic anhydride and 3-methoxyaniline in 14% overall yield. The title compound was purified twice by flash chromatography eluting first with dichloromethane, 3 to 20% ethyl acetate and with heptane, 15 to 70% ethyl acetate. ESI/APCI(+): 455 (M+H). ¹H NMR (DMSO-d₆) δ 2.98 (s, 3H), 3.05 (dt, 1H), 3.39 (m, 2H), 3.72 (s, 3H), 4.05 (dt, 1H), 4.32 (s, 1H), 5.62 (s, 1H), 6.63 (d, 1H), 6.91 (t, 1H), 7.02 (d, 1H), 7.12 (d, 1H), 7.20 (t, 1H), 7.33-7.46 (m, 4H), 7.62 (dd, 1H), 10.42 (s, 1H, NH).

### Example 37

### Preparation of 5-Fluoro-2-(2-methoxyethyl)-N-(3-methoxyphenyl)-1-oxo-3-(thiophen-2-yl)-1,2,3,4-tetrahydroisoquinoline-4-carboxamide (C61)

This compound was obtained following method B, from thiophene-2-carboxaldehyde, 2-methoxyethylamine, 5-fluorohomophthalic anhydride and 3-methoxyaniline in 6% overall yield. The title compound was purified by flash chromatography eluting with dichloromethane, 0 to 20% ethyl acetate and crystallized from ethyl acetate/heptane. ESI/APCI(+): 455 (M+H). ¹H NMR (DMSO-d₆) δ 2.94 (s, 3H), 3.01 (ddd, 1H), 3.34-3.44 (m, 2H), 3.72 (s, 3H), 4.11 (dt, 1H), 4.51 (s, 1H), 5.64 (s, 1H), 6.65 (dd, 1H), 6.92 (dd, 1H), 7.04 (d, 1H), 7.13 (d, 1H), 7.21 (t, 1H), 7.34 (m, 2H), 7.37 (t, 1H), 7.46 (td, 1H), 7.77 (d, 1H), 10.62 (s, 1H, NH).

### Example 38

### Preparation of N-(3-methoxyphenyl)-1-oxo-3-(thiophen-2-yl)-2-(2,2,2-trifluoroethyl)-1,2,3,4-tetrahydroisoquinoline-4-carboxamide (C62)

This compound was obtained following method B, from thiophene-2-carboxaldehyde, 2,2,2-trifluoroethylamine, homophthalic anhydride and 3-methoxyaniline in 23% overall yield. The title compound was purified by flash chromatography eluting with dichloromethane, 3 to 20% ethyl acetate. ESI/APCI(+): 461 (M+H). ¹H NMR (DMSO-d₆) δ 3.71 (s, 3H), 3.83 (m, 1H), 4.34 (s, 1H), 4.64 (m, 1H), 5.58 (s, 1H), 6.62 (dd, 1H), 6.90 (dd, 1H), 7.01 (m, 2H), 7.19 (t, 1H), 7.24 (s, 1H), 7.32 (dd, 1H), 7.37 (d, 1H), 7.45 (td, 1H), 7.52 (td, 1H), 7.96 (d, 1H), 10.46 (s, 1H, NH).

### Example 39

### Preparation of N-(3-methoxyphenyl)-2-(2-(methylthio)ethyl)-1-oxo-3-(thiophen-2-yl)-1,2,3,4-tetrahydroisoquinoline-4-carboxamide (C63)

This compound was obtained following method B, from thiophene-2-carboxaldehyde, 2-methylthioethylamine, homophthalic anhydride and 3-methoxyaniline in 45% overall yield. The title compound was purified by flash chromatography eluting with dichloromethane, 3 to 20% ethyl acetate. ESI/APCI(+): 453 (M+H). ¹H NMR (DMSO-d₆) δ 2.05 (s, 3H), 2.50-2.69 (m, 2H), 3.09 (ddd, 1H), 3.71 (s, 3H), 3.99 (ddd, 1H), 4.28 (s, 1H), 5.61 (s, 1H), 6.63 (dd, 1H), 6.91 (dd, 1H), 6.94 (d, 1H), 7.06 (d, 1H), 7.12-7.52 (m, 6H), 7.92 (d, 1H), 10.35 (s, 1H, NH)

### Example 40

### Preparation of N-(3-methoxyphenyl)-2-(3-morpholinopropyl)-1-oxo-3-(thiophen-2-yl)-1,2,3,4-tetrahydroisoquinoline-4-carboxamide (C64)

This compound was obtained following method B, from thiophene-2-carboxaldehyde, 3-morpholinopropylamine, homophthalic anhydride and 3-methoxyaniline in 74% overall yield. The title compound was purified by flash chromatography eluting with dichloromethane, 1 to 7% methanol (containing 5% ammonia). ESI/APCI(+): 506 (M+H).

¹H NMR (DMSO-d₆) δ 1.21 (m, 3H), 1.87 (m, 2H), 2.88 (m, 2H), 3.08 (m, 1H), 3.66 (m, 6H), 3.71 (s, 3H), 4.02 (m, 1H), 4.32 (s, 1H), 5.52 (s, 1H), 6.65 (dd, 1H), 6.92 (dd, 1H), 7.04 (d, 1H), 7.10 (d, 1H), 7.21 (t, 1H), 7.31 (m, 2H), 7.40 (t, 1H), 7.47 (t, 1H), 7.94 (d, 1H), 10.48 (s, 1H, NH).

### Example 41

### Preparation of N-(3-ethoxyphenyl)-2-(2-methoxyethyl)-1-oxo-3-(thiophen-2-yl)-1,2,3,4-tetrahydroisoquinoline-4-carboxamide (C59)

This compound was obtained following method B, from thiophene-2-carboxaldehyde, 2-methoxyethylamine, homophthalic anhydride and 3-ethoxyaniline in 8.3% overall yield. The title compound was purified twice by flash chromatography eluting with heptane, 10 to 80% ethyl acetate. ESI/APCI(+): 451 (M+H). ¹H NMR (DMSO-d₆) δ 1.30 (t, 3H), 3.01 (s, 3H), 3.41-3.45 (m, 2H), 3.93-3.80 (m, 2H), 4.00-4.10 (m, 2H), 4.27 (s, 1H), 5.59 (s, 1H), 6.60-6.63 (m, 1H), 6.89-6.93 (m, 1H), 7.01 (d, NH), 7.08 (d, 1H), 7.20 (t, 1H), 7.30-7.37 (m, 3H), 7.40-7.51 (m, 2H), 7.91 (d, 1H), 10.73 (s, 1H).

### Example 42

### Preparation of N-(3-chlorophenyl)-2-(2-methoxyethyl)-1-oxo-3-(thiophen-2-yl)-1,2,3,4-tetrahydroisoquinoline-4-carboxamide (C60)

This compound was obtained following method B, from thiophene-2-carboxaldehyde, 2-methoxyethylamine, homophthalic anhydride and 3-chloroaniline in 3% overall yield. The title compound was purified twice by flash chromatography eluting with heptane, 10 to 80% ethyl acetate. ESI/APCI(+): 441 (M+H). ¹H NMR (DMSO-d₆) δ 2.99 (s, 3H), 3.01-3.10 (m, 2H), 3.42 (t, 2H), 4.28 (s, 1H), 5.59 (s, 1H), 6.90-6.92 (m, 1H), 7.00 (d, NH), 7.11 (d, 1H), 7.30-7.41 (m, 3H), 7.44-7.51 (m, 3H), 7.82 (s, 1H), 7.91 (d, 1H), 10.60 (s, 1H).

### Example 43

### Preparation of 2-(2-Ethoxyethyl)-N-(3-methoxyphenyl)-1-oxo-3-(thiophen-2-yl)-1,2,3,4-tetrahydroisoquinoline-4-carboxamide (C65)

This compound was obtained following method B, from thiophene-2-carboxaldehyde, 2-ethoxyethylamine, homophthalic anhydride and 3-methoxyaniline in 43% overall yield. The title compound was purified twice by flash chromatography eluting first with dichloromethane, 3 to 20% ethyl acetate and with heptane, 15 to 70% ethyl acetate. ESI/APCI(+): 451 (M+H). ¹H NMR (DMSO-d₆) δ 0.87 (t, 3H), 3.06 (dt, 1H), 3.19 (m, 2H), 3.47 (m, 2H), 3.71 (s, 3H), 4.05 (dt, 1H), 4.27 (s, 1H), 5.61 (s, 1H), 6.62 (d, 1H), 6.90 (dd, 1H), 7.00 (d, 1H), 7.11 (d, 1H), 7.19 (t, 1H), 7.30-7.35 (m, 2H), 7.40-7.49 (m, 2H), 7.91 (dd, 1H), 10.40 (s, 1H, NH).

### Example 44

### Preparation of 2-(2-Isopropoxyethyl)-N-(3-methoxyphenyl)-1-oxo-3-(thiophen-2-yl)-1,2,3,4-tetrahydroisoquinoline-4-carboxamide (C66)

This compound was obtained following method B, from thiophene-2-carboxaldehyde, 2-isopropoxyethylamine, homophthalic anhydride and 3-methoxyaniline in 43% overall yield. The title compound was purified twice by flash chromatography eluting first with dichloromethane, 3 to 20% ethyl acetate and with heptane, 15 to 70% ethyl acetate. ESI/APCI(+): 465 (M+H). ¹H NMR (DMSO-d₆) δ 0.89 (d, 3H), 0.93 (d, 3H), 3.01 (dt, 1H), 3.19 (m, 2H), 3.37 (t, 1H), 3.34 (t, 1H), 3.71 (s, 3H), 4.01 (dt, 1H), 4.27 (s, 1H), 5.62 (s, 1H), 6.63 (d, 1H), 6.90 (dd, 1H), 7.00 (d, 1H), 7.10 (d, 1H), 7.19 (t, 1H), 7.30-7.35 (m, 3H), 7.43-7.49 (m, 2H), 7.91 (dd, 1H), 10.40 (s, 1H, NH).

### Example 45

### Preparation of 2-((1-ethylpyrrolidin-2-yl)methyl)-N-(3-methoxyphenyl)-1-oxo-3-(thiophen-2-yl)-1,2,3,4-tetrahydroisoquinoline-4-carboxamide (C67)

This compound was obtained following method B, from thiophene-2-carboxaldehyde, (1-ethylpyrrolidin-2-yl)methanamine, homophthalic anhydride and 3-methoxyaniline in 20% overall yield. The title compound was purified twice by flash chromatography eluting first with dichloromethane, 0 to 10% MeOH and with dichloromethane, 1 to 7% methanol (containing 5% ammonia). ESI/APCI(+): 490 (M+H). ¹H NMR (DMSO-d₆) δ 1.13 (m, 3H), 1.76 (m, 3H), 1.98 (m, 1H), 2.65-3.35 (m, 6H), 3.71 (s, 3H), 4.19 (m, 1H), 4.42 (s, 1H), 5.71 (s, 1H), 6.63 (dd, 1H), 6.92 (t, 1H), 7.08 (m, 1H), 7.16-7.25 (m, 2H), 7.32-7.54 (m, 5H), 7.95 (d, 1H), 10.76 (s, 1H, NH).

### Example 46

### Preparation of 2-(((S)-1-ethylpyrrolidin-2-yl)methyl)-N-(3-methoxyphenyl)-1-oxo-3-(thiophen-2-yl)-1,2,3,4-tetrahydroisoquinoline-4-carboxamide (C74)

This compound was obtained following method B, from thiophene-2-carboxaldehyde, (S)-(1-ethylpyrrolidin-2-yl)methanamine, homophthalic anhydride and 3-methoxyaniline in 1% overall yield. The title compound was purified first by flash chromatography eluting with dichloromethane, 1 to 7% methanol (containing 5% ammonia) then by preparative silica gel plate using a mixture of dichloromethane/methanol (containing 5% ammonia) 95/5. ESI/APCI(+): 490 (M+H). ¹H NMR (DMSO-d₆) δ 1.19 (m, 4H), 1.91 (m, 4H), 2.09 (m, 1H), 3.03 (m, 2H), 3.58 (m, 2H), 3.72 (s, 3H), 4.01 (dd, 1H), 4.76 (d, J=4.9 Hz, 1H), 5.53 (d, J=4.9 Hz, 1H), 6.66 (dd, 1H), 6.87 (dd, 1H), 6.95 (m, 1H), 7.08 (d, 1H), 7.21-7.27 (m, 2H), 7.33 (d, 1H), 7.37 (d, 1H), 7.49 (t, 1H), 7.95 (t, 1H), 8.02 (d, 1H), 10.48 (s, 1H, NH).

### Example 47

### Preparation of 6,7-dimethoxy-2-(2-methoxyethyl)-N-(3-methoxyphenyl)-1-oxo-3-(thiophen-2-yl)-1,2,3,4-tetrahydroisoquinoline-4-carboxamide (C75)

This compound was obtained following method B, from thiophene-2-carboxaldehyde, 2-methoxyethanamine, 5,6-dimethoxyhomophthalic anhydride and 3-methoxyaniline in 30% overall yield. The title compound was purified by flash chromatography eluting with heptane, 30 to 100% EtOAc. ESI/APCI (+) 497 (M+H). 1H NMR (DMSO-d₆) δ 3.01 (s, 3H), 3.02 (m, 1H), 3.40 (m, 2H), 3.71 (s, 3H), 3.77 (m, 3H), 3.81 (s, 3H), 4.01 (m, 1H), 4.14 (s, 1H), 5.53 (s, 1H), 6.62 (dd, 1H), 6.91 (dd, 1H), 6.97 (s, 1H), 7.04 (d, 1H), 7.12 (d, 1H), 7.19 (t, 1H), 7.30 (m, 2H), 7.42 (s, 1H), 10.26 (s, 1H, NH).

### Example 48

### Preparation of 2-(2-methoxyethyl)-N-(naphthalen-1-ylmethyl)-1-oxo-3-(thiophen-2-yl)-1,2,3,4-tetrahydroisoquinoline-4-carboxamide (C72)

This compound was obtained following method B, from thiophene-2-carboxaldehyde, 2-methoxyethanamine, homophthalic anhydride and naphthalen-1-ylmethanamine in 21% overall yield. The title compound was purified first by flash chromatography eluting with heptane, 10 to 80% EtOAc and then by preparative HPLC eluting with a gradient of acetonitrile in water (0.1% formic acid). ESI/APCI (+) 427 (M+H). ¹H NMR (DMSO-d₆) δ 3.08-3.15 (m, 2H), 3.17 (s, 3H), 3.43 (t, 2H), 4.14 (s, 1H), 4.65-4.83 (m, 2H), 5.51 (s, 1H), 6.87 (t, 1H), 6.94 (d, 1H), 7.27 (s, 1H), 7.29 (s, 1H), 7.41 (t, 2H), 7.45 (d, 1H), 7.48 (s, 1H), 7.51-7.57 (m, 2H), 7.85 (d, 1H), 7.91-8.00 (m, 3H), 8.60 (t, NH).

### Example 49

### Preparation of N-(4-methoxybenzyl)-2-(2-methoxyethyl)-1-oxo-3-(thiophen-2-yl)-1,2,3,4-tetrahydroisoquinoline-4-carboxamide (C79)

This compound was obtained following method B, from thiophene-2-carboxaldehyde, 2-methoxyethanamine, homophthalic anhydride and 4-methoxybenzylamine in 5% overall yield. The title compound was purified twice by flash chromatography eluting first with dichloromethane, 0 to 5% methanol and then with heptane, 10 to 80% EtOAc. ESI/APCI (+) 451 (M+H). ¹H NMR (DMSO-d₆) δ 3.04-3.13 (m, 2H), 3.17 (s, 3H), 3.40 (t, 2H), 3.72 (s, 3H),

4.08 (s, 1H), 4.13-4.29 (m, 2H), 5.49 (s, 1H), 6.85-6.89 (m, 3H), 6.95 (d, 1H), 7.13 (d, 2H), 7.28 (t, 2H), 7.40-7.51 (m, 2H), 7.90 (d, 1H), 8.24 (t, NH).

### Example 50

### Preparation of 2-(2-methoxyethyl)-1-oxo-N-(pyridin-4-ylmethyl)-3-(thiophen-2-yl)-1,2,3,4-tetrahydroisoquinoline-4-carboxamide (C80)

This compound was obtained following method B, from thiophene-2-carboxaldehyde, 2-methoxyethanamine, homophthalic anhydride and 4-picolylamine in 13% overall yield. The title compound was purified three times by flash chromatography eluting with dichloromethane, 0 to 5% methanol. ESI/APCI (+) 422 (M+H). ¹H NMR (DMSO-d₆) δ 3.05-3.13 (m, 2H), 3.15 (s, 3H), 3.40 (d, 2H), 4.16 (s, 1H), 4.23-4.40 (m, 2H), 5.56 (s, 1H), 6.89 (m, 1H), 6.98 (d, 1H), 7.19 (d, 2H), 7.23 (d, 1H), 7.35 (d, 1H), 7.42-7.52 (m, 2H), 7.91 (d, 1H), 8.46 (d, 2H), 8.52 (t, NH).

### Example 51

### Preparation of N-(benzo[d]thiazol-5-yl)-2-(2-methoxyethyl)-1-oxo-3-(thiophen-2-yl)-1,2,3,4-tetrahydroisoquinoline-4-carboxamide (081)

This compound was obtained following method B, from thiophene-2-carboxaldehyde, 2-methoxyethanamine, homophthalic anhydride and benzo[d]thiazol-5-amine in 4% overall yield. The title compound was purified three times by flash chromatography eluting twice with dichloromethane, 0 to 5% methanol, and once with heptane, 10 to 80% EtOAc. ESI/APCI (+) 464 (M+H). ¹H NMR (DMSO-d₆) δ 2.98 (s, 3H), 3.06-3.10 (m, 2H), 3.43-3.47 (m, 2H), 4.34 (s, 1H), 5.65 (s, 1H), 6.91-6.94 (m, 1H), 7.03 (d, 1H), 7.31 (d, NH), 7.38-7.50 (m, 3H), 7.63-7.65 (m, 1H), 7.93 (d, 1H), 8.08 (d, 1H), 8.47 (d, 1H), 9.37 (s, 1H), 10.66 (s, 1H).

### Example 52

### Preparation of N-(biphenyl-2-yl)-2-(2-methoxyethyl)-1-oxo-3-(thiophen-2-yl)-1,2,3,4-tetrahydroisoquinoline-4-carboxamide (C84)

This compound was obtained following method B, from thiophene-2-carboxaldehyde, 2-methoxyethanamine, homophthalic anhydride and 2-amino-biphenyl in 1% overall yield. The title compound was purified three times by flash chromatography eluting twice with heptane, 10 to 80% EtOAc. ESI/APCI (+) 483 (M+H). ¹H NMR (DMSO-d₆) δ 3.08-3.14 (m, 2H), 3.12 (s, 3H), 3.40-6.46 (m, 2H), 4.21 (s, 1H), 5.48 (s, 1H), 6.87-6.90 (m, 1H), 6.93 (d, 1H), 7.14-7.17 (m, NH), 7.27-7.31 (m, 5H), 7.34-7.46 (m, 6H), 7.70-7.72 (m, 1H), 7.88-7.91 (m, 1H), 9.11 (s, 1H).

### Example 53

### Preparation of 2-(2-methoxyethyl)-1-oxo-3-(thiophen-2-yl)-N-m-tolyl-1,2,3,4-tetrahydroisoquinoline-4-carboxamide (C85)

This compound was obtained following method B, from thiophene-2-carboxaldehyde, 2-methoxyethanamine, homophthalic anhydride and m-toluidine in 1% overall yield. The title compound was purified by flash chromatography eluting with heptane, 10 to 80% EtOAc. ESI/APCI (+) 421 (M+H). ¹H NMR (DMSO-d₆) δ 2.27 (s, 3H), 3.01 (s, 3H), 3.04-3.10 (m, 2H), 3.40-3.45 (m, 2H), 4.27 (s, 1H), 5.58 (s, 1H), 6.86-6.92 (m, 2H), 7.01 (d, NH), 7.16-7.21 (t, 1H), 7.30-7.48 (m, 6H), 7.91 (d, 1H), 10.30 (s, 1H).

### Example 54

### Preparation of 2-(2-methoxyethyl)-1-oxo-3-(thiophen-2-yl)-N-p-tolyl-1,2,3,4-tetrahydroisoquinoline-4-carboxamide (C86)

This compound was obtained following method B, from thiophene-2-carboxaldehyde, 2-methoxyethanamine, homophthalic anhydride and p-toluidine in 1% overall yield. The title compound was purified by flash chromatography eluting with heptane, 10 to 80% EtOAc. ESI/APCI (+) 421 (M+H). ¹H NMR (DMSO-d₆) δ 2.24 (s, 3H), 0.03 (s, 3H), 3.05-3.09 (m, 2H), 3.35-3.45 (m, 2H), 4.25 (s, 1H), 5.58 (s, 1H), 6.89-6.92 (m, 1H), 7.01 (d, NH), 7.10-7.12 (d, 2H), 7.30-7.35 (m, 2H), 7.42-7.50 (m, 4H), 7.90-7.92 (m, 1H), 10.29 (s, 1H).

### Example 55

### Preparation of N-(3,4-dichlorophenyl)-2-(2-methoxyethyl)-1-oxo-3-(thiophen-2-yl)-1,2,3,4-tetrahydroisoquinoline-4-carboxamide (C87)

This compound was obtained following method B, from thiophene-2-carboxaldehyde, 2-methoxyethanamine, homophthalic anhydride and 3,4-dichloroaniline in 2% overall yield. The title compound was purified by flash chromatography eluting with heptane, 10 to 60% EtOAc. ESI/APCI (+) 475 (M+H). ¹H NMR (DMSO-d₆) δ 2.99 (s, 3H), 3.00-3.07 (m, 2H), 3.38-3.44 (m, 2H), 4.28 (s, 1H), 5.59 (s, 1H), 6.90-6.92 (m, 1H), 7.00 (d, NH), 7.30-7.36 (m, 2H), 7.42-7.53 (m, 3H), 7.59-7.61 (d, 1H), 7.91-7.94 (m, 1H), 8.00 (d, 1H), 10.72 (s, 1H).

### Example 56

### Preparation of N-(3,4-difluorophenyl)-2-(2-methoxyethyl)-1-oxo-3-(thiophen-2-yl)-1,2,3,4-tetrahydroisoquinoline-4-carboxamide (C88)

This compound was obtained following method B, from thiophene-2-carboxaldehyde, 2-methoxyethanamine, homophthalic anhydride and 3,4-difluoroaniline in 3% overall yield. The title compound was purified by flash chromatography eluting with heptane, 10 to 60% EtOAc. ESI/APCI (+) 441 (M+H). ¹H NMR (DMSO-d₆) δ 3.00 (s, 3H), 3.03-3.08 (m, 2H), 3.40-3.44 (m, 2H), 4.26 (s, 1H), 5.58 (s, 1H), 6.89-6.93 (m, 1H), 7.00 (d, NH), 7.31-7.37 (m, 3H), 7.40-7.49 (m, 3H), 7.75-7.82 (m, 1H), 7.91-7.93 (m, 1H), 10.65 (s, 1H).

### Example 57

### Preparation of N-(3-benzylphenyl)-2-(2-methoxyethyl)-1-oxo-3-(thiophen-2-yl)-1,2,3,4-tetrahydroisoquinoline-4-carboxamide (C89)

This compound was obtained following method B, from thiophene-2-carboxaldehyde, 2-methoxyethanamine, homophthalic anhydride and 3-benzylaniline in 6% overall yield. The title compound was purified by flash chromatography eluting with heptane, 10 to 60% EtOAc. ESI/APCI (+) 497 (M+H). ¹H NMR (DMSO-d₆) δ 2.94 (s, 3H), 3.06-3.15 (m, 2H), 3.47-3.55 (m, 2H), 3.92 (s, 2H), 4.20 (s, 1H), 5.67 (s, 1H), 6.85-6.87 (m, 1H), 6.94-6.97 (m, 2H), 7.12-7.25 (m, 9H), 7.43-7.52 (m, 4H), 8.01-8.05 (d, 1H).

### Example 58

### Preparation of N-(4-benzylphenyl)-2-(2-methoxyethyl)-1-oxo-3-(thiophen-2-yl)-1,2,3,4-tetrahydroisoquinoline-4-carboxamide (C90)

This compound was obtained following method B, from thiophene-2-carboxaldehyde, 2-methoxyethanamine, homophthalic anhydride and 4-benzylaniline in 2% overall yield. The title compound was purified by flash chromatography eluting with heptane, 10 to 60% EtOAc. ESI/APCI (+) 497 (M+H). ¹H NMR (DMSO-d₆) δ 3.03 (s, 3H), 3.09-3.18 (m, 2H), 3.50-3.58 (m, 2H), 3.91 (s, 2H), 4.17 (s, 1H), 5.69 (s, 1H), 6.86-6.88 (m, 1H), 6.95-6.96 (d, NH), 7.12-7.27 (m, 10H), 7.43-7.53 (m, 4H), 8.03-8.05 (m, 1H).

### Example 59

### Preparation of N-(3-chloro-4-fluorophenyl)-2-(2-methoxyethyl)-1-oxo-3-(thiophen-2-yl)-1,2,3,4-tetrahydroisoquinoline-4-carboxamide (C94)

This compound was obtained following method B, from thiophene-2-carboxaldehyde, 2-methoxyethanamine, homophthalic anhydride and 3-chloro-4-fluoroaniline in 3% overall yield. The title compound was purified by flash chromatography eluting with heptane, 10 to 60% EtOAc. ESI/APCI (+) 459 (M+H). ¹H NMR (DMSO-d₆) δ 3.02-3.11 (m, 2H), 3.41-3.45 (m, 2H), 4.27 (s, 1H), 5.59 (s, 1H), 6.90-6.93 (m, 1H), 7.00 (d, NH), 7.31-7.43 (m, 2H), 7.44-7.49 (m, 4H), 7.92-7.95 (m, 2H), 10.63 (s, 1H).

### Example 60

### Preparation of N-(4-fluorophenyl)-2-(2-methoxyethyl)-1-oxo-3-(thiophen-2-yl)-1,2,3,4-tetrahydroisoquinoline-4-carboxamide (C95)

This compound was obtained following method B, from thiophene-2-carboxaldehyde, 2-methoxyethanamine, homophthalic anhydride and 4-fluoroaniline in 3% overall yield. The title compound was purified by flash chromatography eluting with heptane, 10 to 60% EtOAc. ESI/APCI (+) 425 (M+H). ¹H NMR (DMSO-d₆) δ 3.00 (s, 3H), 3.04-3.10 (m, 2H), 3.41-3.44 (m, 2H), 4.26 (s, 1H), 5.89 (s, 1H), 6.89-6.93 (m, 1H), 7.00 (d, NH), 7.13-7.19 (m, 2H), 7.30-7.36 (m, 2H), 7.41-7.51 (m, 2H), 7.60-7.65 (m, 2H), 7.91-7.93 (m, 1H), 10.45 (s, 1H).

### Example 61

### Preparation of 2-(2-methoxyethyl)-1-oxo-N-(4-(piperidin-1-yl)phenyl)-3-(thiophen-2-yl)-1,2,3,4-tetrahydroisoquinoline-4-carboxamide (C107)

This compound was obtained following method B, from thiophene-2-carboxaldehyde, 2-methoxyethanamine, homophthalic anhydride and 4-(piperidin-1-yl)aniline in 1% overall yield. The title compound was purified by flash chromatography eluting with heptane, 10 to 60% EtOAc. ESI/APCI (+) 490 (M+H). ¹H NMR (DMSO-d₆) δ 1.50-1.51 (m, 2H), 1.58-1.60 (m, 4H), 3.03-3.10 (m, 9H), 3.41-3.45 (m, 2H), 4.22 (s, 1H), 5.56 (s, 1H), 6.86-6.92 (m, 3H), 7.00 (d, NH), 7.29-7.35 (m, 2H), 7.40-7.48 (m, 4H), 7.90-7.92 (d, 1H), 10.11 (s, 1H).

### Example 62

### Preparation of N-(3-acetamidophenyl)-2-(2-methoxyethyl)-1-oxo-3-(thiophen-2-yl)-1,2,3,4-tetrahydroisoquinoline-4-carboxamide (C108)

This compound was obtained following method B, from thiophene-2-carboxaldehyde, 2-methoxyethanamine, homophthalic anhydride and 3′-aminoacetaniline in 2% overall yield. The title compound was purified by flash chromatography eluting with dichloromethane, 0 to 5% methanol. ESI/APCI (+) 425 (M+H). ¹H NMR (DMSO-d₆) δ 2.03 (s, 3H), 3.01 (s, 3H), 3.36-3.46 (m, 2H), 3.99-4.11 (m, 2H), 4.30 (s, 1H), 5.60 (s, 1H), 6.89-6.92 (m, 1H), 7.00-7.02 (d, NH), 7.17-7.21 (m, 2H), 7.29-7.36 (m, 3H), 7.39-7.50 (m, 2H), 7.91-7.98 (m, 2H), 9.95 (s, 1H), 10.42 (s, 1H).

### Example 63

### Preparation of N-(1,3-dimethyl-1H-pyrazol-5-yl)-2-(2-methoxyethyl)-1-oxo-3-(thiophen-2-yl)-1,2,3,4-tetrahydroisoquinoline-4-carboxamide (C109)

This compound was obtained following method B, from thiophene-2-carboxaldehyde, 2-methoxyethanamine, homophthalic anhydride and 1,3-dimethyl-1H-pyrazol-5-amine in 1% overall yield. The title compound was purified by flash chromatography eluting with heptane, 10 to 60% EtOAc. ESI/APCI (+) 425 (M+H). ¹H NMR (DMSO-d₆) δ 2.06 (s, 3H), 3.10 (s, 3H), 3.12-3.17 (m, 1H), 3.35 (s, 3H), 3.39-3.46 (m, 2H), 3.99-4.05 (m, 1H), 4.36 (s, 1H), 5.59 (s, 1H), 5.98 (s, 1H), 6.89-6.92 (t, 1H), 7.01-7.02 (d, 1H), 7.30-7.32 (d, 1H), 7.37-7.53 (m, 3H), 7.91-7.93 (d, 1H), 10.24 (s, NH).

### Example 64

### Preparation of 2-(2-methoxyethyl)-1-oxo-3-(thiophen-2-yl)-N-(3-(trifluoromethyl)phenyl)-1,2,3,4-tetrahydroisoquinoline-4 carboxamide (C110)

This compound was obtained following method B, from thiophene-2-carboxaldehyde, 2-methoxyethanamine, homophthalic anhydride and 3-(trifluoromethyl)aniline in 6% overall yield. The title compound was purified by flash chromatography eluting with heptane, 10 to 60% EtOAc. ESI/APCI (+) 475 (M+H). ¹H NMR (DMSO-d₆) δ 2.93 (s, 3H), 3.03-3.07 (m, 2H), 3.39-3.42 (m, 2H), 4.30 (s, 1H), 5.61 (s, 1H), 6.90-6.93 (m, 1H), 7.00 (s, NH), 7.31-7.34 (m, 2H), 7.36-7.52 (m, 3H), 7.65-7.60 (m, 1H), 7.78-7.80 (d, 1H), 7.92-7.94 (d, 1H), 8.11 (s, 1H), 10.76 (s, 1H).

### Example 65

### Preparation of N-(4-chloro-3-methoxyphenyl)-2-(2-methoxyethyl)-1-oxo-3-(thiophen-2-yl)-1,2,3,4-tetrahydroisoquinoline-4-carboxamide (C111)

This compound was obtained following method B, from thiophene-2-carboxaldehyde, 2-methoxyethanamine, homophthalic anhydride and 4-chloro-3-methoxyaniline in 2% overall yield. The title compound was purified by flash chromatography eluting with heptane, 10 to 60% EtOAc. ESI/APCI (+) 471 (M+H). ¹H NMR (DMSO-d₆) δ 2.92 (s, 3H), 2.99-3.10 (m, 1H), 3.39-3.41 (m, 2H), 3.80 (s, 3H), 4.06-4.10 (m, 1H), 4.27 (s, 1H), 5.59 (s, 1H), 6.89-6.92 (m, 1H), 7.00 (d, NH), 7.14-7.17 (m, 1H), 7.30-7.36 (m, 3H), 7.40-7.54 (m, 3H), 7.90-7.93 (d, 1H), 10.57 (s, 1H).

### Example 66

### Preparation of 2-(2-methoxyethyl)-1-oxo-3-(thiophen-2-yl)-N-(3-(trifluoromethoxy)phenyl)-1,2,3,4-tetrahydroisoquinoline-4-carboxamide (C112)

This compound was obtained following method B, from thiophene-2-carboxaldehyde, 2-methoxyethanamine, homophthalic anhydride and 3-(trifluoromethoxy)aniline in 17% overall yield. The title compound was purified by flash chromatography eluting with heptane, 10 to 60% EtOAc. ESI/APCI (+) 491 (M+H). ¹H NMR (DMSO-d₆) δ 2.95 (s, 3H), 3.02-3.06 (m, 1H), 3.36-3.43 (m, 2H), 4.06-4.12 (m, 1H), 4.29 (s, 1H), 5.59 (s, 1H), 6.89-6.93 (m, 1H), 7.00 (d, NH), 7.04-7.06 (d, 1H), 7.31-7.36 (m, 2H), 7.42-7.52 (m, 4H), 7.82 (s, 1H), 7.91-7.94 (d, 1H), 10.73 (s, 1H).

### Example 67

### Preparation of 2-(2-methoxyethyl)-1-oxo-3-(thiophen-2-yl)-N-(4-(trifluoromethoxy)phenyl)-1,2,3,4-tetrahydroisoquinoline-4-carboxamide (C113)

This compound was obtained following method B, from thiophene-2-carboxaldehyde, 2-methoxyethanamine, homophthalic anhydride and 4-(trifluoromethoxy)aniline in 12% overall yield. The title compound was purified by flash chromatography eluting with heptane, 10 to 60% EtOAc. ESI/APCI (+) 491 (M+H). ¹H NMR (DMSO-d₆) δ 2.98 (s, 3H), 3.04-3.08 (m, 1H), 3.40-3.44 (m, 2H), 4.04-4.10 (m, 1H), 4.28 (s, 1H), 5.59 (s, 1H), 6.89-6.92 (m, 1H), 7.00 (d, NH), 7.30-7.36 (m, 4H), 7.41-7.49 (m, 2H), 7.69-7.73 (d, 2H), 7.91-7.93 (d, 1H), 10.60 (s, 1H).

### Example 68

### Preparation of methyl 2-methoxy-4-(2-(2-methoxyethyl)-1-oxo-3-(thiophen-2-yl)-1,2,3,4-tetrahydroisoquinoline-4-carboxamido)benzoate (C114)

This compound was obtained following method B, from thiophene-2-carboxaldehyde, 2-methoxyethanamine, homophthalic anhydride and methyl 4-amino-2-methoxybenzoate in 10% overall yield. The title compound was purified by flash chromatography eluting with heptane, 10 to 60% EtOAc. ESI/APCI (+) 495 (M+H). ¹H NMR (DMSO-d₆) δ 2.96 (s, 3H), 3.01-3.10 (m, 1H), 3.37-3.45 (m, 2H), 3.74 (s, 3H), 3.77 (s, 3H), 4.06-4.13 (m, 1H), 4.32 (s, 1H), 5.60 (s, 1H), 6.90-6.93 (t, 1H), 7.00-7.01 (d, NH), 7.18-7.21 (d, 1H), 7.30-7.36 (m, 2H), 7.41-7.51 (m, 2H), 7.56 (s, 1H), 7.69-7.72 (d, 1H), 7.91-7.94 (d, 1H), 10.74 (s, 1H).

### Example 69

### Preparation of N-(3-(1H-pyrrol-1-yl)phenyl)-2-(2-methoxyethyl)-1-oxo-3-(thiophen-2-yl)-1,2,3,4-tetrahydroisoquinoline-4-carboxamide (C115)

This compound was obtained following method B, from thiophene-2-carboxaldehyde, 2-methoxyethanamine, homophthalic anhydride and 3-(1H-pyrrol-1-yl)aniline in 15% overall yield. The title compound was purified by flash chromatography eluting with heptane, 10 to 60% EtOAc. ESI/APCI (+) 472 (M+H). ¹H NMR (DMSO-d₆) δ 2.89 (s, 3H), 3.04-3.09 (m, 1H), 3.41-3.45 (m, 2H), 4.01-4.11 (m, 1H), 4.31 (s, 1H), 5.61 (s, 1H), 6.26 (s, 2H), 6.90-6.93 (t, 1H), 7.01-7.02 (d, NH), 7.25-7.27 (m, 3H), 7.30-7.37 (m, 2H), 7.40-7.50 (m, 4H), 7.87 (s, 1H), 7.91-7.94 (d, 1H), 10.59 (s, 1H).

### Example 70

### Preparation of N-(4-(1H-pyrrol-1-yl)phenyl)-2-(2-methoxyethyl)-1-oxo-3-(thiophen-2-yl)-1,2,3,4-tetrahydroisoquinoline-4-carboxamide (C116)

This compound was obtained following method B, from thiophene-2-carboxaldehyde, 2-methoxyethanamine, homophthalic anhydride and 4-(1H-pyrrol-1-yl)aniline in 12% overall yield. The title compound was purified by flash chromatography eluting with heptane, 10 to 60% EtOAc. ESI/APCI (+) 472 (M+H). ¹H NMR (DMSO-d₆) δ 3.02 (s, 3H), 3.05-3.09 (m, 1H), 3.41-3.45 (m, 2H), 4.06-4.11 (m, 1H), 4.28 (s, 1H), 5.60 (s, 1H), 6.23 (s, 2H), 6.90-6.93 (m, 1H), 7.01-7.02 (d, NH), 7.32-7.37 (m, 4H), 7.43-7.55 (m, 4H), 7.66-7.69 (d, 2H), 7.91-7.93 (d, 1H), 10.50 (s, 1H).

### Example 71

### Preparation of N-(2,3-dihydrobenzo[b][1,4]dioxin-6-yl)-2-(2-methoxyethyl)-1-oxo-3-(thiophen-2-yl)-1,2,3,4-tetrahydroisoquinoline-4-carboxamide (C117)

This compound was obtained following method B, from thiophene-2-carboxaldehyde, 2-methoxyethanamine, homophthalic anhydride and 2,3-dihydrobenzo[b][1,4]dioxin-6-amine in 15% overall yield. The title compound was purified by flash chromatography eluting with heptane, 10 to 60% EtOAc. ESI/APCI (+) 465 (M+H). ¹H NMR (DMSO-d₆) δ 3.05 (s, 3H), 3.40-3.44 (m, 2H), 3.99-4.10 (m, 2H), 4.19 (s, 4H), 4.21 (s, 1H), 5.55 (s, 1H), 6.77-6.80 (d, 1H), (t, 1H), 6.95-6.96 (d, 1H), 6.98-7.00 (m, 1H), 7.22-7.23 (d, NH), 7.29-7.34 (m, 2H), 7.39-7.50 (m, 2H), 7.89-7.92 (d, 1H), 10.21 (s, 1H).

### Example 72

### Preparation of 2-(2-methoxyethyl)-N-(4-morpholinophenyl)-1-oxo-3-(thiophen-2-yl)-1,2,3,4-tetrahydroisoquinoline-4-carboxamide (C121)

This compound was obtained following method B, from thiophene-2-carboxaldehyde, 2-methoxyethanamine, homophthalic anhydride and 4-morpholinoaniline in 5% overall yield. The title compound was purified by flash chromatography eluting with heptane, 10 to 60% EtOAc. ESI/APCI (+) 492 (M+H). ¹H NMR (DMSO-d₆) δ 2.36-2.42 (m, 2H), 2.99-3.09 (m, 7H), 3.41-3.45 (m, 2H), 3.70-3.73 (m, 4H), 4.22 (1H), 5.60 (s, 1H), 6.88-6.92 (m, 3H), 7.00-7.01 (d, NH), 7.29-7.35 (m, 2H), 7.42-7.48 (m, 4H), 7.90-7.92 (d, 1H), 10.16 (s, 1H).

### Example 73

### Preparation of 2-(2-methoxyethyl)-1-oxo-N-(3-(pyrrolidin-1-yl)phenyl)-3-(thiophen-2-yl)-1,2,3,4-tetrahydroisoquinoline-4-carboxamide (C122)

This compound was obtained following method B, from thiophene-2-carboxaldehyde, 2-methoxyethanamine, homophthalic anhydride and 3-(pyrrolidin-1-yl)aniline in 10% overall yield. The title compound was purified by flash chromatography eluting with heptane, 10 to 60% EtOAc. ESI/APCI (+) 476 (M+H). ¹H NMR (DMSO-d₆) δ 1.93 (s, 4H), 3.04 (s, 3H), 3.05-3.10 (m, 1H), 3.15-3.19 (t, 4H), 3.40-3.44 (m, 2H), 4.04-4.09 (m, 1H), 4.26 (s, 1H), 5.57 (s, 1H), 6.23-6.25 (d, 1H), 6.81-6.84 (d, 1H), 6.89-6.92 (m, 2H), 7.00-7.09 (m, 2H), 7.29-7.34 (m, 2H), 7.41-7.45 (m, 2H), 7.90-7.92 (m, 1H), 10.86 (s, 1H).

### Example 74

### Preparation of 2-(2-methoxyethyl)-N-(1-methyl-1H-indol-5-yl)-1-oxo-3-(thiophen-2-yl)-1,2,3,4-tetrahydroisoquinoline-4-carboxamide (C123)

This compound was obtained following method B, from thiophene-2-carboxaldehyde, 2-methoxyethanamine, homophthalic anhydride and 1-methyl-1H-indol-5-amine in 14% overall yield. The title compound was purified by flash chromatography eluting with heptane, 10 to 60% EtOAc. ESI/APCI (+) 460 (M+H). ¹H NMR (DMSO-d₆) δ 3.03 (s, 3H), 3.05-3.14 (m, 1H), 3.43-3.48 (m, 2H), 3.75 (s, 3H), 4.03-4.09 (m, 1H), 4.27 (s, 1H), 5.61 (s, 1H), 6.34-6.35 (d, 1H), 6.90-6.93 (t, 1H), 7.02-7.04 (d, NH), 7.27-7.31 (m, 3H), 7.35-7.40 (t, 2H), 7.42-7.48 (m, 2H), 7.85 (s, 1H), 7.91-7.93 (d, 1H), 10.81 (s, 1H).

### Example 75

### Preparation of 2-(2-methoxyethyl)-N-((1R,4Rr)-4-methylcyclohexyl)-1-oxo-3-(thiophen-2-yl)-1,2,3,4-tetrahydroisoquinoline-4-carboxamide (C124)

This compound was obtained following method B, from thiophene-2-carboxaldehyde, 2-methoxyethanamine, homophthalic anhydride and trans-4-methylcyclohexylamine in 19% overall yield. The title compound was purified by flash chromatography eluting with heptane, 10 to 60% EtOAc. ESI/APCI (+) 427 (M+H). ¹H NMR (DMSO-d₆) δ 0.83-0.86 (d, 3H), 0.89-0.97 (m, 2H), 1.11-1.31 (m, 3H), 1.60-1.69 (t, 3H), 1.82-1.86 (t, 1H), 3.07-3.14 (m, 1H), 3.20 (s, 3H), 3.37-3.48 (m, 2H), 3.97 (s, 1H), 5.38 (s, 1H), 6.86-6.89 (m, 1H), 6.95-6.96 (d, 1H), 7.22-7.28 (m, 2H), 7.36-7.47 (m, 2H), 7.87-7.89 (d, 1H), 7.96-7.99 (d, NH).

### Example 76

### Preparation of N-(5-ethyl-1,3,4-oxadiazol-2-yl)-2-(2-methoxyethyl)-1-oxo-3-(thiophen-2-yl)-1,2,3,4-tetrahydroisoquinoline-4-carboxamide (C125)

This compound was obtained following method B, from thiophene-2-carboxaldehyde, 2-methoxyethanamine, homophthalic anhydride and 5-ethyl-1,3,4-oxadiazol-2-amine in 6% overall yield. The title compound was purified by flash chromatography eluting with heptane, 10 to 60% EtOAc. ESI/APCI (+) 427 (M+H). ¹H NMR (DMSO-d₆) δ 1.15-1.23 (m, 3H), 2.73-2.80 (m, 2H), 3.05 (s, 3H), 3.09-3.11 (m, 1H), 3.41-3.44 (m, 2H), 3.99-4.11 (m, 1H), 4.27 (s, 1H), 5.67 (s, 1H), 6.87-6.99 (m, 2H), 7.28-7.49 (m, 4H), 7.82-7.97 (m, 1H), 12.09-12.35 (m, 1H).

### Example 77

### Preparation of 2-(2-methoxyethyl)-N-(5-methylisoxazol-3-yl)-1-oxo-3-(thiophen-2-yl)-1,2,3,4-tetrahydroisoquinoline-4-carboxamide (C126)

This compound was obtained following method B, from thiophene-2-carboxaldehyde, 2-methoxyethanamine, homophthalic anhydride and 5-methyl-isoxazole-3-amine in 12% overall yield. The title compound was purified by flash chromatography eluting with heptane, 10 to 60% EtOAc. ESI/APCI (+) 412 (M+H). ¹H NMR (DMSO-d₆) δ 2.35 (s, 3H), 3.02 (s, 3H), 3.37-3.41 (m, 2H), 4.07-4.11 (m, 2H), 4.31 (s, 1H), 5.60 (s, 1H), 6.58 (s, 1H), 6.88-6.91 (m, 1H), 6.89-6.99 (d, NH), 7.27-7.32 (m, 2H), 7.40-7.46 (m, 2H), 7.89-7.92 (m, 1H), 11.53 (s, 1H).

### Example 78

### Preparation of 2-(2-methoxyethyl)-N-(3-methylisoxazol-5-yl)-1-oxo-3-(thiophen-2-yl)-1,2,3,4-tetrahydroisoquinoline-4-carboxamide (C127)

This compound was obtained following method B, from thiophene-2-carboxaldehyde, 2-methoxyethanamine, homophthalic anhydride and 3-methyl-isoxazole-5-amine in 1% overall yield. The title compound was purified by flash chromatography eluting with heptane, 10 to 60% EtOAc. ESI/APCI (+) 412 (M+H). ¹H NMR (DMSO-d₆) δ 2.16 (s, 3H), 2.99 (s, 3H), 3.01-3.06 (m, 1H), 3.37-3.4 (m, 2H), 4.06-4.12 (m, 1H), 4.32 (s, 1H), 5.61 (s, 1H), 6.08 (s, 1H), 6.88-6.91 (m, 1H), 6.96-6.97 (d, 1H), 7.29-7.34 (m, 2H), 7.41-7.48 (m, 2H), 7.89-7.92 (m, 1H), 12.13 (s, NH).

### Example 79

### Preparation of N-(4-acetamidophenyl)-2-(2-methoxyethyl)-1-oxo-3-(thiophen-2-yl)-1,2,3,4-tetrahydroisoquinoline-4-carboxamide (C129)

This compound was obtained following method B, from thiophene-2-carboxaldehyde, 2-methoxyethanamine, homophthalic anhydride and N-(4-aminophenyl)acetamide in 1% overall yield. The title compound was purified by flash chromatography eluting with dichloromethane, 0 to 8% methanol. ESI/APCI (+) 464 (M+H). ¹H NMR (DMSO-d₆) δ 2.00 (s, 3H), 3.02 (s, 3H), 3.05-3.12 (m, 1H), 3.40-3.44 (m, 2H), 4.04-4.08 (m, 1H), 4.24 (s, 1H), 5.57 (s, 1H), 6.89-6.92 (m, 1H), 7.00-7.01 (d, NH), 7.29-7.35 (m, 2H), 7.39-7.46 (m, 2H), 7.48-7.50 (m, 4H), 7.90-7.92 (m, 1H), 9.87 (s, 1H), 10.30 (s, 1H).

### Example 80

### Preparation of N-(3,5-dimethoxyphenyl)-2-(2-methoxyethyl)-1-oxo-3-(thiophen-2-yl)-1,2,3,4-tetrahydroisoquinoline-4-carboxamide (C130)

This compound was obtained following method B, from thiophene-2-carboxaldehyde, 2-methoxyethanamine, homophthalic anhydride and 3,5-dimethoxyaniline in 4% overall yield. The title compound was purified by flash chromatography eluting with heptane, 10 to 60% AcOEt. ESI/APCI (+) 467 (M+H). ¹H NMR (DMSO-d₆) δ 3.02 (s, 3H), 3.04-3.09 (m, 1H), 3.39-3.43 (m, 2H), 3.70 (m, 6H), 4.05-4.10 (m, 1H), 4.25 (s, 1H), 5.57 (s, 1H), 6.21 (s, 1H), 6.85-6.86 (m, 2H), 6.89-6.92 (m, 1H), 7.00-7.01 (d, NH), 7.30-7.34 (m, 2H), 7.40-7.51 (m, 2H), 7.91-7.93 (m, 1H), 10.37 (s, 1H).

### Example 81

### Preparation of 2-(2-methoxyethyl)-N-(2-methyl-1H-indol-5-yl)-1-oxo-3-(thiophen-2-yl)-1,2,3,4-tetrahydroisoquinoline-4-carboxamide (C131)

This compound was obtained following method B, from thiophene-2-carboxaldehyde, 2-methoxyethanamine, homophthalic anhydride and 2-methylindole-5-amine in 1% overall yield. The title compound was purified by preparative HPLC eluting with a gradient of acetonitrile in water (with 0.1% formic acid). ESI/APCI (+) 460 (M+H). ¹H NMR (DMSO-d₆) δ 2.49 (s, 3H), 3.04 (s, 3H), 3.08-3.13 (m, 1H), 3.43-3.47 (m, 2H), 4.02-4.08 (m, 1H), 4.25 (s, 1H), 5.59 (s, 1H), 6.04 (s, 1H), 6.90-6.92 (m, 1H), 7.02 (d, NH), 7.09-7.19 (m, 2H), 7.29-7.30 (d, 1H), 7.35-7.51 (m, 3H), 7.69 (s, 1H), 7.91-7.94 (d, 1H), 10.05 (s, 1H), 10.30 (s, NH).

### Example 82

### Preparation of N-(1H-indazol-5-yl)-2-(2-methoxyethyl)-1-oxo-3-(thiophen-2-yl)-1,2,3,4-tetrahydroisoquinoline-4-carboxamide (C132)

This compound was obtained following method B, from thiophene-2-carboxaldehyde, 2-methoxyethanamine, homophthalic anhydride and 1H-indazole-5-amine in 1% overall yield. The title compound was purified by preparative HPLC eluting with a gradient of acetonitrile in water (with 0.1% formic acid). ESI/APCI (+) 447 (M+H). ¹H NMR (DMSO-d₆) δ 3.01 (s, 3H), 3.07-3.14 (m, 1H), 3.43-3.47 (m, 2H), 4.05-4.09 (m, 1H), 4.28 (s, 1H), 5.61 (s, 1H), 6.90-6.92 (m, 1H), 7.02 (s, NH), 7.29-7.31 (d, 1H), 7.36-7.38 (m, 1H), 7.43-7.51 (m, 4H), 7.91-7.94 (m, 1H), 7.99 (s, 1H), 8.09 (s, 1H), 10.34 (s, 1H), 12.97 (s, NH).

### Example 83

### Preparation of 2-(2-methoxyethyl)-N,3-bis(3-methoxyphenyl)-1-oxo-1,2,3,4-tetrahydroisoquinoline-4-carboxamide (C133)

This compound was obtained following method B, from m-anisaldehyde, 2-methoxyethanamine, homophthalic anhydride and m-anisidine in 20% overall yield. The title compound was purified by flash chromatography eluting with heptane, 10 to 60% EtOAc. ESI/APCI (+) 461 (M+H). ¹H NMR (DMSO-d₆) δ 2.96-3.03 (m, 1H), 3.01 (s, 3H), 3.38-3.44 (m, 2H), 3.67 (s, 3H), 3.72 (s, 3H), 4.02-4.08 (m, 1H), 4.17 (s, 1H), 5.29 (s, 1H), 6.62-6.65 (d, 1H), 6.71-6.73 (d, 1H), 6.77-6.80 (d, 2H), 7.13-7.25 (m, 4H), 7.32 (s, 1H), 7.38-7.43 (m, 2H), 7.93-7.95 (m, 1H), 10.38 (s, 1H).

### Example 84

### Preparation of 2-(2-methoxyethyl)-3-(2-methoxyphenyl)-N-(3 methoxyphenyl)-1-oxo-1,2,3,4-tetrahydroisoquinoline-4 carboxamide (C134)

This compound was obtained following method B, from o-anisaldehyde, 2-methoxyethanamine, homophthalic anhydride and m-anisidine in 4% overall yield. The title compound was purified by flash chromatography eluting with heptane, 10 to 60% EtOAc. ESI/APCI (+) 461 (M+H). ¹H NMR (DMSO-d₆) δ 3.08 (s, 3H), 3.10-3.15 (m, 1H), 3.42-3.51 (m, 2H), 3.72 (s, 3H), 3.87-3.89 (m, 1H), 3.92 (s, 3H), 4.05 (s, 1H), 5.44 (s, 1H), 6.63-6.66 (d, 2H), 6.74-6.79 (m, 1H), 7.05-7.13 (m, 2H), 7.18-7.29 (m, 3H), 7.34-7.43 (m, 3H), 7.96-7.99 (m, 1H), 10.06 (s, 1H).

### Example 85

### Preparation of ethyl 1-(2-(2-methoxyethyl)-1-oxo-3-(thiophen-2-yl)-1,2,3,4-tetrahydroisoquinoline-4-carbonyl)piperidine-4-carboxylate (C136)

This compound was obtained following method B, from thiophene-2-carboxaldehyde, 2-methoxyethanamine, homophthalic anhydride and ethyl piperidine-4-carboxylate in 8% overall yield. The title compound was purified by preparative HPLC eluting with a gradient of acetonitrile in water (0.1% formic acid). ESI/APCI (+) 471 (M+H). ¹H NMR (DMSO-d₆) δ 1.20 (m, 4H), 1.36 (m, 1H), 1.50-1.79 (m, 2H), 1.84-1.99 (m, 2H), 2.69 (m, 1H), 2.79-2.91 (m, 1H), 2.99 (m, 1H), 3.21 (s, 3H), 3.38 (m, 2H), 4.06 (m, 5H), 4.66 (s, 1H), 6.90 (m, 1H), 7.01 (d, 1H), 7.23 (m, 1H), 7.29 (d, 1H), 7.37-7.47 (m, 2H), 7.88 (d, 1H).

### Example 86

### Preparation of N-(5-tert-butylisoxazol-3-yl)-2-(2-methoxyethyl)-1-oxo-3-(thiophen-2-yl)-1,2,3,4-tetrahydroisoquinoline-4-carboxamide (C142)

This compound was obtained following method B, from thiophene-2-carboxaldehyde, 2-methoxyethanamine, homophthalic anhydride and ethyl 3-amino-5-tert-butylisoxazole in 3% overall yield. The title compound was purified by flash chromatography eluting with heptane, 30 to 90% EtOAc. ESI/APCI (+) 458 (M+H). ¹H NMR (DMSO-d₆) δ 1.25 (s 9H), 3.01 (s, 3H), 3.05-3.10 (m, 1H), 3.32-3.42 (m, 2H), 4.06 (m, 1H), 4.32 (s, 1H), 5.59 (s, 1H), 6.56 (s, 1H), 6.89 (m, 1H), 6.97 (s, 1H), 7.29 (m, 2H), 7.42 (m, 2H), 7.92 (d, 1H), 11.57 (s, NH).

### Example 87

### Preparation of 5,8-difluoro-2-(2-methoxyethyl)-N-(3-methoxyphenyl)-1-oxo-3-(thiophen-2-yl)-1,2,3,4-tetrahydroisoquinoline-4-carboxamide (C91)

This compound was obtained following method B, from thiophene-2-carboxaldehyde, 2-methoxyethanamine, 5,8-difluorohomophthalic anhydride and 3-methoxyaniline in 2% overall yield. The title compound was purified twice by flash chromatography eluting with heptane, 0 to 65% EtOAc. ESI/APCI (+) 474 (M+H). ¹H NMR (DMSO-d₆) δ 2.91 (3H, s), 3.06-3.13 (1H, m), 3.39 (2H, m), 3.72 (3H, s), 4.04 (1H, m), 4.52 (1H, s), 5.63 (1H, s), 6.66 (1H, dd), 6.95 (1H, t), 7.05 (1H, m), 7.14 (1H, d), 7.22-7.37 (4H, m), 7.45 (1H, m), 10.66 (1H, s).

### Example 88

### Preparation of 8-fluoro-7-methoxy-2-(2-methoxyethyl)-N-(3-methoxyphenyl)-1-oxo-3-(thiophen-2-yl)-1,2,3,4-tetrahydroisoquinoline-4-carboxamide (C128)

This compound was obtained following method B, from thiophene-2-carboxaldehyde, 2-methoxyethanamine, 8-fluoro-7-methoxyhomophthalic anhydride and 3-methoxyaniline in 58% overall yield. The title compound was purified twice by flash chromatography eluting with dichloromethane, 2 to 20% EtOAc. ESI/APCI(+): 485 (M+H). ¹H NMR (DMSO-d₆) δ 2.96 (s, 3H), 3.12 (m, 2H), 3.37 (m, 1H), 3.72 (s, 3H), 3.83 (s, 3H), 3.87 (dt, 1H), 4.22 (s, 1H), 5.53 (s, 1H), 6.62 (d, 1H), 6.91 (t, 1H), 7.01 (d, 1H), 7.07 (d, 1H), 7.10 (d, 1H), 7.20-7.28 (m, 2H), 7.31 (m, 2H), 10.36 (s, 1H, NH).

### Example 89

### Preparation of 2-(2-methoxyethyl)-N-(3-methoxyphenyl)-1-oxo-3-(thiophen-2-yl)-7-(trifluoromethyl)-1,2,3,4-tetrahydroisoquinoline-4-carboxamide (C92)

This compound was obtained following method B, from thiophene-2-carboxaldehyde, 2-methoxyethanamine, 7-trifluoromethylhomophthalic anhydride and 3-methoxyaniline in 7% overall yield. The title compound was purified by flash chromatography eluting with heptane, 0 to 65% EtOAc. ESI/APCI (+) 505 (M+H). ¹H NMR (DMSO-d₆) δ 2.96 (3H, s), 3.05-3.14 (1H, m), 3.38-3.44 (2H, m), 3.71 (3H, s), 4.12 (1H, m), 4.46 (1H, s), 5.67 (1H, s), 6.64 (1H, dd), 6.92 (1H, m), 7.03 (1H, d), 7.12-7.34 (3H, m), 7.64 (1H, m), 7.88 (1H, m), 8.15 (1H, s), 10.53 (1H, s).

### Example 90

### Preparation of 8-fluoro-2-(2-methoxyethyl)-N-(3-methoxyphenyl)-7-methyl-1-oxo-3-(thiophen-2-yl)-1,2,3,4-tetrahydroisoquinoline-4-carboxamide (C93)

This compound was obtained following method B, from thiophene-2-carboxaldehyde, 2-methoxyethanamine, 8-fluoro-7-methylhomophthalic anhydride and 3-methoxyaniline in 2% overall yield. The title compound was purified by flash chromatography eluting with heptane, 0 to 65% EtOAc and recrystallised from ethanol. ESI/APCI (+) 469 (M+H). ¹H NMR (DMSO-d₆) δ 2.23 (3H, s), 2.95 (3H, s), 3.08 (1H, m), 3.37 (2H, m), 3.71 (3H, s), 3.98 (1H, m), 4.26 (1H, s), 5.34 (1H, s), 6.64 (1H, dd), 6.92 (1H, m), 7.00-7.05 (2H, m), 7.13 (1H, d), 7.22 (1H, t), 7.31-7.38 (3H, m), 10.42 (1H, s).

### Example 91

### Preparation of 5-methoxy-2-(2-methoxyethyl)-N-(3-methoxyphenyl)-1-oxo-3-(thiophen-2-yl)-1,2,3,4-tetrahydroisoquinoline-4-carboxamide (C148)

This compound was obtained following method B, from thiophene-2-carboxaldehyde, 2-methoxyethanamine, 5-methoxyhomophthalic anhydride and 3-methoxyaniline in 16% overall yield. The title compound was purified by flash chromatography eluting with heptane, 10 to 90% EtOAc. ESI/APCI (+) 467 (M+H). ¹H NMR (DMSO-d₆) δ 2.93 (s, 3H), 3.00 (m, 1H), 3.39 (m, 2H), 3.72 (s, 3H), 3.78 (s, 3H), 4.07-4.15 (m, 1H), 4.48 (s, 1H), 5.53 (s, 1H), 6.63 (d, 1H), 6.90 (m, 1H), 7.00 (d, NH), 7.13-7.24 (m, 3H), 7.29 (d, 1H), 7.39 (m, 2H), 7.52 (d, 1H), 10.43 (s, 1H).

### Example 92

### Preparation of 3-(2,4-dimethylthiazol-5-yl)-2-(2-methoxyethyl)-N-(3-methoxyphenyl)-1-oxo-1,2,3,4-tetrahydroisoquinoline-4-carboxamide (C96)

This compound was obtained following method B, from 2,4-dimethylthiazole-5-carbaldehyde, 2-methoxyethanamine, homophthalic anhydride and 3-methoxyaniline in 8% overall yield. The title compound was purified by flash chromatography eluting with dichloromethane, 15 to 100% EtOAc. ESI/APCI (+) 466 (M+H). ¹H NMR (DMSO-d₆) δ 2.40 (s, 3H), 2.43 (s, 3H), 2.96 (dt, 1H), 3.04 (s, 3H), 3.43 (t, 2H), 3.71 (s, 3H), 3.94 (dt, 1H), 4.09 (s, 1H), 5.56 (s, 1H), 6.62 (dd, 1H), 7.09 (d, 1H), 7.19 (t, 1H), 7.30 (s, 1H), 7.38 (d, 1H), 7.47-7.56 (m, 2H), 7.93 (d, 1H), 10.30 (s, 1H, NH).

### Example 93

### Preparation of 2-(2-methoxyethyl)-N-(3-methoxyphenyl)-1-oxo-3-((tetrahydro-2H-pyran-4-yl)methyl)-1,2,3,4-tetrahydroisoquinoline-4-carboxamide (C97)

This compound was obtained following method B, from 2-(tetrahydro-2H-pyran-4-yl)acetaldehyde, 2-methoxyethanamine, homophthalic anhydride and 3-methoxyaniline in 30% overall yield. The title compound was purified by flash chromatography eluting with dichloromethane, 15 to 100% EtOAc. ESI/APCI(+): 453 (M+H). ¹H NMR (DMSO-d₆) δ 1.08-1.31 (m, 3H), 1.38-1.53 (m, 2H), 1.61 (m, 2H), 3.04 (s, 3H), 3.09 (m, 1H), 3.24 (m, 2H), 3.37-3.46 (m, 2H), 3.70 (s, 3H), 3.95 (m, 2H), 3.95 (s, 1H), 3.99-4.09 (m, 2H), 6.91 (dd, 1H), 7.08 (d, 1H), 7.17 (t, 1H), 7.28 (s, 1H), 7.41 (m, 2H), 7.48 (t, 1H), 7.85 (d, 1H), 10.12 (s, 1H, NH).

### Example 94

### Preparation of 2-(2-methoxyethyl)-N-(3-methoxyphenyl)-3-(2-methylthiazol-4-yl)-1-oxo-1,2,3,4-tetrahydroisoquinoline-4-carboxamide (C98)

This compound was obtained following method B, from 2-methylthiazole-4-carbaldehyde, 2-methoxyethanamine, homophthalic anhydride and 3-methoxyaniline in 50% overall yield. The title compound was purified by flash chromatography eluting with dichloromethane, 15 to 50% EtOAc. ESI/APCI(+): 452 (M+H). ¹H NMR (DMSO-d₆) δ 2.60 (s, 3H), 3.05 (s, 3H), 3.21 (dt, 1H), 3.45 (t, 2H), 3.71 (s, 3H), 4.11 (dt, 1H), 4.50 (s, 1H), 5.37 (s, 1H), 6.62 (dt, 1H), 6.96 (s, 1H), 7.15-7.23 (m, 2H), 7.30-7.45 (m, 4H), 7.88 (d, 1H), 10.45 (s, 1H, NH).

### Example 95

### Preparation of 3-(3,5-dimethylisoxazol-4-yl)-2-(2-methoxyethyl)-N-(3-methoxyphenyl)-1-oxo-1,2,3,4-tetrahydroisoquinoline-4-carboxamide (C99)

This compound was obtained following method B, from 3,5-dimethylisoxazol-4-carbaldehyde, 2-methoxyethanamine, homophthalic anhydride and 3-methoxyaniline in 9% overall yield. The title compound was purified twice by flash chromatography eluting first with dichloromethane, 15 to 50% EtOAc and then with heptane, 30 to 70% EtOAc. ESI/APCI(+): 450 (M+H). ¹H NMR (DMSO-d₆) δ 2.11 (s, 3H), 3.10 (m, 1H), 3.12 (s, 3H), 3.15 (m, 2H), 3.72 (s, 3H), 3.94 (dt, 1H), 4.12 (d, J=5.1 Hz, 1H), 5.19 (d, J=5.1 Hz, 1H), 6.65 (dd, 1H), 7.07 (d, 1H), 7.20 (m, 2H), 7.30 (d, 1H), 7.45 (t, 1H), 7.53 (t, 1H), 7.98 (d, 1H), 10.26 (s, 1H, NH).

### Example 96

### Preparation of 3-(1,3-dimethyl-1H-pyrazol-5-yl)-2-(2-methoxyethyl)-N-(3-methoxyphenyl)-1-oxo-1,2,3,4-tetrahydroisoquinoline-4-carboxamide (C100)

This compound was obtained following method B, from 1,3-dimethyl-1H-pyrazol-5-carbaldehyde, 2-methoxyethanamine, homophthalic anhydride and 3-methoxyaniline in 21% overall yield. The title compound was purified by flash chromatography eluting with dichloromethane, 30 to 100% EtOAc. ESI/APCI(+): 449 (M+H). ¹H NMR (DMSO-d₆) δ 1.93 (s, 3H), 3.07 (s, 3H), 3.17 (m, 1H), 3.42 (m, 2H), 3.72 (s, 3H), 3.86 (s, 3H), 3.88 (dt, 1H), 4.11 (s, 1H), 5.25 (s, 1H), 5.41 (s, 1H), 6.64 (dd, 1H), 7.10 (d, 1H), 7.19 (t, 1H), 7.28 (s, 1H), 7.33 (d, 1H), 7.43-7.53 (m, 2H), 7.95 (d, 1H), 10.21 (s, 1H, NH).

### Example 97

### Preparation of 3-(1,5-dimethyl-1H-pyrazol-3-yl)-2-(2-methoxyethyl)-N-(3-methoxyphenyl)-1-oxo-1,2,3,4-tetrahydroisoquinoline-4-carboxamide (C101)

This compound was obtained following method B, from 1,5-dimethyl-1H-pyrazol-3-carbaldehyde, 2-methoxyethanamine, homophthalic anhydride and 3-methoxyaniline in 33% overall yield. The title compound was purified by flash chromatography eluting with dichloromethane, 30 to 100% EtOAc. ESI/APCI(+), 449 (M+H). ¹H NMR (DMSO-d₆) δ 2.08 (s, 3H), 3.02 (s, 3H), 3.06 (m, 1H), 3.41 (m, 2H), 3.60 (s, 3H), 3.70 (s, 3H), 4.12 (dt, 1H), 4.30 (s, 1H), 5.19 (s, 1H), 5.49 (s, 1H), 6.61 (d, 1H), 7.14 (m, 2H), 7.29-7.44 (m, 4H), 7.87 (d, 1H), 10.41 (s, 1H, NH).

### Example 98

### Preparation of 2-(2-methoxyethyl)-N-(3-methoxyphenyl)-1-oxo-3-(thiazol-2-yl)-1,2,3,4-tetrahydroisoquinoline-4-carboxamide (C102)

This compound was obtained following method B, from thiazol-2-carbaldehyde, 2-methoxyethanamine, homophthalic anhydride and 3-methoxyaniline in 30% overall yield. The title compound was purified by flash chromatography eluting with dichloromethane, 15 to 50% EtOAc. ESI/APCI(+), 438 (M+H). ¹H NMR (DMSO-d₆) δ 3.01 (s, 3H), 3.12 (m, 1H), 3.48 (m, 2H), 3.71 (s, 3H), 4.14 (dt, 1H), 4.52 (s, 3H), 5.68 (s, 1H), 6.62 (d, 1H), 7.14 (t, 1H), 7.19 (t, 1H), 7.31-7.45 (m, 4H), 7.56 (d, 1H), 7.73 (d, 1H), 7.88 (d, 1H), 10.55 (s, 1H, NH).

### Example 100

### Preparation of 2-(2-methoxyethyl)-N-(3-methoxyphenyl)-3-(3-methylisoxazol-5-yl)-1-oxo-1,2,3,4-tetrahydroisoquinoline-4-carboxamide (C104)

This compound was obtained following method B, from 3-methylisoxazol-5-carbaldehyde, 2-methoxyethanamine, homophthalic anhydride and 3-methoxyaniline in 6% overall yield. The title compound was purified by flash chromatography eluting with dichloromethane, 5 to 40% EtOAc. ESI/APCI(+): 436 (M+H). ¹H NMR (DMSO-d₆) δ 2.10 (s, 3H), 2.99 (s, 3H), 3.21 (dt, 1H), 3.41 (m, 2H), 3.71 (s, 3H), 4.04 (dt, 1H), 4.41 (s, 3H), 5.56 (s, 1H), 6.01 (s, 1H), 6.62 (dd, 1H), 7.11 (d, 1H), 7.19 (t, 1H), 7.31 (s, 1H), 7.37 (t, 1H), 7.42-7.52 (m, 2H), 7.89 (d, 1H), 10.46 (s, 1H, NH).

### Example 101

### Preparation of 2-(2-methoxyethyl)-N-(3-methoxyphenyl)-3-(1-methyl-1H-pyrrol-2-yl)-1-oxo-1,2,3,4-tetrahydroisoquinoline-4-carboxamide (C106)

This compound was obtained following method B, from 1-methyl-1H-pyrrol-2-carbaldehyde, 2-methoxyethanamine, homophthalic anhydride and 3-methoxyaniline in 40% overall yield. The title compound was purified by flash chromatography eluting with dichloromethane, 5 to 40% EtOAc. ESI/APCI(+): 434 (M+H). ¹H NMR (DMSO-d₆) δ 3.05 (m, 1H), 3.09 (s, 3H), 3.41 (m, 2H), 3.71 (s, 3H), 3.91 (dt, 1H), 4.10 (s, 1H), 5.32 (s, 2H), 5.73 (t, 1H), 6.63 (m, 2H), 7.09 (d, 1H), 7.19 (t, 1H), 7.28 (m, 2H), 7.42-7.48 (m, 2H), 7.95 (dd, 1H), 10.17 (s, 1H, NH).

### Example 102

### Preparation of 2-(2-methoxyethyl)-N-(3-methoxyphenyl)-1-oxo-3-(phenylethynyl)-1,2,3,4-tetrahydroisoquinoline-4-carboxamide (C119)

This compound was obtained following method B, from phenyl propargylaldehyde, 2-methoxyethanamine, homophthalic anhydride and 3-methoxyaniline in 13% overall yield. The title compound was purified by flash chromatography eluting with dichloromethane, 0 to 20% EtOAc and was recrystallised from a mixture of heptane and ethyl acetate. ESI/APCI(+): 455 (M+H). ¹H NMR (DMSO-d₆) δ 3.05 (3H, s), 3.41-3.57 (3H, m), 3.70 (3 H, s), 3.99 (1H, m), 4.32 (1H, s), 5.33 (1H, s), 6.63 (1H, m), 7.08-7.56 (11H, m), 7.94 (1 H, m), 10.42 (1H, s).

### Example 103

### Preparation of 3-(5-chlorothiophen-2-yl)-2-(2-methoxyethyl)-N-(3-methoxyphenyl)-1-oxo-1,2,3,4-tetrahydroisoquinoline-4-carboxamide (C135)

This compound was obtained following method B, from phenyl 2-chlorothiophene-5-carboxaldehyde, 2-methoxyethanamine, homophthalic anhydride and 3-methoxyaniline in 11% overall yield. The title compound was purified by flash chromatography eluting with heptane, 20 to 80% EtOAc. ESI/APCI(+): 471 (M+H). ¹H NMR (DMSO-d₆) δ 3.00 (s, 3H), 3.08 (m, 1H), 3.41 (m, 2H), 3.71 (s, 3H), 4.04 (m, 1H), 4.24 (s, 1H), 5.52 (s, 1H), 3.34 (d, 1H), 6.92 (m, 2H), 7.10-7.52 (m, 6H), 7.92 (d, 1H), 10.37 (s, 1H).

### Example 104

### Preparation of 3-(benzyloxymethyl)-2-(2-methoxyethyl)-N-(3-methoxyphenyl)-1-oxo-1,2,3,4-tetrahydroisoquinoline-4-carboxamide (C138)

This compound was obtained following method B, from 2-(benzyloxy)acetaldehyde, 2-methoxyethanamine, homophthalic anhydride and 3-methoxyaniline in 1% overall yield. The title compound was purified twice by flash chromatography eluting with dichloromethane, 0 to 10% methanol and then with heptane, 5 to 50% EtOAc. ESI/APCI(+): 475 (M+H). ¹H NMR (DMSO-d₆) δ 2.37 (1H, m), 3.03 (3H, s), 3.47-3.32 (4H, m), 3.69 (3H, s), 3.92 (1H, dt), 4.11 (1H, s), 4.24 (1H, t), 4.39 (1H, d), 4.46 (1H, d), 6.60 (1H, d), 7.09 (1H, m), 7.15 (3H, m), 7.27 (3H, m), 7.39 (2H, m), 7.47 (1H, d), 7.83 (2H, m), 10.23 (1H, s, NH).

### Example 105

### Preparation of 3-(hydroxymethyl)-2-(2-methoxyethyl)-N-(3-methoxyphenyl)-1-oxo-1,2,3,4-tetrahydroisoquinoline-4-carboxamide (C140)

To a suspension of 3-(benzyloxymethyl)-2-(2-methoxyethyl)-N-(3-methoxyphenyl)-1-oxo-1,2,3,4-tetrahydroisoquinoline-4-carboxamide (270 mg, 0.57 mmol) in EtOH (6 mL) was added palladium on carbon (10%) and the mixture was submitted to hydrogenation at atmospheric pressure overnight. After filtration through a pad of celite, the filtrate was concentrated and the residue was purified by flash chromatography eluting with dichloromethane, 2 to 10% methanol to give 165 mg of the title compound, which was then re-purified by preparative layer chromatography on silica gel eluting with a mixture of dichloromethane, 5% methanol. ESI/APCI(+): 385 (M+H). ¹H NMR (DMSO-d₆) δ 3.07 (3H, s), 3.13 (1H, m), 3.24 (1H, m), 3.52-3.38 (3H, m), 3.70 (3H, s), 3.96 (2H, m), 4.14 (1H, s), 5.08 (1H, t), 6.59 (1H, d), 7.10 (1H, d), 7.16 (1H, t), 7.30 (1H, s), 7.37 (2H, m), 7.47 (1H, t), 7.84 (1H, d), 10.21 (1H, s, NH).

### Example 106

### Preparation of 3-cyano-2-(2-methoxyethyl)-N-(3-methoxyphenyl)-1-oxo-1,2,3,4-tetrahydroisoquinoline-4-carboxamide (C143)

To a 0° c. cooled solution of 3-(hydroxymethyl)-2-(2-methoxyethyl)-N-(3-methoxyphenyl)-1-oxo-1,2,3,4-tetrahydroisoquinoline-4-carboxamide (130 mg, 0.34 mmol) in dichloromethane (5 mL) was added Dess-Martin periodinane (0.3M solution in dichloromethane, 1.35 mL, 0.41 mmol), and the mixture was stirred at 0° C. for 2 h. Solutions of saturated aq NaHCO3 (1 mL) and saturated aq Na2S2O3 (1 mL) were then added to the mixture, and this was followed by the addition of dichloromethane (10 mL). The organic layer was separated, and the aqueous phase was further extracted with dichloromethane (2×10 mL). The combined organic extracts were washed with brine, dried (MgSO4), and filtered, and the solvent was removed under reduced pressure to give 2-(2-methoxyethyl)-1-oxo-3-(thiophen-2-yl)-1,2,3,4-tetrahydroisoquinoline-4-carbaldehyde (126 mg, 97%) as an oily yellow solid which was used in the next step without further purification.
To the aldehyde in dry THF (10 mL) was added hydroxylamine hydrochloride (114 mg, 1.65 mmol) and sodium acetate (270 mg, 3.29 mol) and heated at 70° C. (oil bath) for 1.5 hour. The reaction was allowed to cool to room temperature, EtOAc (10 mL) was added, and the organic layer was washed with water (20 mL) and brine (20 mL). The aqueous layers were then re-extracted with EtOAc (2×20 mL), the combined organic layers were then dried over magnesium sulfate and filtered, and the solvent was removed under reduced pressure to give 3-((E)-(hydroxyimino)methyl)-2-(2-methoxyethyl)-N-(3-methoxyphenyl)-1-oxo-1,2,3,4-tetrahydroisoquinoline-4-carboxamide (119 mg, 90%) as a brown solid.
This oxime in acetic anhydride (5 mL) was refluxed for 1.5 h. The reaction mixture was cooled, and the acetic anhydride was removed under reduced pressure. The residue was purified by flash chromatography eluting with heptane, 5 to 70% EtOAc to give the title compound as a white solid (52 mg, 46%). ESI/APCI(+): 380 (M+H). ¹H NMR (DMSO-d₆) δ 3.10 (3H, s), 3.48 (2H, m), 3.70 (5H, m), 4.42 (1H, s), 5.49 (1H, s), 6.63 (1H, d), 7.04 (1H, d), 7.19 (1H, t), 7.23 (1H, s), 7.51 (1H, m), 7.62 (2H, m), 7.95 (1H, d), 10.33 (1H, s, NH).

### Example 107

### Preparation of 2-(2-methoxyethyl)-N-4-(3-methoxyphenyl)-1-oxo-1,2,3,4-tetrahydroisoquinoline-3,4-dicarboxamide (C146)

To a solution of 3-cyano-2-(2-methoxyethyl)-N-(3-methoxyphenyl)-1-oxo-1,2,3,4-tetrahydroisoquinoline-4-carboxamide (43 mg, 0.11 mmol) in absolute ethanol (2 mL) at room temperature were added hydrogen peroxide (30% solution, 0.12 mL) and sodium hydroxide (1M solution, 0.14 mL). The reaction mixture was stirred for 1 h at room temperature before adding water (1 mL). EtOAc (5 mL) was added and the aqueous layer extracted with EtOAc (3×10 mL). The combined organic extracts were concentrated under reduced pressure and the resulting residue was purified by flash chromatography eluting with dichloromethane, 0 to 10% MeOH to give the title compound as a white solid (8.4 mg, 19%). ESI/APCI(+): 398 (M+H). ¹H NMR (DMSO-d₆) δ 3.07 (s, 3H), 3.19 (dt, 1H), 3.42 (t, 2H), 3.70 (s, 3H), 3.88 (dt, 1H), 4.32 (s, 1H), 4.66 (s, 1H), 6.61 (d, 1H), 7.08 (s, 1H), 7.18 (m, 2H), 7.28 (s, 1H), 7.36-7.49 (m, 4H), 7.85 (d, 1H), 10.17 (s, 1H, NH).

### Example 108

### Preparation of 2-(2-methoxyethyl)-N-(4-methyloxazol-2-yl)-1-oxo-3-(thiophen-2-yl)-1,2,3,4-tetrahydroisoquinoline-4-carboxamide (C154)

This compound was obtained following method B, from thiophene-2-carboxaldehyde, 2-methoxyethanamine, homophthalic anhydride and 4-methyl-1,3-oxazol-2-amine in 9% overall yield. The title compound was purified by flash chromatography eluting with heptane, 50 to 100% ethyl acetate and then cristallised from ethanol. ESI/APCI(+): 412 (M+H). ESI/APCI(−): 410 (M−H). ¹H NMR (DMSO d-6) δ 11.71 (1H, s), 7.90 (1H, d), 7.55 (1H, s), 7.45 (2H, m), 7.30 (2H, m), 6.92 (2H, m), 5.62 (1H, s), 4.27 (1H, br s), 4.10 (1H, m), 3.41 (2H, m), 3.19 (1H, m), 3.05 (3H, s), 2.68 (3H, s).

### Example 109

### Preparation of N-(4-cyano-3-methylisoxazol-5-yl)-2-(2-methoxyethyl)-1-oxo-3-(thiophen-2-yl)-1,2,3,4-tetrahydroisoquinoline-4-carboxamide (C155)

This compound was obtained following method B, from thiophene-2-carboxaldehyde, 2-methoxyethanamine, homophthalic anhydride and 5-amino-3-methyl-4-isoxazolecarbonitrile in 42% overall yield. The title compound was purified by flash chromatography eluting with heptane, 10 to 70% ethyl acetate. ESI/APCI(+): 437 (M+H). ESI/APCI(−): 435 (M−H). ¹H NMR (DMSO-d₆) δ 12.95 (1H, s), 7.93 (1H, d), 7.47 (2H, m), 7.36 (1H, m), 7.30 (1H, d), 6.95 (1H, s), 6.90 (1H, m), 5.63 (1H, s), 4.42 (1H, s), 4.15 (1H, m), 3.42 (2H, m), 3.00 (3H, s), 2.99 (1H, m), 2.29 (3H, s).

### Example 110

### Preparation of N-(4,5-dimethylthiazol-2-yl)-2-(2-methoxyethyl)-1-oxo-3-(thiophen-2-yl)-1,2,3,4-tetrahydroisoquinoline-4-carboxamide (C156)

This compound was obtained following method B, from thiophene-2-carboxaldehyde, 2-methoxyethanamine, homophthalic anhydride and 4,5-dimethylthiazol-2-amine hydrobromide
in 24% overall yield. The title compound was purified by flash chromatography eluting with heptane, 30 to 75% ethyl acetate. ESI/APCI(+): 442 (M+H). ESI/APCI(−): 440 (M−H). ¹H NMR (DMSO-d₆) δ 12.47 (1H, s), 7.92 (1H, d), 7.45 (2H, m), 7.32 (2H, m), 6.98 (1H, d), 6.89 (1H, m), 5.62 (1H, s), 4.34 (1H, s), 4.10 (1H, m), 3.39 (2H, m), 3.01 (1H, m), 2.96 (3H, s), 2.21 (3H, s), 2.17 (3H, s).

### Example 111

### Preparation of 2-(2-methoxyethyl)-N-(4-(2-methylthiazol-4-yl)phenyl)-1-oxo-3-(thiophen-2-yl)-1,2,3,4-tetrahydroisoquinoline-4-carboxamide (C157)

This compound was obtained following method B, from thiophene-2-carboxaldehyde, 2-methoxyethanamine, homophthalic anhydride and 4-(2-methylthiazol-4-yl)aniline in 17% overall yield. The title compound was purified by flash chromatography eluting with heptane, 30 to 75% ethyl acetate. ESI/APCI(+): 504 (M+H). ESI/APCI(−): 502 (M−H). ¹H NMR (DMSO-d₆) δ 10.49 (1H, s), 7.91 (3H, m), 7.81 (1H, s), 7.67 (2H, d), 7.46 (2H, m), 7.34 (2H, m), 7.02 (1H, d), 6.91 (1H, m), 5.61 (1H, s), 4.30 (1H, s), 3.44 (2H, m), 3.09 (1H, m), 3.01 (3H, s), 2.69 (3H, s).

### Example 112

### Preparation of 2-(2-methoxyethyl)-1-oxo-N-(6-phenoxypyridin-3-yl)-3-(thiophen-2-yl)-1,2,3,4-tetrahydroisoquinoline-4-carboxamide (C158)

This compound was obtained following method B, from thiophene-2-carboxaldehyde, 2-methoxyethanamine, homophthalic anhydride and 6-phenoxypyridin-3-amine in 17% overall yield. The title compound was purified by flash chromatography eluting with heptane, 30 to 75% ethyl acetate. ESI/APCI(+): 500 (M+H). ESI/APCI(−): 498 (M−H). ¹H NMR (DMSO-d₆) δ 10.57 (1H, s), 8.35 (1H, m), 8.09 (1H, dd), 7.93 (1H, d), 7.40 (6H, m), 7.18 (1H, m), 7.04 (4H, m), 6.92 (1H, m), 5.60 (1H, s), 4.29 (1H, s), 4.09 (1H, m), 3.43 (2H, m), 3.09 (1H, m), 3.03 (3H, s).

### Example 113

### Preparation of N-(4-(furan-2-yl)phenyl)-2-(2-methoxyethyl)-1-oxo-3-(thiophen-2-yl)-1,2,3,4-tetrahydroisoquinoline-4-carboxamide (C159)

This compound was obtained following method B, from thiophene-2-carboxaldehyde, 2-methoxyethanamine, homophthalic anhydride and 4-(furan-2-yl)aniline in 25% overall yield. The title compound was purified by flash chromatography eluting with heptane, 0 to 60% ethyl acetate. ESI/APCI(+): 473 (M+H). ESI/APCI(−): 470 (M−H). ¹H NMR (DMSO-d₆) δ 10.49 (1H, s), 7.93 (1H, d), 7.69 (5H, m), 7.30-7.52 (4H, m), 7.01 (1H, d), 6.91 (1H, m), 6.84 (1H, d), 5.61 (1H, s), 4.29 (1H, s), 4.08 (1H, m), 3.43 (2H, m), 3.07 (1H, m), 3.00 (3H, s).

### Example 114

### Preparation of N-(5-(4-fluorophenyl)isoxazol-3-yl)-2-(2-methoxyethyl)-1-oxo-3-(thiophen-2-yl)-1,2,3,4-tetrahydroisoquinoline-4-carboxamide (C160)

This compound was obtained following method B, from thiophene-2-carboxaldehyde, 2-methoxyethanamine, homophthalic anhydride and 3-amino-5-(4-fluorophenyl)isoxazole in 7% overall yield. The title compound was purified by flash chromatography eluting with dichloromethane, 10 to 60% ethyl acetate. ESI/APCI(+): 473 (M+H). ESI/APCI(−): 470 (M−H).

### Example 115

### Preparation of 2-(2-methoxyethyl)-1-oxo-N-(1H-pyrazol-3-yl)-3-(thiophen-2-yl)-1,2,3,4-tetrahydroisoquinoline-4-carboxamide (C184)

This compound was obtained following method B, from thiophene-2-carboxaldehyde, 2-methoxyethanamine, homophthalic anhydride and 3-aminopyrazole in 16% overall yield. The title compound was purified by flash chromatography eluting with heptane, 10 to 70% ethyl acetate. ESI/APCI(+): 397 (M+H). ESI/APCI(−): 395 (M−H).

### Example 116

### Preparation of N-(5-tert-butyl-1H-pyrazol-3-yl)-2-(2-methoxyethyl)-1-oxo-3-(thiophen-2-yl)-1,2,3,4-tetrahydroisoquinoline-4-carboxamide (C185)

This compound was obtained following method B, from thiophene-2-carboxaldehyde, 2-methoxyethanamine, homophthalic anhydride and 3-amino-5-tert-butylpyrazole in 34% overall yield. The title compound was purified by flash chromatography eluting with heptane, 10 to 70% ethyl acetate and recrystallised from ethanol. ESI/APCI(+): 453 (M+H). ESI/APCI(−): 451 (M−H). ¹H NMR (DMSO-d₆) δ 7.95 (1H, m), 7.46 (2H, m), 7.36 (2H, m), 6.89 (1H, m), 6.80 (1H, m), 6.53 (2H, s), 5.95 (1H, s), 5.34 (1H, s), 5.25 (1H, s), 4.19 (1H, m), 3.36 (2H, m), 2.98 (3H, s), 2.90 (1H, m), 1.30 (9H, s).

### Example 117

### Preparation of 2-(2-methoxyethyl)-1-oxo-N-(6-phenylpyridin-3-yl)-3-(thiophen-2-yl)-1,2,3,4-tetrahydroisoquinoline-4-carboxamide (C186)

This compound was obtained following method B, from thiophene-2-carboxaldehyde, 2-methoxyethanamine, homophthalic anhydride and 6-phenylpyridin-3-amine in 8% overall yield. The title compound was purified by flash chromatography eluting with heptane, 0 to 60% ethyl acetate, preparative thick layer chromatography eluting with 5% methanol in dichloromethane and recrystallised from ethanol. ESI/APCI(+): 484 (M+H). ESI/APCI(−): 482(M−H). ¹H NMR (DMSO-d₆) δ 10.76 (1H, s), 8.84 (1H, s), 8.17 (1H, m), 8.05 (2H, m), 7.94 (2H, t), 7.31-7.53 (7H, m), 7.02 (1H, m), 6.92 (1H, dd), 5.63 (1H, s), 4.35 (1H, s), 4.11 (1H, m), 3.44 (2H, m), 3.05 (1H, m), 3.00 (3H, s).

### Example 118

### Preparation of 2-(2-methoxyethyl)-N-(4-(5-methyl-1,2,4-oxadiazol-3-yl)phenyl)-1-oxo-3-(thiophen-2-yl)-1,2,3,4-tetrahydroisoquinoline-4-carboxamide (C187)

This compound was obtained following method B, from thiophene-2-carboxaldehyde, 2-methoxyethanamine, homophthalic anhydride and 4-(5-methyl-1,2,4-oxadiazol-3-yl)aniline in 6% overall yield. The title compound was purified by flash chromatography eluting with heptane, 0 to 60% ethyl acetate and preparative thick layer chromatography eluting with 75% ethyl acetate in heptane. ESI/APCI(+): 489 (M+H). ESI/APCI(−): 487 (M−H). ¹H NMR (DMSO-d₆) δ 10.71 (1H, s), 7.93 (3H, m), 7.80 (2H, m), 7.30-7.52 (4H, m), 7.01 (1H, m), 6.92 (1H, dd), 5.62 (1H, s), 4.33 (1H, s), 4.09 (1H, m), 3.43 (2H, m), 3.09 (1H, m), 2.98 (3H, s), 2.63 (3H, s).

### Example 119

### Preparation of 2-(2-methoxyethyl)-N-(4-(morpholinomethyl)phenyl)-1-oxo-3-(thiophen-2-yl)-1,2,3,4-tetrahydroisoquinoline-4-carboxamide (C188)

This compound was obtained following method B, from thiophene-2-carboxaldehyde, 2-methoxyethanamine, homophthalic anhydride and 4-(morpholinomethyl)aniline in 43% overall yield. The title compound was purified by flash chromatography eluting with heptane, 0 to 60% ethyl acetate. ESI/APCI(+): 506 (M+H). ESI/APCI(−): 504 (M−H). ¹H NMR (DMSO-d₆) δ 10.35 (1H, s), 7.91 (1H, d), 7.38-7.63 (4H, m), 7.17-7.36 (4H, m), 6.99 (1H, m), 6.89 (1H, m), 5.57 (1H, s), 4.26 (1H, s), 4.05 (1H, m), 3.49-3.64 (6H, m), 3.41 (2H, m), 3.09 (1H, m), 3.00 (3H, s), 2.32 (4H, m).

### Example 120

### Preparation of N-(1,3-dihydroisobenzofuran-5-yl)-2-(2-methoxyethyl)-1-oxo-3-(thiophen-2-yl)-1,2,3,4-tetrahydroisoquinoline-4-carboxamide (C190)

This compound was obtained following method B, from thiophene-2-carboxaldehyde, 2-methoxyethanamine, homophthalic anhydride and 1,3-dihydroisobenzofuran-5-amine in 11% overall yield. The title compound was purified by flash chromatography eluting with heptane, 0 to 60% ethyl acetate. ESI/APCI(+): 449 (M+H). ESI/APCI(−): 447 (M−H). ¹H NMR (DMSO-d₆) δ 10.41 (1H, s), 7.92 (1H, d), 7.61 (1H, s), 7.45 (3H, m), 7.37 (1H, t), 7.30 (1H, d), 7.24 (1H, d), 7.01 (1H, d), 6.90 (1H, dd), 5.59 (1H, s), 4.94 (4H, s), 4.28 (1H, s), 4.07 (1H, m), 3.43 (2H, m), 3.08 (1H, m), 3.02 (3H, s).

### Example 121

### Preparation of 2-(2-methoxyethyl)-N-(4-(6-methylpyrazin-2-yloxy)phenyl)-1-oxo-3-(thiophen-2-yl)-1,2,3,4-tetrahydroisoquinoline-4-carboxamide (C191)

This compound was obtained following method B, from thiophene-2-carboxaldehyde, 2-methoxyethanamine, homophthalic anhydride and 4-(6-methylpyrazin-2-yloxy)aniline in 31% overall yield. The title compound was purified by flash chromatography eluting with heptane, 0 to 60% ethyl acetate. ESI/APCI(+): 515 (M+H). ESI/APCI(−): 513 (M−H). ¹H NMR (DMSO-d₆) δ 10.45 (1H, s), 8.24 (2H, s), 7.92 (1H, d), 7.64 (2H, d), 7.40-7.52 (2H, m), 7.36 (1H, d), 7.30 (1H, d), 7.14 (2H, d), 7.01 (1H, m), 6.91 (1H, dd), 5.59 (1H, s), 4.28 (1H, s), 4.07 (1H, m), 3.44 (2H, m), 3.08 (1H, m), 3.03 (3H, s), 2.30 (3H, s).

### Example 122

### Preparation of N-(6-chlorobenzo[d]thiazol-2-yl)-2-(2-methoxyethyl)-1-oxo-3-(thiophen-2-yl)-1,2,3,4-tetrahydroisoquinoline-4-carboxamide (C202)

This compound was obtained following method B, from thiophene-2-carboxaldehyde, 2-methoxyethanamine, homophthalic anhydride and 6-chlorobenzo[d]thiazol-2-amine in 15% overall yield. The title compound was purified by flash chromatography eluting with heptane, 0 to 60% ethyl acetate and recrystallised from heptane. ESI/APCI(+): 498 (M+H). ESI/APCI(−): 496 (M−H). ¹H NMR (DMSO-d₆) δ 13.14 (1H, s), 8.12 (1H, m), 7.94 (1H, m), 7.76 (1H, d), 7.47 (3H, m), 7.38 (1H, m), 7.30 (1H, d), 7.00 (1H, m), 6.90 (1H, m), 5.70 (1H, s), 4.47 (1H, s), 4.14 (1H, m), 3.40 (2H, m), 2.98 (1H, m), 2.88 (3H, s).

### Example 123

### Preparation of 2-(2-methoxyethyl)-N-(4-(1-methyl-1H-pyrazol-3-yl)phenyl)-1-oxo-3-(thiophen-2-yl)-1,2,3,4-tetrahydroisoquinoline-4-carboxamide (C203)

This compound was obtained following method B, from thiophene-2-carboxaldehyde, 2-methoxyethanamine, homophthalic anhydride and 4-(1-methyl-1H-pyrazol-3-yl)aniline in 23% overall yield. The title compound was purified by flash chromatography eluting with heptane, 0 to 60% ethyl acetate and recrystallised from ethanol. ESI/APCI(+): 487 (M+H). ESI/APCI(−): 485 (M−H). ¹H NMR (DMSO-d₆) δ 10.43 (1H, s), 7.92 (1H, d), 7.59-7.76 (5H, m), 7.29-7.51 (4H, m), 7.01 (1H, m), 6.91 (1H, dd), 6.62 (1H, m), 5.60 (1H, s), 4.28 (1H, s), 4.07 (1H, m), 3.85 (3H, s), 3.43 (2H, m), 3.07 (1H, m), 3.01 (3H, s).

### Example 124

### Preparation of N-(1,3-dimethyl-1H-pyrazol-5-yl)-2-(2-methoxyethyl)-1-oxo-3-(thiophen-2-yl)-1,2,3,4-tetrahydroisoquinoline-4-carboxamide (C204)

This compound was obtained following method B, from thiophene-2-carboxaldehyde, 2-methoxyethanamine, homophthalic anhydride and 1,3-dimethyl-1H-pyrazol-5-amine hydrochloride in 6% overall yield. The title compound was purified by flash chromatography eluting with heptane, 0 to 60% ethyl acetate and preparative thick layer chromatography eluting with 5% methanol in dichloromethane. ESI/APCI(+): 425 (M+H). ESI/APCI(−): 423 (M−H). ¹H NMR (DMSO-d₆) δ 10.21 (1H, s), 8.18 (2H, m), 7.92 (1H, d), 7.30-7.53 (2H m), 7.01 (1H, m), 6.91 (1H, m), 5.98 (1H, s), 5.58 (1H, s), 4.35 (1H, s), 4.02 (1H, m), 3.62 (2H, m), 3.56 (3H, s), 3.14 (1H, m), 3.10 (3H, s), 2.06 (3H, s).

### Example 125

### Preparation of 2-(2-methoxyethyl)-N-(4-(5-methyl-1,3,4-oxadiazol-2-yl)phenyl)-1-oxo-3-(thiophen-2-yl)-1,2,3,4-tetrahydroisoquinoline-4-carboxamide (C205)

This compound was obtained following method B, from thiophene-2-carboxaldehyde, 2-methoxyethanamine, homophthalic anhydride and 4-(5-methyl-1,3,4-oxadiazol-2-yl)aniline in 5% overall yield. The title compound was purified by flash chromatography eluting with heptane, 0 to 60% ethyl acetate and recrystallised from ethanol. ESI/APCI(+): 489 (M+H). ESI/APCI(−): 487 (M−H). ¹H NMR (DMSO-d₆) δ 10.75 (1H, s), 7.93 (3H, m), 7.81 (2H, d), 7.47 (2H, m), 7.36 (1H, d), 7.31 (1H, d), 7.01 (1H, m), 6.91 (1H, m), 5.61 (1H, s), 4.33 (1H, s), 4.08 (1H, m), 3.42 (2H, m), 3.07 (1H, m), 2.98 (3H, s), 2.68 (3H, s).

### Example 126

### Preparation of 2-(2-methoxyethyl)-N-(1-methyl-2-oxoindolin-5-yl)-1-oxo-3-(thiophen-2-yl)-1,2,3,4-tetrahydroisoquinoline-4-carboxamide (C206)

This compound was obtained following method B, from thiophene-2-carboxaldehyde, 2-methoxyethanamine, homophthalic anhydride and 5-amino-1-methylindolin-2-one in 6% overall yield. The title compound was purified by flash chromatography eluting with heptane, 0 to 60% ethyl acetate and recrystallised twice from ethanol. ESI/APCI(+): 476 (M+H). ESI/APCI(−): 474 (M−H). ¹H NMR (DMSO-d₆) δ 10.25 (1H, s), 7.92 (1H, d), 7.29-7.57 (6H, m), 7.01 (1H, m), 6.91 (2H, m), 5.58 (1H, s), 4.24 (1H, s), 4.06 (1H, m), 3.52 (2H, s), 3.44 (2H, m), 3.13 (1H, m), 3.08 (3H, s), 3.05 (3H, s).

### Example 127

### Preparation of 5-amino-3-(−2-(2-methoxyethyl)-1-oxo-3-(thiophen-2-yl)-1,2,3,4-tetrahydroisoquinoline-4-carbonyl)benzo[d]oxazol-2(3H)-one (C207)

This compound was obtained following method B, from thiophene-2-carboxaldehyde, 2-methoxyethanamine, homophthalic anhydride and 5-aminobenzo[d]oxazol-2(3H)-one in 11% overall yield. The title compound was purified by flash chromatography eluting with heptane, 0 to 60% ethyl acetate and recrystallised from ethanol. ESI/APCI(+): 464 (M+H). ESI/APCI(−): 462 (M−H). ¹H NMR (DMSO-d₆) δ NMR:7.94 (1H, m), 7.55 (1H, d), 7.47 (2H, m), 7.32 (2H, m), 7.89 (2H, m), 6.63 (1H, s), 6.42 (1H, d), 5.68 (1H, s), 5.42 (2H, s), 5.31 (1H, s), 4.15 (1H, m), 3.44 (1H, m), 3.31 (1H, m), 2.99 (1H, m), 2.79 (3H, s).

### Example 128

### Preparation of 2-(2-methoxyethyl)-N-(1-methyl-3-phenyl-1H-pyrazol-5-yl)-1-oxo-3-(thiophen-2-yl)-1,2,3,4-tetrahydroisoquinoline-4-carboxamide (C208)

This compound was obtained following method B, from thiophene-2-carboxaldehyde, 2-methoxyethanamine, homophthalic anhydride and 1-methyl-3-phenyl-1H-pyrazol-5-amine in 3% overall yield. The title compound was purified by flash chromatography eluting with heptane, 50 to 100% ethyl acetate. ESI/APCI(+): 487 (M+H). ESI/APCI(−): 485 (M−H). ¹H NMR (DMSO-d₆) δ 10.42 (1H, s), 7.93 (1H, d), 7.72 (2H, d), 7.26-7.55 (7H, m), 7.02 (1H, m), 6.91 (1H, m), 6.66 (1H, s), 5.63 (1H, s), 4.38 (1H, s), 4.04 (1H, m), 3.72 (3H, s), 3.48 (2H, m), 3.17 (1H, m), 3.10 (3H, s).

### Example 129

### Preparation of methyl 4-(2-(2-methoxyethyl)-1-oxo-3-(thiophen-2-yl)-1,2,3,4-tetrahydroisoquinoline-4-carboxamido)-1-methyl-1H-pyrrole-2-carboxylate (C209)

This compound was obtained following method B, from thiophene-2-carboxaldehyde, 2-methoxyethanamine, homophthalic anhydride and methyl 4-amino-1-methyl-1H-pyrrole-2-carboxylate hydrochloride in 22% overall yield. The title compound was purified by flash chromatography eluting with heptane, 0 to 60% ethyl acetate. ESI/APCI(+): 468 (M+H). ESI/APCI(−): 466 (M−H). ¹H NMR (DMSO-d₆) δ 10.25 (1H, s), 7.90 (1H, d), 7.28-7.89 (5H, m), 6.98 (1H, s), 6.88 (1H, m), 6.76 (1H, m), 5.54 (1H, s), 4.15 (1H, s), 4.03 (1H, s), 3.79 (3H, s), 3.72 (3H, s), 3.42 (2H, m), 3.10 (1H, m), 3.06 (3H, s).

### Example 130

### Preparation of 2-(2-methoxyethyl)-N-(3-(oxazol-5-yl)phenyl)-1-oxo-3-(thiophen-2-yl)-1,2,3,4-tetrahydroisoquinoline-4-carboxamide (C210)

This compound was obtained following method B, from thiophene-2-carboxaldehyde, 2-methoxyethanamine, homophthalic anhydride and 3-(oxazol-5-yl)aniline in 9% overall yield. The title compound was purified by flash chromatography eluting with heptane, 50 to 60% ethyl acetate and recrystallised from ethanol. ESI/APCI(+): 474 (M+H). ESI/APCI(−): 472 (M−H). ¹H NMR (DMSO-d₆) δ 10.57 (1H, s), 8.43 (1H, s), 8.03 (1H, s), 7.92 (1H, m), 7.63 (1H, s), 7.56 (1H, m), 7.40-7.49 (4H, m), 7.35 (1H, d), 7.31 (1H, d), 7.00 (1H, m), 6.91 (1H, t), 5.61 (1H, s), 4.30 (1H, s), 4.08 (1H, m), 3.42 (2H, m), 3.07 (1H, m), 2.98 (3H, s).

### Example 131

### Preparation of 2-(2-methoxyethyl)-N-(3-methoxyphenyl)-N-methyl-1-oxo-3-(thiophen-2-yl)-1,2,3,4-tetrahydroisoquinoline-4-carboxamide (C151)

This compound was obtained following method B, from thiophene-2-carboxaldehyde, 2-methoxyethanamine, homophthalic anhydride and 3-methoxy-N-methylaniline in 10% overall yield. The title compound was purified by flash chromatography eluting with heptane, 10 to 90% ethyl acetate. ESI/APCI(+): 451 (M+H). ESI/APCI(−): 449 (M−H). ¹H NMR (DMSO-d₆) δ 2.97-3.01 (m, 1H), 3.21 (s, 3H), 3.32 (s, 3H), 3.48-3.50 (m, 2H), 3.85 (s, 3H), 4.07 (s, 1H), 4.10-4.13 (m, 1H), 5.41 (s, 1H), 6.28 (s, 1H), 6.72-6.74 (t, 1H), 7.01 (s, 1H), 7.07-7.10 (m, 1H), 7.17-7.22 (m, 3H), 7.38-7.43 (t, 2H), 7.50-7.56 (t, 1H), 7.88-7.91 (m, 1H).

### Example 132

### Preparation of N-(4-(1H-pyrrol-1-yl)phenyl)-8-fluoro-7-methoxy-2-(2-methoxyethyl)-1-oxo-3-(thiophen-2-yl)-1,2,3,4-tetrahydroisoquinoline-4-carboxamide (C150)

This compound was obtained following method B, from thiophene-2-carboxaldehyde, 2-methoxyethanamine, 8-fluoro-7-methoxyisochroman-1,3-dione and 4-(1H-pyrrol-1-yl)aniline
in 17% overall yield. The title compound was purified by flash chromatography eluting with heptane, 5 to 70% ethyl acetate and precipitated from ethyl acetate. ESI/APCI(+): 520 (M+H). ESI/APCI(−): 518 (M−H). ¹H NMR (DMSO-d₆) δ 10.43 (1H, s, NH), 7.67 (2H, d), 7.52 (2H, d), 7.32 (3H, m), 7.26 (1H, t), 7.09 (1H, d), 7.02 (1H, d), 6.91 (1H, dd), 6.23 (2H, t), 5.56 (1H, s), 4.24 (1H, s), 3.94 (1H, dt), 3.84 (3H, s), 3.39 (2H, m), 3.09 (1H, dt), 2.98 (3H, s).

### Example 133

### Preparation of N-(4-(1H-pyrrol-1-yl)phenyl)-3-(5-chlorothiophen-2-yl)-2-(2-methoxyethyl)-1-oxo-1,2,3,4-tetrahydroisoquinoline-4-carboxamide (C153)

This compound was obtained following method B, from 5-chlorothiophene-2-carbaldehyde, 2-methoxyethanamine, homophthalic anhydride and 4-(1H-pyrrol-1-yl)aniline in 10% overall yield. The title compound was purified by flash chromatography eluting with heptane, 5 to 70% ethyl acetate and precipitated from ethyl acetate. ESI/APCI(+): 506 (M+H). ESI/APCI(−): 504 (M−H). ¹H NMR (DMSO-d₆) δ 10.49 (1H, s, NH), 7.92 (1H, d), 7.66 (2H, d), 7.52 (3H, m), 7.45 (1H, t), 7.40 (1H, d), 7.32 (2H, s), 6.93 (1H, d), 6.91 (1H, d), 6.24 (2H, s), 5.56 (1H, s), 4.27 (1H, s), 4.03 (1H, dt), 3.42 (2H, m), 3.08 (1H, dt), 3.03 (3H, s).

### Example 134

### Preparation of N-(3-methoxyphenyl)-1-oxo-3-(thiophen-2-yl)-2-trimethylsilyl-1,2,3,4-tetrahydroisoquinoline-4-carboxamide (C161)

This compound was obtained following method B, from thiophene-2-carbaldehyde, trimethylsilylamine, homophthalic anhydride and m-anisidine in 39% overall yield. The title compound was purified by flash chromatography eluting with heptane, 5 to 70% ethyl acetate. ESI/APCI(+): 379 (M+H). ESI/APCI(−): 377 (M−H). ¹H NMR (DMSO-d₆) δ 10.38 (1H, s, NH), 8.53 (1H, d, NH), 7.89 (1H, d), 7.47 (1H, t), 7.39 (1H, t), 7.31 (3H, m), 7.19 (1H, t), 7.09 (1H, d), 7.00 (1H, s), 6.91 (1H, t), 6.62 (1H, d), 5.29 (1H, s), 4.33 (1H, s), 3.34 (3H, s).

### Example 135

### Preparation of 2-(2-methoxyethyl)-1-oxo-N-(pyrimidin-4-yl)-3-(thiophen-2-yl)-1,2,3,4-tetrahydroisoquinoline-4-carboxamide (C162)

This compound was obtained following method B, from thiophene-2-carbaldehyde, 2-methoxyethanamine, homophthalic anhydride and 4-aminopyrimidine in 9% overall yield. The title compound was purified by flash chromatography eluting with dichloromethane, 0 to 5% methanol. ESI/APCI(+): 409 (M+H). ESI/APCI(−): 407 (M−H). ¹H NMR (DMSO-d₆) δ 11.61 (1H, s, NH), 8.93 (1H, s), 8.64 (1H, d), 7.99 (1H, d), 7.90 (1H, d), 7.41 (2H, m), 7.32 (1H, d), 7.28 (1H, d), 7.00 (1H, d), 6.88 (1H, dd), 5.63 (1H, s), 4.48 (1H, s), 4.09 (1H, dt), 3.39 (2H, m), 2.97 (1H, m), 2.91 (3H, s).

### Example 136

### Preparation of 2-(2-methoxyethyl)-1-oxo-N-(pyrazin-2-yl)-3-(thiophen-2-yl)-1,2,3,4-tetrahydroisoquinoline-4-carboxamide (C163)

This compound was obtained following method B, from thiophene-2-carbaldehyde, 2-methoxyethanamine, homophthalic anhydride and 2-aminopyrazine in 8% overall yield. The title compound was purified by flash chromatography eluting with dichloromethane, 0 to 5% methanol. ESI/APCI(+): 409 (M+H). ESI/APCI(−): 407 (M−H). ¹H NMR (DMSO-d₆) δ 11.45 (1H, s, NH), 9.28 (1H, s), 8.44 (1H, s), 8.38 (1H, s), 7.91 (1H, d), 7.41 (2H, m), 7.28 (2H, m),

### 7.01 (1H, s), 6.89 (1H, m), 5.63 (1H, s), 4.48 (1H, s), 4.10 (1H, m), 3.40 (2H, m), 2.97 (1H, m), 2.88 (3H, s).

### Example 137

### Preparation of N-(benzo[d]thiazol-5-yl)-2-(2-methoxyethyl)-1-oxo-3-(thiophen-2-yl)-1,2,3,4-tetrahydroisoquinoline-4-carboxamide (C164)

This compound was obtained following method B, from thiophene-2-carbaldehyde, 2-methoxyethanamine, homophthalic anhydride and benzo[d]thiazol-5-amine in 37% overall yield. The title compound was purified by flash chromatography eluting with heptane, 5 to 70% ethyl acetate and precipitated in ethyl acetate. ESI/APCI(+): 464 (M+H). ESI/APCI(−) δ 462 (M−H). ¹H NMR (DMSO-d₆) δ 10.64 (1H, s, NH), 9.35 (1H, d), 8.47 (1H, s), 8.08 (1H, dd), 7.93 (1H, d), 7.62 (1H, d), 7.44 (4H, m), 7.37 (1H, d), 7.31 (1H, s), 7.03 (1H, s), 6.92 (1H, m), 5.64 (1H, s), 4.34 (1H, s), 4.08 (1H, dt), 3.45 (2H, m), 3.05 (1H, dt), 2.98 (3H, s).

### Example 138

### Preparation of N-(4-isopropylphenyl)-2-(2-methoxyethyl)-1-oxo-3-(thiophen-2-yl)-1,2,3,4-tetrahydroisoquinoline-4-carboxamide (C165)

This compound was obtained following method B, from thiophene-2-carbaldehyde, 2-methoxyethanamine, homophthalic anhydride and 4-isopropylaniline in 36% overall yield. The title compound was purified by flash chromatography eluting with heptane, 5 to 70% ethyl acetate. ESI/APCI(+): 449 (M+H). ESI/APCI(−): 447 (M−H). ¹H NMR (DMSO-d₆) δ 10.28 (1H, s, NH), 7.91 (1H, d), 7.42 (4H, m), 7.26 (2H, m), 7.16 (2H, d), 7.01 (1H, s), 6.90 (1H, m), 5.58 (1H, s), 4.26 (1H, s), 4.04 (1H, dt), 3.43 (2H, m), 3.07 (1H, dt), 3.03 (3H, s), 2.49 (1H, m), 1.17 (3H, d), 1.15 (3H, d).

### Example 139

### Preparation of N-(3-(hydroxymethyl)phenyl)-2-(2-methoxyethyl)-1-oxo-3-(thiophen-2-yl)-1,2,3,4-tetrahydroisoquinoline-4-carboxamide (C166)

This compound was obtained following method B, from thiophene-2-carbaldehyde, 2-methoxyethanamine, homophthalic anhydride and (3-aminophenyl)methanol in 50% overall yield. The title compound was purified by flash chromatography eluting with heptane, 30 to 100% ethyl acetate. ESI/APCI(+): 437 (M+H). ESI/APCI(−): 435 (M−H). ¹H NMR (DMSO-d₆) δ 10.35 (1H, s, NH), 7.91 (1H, d), 7.56 (1H, s), 7.42 (3H, m), 7.30 (2H, m), 7.22 (1H, t), 6.97 (2H, m), 6.91 (1H, m), 5.59 (1H, s), 5.20 (1H, t, OH), 4.45 (2H, d), 4.27 (1H, s), 4.04 (1H, dt), 3.43 (2H, m), 3.07 (1H, dt), 3.01 (3H, s).

### Example 140

### Preparation of N-(4-(1H-1,2,4-triazol-1-yl)phenyl)-2-(2-methoxyethyl)-1-oxo-3-(thiophen-2-yl)-1,2,3,4-tetrahydroisoquinoline-4-carboxamide (C167)

This compound was obtained following method B, from thiophene-2-carbaldehyde, 2-methoxyethanamine, homophthalic anhydride and 4-(1H-1,2,4-triazol-1-yl)aniline in 22% overall yield. The title compound was purified by flash chromatography eluting with dichloromethane, 30 to 100% ethyl acetate. ESI/APCI(+): 474 (M+H). ESI/APCI(−): 472 (M−H). ¹H NMR (DMSO-d₆) δ 10.62 (1H, s, NH), 9.21 (1H, s), 8.20 (1H, s), 7.92 (1H, d), 7.76 (4H, m), 7.44 (2H, m), 7.36 (1H, d), 7.31 (1H, d), 7.01 (1H, s), 6.90 (1H, dd), 5.62 (1H, s), 4.31 (1H, s), 4.06 (1H, dt), 3.43 (2H, m), 3.03 (1H, dt), 3.01 (3H, s).

### Example 141

### Preparation of 2-(2-methoxyethyl)-N-(2-methylquinolin-6-yl)-1-oxo-3-(thiophen-2-yl)-1,2,3,4-tetrahydroisoquinoline-4-carboxamide (C168)

This compound was obtained following method B, from thiophene-2-carbaldehyde, 2-methoxyethanamine, homophthalic anhydride and 2-methylquinolin-6-amine in 29% overall yield. The title compound was purified by flash chromatography eluting with dichloromethane, 30 to 100% ethyl acetate. ESI/APCI(+): 472 (M+H). ESI/APCI(−): 470 (M−H). ¹H NMR (DMSO-d₆) δ 10.67 (1H, s, NH), 8.30 (1H, s), 8.13 (1H, d), 7.93 (1H, d), 7.87 (1H, d), 7.76 (1H, dd), 50-7.31 (5H, m), 7.02 (1H, d), 6.91 (1H, dd), 5.65 (1H, s), 4.35 (1H, s), 4.06 (1H, dt), 3.42 (2H, t), 3.09 (1H, dt), 2.95 (3H, s), 20.61 (3H, s).

### Example 142

### Preparation of N-(3,5-dimethylisoxazol-4-yl)-2-(2-methoxyethyl)-1-oxo-3-(thiophen-2-yl)-1,2,3,4-tetrahydroisoquinoline-4-carboxamide (C169)

This compound was obtained following method B, from thiophene-2-carbaldehyde, 2-methoxyethanamine, homophthalic anhydride and (4-amino-3-methylisoxazol-5-yl)methylium
in 51% overall yield. The title compound was purified by flash chromatography eluting with dichloromethane, 5 to 50% ethyl acetate. ESI/APCI(+): 426 (M+H). ESI/APCI(−): 424 (M−H). ¹H NMR (DMSO-d₆) δ 9.95 (1H, s, NH), 7.91 (1H, d), 7.50 (1H, t), 7.42 (1H, t), 7.26 (1H, t), 7.31 (1H, d), 7.01 (1H, d), 6.90 (1H, dd), 5.59 (1H, s), 4.25 (1H, s), 3.94 (1H, dt), 3.40 (2H, m), 3.18 (1H, m), 3.15 (3H, s), 2.23 (3H, s), 2.03 (3H, s).

### Example 143

### Preparation of N-(benzo[d]thiazol-6-yl)-2-(2-methoxyethyl)-1-oxo-3-(thiophen-2-yl)-1,2,3,4-tetrahydroisoquinoline-4-carboxamide (C170)

This compound was obtained following method B, from thiophene-2-carbaldehyde, 2-methoxyethanamine, homophthalic anhydride and benzo[d]thiazol-6-amine in 49% overall yield. The title compound was purified by flash chromatography eluting with dichloromethane, 5 to 50% ethyl acetate. ESI/APCI(+): 464 (M+H). ESI/APCI(−): 462 (M−H). ¹H NMR (DMSO-d₆) δ 10.67 (1H, s, NH), 9.25 (1H, s), 8.54 (1H, s), 8.02 (1H, d), 7.93 (1H, d), 7.61 (1H, d), 7.38 (3H, m), 7.31 (1H, d), 7.02 (1H, s), 6.91 (1H, dd), 5.64 (1H, s), 4.34 (1H, s), 4.05 (1H, dt), 3.43 (2H, t), 3.08 (1H, dt), 2.97 (3H, s).

### Example 144

### Preparation of 2-(2-methoxyethyl)-1-oxo-N-(quinoxalin-6-yl)-3-(thiophen-2-yl)-1,2,3,4-tetrahydroisoquinoline-4-carboxamide (C171)

This compound was obtained following method B, from thiophene-2-carbaldehyde, 2-methoxyethanamine, homophthalic anhydride and quinoxalin-6-amine in 18% overall yield. The title compound was purified by flash chromatography eluting with dichloromethane, 30 to 100% ethyl acetate. ESI/APCI(+): 459 (M+H). ESI/APCI(−): 457 (M−H). ¹H NMR (DMSO-d₆) δ 10.98 (1H, s, NH), 8.88 (1H, s), 8.82 (1H, s), 8.50 (1H, s), 8.07 (1H, t), 7.94 (2H, m), 7.45 (2H, m), 7.39 (1H, d), 7.32 (1H, d), 7.03 (1H, s), 6.92 (1H, t), 5.67 (1H, s), 4.40 (1H, s), 4.08 (1H, dt), 3.43 (2H, t), 3.05 (1H, dt), 2.94 (3H, s).

### Example 145

### Preparation of N-(3-(1H-pyrazol-1-yl)phenyl)-2-(2-methoxyethyl)-1-oxo-3-(thiophen-2-yl)-1,2,3,4-tetrahydroisoquinoline-4-carboxamide (C172)

This compound was obtained following method B, from thiophene-2-carbaldehyde, 2-methoxyethanamine, homophthalic anhydride and 3-(1H-pyrazol-1-yl)aniline in 50% overall yield. The title compound was purified by flash chromatography eluting with heptane, 15 to 70% ethyl acetate. ESI/APCI(+): 473 (M+H). ESI/APCI(−): 471 (M−H). ¹H NMR (DMSO-d₆) δ 10.53 (1H, s, NH), 8.42 (1H, s), 7.92 (1H, d), 7.80 (2H, m), 7.70 (3H, m), 7.43 (2H, m), 7.35 (1H, d), 7.30 (1H, d), 7.01 (1H, s), 6.90 (1H, t), 6.51 (1H, d), 5.62 (1H, s), 4.30 (1H, s), 4.06 (1H, dt), 3.43 (2H, t), 3.03 (1H, dt), 3.02 (3H, s).

### Example 146

### Preparation of 2-(2-methoxyethyl)-1-oxo-N-(4-(pyridin-3-yl)phenyl)-3-(thiophen-2-yl)-1,2,3,4-tetrahydroisoquinoline-4-carboxamide (C173)

This compound was obtained following method B, from thiophene-2-carbaldehyde, 2-methoxyethanamine, homophthalic anhydride and 4-(pyridin-3-yl)aniline in 13% overall yield. The title compound was purified by flash chromatography eluting with dichloromethane, 1 to 7% methanol. ESI/APCI(+): 484 (M+H). ESI/APCI(−): 482 (M−H). ¹H NMR (DMSO-d₆) δ 10.54 (1H, s, NH), 8.89 (1H, s), 8.52 (1H, d), 8.04 (1H, d), 7.93 (1H, d), 7.74 (4H, s), 7.42 (3H, m), 7.37 (1H, d), 7.31 (1H, d), 7.03 (1H, s), 6.91 (1H, t), 5.63 (1H, s), 4.32 (1H, s), 4.07 (1H, dt), 3.43 (2H, t), 3.07 (1H, dt), 3.03 (3H, s).

### Example 147

### Preparation of N-(4-((1H-pyrazol-1-yl)methyl)phenyl)-2-(2-methoxyethyl)-1-oxo-3-(thiophen-2-yl)-1,2,3,4-tetrahydroisoquinoline-4-carboxamide (C174)

This compound was obtained following method B, from thiophene-2-carbaldehyde, 2-methoxyethanamine, homophthalic anhydride and 4-((1H-pyrazol-1-yl)methyl)aniline in 52% overall yield. The title compound was purified by flash chromatography eluting with heptane, 5 to 70% ethyl acetate. ESI/APCI(+): 487 (M+H). ESI/APCI(−): 485 (M−H). ¹H NMR (DMSO-d₆) δ 10.40 (1H, s, NH), 7.90 (1H, d), 7.76 (1H, s), 7.54 (2H, d), 7.44 (3H, m), 7.32 (1H, d), 7.29 (1H, d), 7.16 (2H, d), 6.99 (1H, s), 6.89 (1H, t), 6.24 (1H, d), 5.57 (1H, s), 5.26 (2H, s), 4.26 (1H, s), 4.03 (1H, dt), 3.40 (2H, m), 3.02 (1H, dt), 2.97 (3H, s).

### Example 148

### Preparation of 2-(2-methoxyethyl)-1-oxo-N-(4-(pyrimidin-2-yl)phenyl)-3-(thiophen-2-yl)-1,2,3,4-tetrahydroisoquinoline-4-carboxamide (C175)

This compound was obtained following method B, from thiophene-2-carbaldehyde, 2-methoxyethanamine, homophthalic anhydride and 4-(pyrimidin-2-yl)aniline in 31% overall yield. The title compound was purified by flash chromatography eluting with dichloromethane, 15 to 70% ethyl acetate and precipitation in ethyl acetate. ESI/APCI(+): 485 (M+H). ESI/APCI(−): 483 (M−H). ¹H NMR (DMSO-d₆) δ 10.63 (1H, s, NH), 8.87 (2H, d), 8.35 (2H, d), 7.92 (1H, d), 7.75 (2H, dd), 7.53-7.37 (4H, m), 7.31 (1H, d), 7.02 (1H, s), 6.91 (1H, d), 5.63 (1H, s), 4.33 (1H, s), 4.07 (1H, dt), 3.38 (2H, m), 3.03 (1H, dt), 2.99 (3H, s).

### Example 149

### Preparation of 2-(2-methoxyethyl)-N-(1-methylindolin-5-yl)-1-oxo-3-(thiophen-2-yl)-1,2,3,4-tetrahydroisoquinoline-4-carboxamide (C176)

This compound was obtained following method B, from thiophene-2-carbaldehyde, 2-methoxyethanamine, homophthalic anhydride and 1-methylindolin-5-amine in 42% overall yield. The title compound was purified by flash chromatography eluting with dichloromethane, 5 to 50% ethyl acetate and precipitation in dichloromethane. ESI/APCI(+): 462 (M+H). ESI/APCI(−): 460 (M−H). ¹H NMR (DMSO-d₆) δ 10.15 (1H, s, NH), 7.90 (1H, d), 7.41 (2H, m), 729 (2H, m), 7.00 (1H, s), 6.89 (2H, m), 6.84 (1H, s), 6.75 (1H, d), 5.56 (1H, s), 4.24 (1H, s), 4.04 (1H, dt), 3.41 (2H, t), 3.20 (2H, t), 3.09 (1H, m), 3.05 (3H, s), 2.77 (2H, t), 2.63 (3H, s).

### Example 150

### Preparation of N-(4-tert-butylthiazol-2-yl)-2-(2-methoxyethyl)-1-oxo-3-(thiophen-2-yl)-1,2,3,4-tetrahydroisoquinoline-4-carboxamide (C177)

This compound was obtained following method B, from thiophene-2-carbaldehyde, 2-methoxyethanamine, homophthalic anhydride and 4-tert-butylthiazol-2-amine in 25% overall yield. The title compound was purified by flash chromatography eluting with heptane, 5 to 50% ethyl acetate and precipitation in ethyl acetate. ESI/APCI(+): 470 (M+H). ESI/APCI(−): 468 (M−H). ¹H NMR (DMSO-d₆) δ 12.74 (1H, s, NH), 7.91 (1H, d), 7.40 (2H, m), 7.28 (2H, m), 6.80 (1H, s), 6.88 (1H, td), 6.75 (1H, s), 5.65 (1H, s), 4.39 (1H, s), 4.09 (1H, dt), 3.36 (2H, m), 2.94 (1H, dt), 2.88 (3H, s), 1.27 (9H, s).

### Example 151

### Preparation of 2-(2-methoxyethyl)-N-(4-(oxazol-5-yl)phenyl)-1-oxo-3-(thiophen-2-yl)-1,2,3,4-tetrahydroisoquinoline-4-carboxamide (C178)

This compound was obtained following method B, from thiophene-2-carbaldehyde, 2-methoxyethanamine, homophthalic anhydride and 4-(oxazol-5-yl)aniline in 29% overall yield. The title compound was purified by flash chromatography eluting with dichloromethane, 5 to 70% ethyl acetate. ESI/APCI(+): 474 (M+H). ESI/APCI(−): 472 (M−H). ¹H NMR (DMSO d-6) δ 10.56 (1H, s, NH), 8.40 (1H, s), 7.92 (1H, d), 7.71 (2H, d), 7.67 (2H, d), 7.52 (1H, s), 7.41 (2H, m), 7.35 (1H, d), 7.30 (1H, d), 7.01 (1H, s), 6.90 (1H, d), 5.61 (1H, s), 4.30 (1H, s), 4.05 (1H, dt), 3.42 (2H, t), 3.03 (1H, dt), 3.00 (3H, s).

### Example 152

### Preparation of 2-(2-methoxyethyl)-1-oxo-3-(thiophen-2-yl)-N-(3-(trifluoromethyl)-1,2,4-thiadiazol-5-yl)-1,2,3,4-tetrahydroisoquinoline-4-carboxamide (C179)

This compound was obtained following method B, from thiophene-2-carbaldehyde, 2-methoxyethanamine, homophthalic anhydride and 3-(trifluoromethyl)-1,2,4-thiadiazol-5-amine in 23% overall yield. The title compound was purified by flash chromatography eluting with heptane, 5 to 50% ethyl acetate. ESI/APCI(+): 483 (M+H). ESI/APCI(−): 481 (M−H). ¹H NMR (DMSO-d₆) δ 13.98 (1H, s, NH), 7.93 (1H, d), 7.44 (2H, m), 7.35 (1H, d), 7.31 (1H, d), 6.97 (1H, s), 6.90 (1H, t), 5.68 (1H, s), 4.55 (1H, s), 4.09 (1H, dt), 3.33 (2H, m), 2.95 (1H, dt), 2.85 (3H, s).

### Example 153

### Preparation of 2-(2-methoxyethyl)-N-(1-methyl-1H-pyrazol-3-yl)-1-oxo-3-(thiophen-2-yl)-1,2,3,4-tetrahydroisoquinoline-4-carboxamide (C180)

This compound was obtained following method B, from thiophene-2-carbaldehyde, 2-methoxyethanamine, homophthalic anhydride and 1-methyl-1H-pyrazol-3-amine in 38% overall yield. The title compound was purified by flash chromatography eluting with dichloromethane, 30 to 100% ethyl acetate. ESI/APCI(+): 411 (M+H). ¹H NMR (DMSO-d₆) δ 10.90 (1H, s, NH), 7.89 (1H, d), 7.52 (1H, s), 7.41 (2H, m), 7.27 (2H, m), 6.99 (1H, s), 6.88 (1H, t), 6.37 (1H, s), 5.56 (1H, s), 4.26 (1H, s), 4.01 (1H, dt), 3.74 (3H, s), 3.38 (2H, m), 3.06 (1H, dt), 3.03 (3H, s).

### Example 154

### Preparation of N-(4-(hydroxymethyl)phenyl)-2-(2-methoxyethyl)-1-oxo-3-(thiophen-2-yl)-1,2,3,4-tetrahydroisoquinoline-4-carboxamide (C181)

This compound was obtained following method B, from thiophene-2-carbaldehyde, 2-methoxyethanamine, homophthalic anhydride and (4-aminophenyl)methanol in 4% overall yield. The title compound was purified by flash chromatography eluting with heptane, 30 to 100% ethyl acetate. ESI/APCI(+): 437 (M+H). ESI/APCI(−): 435 (M−H). ¹H NMR (DMSO-d₆) δ 10.34 (1H, s, NH), 7.91 (1H, d), 7.54 (2H, d), 7.42 (2H, m), 7.34 (1H, d), 7.29 (1H, d), 7.24 (2H, d), 7.01 (1H, s), 6.90 (1H, t), 5.60 (1H, s), 5.09 (1H, t, OH), 4.42 (2H, d), 4.28 (1H, s), 4.04 (1H, dt), 3.42 (2H, m), 3.06 (1H, dt), 3.02 (3H, s).

### Example 155

### Preparation of 2-(2-methoxyethyl)-1-oxo-N-(5-phenyl-1H-pyrazol-3-yl)-3-(thiophen-2-yl)-1,2,3,4-tetrahydroisoquinoline-4-carboxamide (C182)

This compound was obtained following method B, from thiophene-2-carbaldehyde, 2-methoxyethanamine, homophthalic anhydride and 5-phenyl-1H-pyrazol-3-amine in 31% overall yield. The title compound was purified by flash chromatography eluting with dichloromethane, 5 to 70% ethyl acetate. ESI/APCI(+): 473 (M+H). ESI/APCI(−): 471 (M−H). ¹H NMR (DMSO-d₆) δ 7.96 (3H, m), 7.52-7.39 (7H, m), 6.92 (1H, t), 6.86 (1H, d), 6.79 (2H, s), 5.98 (1H, s), 5.90 (1H, s), 5.46 (1H, s), 4.17 (1H, dt), 3.33 (2H, m), 3.00 (3H, s), 2.87 (1H, dt).

### Example 156

### Preparation of N-(5-(furan-2-yl)-1H-pyrazol-3-yl)-2-(2-methoxyethyl)-1-oxo-3-(thiophen-2-yl)-1,2,3,4-tetrahydroisoquinoline-4-carboxamide (C183)

This compound was obtained following method B, from thiophene-2-carbaldehyde, 2-methoxyethanamine, homophthalic anhydride and 5-(furan-2-yl)-1H-pyrazol-3-amine in 20% overall yield. The title compound was purified by flash chromatography eluting with dichloromethane, 5 to 40% ethyl acetate. ESI/APCI(+): 463 (M+H). ESI/APCI(−): 461 (M−H). ¹H NMR (DMSO-d₆) δ 7.95 (1H, dd), 7.84 (1H, s), 7.52-7.42 (3H, m), 7.36 (1H, dd), 6.99 (1H, d), 6.92 (2H, m), 6.81 (2H, s), 6.66 (1H, d), 5.90 (1H, s), 5.70 (1H, s), 5.37 (1H, s), 4.14 (1H, dt), 3.31 (2H, m), 3.01 (3H, s), 2.86 (1H, dt).

### Example 157

### Preparation of 2-(2-methoxyethyl)-1-oxo-N-(4-phenoxyphenyl)-3-(thiophen-2-yl)-1,2,3,4-tetrahydroisoquinoline-4-carboxamide (C192)

This compound was obtained following method B, from thiophene-2-carbaldehyde, 2-methoxyethanamine, homophthalic anhydride and 4-phenoxyaniline in 42% overall yield. The title compound was purified by flash chromatography eluting with heptane, 5 to 50% ethyl acatetate and repurified by flash chromatography eluting with dichloromethane, 2 to 20% ethyl acetate. ESI/APCI(+): 499 (M+H). ESI/APCI(−): 497 (M−H). ¹H NMR (DMSO-d₆) δ 10.41 (1H, s, NH), 7.91 (1H, d), 7.61 (2H, d), 7.51-7.30 (6H, m), 7.07 (1H, t), 7.01-6.91 (6H, m), 5.59 (1H, s), 4.27 (1H, s), 4.05 (1H, dt), 3.42 (2H, t), 3.08 (1H, dt), 3.04 (3H, s).

### Example 158

### Preparation of ethyl 5-(-2-(2-methoxyethyl)-1-oxo-3-(thiophen-2-yl)-1,2,3,4-tetrahydroisoquinoline-4-carboxamido)-1,3,4-oxadiazole-2-carboxylate (C193)

This compound was obtained following method B, from thiophene-2-carbaldehyde, 2-methoxyethanamine, homophthalic anhydride and ethyl 5-amino-1,3,4-oxadiazole-2-carboxylate in 2% overall yield. The title compound was purified by flash chromatography eluting with dichloromethane, 1 to 10% methanol and repurified by flash chromatography eluting with dichloromethane, 2 to 20% ethyl acetate. ESI/APCI(+): 471 (M+H). ESI/APCI(−): 469 (M−H). ¹H NMR (DMSO-d₆) δ 12.80 (1H, s, NH), 7.93 (1H, d), 7.51-7.42 (2H, m), 7.34 (1H, m), 7.30 (1H, m), 6.96 (1H, s), 6.89 (1H, m), 5.65 (1H, s), 4.41-4.35 (3H, m), 4.07 (1H, dt), 3.41 (2H, m), 3.05 (1H, m), 3.01 (3H, s).

### Example 159

### Preparation of 2-(2-methoxyethyl)-1-oxo-N-(3-phenyl-1,2,4-thiadiazol-5-yl)-3-(thiophen-2-yl)-1,2,3,4-tetrahydroisoquinoline-4-carboxamide (C194)

This compound was obtained following method B, from thiophene-2-carbaldehyde, 2-methoxyethanamine, homophthalic anhydride and 3-phenyl-1,2,4-thiadiazol-5-amine in 30% overall yield. The title compound was purified by flash chromatography eluting with heptane, 5 to 50% ethyl acetate and repurified by flash chromatography eluting with dichloromethane, 2 to 20% ethyl acetate. ESI/APCI(+): 491 (M+H). ESI/APCI(−): 489 (M−H). ¹H NMR (DMSO-d₆) δ 13.78 (1H, s, NH), 8.18 (2H, d), 7.94 (1H, d), 7.54-7.47 (5H, m), 7.37 (1H, d), 7.31 (1H, d), 7.00 (1H, s), 6.91 (1H, t), 5.72 (1H, s), 4.57 (1H, s), 4.12 (1H, dt), 3.36 (2H, m), 2.95 (1H, dt), 2.86 (3H, s).

### Example 160

### Preparation of N-(benzofuran-5-yl)-2-(2-methoxyethyl)-1-oxo-3-(thiophen-2-yl)-1,2,3,4-tetrahydroisoquinoline-4-carboxamide (C195)

This compound was obtained following method B, from thiophene-2-carbaldehyde, 2-methoxyethanamine, homophthalic anhydride and benzofuran-5-amine in 37% overall yield. The title compound was purified by flash chromatography eluting with heptane, 15 to 70% ethyl acetate and repurified by flash chromatography eluting with dichloromethane, 2 to 20% ethyl acetate. ESI/APCI(+): 447 (M+H). ESI/APCI(−): 445 (M−H). ¹H NMR (DMSO-d₆) δ 10.39 (1H, s, NH), 7.99 (1H, s), 7.94 (2H, m), 7.56-7.36 (5H, m), 7.30 (1H, d), 7.02 (1H, s), 6.92 (2H, s), 5.61 (1H, s), 4.29 (1H, s), 4.04 (1H, dt), 3.43 (2H, t), 3.04 (1H, dt), 3.01 (3H, s).

### Example 161

### Preparation of 2-(2-methoxyethyl)-1-oxo-N-(5-methyl-1H-pyrazol-3-yl)-3-(thiophen-2-yl)-1,2,3,4-tetrahydroisoquinoline-4-carboxamide (C196)

This compound was obtained following method B, from thiophene-2-carbaldehyde, 2-methoxyethanamine, homophthalic anhydride and 5-methyl-1H-pyrazol-3-amine in 17% overall yield. The title compound was purified by flash chromatography eluting with dichloromethane, 5 to 40% ethyl acetate and repurified by flash chromatography eluting with heptane, 15 to 70% ethyl acetate. ESI/APCI(+): 411 (M+H). ESI/APCI(−): 409 (M−H). ¹H NMR (DMSO-d₆) δ 7.93 (1H, d), 7.47 (2H, m), 7.36 (1H, d), 7.32 (1H, d), 6.89 (2H, m), 6.58 (2H, d), 5.82 (1H, s), 5.31 (1H, s), 5.26 (1H, s), 4.13 (1H, dt), 3.35 (2H, m), 3.02 (3H, s), 2.87 (1H, dt).

### Example 162

### Preparation of N-(3-cyclopropyl-1-methyl-1H-pyrazol-5-yl)-2-(2-methoxyethyl)-1-oxo-3-(thiophen-2-yl)-1,2,3,4-tetrahydroisoquinoline-4-carboxamide (C197)

This compound was obtained following method B, from thiophene-2-carbaldehyde, 2-methoxyethanamine, homophthalic anhydride and 5-amino-3-cyclopropyl-1-methylene-1H-pyrazol-1-ium in 13% overall yield. The title compound was purified by flash chromatography eluting with dichloromethane, 5 to 70% ethyl acetate. ESI/APCI(+): 451 (M+H). ESI/APCI(−): 449 (M−H). ¹H NMR (DMSO-d₆) δ 10.21 (1H, s, NH), 7.91 (1H, d), 7.50-7.42 (2H, m), 7.36 (1H, d), 7.30 (1H, d), 7.00 (1H, s), 6.90 (1H, t), 5.89 (1H, s), 5.57 (1H, s), 4.34 (1H, s), 3.98 (1H, dt), 3.56 (3H, s), 3.37 (2H, m), 3.13 (1H, m), 3.10 (3H, s), 1.72 (1H, m), 0.77 (2H, m), 0.54 (2H, m).

### Example 163

### Preparation of N-(1,1-dioxobenzo[b]thiophen-6-yl)-2-(2-methoxyethyl)-1-oxo-3-(thiophen-2-yl)-1,2,3,4-tetrahydroisoquinoline-4-carboxamide (C198)

This compound was obtained following method B, from thiophene-2-carbaldehyde, 2-methoxyethanamine, homophthalic anhydride and 1,1-dioxobenzo[b]thiophen-6-amine in 19% overall yield. The title compound was purified by flash chromatography eluting with dichloromethane, 5 to 50% ethyl acetate and recrystallised from ethanol. ESI/APCI(+): 495 (M+H). ESI/APCI(−): 493 (M−H). ¹H NMR (DMSO-d₆) δ 10.90 (1H, s, NH), 8.11 (1H, s), 7.91 (1H, d), 7.73 (1H, d), 7.55 (2H, t), 7.47 (1H, t), 7.42 (1H, t), 7.35 (1H, d), 7.31 (1H, d), 7.27 (1H, d), 7.00 (1H, s), 6.90 (1H, s), 5.61 (1H, s), 4.32 (1H, s), 4.06 (1H, dt), 3.40 (2H, t), 3.01 (1H, dt), 2.97 (3H, s).

### Example 164

### Preparation of N-(6-fluorobenzo[d]thiazol-2-yl)-2-(2-methoxyethyl)-1-oxo-3-(thiophen-2-yl)-1,2,3,4-tetrahydroisoquinoline-4-carboxamide (C199)

This compound was obtained following method B, from thiophene-2-carbaldehyde, 2-methoxyethanamine, homophthalic anhydride and 6-fluorobenzo[d]thiazol-2-amine in 31% overall yield. The title compound was purified by flash chromatography eluting with heptane, 15 to 70% ethyl acetate and recrystallised from ethanol. ESI/APCI(+): 482 (M+H). ESI/APCI(−): 480 (M−H). ¹H NMR (DMSO-d₆) δ 13.07 (1H, s, NH), 7.93 (1H, d), 7.88 (1H, dd), 7.77 (1H, dd), 7.52 (2H, m), 7.37 (1H, d), 7.33 (2H, m), 7.00 (1H, s), 6.90 (1H, t), 5.70 (1H, s), 4.48 (1H, s), 4.11 (1H, dt), 3.35 (2H, m), 2.95 (1H, dt), 2.89 (3H, s).

### Example 165

### Preparation of N-(3-(1H-1,2,4-triazol-1-yl)phenyl)-2-(2-methoxyethyl)-1-oxo-3-(thiophen-2-yl)-1,2,3,4-tetrahydroisoquinoline-4-carboxamide (C200)

This compound was obtained following method B, from thiophene-2-carbaldehyde, 2-methoxyethanamine, homophthalic anhydride and 3-(1H-1,2,4-triazol-1-yl)aniline in 32% overall yield. The title compound was purified by flash chromatography eluting with dichloromethane, 30 to 100% ethyl acetate and recrystallised from ethanol. ESI/APCI(+): 474 (M+H). ESI/APCI(−): 472 (M−H). ¹H NMR (DMSO-d₆) δ 10.69 (1H, s, NH), 9.26 (1H, s), 8.22 (1H, s), 8.20 (1H, s), 7.92 (1H, d), 7.59 (1H, t), 7.53 (1H, d), 7.50 (3H, m), 7.35 (1H, d), 7.31 (1H, d), 7.00 (1H, s), 6.90 (1H, t), 5.62 (1H, s), 4.32 (1H, s), 4.05 (1H, dt), 3.41 (2H, t), 3.03 (1H, dt), 2.98 (3H, s).

### Example 166

### Preparation of N-(4-(3,5-dimethyl-1H-pyrazol-1-yl)phenyl)-2-(2-methoxyethyl)-1-oxo-3-(thiophen-2-yl)-1,2,3,4-tetrahydroisoquinoline-4-carboxamide (C201)

This compound was obtained following method B, from thiophene-2-carbaldehyde, 2-methoxyethanamine, homophthalic anhydride and 4-(3,5-dimethyl-1H-pyrazol-1-yl)aniline in 9% overall yield. The title compound was purified by flash chromatography eluting with heptane, 30 to 100% ethyl acetate and precipitated in a mixture of heptane and ethyl acetate. ESI/APCI(+): 501 (M+H). ESI/APCI(−): 499 (M−H). ¹H NMR (DMSO-d₆) δ 10.54 (1H, s, NH), 7.91 (1H, d), 7.72 (1H, s), 7.69 (1H, s), 7.52-7.36 (5H, m), 7.30 (1H, d), 7.02 (1H, s), 6.90 (1H, t), 6.03 (1H, s), 5.61 (1H, s), 4.30 (1H, s), 4.05 (1H, dt), 3.43 (2H, t), 3.02 (1H, dt), 2.98 (3H, s).

### Example 167

### Preparation of 2-(2-methoxyethyl)-N-(4-(2-methyl-1H-imidazol-1-yl)phenyl)-1-oxo-3-(thiophen-2-yl)-1,2,3,4-tetrahydroisoquinoline-4-carboxamide (211)

This compound was obtained following method B, from thiophene-2-carbaldehyde, 2-methoxyethanamine, homophthalic anhydride and 4-(2-methyl-1H-imidazol-1-yl)aniline in 5% overall yield. The title compound was purified by flash chromatography eluting with heptane, 30 to 100% ethyl acetate and by thick layer chromatography eluting with 5% methanol in dichloromethane. ESI/APCI(+): 498 (M+H). ESI/APCIH: 496 (M−H).

### Example 168

### Preparation of N-(3-((1H-imidazol-1-yl)methyl)phenyl)-2-(2-methoxyethyl)-1-oxo-3-(thiophen-2-yl)-1,2,3,4-tetrahydroisoquinoline-4-carboxamide (C212)

This compound was obtained following method B, from thiophene-2-carbaldehyde, 2-methoxyethanamine, homophthalic anhydride and 3-((1H-imidazol-1-yl)methyl)aniline in 3% overall yield. The title compound was purified by flash chromatography eluting with heptane, 0 to 100% ethyl acetate and by thick layer chromatography eluting with 5% methanol in dichloromethane. ESI/APCI(+): 487 (M+H). ESI/APCIH: 485 (M−H).

### Example 169

### Preparation of 2-(2-methoxyethyl)-N-(4-methyl-3-(methylsulfonamido)phenyl)-1-oxo-3-(thiophen-2-yl)-1,2,3,4-tetrahydroisoquinoline-4-carboxamide (C213)

This compound was obtained following method B, from thiophene-2-carbaldehyde, 2-methoxyethanamine, homophthalic anhydride and N-(5-amino-2-methylphenyl)methanesulfonamide in 14% overall yield. The title compound was purified by flash chromatography eluting with heptane, 0 to 60% ethyl acetate. ESI/APCI(+): 485 (M+H). ESI/APCI(−): 482 (M−H). ¹H NMR (DMSO-d₆) δ 10.41 (1H, s), 9.04 (1H, s), 7.91 (1H, d), 7.57 (1H, s), 7.45 (4H, m), 7.31 (2H, m), 7.17 (1H, d), 7.00 (1H, m), 6.90 (1H, m), 5.58 (1H, s), 4.26 (1H, s), 4.08 (1H, m), 3.42 (2H, m), 3.06 (1H, m), 3.00 (3H, s), 2.95 (3H, s), 2.36 (3H, s).

### Example 170

### Preparation of 2-(2-methoxyethyl)-N-(4-(4-methyl-4H-1,2,4-triazol-3-yl)phenyl)-1-oxo-3-(thiophen-2-yl)-1,2,3,4-tetrahydroisoquinoline-4-carboxamide (C214)

This compound was obtained following method B, from thiophene-2-carbaldehyde, 2-methoxyethanamine, homophthalic anhydride and 4-(4-methyl-4H-1,2,4-triazol-3-yl)aniline in 11% overall yield. The title compound was purified by flash chromatography eluting with heptane, 0 to 60% ethyl acetate and by thick layer chromatography eluting with 5% methanol in dichloromethane. ESI/APCI(+): 488 (M+H). ESI/APCIH: 486 (M−H).

### Example 171

### Preparation of N-(4-((1H-imidazol-1-yl)methyl)phenyl)-2-(2-methoxyethyl)-1-oxo-3-(thiophen-2-yl)-1,2,3,4-tetrahydroisoquinoline-4-carboxamide (C215)

This compound was obtained following method B, from thiophene-2-carbaldehyde, 2-methoxyethanamine, homophthalic anhydride and 4-((1H-imidazol-1-yl)methyl)aniline in 30% overall yield. The title compound was purified by flash chromatography eluting with ethyl acetate, 1 to 10% methanol and by preparative HPLC. ESI/APCI(+): 487 (M+H). ESI/APCI(−): 485 (M−H). ¹H NMR (DMSO-d₆) δ 10.44 (1H, s, NH), 7.92 (1H, d), 7.70 (1H, d), 7.58 (2H, m), 7.51 (2H, m), 7.35 (1H, d), 7.29 (1H, d), 7.23 (2H, m), 7.13 (1H, s), 7.00 (1H, s), 6.91 (2H, m), 5.57 (1H, s), 5.12 (2H, s), 4.27 (1H, s), 4.02 (1H, dt), 3.41 (2H, t), 3.09 (1H, m), 2.99 (3H, s).

### Example 172

### Preparation of 2-(2-methoxyethyl)-1-oxo-N-(4-(piperidin-1-ylmethyl)phenyl)-3-(thiophen-2-yl)-1,2,3,4-tetrahydroisoquinoline-4-carboxamide (C216)

This compound was obtained following method B, from thiophene-2-carbaldehyde, 2-methoxyethanamine, homophthalic anhydride and 4-(piperidin-1-ylmethyl)aniline in 14% overall yield. The title compound was purified by flash chromatography eluting with ethyl acetate, 1 to 10% methanol and by preparative HPLC. ESI/APCI(+): 504 (M+H). ESI/APCI(−): 502 (M−H).

### Example 173

### Preparation of N-(4-((1H-1,2,4-triazol-1-yl)methyl)phenyl)-2-(2-methoxyethyl)-1-oxo-3-(thiophen-2-yl)-1,2,3,4-tetrahydroisoquinoline-4-carboxamide (C217)

This compound was obtained following method B, from thiophene-2-carbaldehyde, 2-methoxyethanamine, homophthalic anhydride and 4-((1H-1,2,4-triazol-1-yl)methyl)aniline in 20% overall yield. The title compound was purified by flash chromatography eluting with ethyl acetate, 1 to 10% methanol and by preparative HPLC. ESI/APCI(+): 488 (M+H). ESI/APCI(−): 486 (M−H).

### Example 174

### Preparation of 2-(2-methoxyethyl)-1-oxo-N-(4-(tetrahydro-2H-pyran-4-yloxy)phenyl)-3-(thiophen-2-yl)-1,2,3,4-tetrahydroisoquinoline-4-carboxamide (C218)

This compound was obtained following method B, from thiophene-2-carbaldehyde, 2-methoxyethanamine, homophthalic anhydride and 4-(tetrahydro-2H-pyran-4-yloxy)aniline in 30% overall yield. The title compound was purified twice by flash chromatography eluting first with dichloromethane, 15 to 70% ethyl acetate and heptane, 30 to 100% ethyl acetate. ESI/APCI(+): 507 (M+H). ESI/APCI(−): 505 (M−H). ¹H NMR (DMSO-d₆) δ 10.21 (1H, s, NH), 7.90 (1H, dd), 7.50 (3H, m), 7.40 (1H, dt), 7.32 (1H, d), 7.29 (1H, dd), 7.00 (1H, d), 6.94 (1H, s), 6.92 (2H, m), 5.57 (1H, s), 4.49 (1H, septuplet), 4.23 (1H, s), 4.02 (1H, dt), 3.80 (2H, dt), 3.41 (4H, m), 3.05 (1H, dt), 3.03 (3H, s), 1.95 (2H, m), 1.59 (2H, dtd).

### Example 175

### Preparation of N-(benzo[d]oxazol-6-yl)-2-(2-methoxyethyl)-1-oxo-3-(thiophen-2-yl)-1,2,3,4-tetrahydroisoquinoline-4-carboxamide (C219)

This compound was obtained following method B, from thiophene-2-carbaldehyde, 2-methoxyethanamine, homophthalic anhydride and benzo[d]oxazol-6-amine in 24% overall yield. The title compound was purified by flash chromatography eluting with dichloromethane, 5 to 70% ethyl acetate. ESI/APCI(+): 448 (M+H). ESI/APCI(−): 446 (M−H). ¹H NMR (DMSO-d₆) δ 10.69 (1H, s, NH), 8.65 (1H, s), 8.22 (1H, d), 7.92 (1H, d), 7.74 (1H, d), 7.52 (3H, m), 7.39 (1H, d), 7.31 (1H, d), 7.02 (1H, d), 6.91 (1H, dd), 5.62 (1H, s), 4.32 (1H, s), 4.05 (1H, dt), 3.42 (2H, t), 3.03 (1H, dt), 2.97 (3H, s).

### Example 176

### Preparation of N-(3,4-dimethylisoxazol-5-yl)-2-(2-methoxyethyl)-1-oxo-3-(thiophen-2-yl)-1,2,3,4-tetrahydroisoquinoline-4-carboxamide (C220)

This compound was obtained following method B except that the mixture was stirred at 50° C. for 5 days, from thiophene-2-carbaldehyde, 2-methoxyethanamine, homophthalic anhydride and 3,4-dimethylisoxazol-5-amine in 24% overall yield. The title compound was purified by flash chromatography eluting with heptane, 15 to 70% ethyl acetate and by preparative thick layer chromatography eluting with 50% ethyl acetate in heptane. ESI/APCI(+): 426 (M+H). ESI/APCI(−): 424 (M−H). ¹H NMR (DMSO-d₆) δ 10.99 (1H, s, NH), 7.93 (1H, d), 7.50-7.42 (2H, m), 7.38 (1H, d), 7.31 (1H, d), 7.00 (1H, s), 6.92 (1H, t), 5.62 (1H, s), 4.31 (1H, s), 4.07 (1H, dt), 3.46 (2H, t), 3.12 (3H, s), 3.08 (1H, m).

### Example 177

### Preparation of N-(3-cyano-4-(1H-pyrrol-1-yl)phenyl)-2-(2-methoxyethyl)-1-oxo-3-(thiophen-2-yl)-1,2,3,4-tetrahydroisoquinoline-4-carboxamide (C222)

This compound was obtained following method B, from thiophene-2-carbaldehyde, 2-methoxyethanamine, homophthalic anhydride and 5-amino-2-(1H-pyrrol-1-yl)benzonitrile in 17% overall yield. The title compound was purified by flash chromatography eluting with heptane, 30 to 80% ethyl acetate. ESI/APCI(+): 497 (M+H). ESI/APCI(−): 495 (M−H). ¹H NMR (DMSO-d₆) δ 10.91 (1H, s), 8.12 (1H, d), 8.02 (1H, d), 7.85 (1H, dd), 7.59 (2H, m), 7.49 (1H, t), 7.39 (1H, d), 7.29 (1H, d), 7.20 (2H, m), 6.85 (2H, m), 6.32 (2H, m), 5.53 (1H, d, J=5.3 Hz), 4.78 (1H, d, J=5.3 Hz), 4.01 (1H, m), 3.53 (2H, m), 3.25 (3H, s), 3.08 (1H, m).

### Example 178

### Preparation of N-(5-chloropyridin-2-yl)-2-(2-methoxyethyl)-1-oxo-3-(thiophen-2-yl)-1,2,3,4-tetrahydroisoquinoline-4-carboxamide (C223)

This compound was obtained following method B, from thiophene-2-carbaldehyde, 2-methoxyethanamine, homophthalic anhydride and 2-amino-5-chloropyridine in 3% overall yield. The title compound was purified by flash chromatography eluting with heptane, 30 to 80% ethyl acetate and triturated in diisopropylether. ESI/APCI(+): 442 (M+H). ESI/APCI(−): 440 (M−H). ¹H NMR (DMSO-d₆) δ 11.31 (1H, s), 8.41 (1H, s), 8.06 (1H, d), 7.90 (2H, m), 7.45 (2H, m), 7.30 (2H, m), 7.01 (1H, d), 6.86 (1H, t), 5.61 (1H, s), 4.44 (1H, s), 4.10 (1H, m), 3.43 (2H, m), 3.01 (1H, m), 2.93 (3H, s).

### Example 179

### Preparation of 3-(furan-2-yl)-2-(2-methoxyethyl)-N-(3-methoxyphenyl)-1-oxo-1,2,3,4-tetrahydroisoquinoline-4-carboxamide (C6)

2-Furfuraldehyde (197 μL, 2.02 mmol) and MgSO₄ (640 mg, 5.32 mmol) were added to a solution of 2-methoxyethylamine (167 μL, 2.02 mmol) in chloroform (8 mL). The reaction mixture was stirred at RT overnight. The solid was filtered. The filtrate was washed with H₂O and brine, dried over Na₂SO₄, filtered and concentrated to give the desired imine which was used in the next step without further purification.

Homophthalic anhydride (140 mg, 0.86 mmol) was added to a solution of previous imine (132 mg, 0.86 mmol) in chloroform (5 mL). The reaction mixture was stirred at RT for 2.5 h and then concentrated to give the desired acid which was used in the next step without further purification.

To a solution of previous carboxylic acid (0.86 mmol) in DMF (3 mL) were added m-anisidine (126 μL, 1.12 mmol), diisopropylethylamine (449 μL, 2.57 mmol) and HATU (525 mg, 1.38 mmol). After 15 h at RT, the reaction mixture was concentrated. The residue was dissolved in CH₂Cl₂ and washed with sat. NaHCO₃. The organic phase was washed with water and brine, dried over Na₂SO₄, filtered, concentrated and purified twice by flash chromatography on silica gel eluting first with heptane, 30 to 80% ethyl acetate and with heptane, 40 to 65% ethyl acetate to give title compound (45 mg, 12%). ESI/APCI(+): 421 (M+H). ¹H NMR (DMSO-d₆) δ 3.14 (s, 3H), 3.13-3.22 (m, 1H), 3.46 (m, 2H), 3.71 (s, 3H), 4.06-4.14 (m, 1H), 4.36 (s, 1H), 5.39 (s, 1H), 6.03 (m, 1H), 6.61 (m, 1H), 6.61-6.65 (m, 1H), 7.1-7.6 (m, 7H), 7.88 (m, 1H), 10.40 (s, 1H).

### Example 180

### Preparation of 3-(2-fluorophenyl)-2-(2-methoxyethyl)-N-(3-methoxyphenyl)-1-oxo-1,2,3,4-tetrahydroisoquinoline-4-carboxamide (C7) (Method C)

A mixture of 2-methoxyethylamine (200 μL, 2.33 mmol) and 2-fluorobenzaldehyde (245 μL, 2.33 mmol) was heated in sealed tube for 6 h at 60° C. The crude material was dried overnight under vacuum and was used in the next step without further purification.

Homophthalic anhydride (378 mg, 2.33 mmol) was added to a solution of previous imine (2.33 mmol) in chloroform (5 mL). The reaction mixture was stirred at RT for 3 h and then concentrated to give the desired acid which was used in the next step without further purification.

To a solution of previous carboxylic acid (2.33 mmol) in DMF (5 mL) were added m-anisidine (3154, 2.80 mmol), diisopropylethylamine (4894, 2.80 mmol) and HATU (1.06 g, 2.80 mmol). After 2 h at RT, the reaction mixture was concentrated. The residue was dissolved in CH₂Cl₂ and washed with sat. NaHCO₃. The organic phase was washed with water and brine, dried over Na₂SO₄, filtered, concentrated and purified by flash chromatography on silica gel eluting with heptane, 30 to 65% ethyl acetate to give title compound (535 mg, 56%). ESI/APCI(+): 449 (M+H). ¹H NMR (DMSO-d₆) δ 3.05 (s, 3H), 3.20-3.29 (m, 1H), 3.42-3.54 (m, 2H), 3.72 (s, 3H), 3.88-3.96 (m, 1H), 4.12 (s, 1H), 5.55 (s, 1H), 6.63-6.65 (m, 1H), 6.78-6.84 (t, 1H), 7.0-7.5 (m, 9H), 7.98-8.00 (m, 1H), 10.24 (s, 1H).

### Example 181

### Preparation of 3-(3-Fluorophenyl)-2-(2-methoxyethyl)-N-(3-methoxyphenyl)-1-oxo-1,2,3,4-tetrahydroisoquinoline-4-carboxamide (C8)

This compound was obtained following method C, from 3-fluorobenzaldehyde, 2-methoxyethylamine, homophthalic anhydride and 3-methoxyaniline in 33% overall yield. The title compound was purified by flash chromatography eluting with heptane, 30 to 80% ethyl acetate and crystallization from ethyl acetate-heptane. ESI/APCI(+): 449 (M+H). ¹H NMR (DMSO-d₆) δ 2.99 (s, 3H), 3.02-3.11 (m, 1H), 3.39-3.44 (m, 2H), 3.72 (s, 3H), 4.00-4.08 (m, 1H), 4.20 (s, 1H), 5.38 (s, 1H), 6.63-6.67 (m, 1H), 6.9-7.5 (m, 10H), 7.93-7.97 (m, 1H), 10.37 (s, 1H).

### Example 182

### Preparation of 3-(4-Fluorophenyl)-2-(2-methoxyethyl)-N-(3-methoxyphenyl)-1-oxo-1,2,3,4-tetrahydroisoquinoline-4-carboxamide (C9)

This compound was obtained following method C, from 4-fluorobenzaldehyde, 2-methoxyethylamine, homophthalic anhydride and 3-methoxyaniline in 10% overall yield. The title compound was purified by flash chromatography eluting with heptane, 30 to 65% ethyl acetate and crystallization from ethyl acetate-heptane. ESI/APCI(+): 449 (M+H). ¹H NMR (DMSO-d₆) δ 2.99 (s, 3H), 3.01-3.08 (m, 1H), 3.39-3.44 (m, 2H), 3.72 (s, 3H), 3.99-4.07 (m, 1H), 4.15 (s, 1H), 5.35 (s, 1H), 6.62-6.66 (m, 1H), 7.0-7.5 (m, 10H), 7.93-7.96 (m, 1H), 10.38 (s, 1H).

### Example 183

### Preparation of 3-(2,6-Difluorophenyl)-2-(2-methoxyethyl)-N-(3-methoxyphenyl)-1-oxo-1,2,3,4-tetrahydroisoquinoline-4-carboxamide (C10)

This compound was obtained following method C, from 2,6-difluorobenzaldehyde, 2-methoxyethylamine, homophthalic anhydride and 3-methoxyaniline in 27% overall yield. The title compound was purified twice by flash chromatography eluting first with dichloromethane, 0 to 50% ethyl acetate and with heptane, 30 to 65% ethyl acetate. ESI/APCI(+): 467 (M+H). ¹H NMR (DMSO-d₆) δ 3.05 (s, 3H), 3.23-3.32 (m, 1H), 3.38-3.45 (m, 1H), 3.48-3.55 (m, 1H), 3.71 (s, 3H), 3.83-3.91 (m, 1H), 4.20 (d, 1H, J=2.5 Hz), 5.35 (d, 1H, J=2.7 Hz), 6.63-6.67 (m, 1H), 7.0-7.6 (m, 9H), 7.96-7.99 (m, 1H), 10.24 (s, 1H).

### Example 184

### Preparation of 2-(2-Methoxyethyl)-N-(3-methoxyphenyl)-1-oxo-3-(thiophen-3-yl)-1,2,3,4-tetrahydroisoquinoline-4-carboxamide (C11)

This compound was obtained following method C, from 3-thiophenecarboxaldehyde, 2-methoxyethylamine, homophthalic anhydride and 3-methoxyaniline in 14% overall yield. The title compound was purified by flash chromatography eluting with heptane, 20 to 80% ethyl acetate. ESI/APCI(+): 437 (M+H). ¹H NMR (DMSO-d₆) δ 3.01 (s, 3H), 3.02-3.11 (m, 1H), 3.39-3.43 (m, 2H), 3.71 (s, 3H), 4.04-4.12 (m, 1H), 4.23 (s, 1H), 5.36 (s, 1H), 6.62-6.65 (m, 1H), 6.89-6.92 (m, 1H), 7.1-7.5 (m, 8H), 7.91-7.94 (m, 1H), 10.40 (s, 1H).

### Example 185

### Preparation of 2-(2-Methoxyethyl)-N-(3-methoxyphenyl)-3-(5-methylthiophen-2-yl)-1-oxo-1,2,3,4-tetrahydroisoquinoline-4-carboxamide (C12)

This compound was obtained following method C, from 5-methylthiophene-2-carboxaldehyde, 2-methoxyethylamine, homophthalic anhydride and 3-methoxyaniline in 9% overall yield. The title compound was purified twice by flash chromatography eluting first with heptane, 25 to 80% ethyl acetate and then with heptane, 35 to 65% ethyl acetate. ESI/APCI(+): 451 (M+H). ¹H NMR (DMSO-d₆) δ 2.27 (s, 3H), 3.01 (s, 3H), 3.04 (m, 1H), 3.38-3.45 (m, 2H), 3.71 (s, 3H), 4.06 (m, 1H), 4.23 (s, 1H), 5.47 (s, 1H), 6.57 (m, 1H), 6.63 (m, 1H), 6.80 (m, 1H), 7.1-7.6 (m, 6H), 7.92 (m, 1H), 10.37 (s, 1H).

### Example 186

### Preparation of 3-(3-Chlorothiophen-2-yl)-2-(2-methoxyethyl)-N-(3-methoxyphenyl)-1-oxo-1,2,3,4-tetrahydroisoquinoline-4-carboxamide (C17)

This compound was obtained following method C, from 3-chlorothiophene-2-carboxaldehyde, 2-methoxyethylamine, homophthalic anhydride and 3-methoxyaniline in 5% overall yield. The title compound was purified by flash chromatography eluting with heptane, 25 to 805% ethyl acetate and crystallization from ethyl acetate-heptane. ESI/APCI(+): 471 (M+H). ¹H NMR (DMSO-d₆) δ 3.07 (s, 3H), 3.12-3.23 (m, 1H), 3.47 (m, 2H), 3.71 (s, 3H), 3.96 (m, 1H), 4.19 (s, 1H), 5.62 (s, 1H), 6.65 (m, 1H), 7.0-7.6 (m, 8H), 7.97 (m, 1H), 10.29 (s, 1H). Example 187

### Preparation of 2-(2-Methoxyethyl)-N-(3-methoxyphenyl)-1-oxo-3-(pyridin-2-yl)-1,2,3,4-tetrahydroisoquinoline-4-carboxamide (C26)

This compound was obtained following method C, from pyridine-2-carboxaldehyde, 2-methoxyethylamine, homophthalic anhydride and 3-methoxyaniline in 21% overall yield. The title compound was purified by flash chromatography eluting with heptane, 50 to 100% ethyl acetate and crystallization from ethyl acetate-heptane. ESI/APCI(+): 432 (M+H). ¹H NMR (DMSO-d₆) δ 3.01 (s, 3H), 3.13-3.22 (m, 1H), 3.46 (m, 2H), 3.72 (s, 3H), 4.10 (m, 1H), 4.50 (s, 1H), 5.36 (s, 1H), 6.64 (m, 1H), 7.1-7.4 (m, 8H), 7.72 (m, 1H), 7.91 (m, 1H), 8.49 (m, 1H), 10.44 (s, 1H).

### Example 188

### Preparation of 2-(2-Methoxyethyl)-N-(3-methoxyphenyl)-1-oxo-3-(pyridin-4-yl)-1,2,3,4-tetrahydroisoquinoline-4-carboxamide (C30)

This compound was obtained following method C, from pyridine-4-carboxaldehyde, 2-methoxyethylamine, homophthalic anhydride and 3-methoxyaniline in 32% overall yield. The title compound was purified by flash chromatography eluting with dichloromethane, 0 to 10% methanol and crystallization from ethyl acetate-heptane. ESI/APCI(+): 432 (M+H). ¹H NMR (DMSO-d₆) δ 2.96 (s, 3H), 3.15 (m, 1H), 3.41 (m, 2H), 3.72 (s, 3H), 4.02 (m, 1H), 4.22 (s, 1H), 5.40 (s, 1H), 6.65 (m, 1H), 7.1-7.4 (m, 8H), 7.95 (m, 1H), 8.48 (m, 2H), 10.41 (s, 1H).

### Example 189

### Preparation of 2-(2-Methoxyethyl)-N-(3-methoxyphenyl)-1-oxo-3-phenyl-1,2,3,4-tetrahydroisoquinoline-4-carboxamide (C28)

This compound was obtained following method C, from benzaldehyde, 2-methoxyethylamine, homophthalic anhydride and 3-methoxyaniline in 30% overall yield. The title compound was purified by flash chromatography eluting with heptane, 30 to 60% ethyl acetate and crystallization from ethyl acetate-heptane. ESI/APCI(+): 431 (M+H). ¹H NMR (DMSO-d₆) δ 2.99 (m, 1H), 3.00 (s, 3H), 3.42 (m, 2H), 3.72 (s, 3H), 4.06 (m, 1H), 4.18 (s, 1H), 5.34 (s, 1H), 6.64 (m, 1H), 7.1-7.4 (m, 11H), 7.95 (m, 1H), 10.37 (s, 1H).

### Example 190

### Preparation of 2-(2-Methoxyethyl)-N-(3-methoxyphenyl)-1-oxo-3-(1H-pyrrol-2-yl)-1,2,3,4-tetrahydroisoquinoline-4-carboxamide (C29)

This compound was obtained following method C, from pyrrole-2-carboxaldehyde, 2-methoxyethylamine, homophthalic anhydride and 3-methoxyaniline in 13% overall yield. The title compound was purified twice by flash chromatography eluting first with heptane, 30 to 65% ethyl acetate and with dichloromethane 0 to 10% methanol and crystallization from ethyl acetate-heptane. ESI/APCI(+): 420. (M+H). ¹H NMR (DMSO-d₆) δ 3.03 (s, 3H), 3.10 (m, 1H), 3.42 (m, 2H), 3.71 (s, 3H), 4.07 (m, 1H), 4.24 (s, 1H), 5.25 (s, 1H), 5.40 (s, 1H), 5.79 (m, 1H), 6.64 (m, 2H), 7.1-7.5 (m, 6H), 7.91 (m, 1H), 10.19 (s, 1H), 10.70 (s, 1H).

### Example 191

### Preparation of 3-(1H-imidazol-2-yl)-2-(2-methoxyethyl)-N-(3-methoxyphenyl)-1-oxo-1,2,3,4-tetrahydroisoquinoline-4-carboxamide (C35)

This compound was obtained following method C, from imidazole-2-carboxaldehyde, 2-methoxyethylamine, homophthalic anhydride and 3-methoxyaniline in 15% overall yield. The title compound was purified by flash chromatography eluting with dichloromethane, 0 to 10% methanol and crystallization from ethyl acetate-heptane. ESI/APCI(+): 421 (M+H). ¹H NMR (DMSO-d₆) δ 3.12 (s, 3H), 3.20-3.34 (m, 1H), 3.44-3.48 (m, 2H), 3.71 (s, 3H), 4.10-4.18 (m, 1H), 4.24 (d, 1H, J=1.3 Hz), 5.36 (d, 1H, J=1.3 Hz), 6.61-6.65 (m, 1H), 6.75 (s, 1H), 6.96 (s, 1H), 7.1-7.5 (m, 6H), 7.87-7.89 (m, 1H), 10.36 (s, 1H), 11.82 (s, 1H).

### Example 192

### Preparation of 2-(2-Methoxyethyl)-N-(3-methoxyphenyl)-1-oxo-3-(pyridin-3-yl)-1,2,3,4-tetrahydroisoquinoline-4-carboxamide (C53)

This compound was obtained following method C, from pyridine-3-carboxaldehyde, 2-methoxyethylamine, homophthalic anhydride and 3-methoxyaniline in 49% overall yield. The title compound was purified by flash chromatography eluting with dichloromethane, 0 to 10% methanol and crystallization from ethyl acetate-heptane. ESI/APCI(+): 432 (M+H). ¹H NMR (DMSO-d₆) δ 2.96 (s, 3H), 3.17 (m, 1H), 3.41 (m, 2H), 3.72 (s, 3H), 3.98 (m, 1H), 4.19 (s, 1H), 5.44 (s, 1H), 6.65 (m, 1H), 7.1-7.5 (m, 9H), 7.97 (m, 1H), 8.43 (m, 1H), 8.52 (s, 1H), 10.38 (s, 1H).

### Example 193

### Preparation of 2-(2-Methoxyethyl)-N-(3-methoxyphenyl)-3-(4-methyl-1H-imidazol-5-yl)-1-oxo-1,2,3,4-tetrahydroisoquinoline-4-carboxamide (C54)

This compound was obtained following method C, from 5-methylimidazole-4-carboxaldehyde, 2-methoxyethylamine, homophthalic anhydride and 3-methoxyaniline in 14% overall yield. The title compound was purified by flash chromatography eluting with dichloromethane, 0 to 10% methanol and crystallization from ethyl acetate-heptane. ESI/APCI(+): 435 (M+H). ¹H NMR (DMSO-d₆) δ 2.03 (s, 3H), 2.89-2.98 (m, 1H), 2.96 (s, 3H), 3.38-3.49 (m, 2H), 3.71 (s, 3H), 3.95-4.04 (m, 1H), 4.32 (d, 1H, J=4.2 Hz), 5.15 (d, 1H, J=4.2 Hz), 6.61-6.64 (m, 1H), 7.1-7.5 (m, 7H), 7.91-7.94 (m, 1H), 10.27 (s, 1H), 11.70 (s, 1H).

### Example 194

### Preparation of 3-(Benzo[b]thiophen-2-yl)-2-(2-methoxyethyl)-N-(3-methoxyphenyl)-1-oxo-1,2,3,4-tetrahydroisoquinoline-4-carboxamide (C27)

This compound was obtained following method C, from benzothiophene-2-carboxaldehyde, 2-methoxyethylamine, homophthalic anhydride and 3-methoxyaniline in 6% overall yield. The title compound was purified by flash chromatography eluting with heptane, 25 to 80% ethyl acetate and crystallization from ethyl acetate-heptane. ESI/APCI(+): 487 (M+H). ¹H NMR (DMSO-d₆) δ 3.01 (s, 3H), 3.15 (m, 1H), 3.46 (m, 2H), 3.72 (s, 3H), 4.14 (m, 1H), 4.38 (s, 1H), 5.71 (s, 1H), 6.65 (m, 1H), 7.1-7.5 (m, 9H), 7.51-7.76 (m, 2H), 7.95 (m, 1H), 10.47 (s, 1H).

### Example 195

### Preparation of 3-tert-Butyl-2-(2-methoxyethyl)-N-(3-methoxyphenyl)-1-oxo-1,2,3,4-tetrahydroisoquinoline-4-carboxamide (C57)

This compound was obtained following method C, from trimethylacetaldehyde, 2-methoxyethylamine, homophthalic anhydride and 3-methoxyaniline in 26% overall yield. The title compound was purified by flash chromatography eluting with heptane, 20 to 70% ethyl acetate and crystallization from ethyl acetate-heptane. ESI/APCI(+): 411 (M+H). ¹H NMR (DMSO-d₆) δ 0.78 (s, 9H), 3.02 (m, 1H), 3.08 (s, 3H), 3.45 (m, 2H), 3.71 (s, 3H), 3.77 (s, 1H), 4.16 (s, 1H), 4.24 (m, 1H), 6.62 (m, 1H), 7.1-7.5 (m, 6H), 7.82 (m, 1H), 10.06 (s, 1H).

### Example 196

### Preparation of 3-Ethyl-2-(2-methoxyethyl)-N-(3-methoxyphenyl)-1-oxo-1,2,3,4-tetrahydroisoquinoline-4-carboxamide (C58)

This compound was obtained following method C, from propionaldehyde, 2-methoxyethylamine, homophthalic anhydride and 3-methoxyaniline in 30% overall yield. The title compound was purified by flash chromatography eluting with heptane, 30 to 60% ethyl acetate and crystallization from ethyl acetate-heptane. ESI/APCI(+): 383 (M+H). ¹H NMR (DMSO-d₆) δ 0.88 (t, 3H), 1.23-1.40 (m, 1H), 1.54-1.65 (m, 1H), 3.02 (s, 3H), 3.06-3.15 (m, 1H), 3.34-3.45 (m, 2H), 3.70 (s, 3H), 3.92 (t, 1H), 3.99 (s, 1H), 4.03 (m, 1H), 6.61 (m, 1H), 7.1-7.5 (m, 6H), 7.85 (m, 1H), 10.16 (s, 1H).

### Example 197

### Preparation of 3-Benzyl-2-(2-methoxyethyl)-N-(3-methoxyphenyl)-1-oxo-1,2,3,4-tetrahydroisoquinoline-4-carboxamide (C68)

This compound was obtained following method C, from phenylacetaldehyde, 2-methoxyethylamine, homophthalic anhydride and 3-methoxyaniline in 15% overall yield. The title compound was purified by flash chromatography eluting with heptane, 20 to 90% ethyl acetate and crystallization from ethyl acetate-heptane. ESI/APCI(+): 445 (M+H). ¹H NMR (DMSO-d₆) δ 2.57 (m, 1H), 2.66-2.80 (m, 2H), 2.98 (s, 3H), 3.29 (m, 2H), 3.69 (s, 3H), 3.78 (m, 1H), 3.94 (s, 1H), 4.29 (t, 1H), 6.60 (m, 1H), 7.0-7.6 (m, 11H), 7.90 (m, 1H), 10.10 (s, 1H).

### Example 198

### Preparation of 2-(2-methoxyethyl)-N-(3-methoxyphenyl)-3-methyl-1-oxo-1,2,3,4-tetrahydroisoquinoline-4-carboxamide (C69)

This compound was obtained following method C, from acetaldehyde, 2-methoxyethylamine, homophthalic anhydride and 3-methoxyaniline in 32% overall yield. The title compound was purified by flash chromatography eluting with heptane, 30 to 70% ethyl acetate and crystallization from ethyl acetate-heptane. ESI/APCI(+): 369 (M+H). ¹H NMR (DMSO-d₆) δ 1.12 (d, 3H, J=6.5 Hz), 3.01 (s, 3H), 3.21-3.30 (m, 1H), 3.30-3.47 (m, 2H), 3.70 (s, 3H), 3.87 (s, 1H), 3.89-3.97 (m, 1H), 4.15-4.21 (q, 1H), 6.60-6.63 (m, 1H), 7.0-7.6 (m, 6H), 7.86-7.89 (m, 1H), 10.19 (s, 1H).

### Example 199

### Preparation of 2-(2-methoxyethyl)-N-(3-methoxyphenyl)-1-oxo-3-((trimethylsilyl)ethynyl)-1,2,3,4-tetrahydroisoquinoline-4-carboxamide (C70)

This compound was obtained following method C, from 3-trimethylsilylpropynal, 2-methoxyethylamine, homophthalic anhydride and 3-methoxyaniline in 28% overall yield. The title compound was purified by flash chromatography eluting with heptane, 20 to 80% ethyl acetate and crystallization from ethyl acetate-heptane. ESI/APCI(+): 451 (M+H). ¹H NMR (DMSO-d₆) δ 0.00 (s, 9H), 3.04 (s, 3H), 3.29-3.51 (m, 4H), 3.70 (s, 3H), 3.91 (m, 1H), 4.19 (d, 1H, J=2.1 Hz), 5.08 (d, 1H, J=2.2 Hz), 6.63 (m, 1H), 7.0-7.6 (m, 6H), 7.91 (m, 1H), 10.36 (s, 1H).

### Example 200

### Preparation of 3-ethynyl-2-(2-methoxyethyl)-N-(3-methoxyphenyl)-1-oxo-1,2,3,4-tetrahydroisoquinoline-4-carboxamide (C83)

Potassium carbonate (46 mg, 0.33 mmol) was added to a solution of 2-(2-methoxyethyl)-N-(3-methoxyphenyl)-1-oxo-3-((trimethylsilyl)ethynyl)-1,2,3,4-tetrahydroisoquinoline-4-carboxamide (50 mg, 0.11 mmol) in methanol (1 mL) and acetonitrile (1 mL). The mixture was stirred at room temperature for 1.5 h. It was the diluted with ethyl acetate and the organic layer was washed with brine, dried over sodium sulfate, filtered and concentrated in vacuo. The residue was purified by flash chromatography eluting with heptane, 30 to 70% ethyl acetate. The resulting compound was recrystallised from heptane/ethyl acetate to give 21 mg (50%) of title compound as a white solid. ESI/APCI(+): 379 (M+H). ESI/APCI(−): 377 (M−H). ¹H NMR (DMSO-d₆) δ 2.11 (3H, s), 3.02 (3H, s), 3.2-3.4 (3H, m), 3.51 (1H, m), 3.70 (3H, s), 3.98 (1H, m), 4.20 (1H, s), 5.07 (1H, s), 6.62 (1H, d), 7.0-7.2 (2H, m), 7.29 (1H, s), 7.4-7.6 (3H, m), 7.91 (1H, d), 10.36 (1H, s).

### Example 201

### Preparation of 2-(2-methoxyethyl)-N-(3-methoxyphenyl)-3-(5-methylisoxazol-3-yl)-1-oxo-1,2,3,4-tetrahydroisoquinoline-4-carboxamide (C103)

This compound was obtained following method C, from 5-methylisoxazol-3-carbaldehyde, 2-methoxyethanamine, homophthalic anhydride and 3-methoxyaniline in 28% overall yield. The title compound was purified by flash chromatography eluting with dichloromethane, 5 to 40% EtOAc. ESI/APCI(+): 436 (M+H). ¹H NMR (DMSO-d₆) δ 2.30 (s, 3H), 2.98 (s, 3H), 3.14 (m, 1H), 3.40 (m, 2H), 3.71 (s, 3H), 4.08 (dt, 1H), 4.32 (s, 3H), 5.43 (s, 1H), 5.92 (s, 1H), 6.62 (dd, 1H), 7.11 (d, 1H), 7.32-7.48 (m, 4H), 7.88 (d, 1H), 10.41 (s, 1H, NH).

### Example 202

### Preparation of 2-(2-methoxyethyl)-N-(3-methoxyphenyl)-3-(1-methyl-1H-pyrazol-5-yl)-1-oxo-1,2,3,4-tetrahydroisoquinoline-4-carboxamide (C105)

This compound was obtained following method C, from 1-methyl-1H-pyrazol-5-carbaldehyde, 2-methoxyethanamine, homophthalic anhydride and 3-methoxyaniline in 25% overall yield. The title compound was purified by flash chromatography eluting with dichloromethane, 5 to 40% EtOAc. ESI/APCI(+): 435 (M+H). ¹H NMR (DMSO-d₆) δ 3.05 (s, 3H), 3.20 (m, 1H), 3.37 (m, 2H), 3.72 (s, 3H), 3.87 (dt, 1H), 3.96 (s, 3H), 4.12 (s, 1H), 5.46 (d, 1H), 5.49 (s, 1H), 6.64 (d, 1H), 7.10 (d, 1H), 7.17 (d, 1H), 7.20 (t, 1H), 7.29 (s, 1H), 7.32 (d, 1H), 7.45-7.50 (m, 2H), 7.96 (dd, 1H), 10.22 (s, 1H, NH).

### Example 203

### Preparation of 3-cyclopentyl-2-(2-methoxyethyl)-N-(3-methoxyphenyl)-1-oxo-1,2,3,4-tetrahydroisoquinoline-4-carboxamide (C118)

This compound was obtained following method C, from cyclopentylcarbaldehyde, 2-methoxyethanamine, homophthalic anhydride and 3-methoxyaniline in 44% overall yield. The title compound was purified twice by flash chromatography eluting with dichloromethane, 0 to 30% EtOAc and was recrystallised from heptane. ESI/APCI(+): 423 (M+H). ¹H NMR (DMSO-d₆) δ 1.35-1.67 (m, 8H), 2.96 (m, 4H), 3.12 (m, 1H), 3.36 (m, 3 H), 3.60 (m, 1H), 3.70 (s, 1H), 3.84 (d, 1H), 3.99 (s, 1H), 4.22 (dt, 1H), 6.61 (d, 1H), 7.11-7.22 (m, 2H), 7.31-7.50 (m, 4H), 7.84 (d, 1H), 10.14 (s, 1H).

### Example 204

### Preparation of 2-(2-methoxyethyl)-N-(3-methoxyphenyl)-3-(2-methylprop-1-enyl)-1-oxo-1,2,3,4-tetrahydroisoquinoline-4-carboxamide (C120)

This compound was obtained following method C, from 3-methylbutenal, 2-methoxyethanamine, homophthalic anhydride and 3-methoxyaniline in 10% overall yield. The title compound was purified twice by flash chromatography eluting first with dichloromethane, 0 to 20% EtOAc and then with heptane, 50 to 90% EtOAc. ESI/APCI(+): 409 (M+H). ¹H NMR (DMSO-d₆) δ 1.63 (s, 3H), 1.79 (s, 3H), 3.02 (m, 1H), 3.07 (s, 3H), 3.42 (m, 2H), 3.70 (s, 3H), 3.85 (s, 1H), 3.94 (m, 1H), 4.80 (d, 1H), 4.94 (d, 1H), 6.62 (dd, 1H), 7.09 (d, 1H), 7.19 (t, 1H), 7.29 (s, 1H), 7.34 (d, 1H), 7.42 (t, 1 H), 7.50 (t, 1H), 7.90 (d, 1H), 10.20 (s, 1H).

### Example 205

### Preparation of 2-(2-methoxyethyl)-N-(3-methoxyphenyl)-1-oxo-3-(2,2,2-trifluoroethyl)-1,2,3,4-tetrahydroisoquinoline-4-carboxamide (C71)

This compound was obtained following method C, from 3,3,3-trifluoropropanal, 2-methoxyethanamine, homophthalic anhydride and 3-methoxyaniline in 6% overall yield. The title compound was purified first by flash chromatography eluting with heptane, 20 to 90% EtOAc and then by preparative HPLC with a gradient of acetonitrile (20-95%) in water (0.1% formic acid). ESI/APCI(+): 437 (M+H), 459 (M+Na). ESI/APCI(−): 435 (M−H). ¹H NMR (DMSO-d₆) δ 2.3-2.6 (2H, m), 3.00 (3H, s), 3.13 (1H, m), 3.41 (2H, m), 3.71 (3H, s), 4.02 (2H, m), 4.46 (1H, m), 6.62 (1H, d), 7.0-7.3 (3H, m), 7.4-7.6 (3H, m), 7.88 (1H, d), 10.25 (1H, s).

### Example 206

### Preparation of 2-(2-methoxyethyl)-N-(3-methoxyphenyl)-3-(methylthiomethyl)-1-oxo-1,2,3,4-tetrahydroisoquinoline-4-carboxamide (C82)

This compound was obtained following method C, from methylthioacetaldehyde dimethyl acetal, 2-methoxyethanamine, homophthalic anhydride and 3-methoxyaniline in 9% overall yield. Methylthioacetaldehyde dimethyl acetal was initially heated to reflux in 1% HCl for 30 min. After cooling to room temperature, the mixture was neutralized with sodium hydrogen carbonate saturated solution and the organic layer was extracted with dichloromethane. Combined organic leyers were concentrated in vacuo to give methylthioacetaldehyde.
The title compound was purified by flash chromatography eluting with heptane, 30 to 80% EtOAc and recrystallised from heptane/EtOAc. ESI/APCI(+): 415 (M+H). ESI/APCI(−): 413 (M−H). ¹H NMR (DMSO-d₆) δ 2.11 (3H, s), 2.34 (1H, m), 2.79 (1H, m), 3.00 (3H, s), 3.44 (3H, m), 3.70 (3H, s), 3.96 (1H, m), 4.15 (1H, m), 4.23 (1H, s), 6.61 (1H, d), 7.17 (2H, m), 7.29 (1H, s), 7.4-7.55 (3H, m), 7.90 (1-H, d), 10.25 (1H, s).

### Example 207

### Preparation of 2-(2-methoxyethyl)-N-(3-methoxyphenyl)-3-(methylsulfonylmethyl)-1-oxo-1,2,3,4-tetrahydroisoquinoline-4-carboxamide (C149)

To 2-(2-methoxyethyl)-N-(3-methoxyphenyl)-3-(methylthiomethyl)-1-oxo-1,2,3,4-tetrahydroisoquinoline-4-carboxamide (20 mg, 0.05 mmol) in THF (1 mL) was added 3-chloroperoxybenzoic acid (26 mg, 0.15 mmol). The mixture was stirred at room temperature for 1 h. THF was removed and the residue was dissolved in ethyl acetate. The organic layer was washed with sodium sulfite solution and brine. The residue was purified by preparative thick layer chromatography to give 14 mg (62%) of title compounds as a white solid. ¹H NMR (DMSO-d₆) δ 10.24 (1H, s), 7.89 (1H, d), 7.56 (2H, m), 7.47 (1H, m), 7.27 (1H, s), 7.16 (1H, t), 713 (1H, t), 6.62 (1H, d), 4.61 (1H, t), 4.20 (1H, s), 3.95 (1H, m), 3.70 (3H, s), 3.51 (3H, m), 3.40 (1H, m), 3.18 (1H, m), 3.10 (3H, s), 3.07 (3H, s).

### Example 208

### Preparation of 4-(2-(2-methoxyethyl)-1-oxo-3-(thiophen-2-yl)-1,2,3,4-tetrahydroisoquinoline-4-carboxamido)benzoic acid (C258)

Methyl 4-(2-(2-methoxyethyl)-1-oxo-3-(thiophen-2-yl)-1,2,3,4-tetrahydroisoquinoline-4-carboxamido)benzoate (470 mg, 1.01 mmol) was dissolved in methanol (8 mL) and 3N sodium hydroxide solution (8 mL) was added. The mixture was stirred 4 hours at 50° C. The methanol was evaporated in vacuo and the remaining aqueous phase was acidified with a few drops of concentrated HCl. The precipitate was filtered and washed with a small amount of water before drying to give 244 mg (53%) of title compound. ESI/APCI(+) δ 451 (M+H).

### Example 209

### Preparation of N-(4-(benzylcarbamoyl)phenyl)-2-(2-methoxyethyl)-1-oxo-3-(thiophen-2-yl)-1,2,3,4-tetrahydroisoquinoline-4-carboxamide (C137)

To a solution of 4-(2-(2-methoxyethyl)-1-oxo-3-(thiophen-2-yl)-1,2,3,4-tetrahydroisoquinoline-4-carboxamido)benzoic acid (50 mg, 0.11 mmol) in dry dichloromethane (3 mL) was added HATU (50 mg, 0.13 mmol), diisopropylethylamine (17 mg, 0.13 mmol) and benzylamine (15 μL, 0.13 mmol) and the mixture was stirred overnight at room temperature. The solution was washed with water, the dichloromethane evaporated and the residue purified by flash chromatography eluting with heptane, 10% to 60% ethyl acetate to give title compound (10 mg, 17%). ESI/APCI(+): 540 (M+H). ¹H NMR (DMSO-d₆) δ 2.80 (s, 3H), 3.02-3.11 (m, 1H), 3.41 (m, 2H), 4.07 (m, 1H), 4.31 (s, 1H), 4.45 (d, 2H), 5.61 (s, 1H), 6.91 (m, 1H), 7.00 (d, NH), 7.33 (m, 6H), 7.40-7.51 (m, 3H), 7.68 (d, 2H), 7.90 (m, 3H), 8.93 (m, 1H), 10.64 (s, NH).

### Example 210

### Preparation of N-(4-(dimethylcarbamoyl)phenyl)-2-(2-methoxyethyl)-1-oxo-3-(thiophen-2-yl)-1,2,3,4-tetrahydroisoquinoline-4-carboxamide (C139)

To a solution of 4-(2-(2-methoxyethyl)-1-oxo-3-(thiophen-2-yl)-1,2,3,4-tetrahydroisoquinoline-4-carboxamido)benzoic acid (150 mg, 0.33 mmol) in dry dichloromethane (10 mL) was added HATU (151 mg, 0.40 mmol) and dimethylamine (2M solution in THF, 1.7 mL) and the mixture was stirred overnight at room temperature. The solution was washed with water, the dichloromethane evaporated and the residue purified by flash chromatography eluting with heptane, 10% to 60% ethyl acetate to give title compound (46 mg, 29%). ESI/APCI(+): 487 (M+H). ¹H NMR (DMSO-d₆) δ 2.93 (s, 6H), 3.00 (s, 3H), 3.08 (m, 1H), 3.40 (m, 2H), 4.01-4.11 (m, 1H), 4.29 (s, 1H), 5.60 (s, 1H), 6.91 (m, 1H), 7.00 (d, NH), 7.30-7.51 (m, 6H), 7.65-7.66 (d, 2H), 7.93 (d, 1H), 10.54 (s, 1H).

### Example 211

### Preparation of 2-(2-methoxyethyl)-N-(4-(methylcarbamoyl)phenyl)-1-oxo-3-(thiophen-2-yl)-1,2,3,4-tetrahydroisoquinoline-4-carboxamide (C141)

To a solution of 4-(2-(2-methoxyethyl)-1-oxo-3-(thiophen-2-yl)-1,2,3,4-tetrahydroisoquinoline-4-carboxamido)benzoic acid (50 mg, 0.11 mmol) in dry dichloromethane (3 mL) was added HATU (50 mg, 0.13 mmol), diisopropylethylamine (17 mg, 0.13 mmol) and methylamine (2N solution in THF, 70 μL) and the mixture was stirred overnight at room temperature. The solution was washed with water, the dichloromethane evaporated and the residue purified by flash chromatography eluting with heptane, 10% to 60% ethyl acetate to give title compound (4 mg, 7%). ESI/APCI(+): 464 (M+H). ¹H NMR (DMSO-d₆) δ 2.75 (d, 3H), 2.97 (s, 3H), 3.02-3.11 (m, 1H), 3.42 (m, 2H), 4.08 (m, 1H), 4.31 (s, 1H), 5.60 (s, 1H), 6.91 (m, 1H), 7.00 (s, NH), 7.33 (m, 2H), 7.40-7.51 (m, 2H), 7.66 (d, 2H), 7.80 (d, 2H), 7.92 (d, 1H), 8.32 (d, NH), 10.60 (s, 1H).

### Example 212

### Preparation of 7-amino-2-(2-methoxyethyl)-N-(3-methoxyphenyl)-1-oxo-3-(thiophen-2-yl)-1,2,3,4-tetrahydroisoquinoline-4-carboxamide (C147)

To a stirred solution of 2-(2-methoxyethyl)-N-(3-methoxyphenyl)-7-nitro-1-oxo-3-(thiophen-2-yl)-1,2,3,4-tetrahydroisoquinoline-4-carboxamide (60 mg, 0.12 mmol) in tetrahydrofuran (8 mL) was added palladium (10% on carbon, 155 mg, 0.144 mmol). The mixture was stirred under hydrogen atmosphere for 4 hours. The mixture filtered over celite and the filtrate was concentrated under reduced pressure The residue was purified by flash chromatography eluting with heptane, 20 to 100% ethyl acetate to give title compound (5.0 mg, 9%) as a white solid. ESI/APCI(+): 452 (M+H). ESI/APCI(−): 450 (M−H). ¹H NMR (DMSO-d₆) δ 2.98 (m, 1H), 3.04 (s, 3H), 3.63 (m, 2H), 3.71 (s, 3H), 3.99 (s, 1H), 4.02 (m, 1H), 5.30 (s, 2H), 5.46 (s, 1H), 6.63 (m, 2H), 6.90-7.01 (m, 3H), 7.10-7.23 (m, 3H), 7.29 (m, 2H), 10.13 (s, 1H).

### Example 213

### Preparation of N-(4-(1H-pyrrol-1-yl)phenyl)-3-ethynyl-2-(2-methoxyethyl)-1-oxo-1,2,3,4-tetrahydroisoquinoline-4-carboxamide (C152)

3-Trimethylsilylpropynal (732 μL, 5 mmol) and 2-methoxyethylamine (436 μL, 5 mmol) were diluted in chloroform (5 mL) and magnesium sulfate (603 mg, 5 mmol) was added. The mixture was stirred at room temperature overnight. Homophthalic anhydride (810 mg, 5 mmol) was then added and the mixture was heated to 60° C. for 3 h. Dichloromethane was added and the mixture was filtered, the filtrate was concentrated to give 2-(2-methoxyethyl)-1-oxo-3-((trimethylsilyl)ethynyl)-1,2,3,4-tetrahydroisoquinoline-4-carboxylic acid as a beige sticky solid. ESI/APCI(+): 346 (M+H).

2-(2-Methoxyethyl)-1-oxo-3-((trimethylsilyl)ethynyl)-1,2,3,4-tetrahydroisoquinoline-4-carboxylic acid (172 mg, 0.5 mmol) was dissolved in dichloromethane (4 mL) and HATU (228 mg, 0.6 mmol), 4-(1H-pyrrol-1-yl)aniline (94 mg, 0.6 mmol) and diisopropylamine (171 μL, 1 mmol) were added. The mixture was stirred at room temperature for 5 h. After addition of dichloromethane and washing with 1N HCl, mixture was concentrated and the residue was purified by flash chromatography eluting with a gradient of ethyl acetate (20-75%) in heptane to give 70 mg of unpure N-(4-(1H-pyrrol-1-yl)phenyl)-2-(2-methoxyethyl)-1-oxo-3-((trimethylsilyl)ethynyl)-1,2,3,4-tetrahydroisoquinoline-4-carboxamide which was used in the next step without further purification.

N-(4-(1H-pyrrol-1-yl)phenyl)-2-(2-methoxyethyl)-1-oxo-3-((trimethylsilyl)ethynyl)-1,2,3,4-tetrahydroisoquinoline-4-carboxamide (138 mg, 0.3 mmol) was dissolved in methanol (3 mL) and potassium carbonate in water was added. The mixture was stirred at room temperature for 2 h, until TLC indicates complete deprotection. The solvent was evaporated, the residue was dissolved in dichloromethane. The organic layer was washed with 1N HCl and concentrated. The residue was purified by flash chromatography eluting with a gradient of ethyl acetate (20-70%) in heptane and recrystallised from ethanol to give 23 mg(18%) of title compound as a colourless solid.

ESI/APCI(+): 414 (M+H). ESI/APCI(−): 412 (M−H). ¹H NMR (DMSO-d₆) δ 10.42 (1H, s), 7.94 (1H, d), 7.64 (2H, d), 7.53 (3H, m), 7.46 (2H, m), 7.30 (2H, t), 6.23 (2H, t), 5.09 (1H, s), 4.21 (1H, s), 4.00 (1H, m), 3.48 (1H, m), 3.41 (2H, m), 3.28 (2H, m), 3.05 (3H, s).

### Example 214

### Preparation of 2-(2-methoxyethyl)-4-((3-methoxyphenylamino)methyl)-3-(thiophen-2-yl)isoquinolin-1(2H)-one (C224)

To a 0° C. cooled solution of thiophen-2-carbonyl chloride (2 mL, 18.7 mmol) in 50 mL of dichloromethane was added triethylamine (2.6 mL, 18.7 mmol) followed by 2-methoxyethylamine (1.63 mL, 18.7 mmol) and the mixture was stirred at room temperature for 1 hour. 50 mL of water was added and the solution was extracted twice with dichloromethane. The combined organic layers were dried over MgSO₄ and evaporated to give 3.24 g of N-(2-methoxyethyl)thiophene-2-carboxamide as white crystals in 93% yield.

A mixture of N-(2-methoxyethyl)thiophene-2-carboxamide (1.18 g, 6.37 mmol) and thionyl chloride (10 eq) was refluxed for 2.5 hours under nitrogen. After cooling, the mixture was concentrated under reduced pressure and the residue was dissolved in 10 mL of toluene and homophthalic anhydride (1.03 g, 6.37 mmol) was added. The mixture was then refluxed for 3 hours. The solvent was removed and the residue was purified by flash chromatography eluting with heptane, 5 to 70% ethyl acetate to give 0.89 g of 2-(2-methoxyethyl)-3-(thiophen-2-yl)isoquinolin-1(2H)-one as a yellow oil (50%).

To a cooled (0° C.) solution of DMF (64 μL, 0.84 mmol) in 1 mL of dry dichloromethane was added POCl₃ (78 μL, 0.84 mmol) dropwise. The ice bath was removed and the reaction was stirred for 30 min. 2-(2-Methoxyethyl)-3-(thiophen-2-yl)isoquinolin-1(2H)-one (120 mg, 0.42 mmol) was added to the above solution at 0° C. The reaction mixture was left stirring at 50° C. for 20 hours, poured onto an ice cold 1N NaOH solution and stirred at RT for an additional hour. The organic layer was separated and the aqueous layer was extracted with dichloromethane. The combined organic extracts were evaporated and the residue was purified by flash chromatography eluting with dichloromethane, 0 to 10% ethyl acetate to give 2-(2-methoxyethyl)-1-oxo-3-(thiophen-2-yl)-1,2-dihydroisoquinoline-4-carbaldehyde (84 mg, 64%) as a purple solid.

To a solution of the previous aldehyde (50 mg, 0.16 mmol) in dry toluene (1.5 mL) was added 3-methoxyaniline and the mixture was heated under microwave irradiation at 160° C. for 15 min. The solution was concentrated in the rotavap and the residue was diluted with 3 mL of THF. To this mixture was added sodium triacetoxyborohydride (118 mg, 0.56 mmol) and the solution was stirred at RT for 3 hours. The reaction was quenched with 1N NaOH and extracted with dichloromethane. After evaporation of organic solvent, the residue was purified by flash chromatography eluting with heptane, 20 to 70% ethyl acetate to give title compound (43.4 mg, 64%). ESI/APCI(+): 421 (M+H). ¹H NMR (DMSO-d₆) δ 3.14 (s, 3H), 3.48 (t, 2H), 3.64 (s, 3H), 3.55-4.02 (m, 4H), 5.73 (t, 1H, NH), 6.11-6.17 (m, 3H), 6.94 (t, 1H), 7.19 (dd, 1H), 7.38 (d, 1H), 7.60 (t, 1H), 7.70 (d, 1H), 7.79 (m, 2H), 8.34 (d, 1H).

### Example 215

### Preparation of 2-(2-methoxyethyl)-1-oxo-3-(thiophen-2-yl)-1,2,3,4-tetrahydroisoquinoline-4-carboxylic acid (C36)

Homophthalic anhydride (580 mg, 3.58 mmol) was added in one portion to a stirred solution of 2-methoxy-N-(thiophen-2-ylmethylene)ethanamine (605 mg, 3.58 mmol) and triethylamine (0.72 mL) in dichloromethane (10 mL). The stirring was continued at RT overnight then 1N NaOH was added and the phases were separated. The aqueous layer was acidified with 6N HCl then extracted with dichloromethane. The organic layer was dried with MgSO₄ and evaporated to dryness to give title compound (1.17 g, 99%). ESI/APCI (+) 332 (M+H). ¹H NMR (DMSO-d₆) δ 3.19 (s, 3H), 3.36 (m, 2H), 3.51 (m, 2H), 3.81 (s, 1H), 5.68 (s, 1H), 6.84 (m, 2H), 7.24 (m, 2H), 7.31 (t, 1H), 7.36 (t, 1H), 7.83 (dd, 1H)

### Example 216

### Preparation of 2-(2-methoxyethyl)-N-(3-methoxyphenyl)-1-oxo-3-(thiophen-2-yl)-1,2-dihydroisoquinoline-4-carboxamide (C32)

A mixture of 2-(2-methoxyethyl)-1-oxo-3-(thiophen-2-yl)-1,2,3,4-tetrahydroisoquinoline-4-carboxylic acid (1.10 g, 3.32 mmol), thionyl chloride (6 mL) and chloroform (6 mL) was heated to reflux for 1 hour. After cooling the volatiles were removed and the residue was dissolved in chloroform (10 mL) under a protective atmosphere of nitrogen. Methanol (10 mL) was added and the mixture was stirred at RT for 20 hours. The solvent was removed and the residue was purified by flash chromatography eluting with dichloromethane, 0 to 20% ethyl acetate to give methyl 2-(2-methoxyethyl)-1-oxo-3-(thiophen-2-yl)-1,2,3,4-tetrahydroisoquinoline-4-carboxylate (250 mg, 22%).
LiHMDS (1.0 M in THF, 2.03 mL) was added slowly to a stirred solution of previous ester (200 mg, 0.58 mmol) in dry THF (5 mL) at −78° C. The reaction mixture was stirred at −78° C. for 30 min, and then a solution of phenylselenyl chloride (388 mg, 2.03 mmol) in THF (2 mL) was added and the mixture was stirred at −78° C. for 1 h. The reaction mixture was stirred at RT overnight. The reaction was quenched by slow addition of 1 N HCl (10 mL) at 0° C. Dichloromethane was used to extract the product. The combined organic layers were washed with water and brine. The resulting organic solution was dried over anhydrous MgSO₄, filtered, and concentrated to afford the crude selenide derivative. The residue was dissolved in THF (10 mL) and acetic acid (1.0 mL) and hydrogen peroxide (30%, 2 mL) were added sequentially to the stirred solution at 0° C. The reaction mixture was stirred at RT overnight. A saturated solution of sodium hydrogen carbonate (30 mL) was added to the reaction mixture at 0° C. and dichloromethane was used to extract the product. The combined organic layers were washed with H₂O. The resulting organic solution was concentrated under reduced pressure. The residue was purified by flash column chromatography on silica gel, eluting with chloroform, to yield in the first fraction methyl 2-(2-methoxyethyl)-1-oxo-3-(thiophen-2-yl)-1,2-dihydroisoquinoline-4-carboxylate (30 mg, 15%). In another fraction was isolated methyl 4-hydroxy-2-(2-methoxyethyl)-1-oxo-3-(thiophen-2-yl)-1,2,3,4-tetrahydroisoquinoline-4-carboxylate (65 mg, 31%). ESI/APCI(+): 362 (M+H)

To a solution of methyl 2-(2-methoxyethyl)-1-oxo-3-(thiophen-2-yl)-1,2-dihydroisoquinoline-4-carboxylate (30 mg, 0.09 mmol) in THF (3 mL) was added 1 mL of 6N NaOH and the solution was stirred at RT for 1 hour then refluxed for 3 hours. Additional 1 mL of 6N NaOH was added and the reflux was continued for 3 hours. After cooling, THF was removed and the remaining aqueous layer was diluted with water, acidified with 6N HCl to pH 3-4 and extracted with dichloromethane. The organic layer was dried over MgSO4 and evaporated to dryness to give 29 mg of crude acid, which was used in the next step without any further purification.

To a solution of the acid in dry DMF (1 mL) was added m-anisidine (114, 0.109 mmol), diisopropylethylamine amine (194, 0.109 mmol) and HATU (41 mg, 0.109 mmol) and the mixture was stirred at RT for 1 hour. DMF was removed on rotavap and dichloromethane was added. The solution was washed with 1H HCl (sat. aq. Sol.), evaporated and the residue was purified by flash chromatography eluting with heptane, 15 to 70% ethyl acetate to yield 23 mg of the activated ester instead of the desired amide.

This activated ester, m-anisidine (45 μL, 0.402 mmol) and diisopropylethylamine amine (69 μL, 0.402 mmol) in 1.5 mL of dry dioxane were heated under microwave irradiation for 6 h at 150° C. The solution was concentrated in vacuum and the residue was purified by flash column chromatography eluting with heptane, 15 to 70% ethyl acetate to give title compound (15.4 mg, 53%) as light yellow solid. ESI/APCI (+) 435 (M+H). ¹H NMR (DMSO-d₆) δ 3.14 (s, 3H), 3.48 (t, 2H), 3.69 (s, 3H), 4.02 (t, 2H), 6.61 (dd, 1H), 6.69 (d, 1H), 7.16 (m, 3H), 7.34 (d, 1H), 7.56 (d, 1H), 7.63 (t, 1H), 7.75 (d, 1H), 7.80 (t, 1H), 8.34 (d, 1H), 10.34 (s, 1H, NH).

### Example 217

### Preparation of 3-(furan-2-yl)-2-(2-methoxyethyl)-N-(3-methoxyphenyl)-1-oxo-1,2-dihydroisoquinoline-4-carboxamide (C38)

This compound was obtained in a similar manner as for example 72 from 2-(2-methoxyethyl)-1-oxo-3-(furan-2-yl)-1,2,3,4-tetrahydroisoquinoline-4-carboxylic acid in 1% overall yield. ESI/APCI (+) 419 (M+H). ¹H NMR (DMSO-d₆) δ 3.15 (s, 3H), 3.48 (t, 2H), 3.71 (s, 3H), 4.01 (t, 2H), 6.60 (s, 1H), 6.65 (dd, 1H), 6.79 (d, 1H), 7.03 (m, 2H), 7.19 (d, 1H), 7.62 (t, 1H), 7.69 (t, 1H), 7.90 (s, 1H), 8.35 (d, 1H), 10.41 (s, 1H, NH).

### Example 218

### Preparation of 4-(hydroxymethyl)-2-(2-methoxyethyl)-3-(thiophen-2-yl)-3,4-dihydroisoquinolin-1(2H)-one (C33)

To a suspension of lithium aluminium hydride (38 mg, 1.01 mmol) in dry THF (4 mL) at 0° C. was added a solution of methyl 2-(2-methoxyethyl)-1-oxo-3-(thiophen-2-yl)-1,2,3,4-tetrahydroisoquinoline-4-carboxylate (175 mg, 0.506 mmol) in THF (4 mL) and the mixture was stirred under a nitrogen atmosphere at 0° C. for 1 hour. Water was carefully added at 0° C. and the solution was diluted with dichloromethane. After filtration through celite and evaporation of the solvent, the residue was purified by flash chromatography eluting with heptane, 15 to 70% ethyl acetate to give title compound (116 mg, 72%) as a white solid. ESI/APCI (+) 318 (M+H). ¹H NMR (DMSO-d₆) δ 3.00 (dt, 1H), 3.16 (t, 1H), 3.26 (s, 3H), 3.49-3.57 (m, 4H), 4.19 (dt, 1H), 5.13 (t, 1H, OH), 5.34 (s, 1H), 6.85 (m, 2H), 7.27 (m, 2H), 7.36 (t, 1H), 7.45 (t, 1H), 7.88 (d, 1H).

### Example 219

### Preparation of 4-(benzyloxymethyl)-2-(2-methoxyethyl)-3-(thiophen-2-yl)-3,4-dihydroisoquinolin-1(2H)-one (C76)

To a solution of 4-(hydroxymethyl)-2-(2-methoxyethyl)-3-(thiophen-2-yl)-3,4-dihydroisoquinolin-1(2H)-one (55 mg, 0.17 mmol) in dry DMF (2 mL) was added sodium hydride (60%, 13.8 mg, 0.35 mmol) and the reaction was stirred at 0° C. for 15 min. Benzyl bromide (25 μL, 0.21 mmol) was then added added and the stirring was continued at 0° C. for 1 hour. Water was added carefully and the reaction mixture was extracted with CH₂Cl₂. The organic solvent was washed with brine,
evaporated and the residue was purified by flash chromatography eluting with heptane, 5 to 50% ethyl acetate to give the title compound (60 mg, 84%) as an oil. ESI/APCI (+) 408 (M+H). ¹H NMR (DMSO-d₆) δ 2.97 (dt, 1H), 3.22 (s, 3H), 3.44-3.56 (m, 4H), 3.62 (t, 1H), 4.20 (dt, 1H), 4.57 (s, 2H), 5.34 (s, 1H), 6.85-6.90 (m, 2H), 7.27 (dd, 1H), 7.30-7.38 (m, 6H), 7.40 (dd, 1H), 7.47 (td, 1H), 7.89 (dd, 1H).

### Example 220

### Preparation of 6-fluoro-2-(2-methoxyethyl)-N-(3-methoxyphenyl)-1-oxo-3-(thiophen-2-yl)-1,2,3,4-tetrahydroisoquinoline-4-carboxamide (C77)

This compound was obtained following method B, from thiophene-2-carboxaldehyde, 2-methoxyethylamine, 5-fluorohomophthalic anhydride and 3-methoxyaniline in 11% overall yield. The title compound was purified by flash chromatography eluting with dichloromethane, 1 to 15% EtOAc. ESI/APCI (+) 455 (M+H). ¹H NMR (DMSO-d₆) δ 2.97 (s, 3H), 3.03 (dt, 1H), 3.39 (m, 2H), 3.71 (s, 3H), 4.06 (dt, 1H), 4.31 (s, 1H), 5.60 (s, 1H), 6.63 (dd, 1H), 6.91 (dd, 1H), 7.02 (d, 1H), 7.11 (d, 1H), 7.20 (t, 1H), 7.26-7.34 (m, 4H), 7.94 (dd, 1H), 10.43 (s, 1H, NH).

### Example 221

### Preparation of 2-(2-methoxyethyl)-4-((3-methoxyphenylamino)methyl)-3-(thiophen-2-yl)-3,4-dihydroisoquinolin-1(2H)-one (C73)

To a solution of 4-(hydroxymethyl)-2-(2-methoxyethyl)-3-(thiophen-2-yl)-3,4-dihydroisoquinolin-1(2H)-one (110 mg, 0.35 mmol) in 3 mL of dichloromethane was added triethyl amine (72 μL, 0.52 mmol), tosyl chloride (72 mg, 0.38 mmol) and dimethylaminopyridine (8 mg, 0.07 mmol) and the mixture was stirred at room temperature for 20 hours. 10 mL of NH₄Cl (sat. aq. Sol.) was added and the solution was extracted with CH₂Cl₂ (2×10 mL). The combined organic extracts was evaporated and the residue was purified by flash chromatography eluting with heptane, 15 to 70% ethylacetate to give title compound (157 mg, 96%) as a white solid.

A mixture of 2-(2-methoxyethyl)-1-oxo-3-(thiophen-2-yl)-1,2,3,4-tetrahydroisoquinolin-4-yl)-methyl 4-methylbenzenesulfonate (50 mg, 0.11 mmol), m-anisidine (5 μL, 0.53 mmol) and potassium carbonate (73 mg, 0.53 mmol) in 1.5 mL of dry dioxane containing 3 drops of DMF was subjected to microwave irradiation at 160° C. for 11 hours. 5 mL of water was added and the solution was extracted with dichloromethane (2×5 mL). The organic solvent was evaporated and the residue was purified by flash chromatography eluting with dichloromethane, 1 to 10% ethylacetate followed by preparative plate using a mixture of dichloromethane and 10% ethylacetate as eluent to give title compound (27 mg, 60%) as a brown solid. ESI/APCI (+) 423 (M+H). ¹H NMR (DMSO-d₆) δ 3.01 (dt, 1H), 3.16 (m, 1H), 3.20 (s, 3H), 3.37 (m, 2H), 3.53 (t, 2H), 3.68 (s, 3H), 4.20 (dt, 1H), 5.32 (s, 1H), 5.90 (brs, 1H, NH), 6.15 (d, 1H), 6.20 (s, 1H), 6.25 (d, 1H), 6.85 (m, 2H), 6.98 (t, 1H), 7.25 (m, 2H), 7.37 (t, 1H), 7.42 (t, 1H), 7.90 (d, 1H).

### Example 222

### Preparation of 4-((4-chlorophenylamino)methyl)-2-(2-methoxyethyl)-3-(thiophen-2-yl)-3,4-dihydroisoquinolin-1(2H)-one (C78)

To a 0° C. cooled solution of 4-(hydroxymethyl)-2-(2-methoxyethyl)-3-(thiophen-2-yl)-3,4-dihydroisoquinolin-1(2H)-one (108 mg, 0.34 mmol) in dichloromethane (5 mL) was added Dess-Martin periodinane (0.3M solution in CH₂Cl₂, 1.36 mL, 0.41 mmol), and the mixture was stirred at 0° C. for 2 h. Solutions of saturated aq NaHCO₃ (1 mL) and saturated aq Na₂S₂O₃ (1 mL) were then added to the mixture, and this was followed by the addition of dichloromethane (10 mL). The organic layer was separated, and the aqueous phase was further extracted with dichloromethane (2×10 mL). The combined organic extracts were washed with brine, dried (MgSO₄), and filtered, and the solvent was removed in vacuo to give a residue which was purified by flash chromatography eluting with heptane, 30 to 100% ethylacetate to provide 2-(2-methoxyethyl)-1-oxo-3-(thiophen-2-yl)-1,2,3,4-tetrahydroiso-quinoline-4-carbaldehyde (92.4 mg, 86%) as a viscous red oil. ESI/APCI (+) 316 (M+H). ESI/APCI (−) 314 (M−H).

To a 4 mL dichloroethane solution of 4-Chlroaniline (18 mg, 0.14 mmol) were added the aldehyde (45 mg, 0.14 mmol), acetic acid (10 μL, 0.17 mmol), and sodium triacetoxyborohydride (66 mg, 0.31 mmol), and the resulting mixture was stirred at room temperature for 24 hours. The reaction mixture was then diluted with dichloromethane, washed with saturated NaHCO₃, and dried over magnesium sulfate. Filtration and evaporation gave the crude product, which was purified by flash column chromatography eluting with dichloromethane, 1 to 10% ethylacetate to give the title compound (18 mg, 29%) as a white solid. ESI/APCI(+) 427 (M+H). ¹H NMR (DMSO-d₆) δ 3.01 (dt, 1H), 3.17 (m, 1H), 3.19 (s, 3H), 3.42 (m, 2H), 3.53 (m, 2H), 4.20 (dt, 1H), 5.32 (s, 1H), 6.08 (t, 1H, NH), 6.65 (d, 2H), 6.87 (m, 2H), 7.10 (d, 2H), 7.25 (m, 2H), 7.39 (t, 1H), 7.46 (t, 1H), 7.90 (d, 1H).

### Example 223

### Preparation of N-(4-cyano-3-(trifluoromethyl)phenyl)-2-(2-methoxyethyl)-1-oxo-3-(thiophen-2-yl)-1,2,3,4-tetrahydroisoquinoline-4-carboxamide (C221)

A solution of 2-(2-methoxyethyl)-1-oxo-3-(thiophen-2-yl)-1,2,3,4-tetrahydroisoquinoline-4-carboxylic acid (100 mg, 0.30 mmol) and thionyl chloride (1.5 mL) in chloroform (1.5 mL) was refluxed for 1 hour. After cooling, the volatiles were removed under reduced pressure. The residue was dissolved in chloroform (1.5 mL) under nitrogen atmosphere and triethylamine (1004) was added. To this solution was added 4-amino-2-(trifluoromethyl)benzonitrile (67 mg, 0.36 mmol) and the stirring was continued at 50° C. for 5 days. The mixture was evaporated under reduced pressure and the crude material was purified by flash chromatography on silica gel using a gradient of ethyl acetate (15-70%) in heptane followed by precipitation from a mixture of dichloromethane/heptane afford 23.3 mg (15%) of the titled compound as a tan solid. ESI/APCI(+): 500 (M+H). ESI/APCI(−) δ 498 (M−H). ¹H NMR (DMSO-d₆) δ 10.38 (1H, s, NH), 8.31 (1H, s), 8.14 (1H, d), 8.04 (1H, d), 7.94 (1H, d), 7.52-7.42 (2H, m), 7.37 (1H, d), 7.32 (1H, d), 7.02 (1H, s), 6.93 (1H, t), 5.62 (1H, s), 4.39 (1H, s), 4.14 (1H, dt), 3.40 (2H, t), 3.08 (1H, dt), 2.91 (3H, s).
Many other examples can be prepared according to the procedures described herein. A few other examples include:
6-chloro-2-(2-methoxyethyl)-N-(3-methoxyphenyl)-1-oxo-3-(thiophen-2-yl)-1,2,3,4-tetrahydroisoquinoline-4-carboxamide (C55), N-(2-amino-4-chlorophenyl)-2-(2-methoxyethyl)-1-oxo-3-(thiophen-2-yl)-1,2,3,4-tetrahydroisoquinoline-4-carboxamide (C144), N-(2-amino-5-chlorophenyl)-2-(2-methoxyethyl)-1-oxo-3-(thiophen-2-yl)-1,2,3,4-tetrahydroisoquinoline-4-carboxamide (C145), N-(1,3-dimethyl-1H-pyrazol-5-yl)-2-(2-methoxyethyl)-1-oxo-3-(thiophen-2-yl)-1,2,3,4-tetrahydroisoquinoline-4-carboxamide (C189), N-cyclohexyl-2-(2-methoxyethyl)-1-oxo-3-(thiophen-2-yl)-1,2,3,4-tetrahydroisoquinoline-4-carboxamide (C259), 2-(2-methoxyethyl)-4-(4-methylpiperidine-1-carbonyl)-3-(thiophen-2-yl)-3,4-dihydroisoquinolin-1(2H)-one (C260), 2-(2-methoxyethyl)-N-(3-(4-methylpiperidin-1-yl)propyl)-1-oxo-3-(thiophen-2-yl)-1,2,3,4-tetrahydroisoquinoline-4-carboxamide (C261), N-(2,5-dimethylphenyl)-N-ethyl-2-(2-methoxyethyl)-1-oxo-3-(thiophen-2-yl)-1,2,3,4-tetrahydroisoquinoline-4-carboxamide (C262), 2-(2-methoxyethyl)-1-oxo-N-(pyridin-3-yl)-3-(thiophen-2-yl)-1,2,3,4-tetrahydroisoquinoline-4-carboxamide (C263), 2-cyclohexyl-N-(3-methoxyphenyl)-3-(4-methoxyphenyl)-1-oxo-1,2,3,4-tetrahydroisoquinoline-4-carboxamide (C225), 3-(4-fluorophenyl)-N-(6-methoxybenzo[d]thiazol-2-yl)-2-(2-methoxyethyl)-1-oxo-1,2,3,4-tetrahydroisoquinoline-4-carboxamide (C226), 3-(4-fluorophenyl)-2-(2-methoxyethyl)-1-oxo-N-(quinolin-3-yl)-1,2,3,4-tetrahydroisoquinoline-4-carboxamide (C227), 3-(4-fluorophenyl)-N-(furan-2-ylmethyl)-2-(2-methoxyethyl)-1-oxo-1,2,3,4-tetrahydroisoquinoline-4-carboxamide (C228), N-((1-ethylpyrrolidin-2-yl)methyl)-3-(4-fluorophenyl)-2-(2-methoxyethyl)-1-oxo-1,2,3,4-tetrahydroisoquinoline-4-carboxamide (C229), 3-(4-fluorophenyl)-2-(2-methoxyethyl)-1-oxo-N-(pyridin-2-ylmethyl)-1,2,3,4-tetrahydroisoquinoline-4-carboxamide (C230), 3-(3,4-dimethoxyphenyl)-N-(3-methoxyphenyl)-2-methyl-1-oxo-1,2,3,4-tetrahydroisoquinoline-4-carboxamide (C231), 2-(4-fluorobenzyl)-N-(3-methoxyphenyl)-3-(4-methoxyphenyl)-1-oxo-1,2,3,4-tetrahydroisoquinoline-4-carboxamide (C232), 3-(3,4-dimethoxyphenyl)-N-(3-methoxyphenyl)-2-methyl-1-oxo-1,2,3,4-tetrahydroisoquinoline-4-carboxamide (C233), N-(3-methoxyphenyl)-3-(4-methoxyphenyl)-2-methyl-1-oxo-1,2,3,4-tetrahydroisoquinoline-4-carboxamide (C234), N-(3-methoxyphenyl)-2-methyl-1-oxo-3-(thiophen-2-yl)-1,2,3,4-tetrahydroisoquinoline-4-carboxamide (C235), 3-(2-fluorophenyl)-N-(3-methoxyphenyl)-2-methyl-1-oxo-1,2,3,4-tetrahydroisoquinoline-4-carboxamide (C236), N-(3-methoxyphenyl)-2-methyl-1-oxo-3-(thiophen-2-yl)-1,2,3,4-tetrahydroisoquinoline-4-carboxamide (C237), 2-(2-methoxyethyl)-N-(2-methoxyphenyl)-1-oxo-3-(thiophen-2-yl)-1,2,3,4-tetrahydroisoquinoline-4-carboxamide (C238), 2-(2-methoxyethyl)-1-oxo-N-(pyridin-2-yl)-3-(thiophen-2-yl)-1,2,3,4-tetrahydroisoquinoline-4-carboxamide (C239), 2-(2-methoxyethyl)-1-oxo-N-((tetrahydrofuran-2-yl)methyl)-3-(thiophen-2-yl)-1,2,3,4-tetrahydroisoquinoline-4-carboxamide (C240), N,2-bis(2-methoxyethyl)-1-oxo-3-(thiophen-2-yl)-1,2,3,4-tetrahydroisoquinoline-4-carboxamide (C241), N-(furan-2-ylmethyl)-2-(2-methoxyethyl)-1-oxo-3-(thiophen-2-yl)-1,2,3,4-tetrahydroisoquinoline-4-carboxamide (C242), N-((1-ethylpyrrolidin-2-yl)methyl)-2-(2-methoxyethyl)-1-oxo-3-(thiophen-2-yl)-1,2,3,4-tetrahydroisoquinoline-4-carboxamide (C243), 2-(2-methoxyethyl)-1-oxo-N-(pyridin-2-ylmethyl)-3-(thiophen-2-yl)-1,2,3,4-tetrahydroisoquinoline-4-carboxamide (C244), 2-(2-methoxyethyl)-1-oxo-N-(pyridin-3-ylmethyl)-3-(thiophen-2-yl)-1,2,3,4-tetrahydroisoquinoline-4-carboxamide (C245), ethyl 2-(2-(2-methoxyethyl)-1-oxo-3-(thiophen-2-yl)-1,2,3,4-tetrahydroisoquinoline-4-carboxamido)benzoate (C246), N-(3-cyanophenyl)-2-(2-methoxyethyl)-1-oxo-3-(thiophen-2-yl)-1,2,3,4-tetrahydroisoquinoline-4-carboxamide (C247), N-(3-methoxybenzyl)-2-(2-methoxyethyl)-1-oxo-3-(thiophen-2-yl)-1,2,3,4-tetrahydroisoquinoline-4-carboxamide (C248), N-(2-chloropyridin-3-yl)-2-(2-methoxyethyl)-1-oxo-3-(thiophen-2-yl)-1,2,3,4-tetrahydroisoquinoline-4-carboxamide (C249), N-(2-carbamoylphenyl)-2-(2-methoxyethyl)-1-oxo-3-(thiophen-2-yl)-1,2,3,4-tetrahydroisoquinoline-4-carboxamide (C264), 2-(2-methoxyethyl)-N-(4-methoxyphenyl)-1-oxo-3-(thiophen-2-yl)-1,2,3,4-tetrahydroisoquinoline-4-carboxamide (C250), N-(2-cyanophenyl)-2-(2-methoxyethyl)-1-oxo-3-(thiophen-2-yl)-1,2,3,4-tetrahydroisoquinoline-4-carboxamide (C251), N-(2-chlorophenyl)-2-(2-methoxyethyl)-1-oxo-3-(thiophen-2-yl)-1,2,3,4-tetrahydroisoquinoline-4-carboxamide (C252), 2-(2-methoxyethyl)-4-(4-(3-methoxyphenyl)piperazine-1-carbonyl)-3-(thiophen-2-yl)-3,4-dihydroisoquinolin-1(2H)-one (C253), N-(3-acetylphenyl)-2-(2-methoxyethyl)-1-oxo-3-(thiophen-2-yl)-1,2,3,4-tetrahydroisoquinoline-4-carboxamide (C254), N-benzyl-2-(2-methoxyethyl)-1-oxo-3-(thiophen-2-yl)-1,2,3,4-tetrahydroisoquinoline-4-carboxamide (C255), 2-isobutyl-N-(3-methoxyphenyl)-1-oxo-3-(thiophen-2-yl)-1,2,3,4-tetrahydroisoquinoline-4-carboxamide (C256), 2-cyclopentyl-N-(3-methoxyphenyl)-1-oxo-3-(thiophen-2-yl)-1,2,3,4-tetrahydroisoquinoline-4-carboxamide (C265), 2-cyclopentyl-1-oxo-N-(pyridin-3-yl)-3-(thiophen-2-yl)-1,2,3,4-tetrahydroisoquinoline-4-carboxamide (C257), N-(2-methoxybenzyl)-2-(2-methoxyethyl)-1-oxo-3-(thiophen-2-yl)-1,2,3,4-tetrahydroisoquinoline-4-carboxamide (C266), N-(2-ethoxybenzyl)-2-(2-methoxyethyl)-1-oxo-3-(thiophen-2-yl)-1,2,3,4-tetrahydroisoquinoline-4-carboxamide (C267), 2-isobutyl-N-isopropyl-1-oxo-3-(thiophen-2-yl)-1,2,3,4-tetrahydroisoquinoline-4-carboxamide (C268), 2-isobutyl-N-(2-morpholinoethyl)-1-oxo-3-(thiophen-2-yl)-1,2,3,4-tetrahydroisoquinoline-4-carboxamide (C269), 2-isobutyl-N-(4-ethylbenzyl)-1-oxo-3-(thiophen-2-yl)-1,2,3,4-tetrahydroisoquinoline-4-carboxamide (C270), 2-cyclopentyl-N-(3-morpholinopropyl)-1-oxo-3-(thiophen-2-yl)-1,2,3,4-tetrahydroisoquinoline-4-carboxamide (C271), 2-cyclopentyl-N-(2-(4-methylpiperazin-1-yl)ethyl)-1-oxo-3-(thiophen-2-yl)-1,2,3,4-tetrahydroisoquinoline-4-carboxamide (C272), 3-(4-fluorophenyl)-N-(6-methoxybenzo[d]thiazol-2-yl)-2-(2-methoxyethyl)-1-oxo-1,2,3,4-tetrahydroisoquinoline-4-carboxamide (C273).

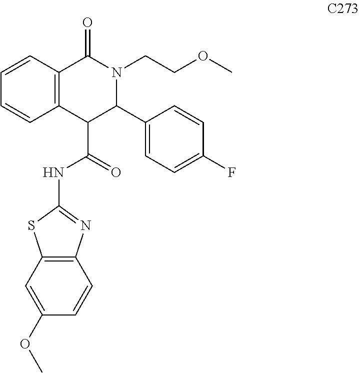

### Part B

### Methodology for Determination of Anti-Flaviviral and Cytostatic Activity

### Cells and Viruses

Madin-Darbey Bovine Kidney (MDBK) cells were maintained in Dulbecco's modified Eagle medium (DMEM) supplemented with BVDV-free 5% fetal calf serum (DMEME-FCS) at 37° C. in a humidified, 5% CO₂ atmosphere. BVDV-1 (strain PE515) was used to assess the antiviral activity in MDBK cells. Vero cells were maintained in the same way as MDBK cells. Vero cells were infected with Coxsackie B3 virus (strain Nancy).

### Determination of Cytostatic Effect on MDBK Cells

The effect of the drugs on exponentially growing MDBK cells was assessed as follows. Cells were seeded at a density of 5000 cell/well in 96 well plates in MEM medium (Gibco) supplemented with 10% fetal calf serum, 2 mM L-glutamine (Life Technologies) and bicarbonate (Life Technologies). Cells were cultured for 24 hr after which serial dilutions of the test compounds were added. Cultures were then again further incubated for 3 days after which the effect on cell growth was quantified by means of the MTS method (Promega). The concentration that results in 50% inhibition of cell growth is defined as the 50% cytostatic concentration (CC₅₀)

### Anti-HCV Assay/Replicon Assay

Huh-5-2 cells [a cell line with a persistent HCV replicon 1389Iuc-ubi-neo/NS3-3′/5.1, replicon with firefly luciferase-ubiquitin-neomycin phosphotransferase fusion protein and EMCV-IRES driven NS3-5B HCV polyprotein] was cultured in RPMI medium (Gibco) supplemented with 10% fetal calf serum, 2 mM L-glutamine (Life Technologies), 1× non-essential amino acids (Life Technologies), 100 IU/ml penicillin and 100 ug/ml streptomycin and 250 ug/ml G418 (Geneticin, Life Technologies). Cells were seeded at a density of 7000 cells per well in 96 well View Plate™ (Packard) in medium containing the same components as described above, except for G418. Cells were allowed to adhere and proliferate for 24 hr. At that time, culture medium was removed and serial dilutions of the test compounds were added in culture medium lacking G418. Interferon alfa 2a (500 IU) was included as a positive control. Plates were further incubated at 37° C. and 5% CO₂ for 72 hours. Replication of the HCV replicon in Huh-5 cells results in luciferase activity in the cells. Luciferase activity is measured by adding 50 μl of 1×Glo-lysis buffer (Promega) for 15 minutes followed by 50 ul of the Steady-Glo Luciferase assay reagent (Promega). Luciferase activity is measured with a luminometer and the signal in each individual well is expressed as a percentage of the untreated cultures. Parallel cultures of Huh-5-2 cells, seeded at a density of 7000 cells/well of classical 96-well cell culture plates (Becton-Dickinson) are treated in a similar fashion except that no Glo-lysis buffer or Steady-Glo Luciferase reagent is added. Instead the density of the culture is measured by means of the MTS method (Promega).

### Quantitative Analysis of HCV RNA by Taqman Real-Time RT-PCR

Replicon cells were plated at 7.5×10³ cells per well in a 96-well plate plates at 37° C. and 5% CO₂ in Dulbecco's modified essential medium containing 10% fetal calf serum, 1% nonessential amino acids and 1 mg/ml Geneticin. After allowing 24 h for cell attachment, different dilutions of compound were added to the cultures. Plates were incubated for 5 days, at which time RNA was extracted using the Qiamp Rneazyi Kit (Qiagen, Hilden, Germany). A 504 PCR reaction contained TaqMan EZ buffer (50 mmol/L Bicine, 115 mmol/L potassium acetate, 0.01 mmol/L EDTA, 60 nmol/L 6-carboxy-X-rhodamine, and 8% glycerol, pH 8.2, Perkin Elmer Corp./Applied Biosystems), 300 μmol/L deoxyadenosine triphosphate, 300 μmol/L deoxyguanosine triphosphate, 300 μmol/L deoxycytidine triphosphate, 600 μmol/L deoxyuridine triphosphate, 200 μmol/L forward primer [5′-ccg gcT Acc Tgc ccA TTc], 200 μmol/L reverse primer [ccA GaT cAT ccT gAT cgA cAA G], 100 μmol/L TaqMan probe [6-FAM-AcA Tcg cAT cgA gcg Agc Acg TAc-TAMRA], 3 mmol/L manganese acetate, 0.5 U AmpErase uracil-N-glycosylase, 7.5 U rTth DNA polymerase, and 10 μl of RNA elution. After initial activation of uracil-N-glycosylase at 50° C. for 2 minutes, RT was performed at 60° C. for 30 minutes, followed by inactivation of uracil-N-glycosylase at 95° C. for 5 minutes. Subsequent PCR amplification consisted of 40 cycles of denaturation at 94° C. for 20 seconds and annealing and extension at 62° C. for 1 minute in an ABI 7700 sequence detector. For each PCR run, negative template and positive template samples were used. The cycle threshold value (Ct-value) is defined as the number of PCR cycles for which the signal exceeds the baseline, which defines a positive value. The sample was considered to be positive if the Ct-value was <50. Results are expressed as genomic equivalents (GE).

### Example 224

### Effect of the Compounds of the Invention in HCV-HUH-5-2 Replicon Cells

Table 2 shows the effect in the anti-HCV replicon assay of some example compounds of the invention.

| | CC50 | IC50 | |
|---|---|---|---|
| | (μg/ml) | (μg/ml) | SI |
| C1 (cis) | 83.7 | 0.3 | 279 |
| C2 (trans) | 41.2 | 1.3 | 31 |
| Mixture of C1 and C2 | 67.3 | 0.98 | 68 |
| C3 | 65.13 | 40.89 | 2 |
| C4 | 125 | 41.55 | 3 |
| C5 | 33.4 | 3.6 | 9 |
| C6 | 69.6 | 6.3 | 11 |
| C8 | 54.5 | 21.81 | 2 |
| C9 | 50.71 | 13.38 | 4 |
| C10 | 49.92 | 32.13 | 2 |
| C11 | 37.9 | 1.2 | 30 |
| C12 | 69.7 | 1.2 | 67 |
| C13 | 33.29 | 2.11 | 16 |
| C15 | 20.16 | 11.81 | 2 |
| C16 | 10.4 | 1.1 | 9 |
| C17 | 50.4 | 3.8 | 13 |
| C18 | 78.16 | 2.65 | 29 |
| C19 | 12.1 | 5.2 | 2 |
| C20 | 42 | 0.4 | 105 |
| C21 | 31.1 | 0.2 | 156 |
| C22 | 36.93 | 4.09 | 9 |
| C23 | 118.25 | 42.97 | 3 |
| C24 | 125 | 35.73 | 3 |
| C25 | 5.28 | 0.98 | 5 |
| C27 | 63.79 | 17.41 | 4 |
| C28 | 74.9 | 38.75 | 2 |
| C29 | 54.55 | 14.75 | 4 |
| C30 | 70.31 | 36.67 | 2 |
| C31 | 66.5 | 1.1 | 62 |
| C32 | 73.74 | 37.27 | 2 |
| C34 | 40.78 | 16.54 | 2 |
| C36 | 82.69 | 47.07 | 2 |
| C37 | 92.7 | 1.5 | 62 |
| C38 | 85.43 | 41.38 | 2 |
| C39 | 80 | 1.3 | 62 |
| C40 | 88.08 | 7.76 | 11 |
| C41 | 107.05 | 0.5 | 214 |
| C42 | 33.1 | 0.5 | 67 |
| C43 | 70.8 | 0.3 | 236 |
| C44 | 28.37 | 4.17 | 7 |
| C45 | 72.6 | 0.1 | 726 |
| C46 | 86.3 | 1.4 | 62 |
| C47 | 98.37 | 0.4 | 246 |
| C48 | 102.05 | 0.3 | 340 |
| C49 | 96.8 | 0.1 | 968 |
| C50 | 76.4 | 0.7 | 109 |
| C51 | 63.15 | 0.5 | 126 |
| C52 | 81.2 | 0.4 | 203 |
| C53 | 80.83 | 52.91 | 2 |
| C55 | 88.04 | 42.99 | 2 |
| C56 | 43.97 | 0.98 | 45 |
| C57 | 76.23 | 39.46 | 2 |
| C58 | 84.96 | 1.81 | 47 |
| C59 | 88.14 | 0.3 | 294 |
| C60 | 86.91 | 0.98 | 89 |
| C61 | 62.56 | 0.22 | 284 |
| C62 | 16.35 | 8.81 | 2 |
| C63 | 81.57 | 3.21 | 25 |
| C64 | 47.77 | 23.87 | 2 |
| C65 | 90.18 | 1.76 | 51 |
| C66 | 39.22 | 1.99 | 20 |
| C67 | 21.15 | 10.44 | 2 |
| C68 | 125 | 70.63 | 2 |
| C69 | 125 | 8.7 | 14 |
| C70 | 125 | 3.33 | 38 |
| C71 | 125 | 12.57 | 10 |
| C72 | 71.16 | 35.82 | 2 |
| C74 | 84.41 | 35.96 | 2 |
| C75 | 125 | 43.13 | 3 |
| C76 | 42.23 | 22.03 | 2 |
| C77 | 125 | 11.24 | 11 |
| C78 | 125 | 50.49 | 2 |
| C79 | 125 | 68.17 | 2 |
| C81 | 65 | 0.16 | 406 |
| C82 | 72 | 12 | 6 |
| C83 | 70 | 0.4 | 175 |
| C84 | 50 | 12 | 4 |
| C85 | 75 | 0.18 | 417 |
| C86 | 75 | 0.25 | 300 |
| C87 | 4.34 | 0.15 | 29 |
| C88 | 60 | 2.7 | 22 |
| C89 | 0.62 | 0.11 | 6 |
| C91 | 45.1 | 0.32 | 141 |
| C92 | 68.87 | 7.04 | 10 |
| C93 | 32.88 | 0.17 | 193 |
| C94 | 17.08 | 0.25 | 68 |
| C95 | 76.26 | 2.21 | 35 |
| C97 | 79.99 | 51.59 | 2 |
| C98 | 80.89 | 50.29 | 2 |
| C100 | 86.74 | 56.82 | 2 |
| C102 | 72.75 | 26.84 | 3 |
| C103 | 75.17 | 40.38 | 2 |
| C104 | 79.94 | 10.05 | 8 |
| C105 | 84.48 | 50.49 | 2 |
| C106 | 84.79 | 44.44 | 2 |
| C107 | 23.47 | 0.12 | 196 |
| C108 | 77.33 | 3.71 | 21 |
| C109 | 83.5 | 43.43 | 2 |
| C110 | 79.32 | 1.45 | 55 |
| C111 | 86.69 | 0.17 | 510 |
| C112 | 75.66 | 0.31 | 244 |
| C113 | 66.9 | 0.19 | 352 |
| C114 | 58.68 | 0.47 | 125 |
| C115 | 76.62 | 0.29 | 264 |
| C116 | 72.44 | 0.04 | 1811 |
| C117 | 81.88 | 0.36 | 227 |
| C118 | 98.25 | 8.32 | 12 |
| C119 | 94 | 4.63 | 20 |
| C120 | 95.38 | 3.28 | 29 |
| C121 | 81.16 | 1.06 | 77 |
| C122 | 81.31 | 0.37 | 222 |
| C123 | 87.86 | 0.25 | 351 |
| C124 | 86.68 | 10.72 | 8 |
| C125 | 53.61 | 7.3 | 7 |
| C126 | 58.11 | 0.9 | 65 |
| C127 | 87.21 | 10.29 | 8 |
| C128 | 52.73 | 0.4 | 132 |
| C129 | 97.01 | 6.66 | 15 |
| C130 | 91.8 | 0.8 | 115 |
| C131 | 69.65 | 1.12 | 62 |
| C132 | 81.28 | 1 | 81 |
| C135 | 56.74 | 0.52 | 109 |
| C137 | 81.1 | 0.032 | 2568 |
| C139 | 75.28 | 2.74 | 27 |
| C141 | 73.81 | 0.63 | 117 |
| C142 | 8.95 | 0.04 | 224 |
| C143 | 83.24 | 24.29 | 3.4 |
| C144 | 45.24 | 5.16 | 8.8 |
| C147 | 77.83 | 0.14 | 537 |
| C148 | 52.15 | 24.55 | 2 |
| C150 | >125 | 0.08 | >1562 |
| C151 | 52 | 22.84 | 2.3 |
| C152 | 30 | 0.2 | 150 |
| C153 | 60.9 | 0.104 | 584 |
| C154 | 53.44 | 3.68 | 15 |
| C155 | 76.44 | 5.65 | 14 |
| C156 | 86.32 | 37.42 | 2 |
| C157 | 71.34 | 0.04 | 2017 |
| C158 | 6.1 | 0.04 | 146 |
| C159 | >100 | 0.028 | >3518 |
| C160 | 41.38 | 0.12 | 332 |
| C162 | 80.27 | 3.56 | 23 |
| C163 | 81.01 | 5.23 | 15 |
| C164 | 20.51 | 0.13 | 157 |
| C165 | 33.09 | 0.4 | 82 |
| C166 | 64.33 | 0.38 | 169 |
| C167 | 60.03 | 0.12 | 519 |
| C168 | >100 | 0.037 | >2739 |
| C170 | 37.66 | 0.12 | 313 |
| C171 | 72.59 | 0.12 | 602 |
| C172 | 14.47 | 0.09 | 167 |
| C173 | 9.59 | 0.09 | 110 |
| C174 | 42.98 | 0.15 | 295 |
| C175 | 10.4 | 0.057 | 181 |
| C176 | 71.65 | 0.48 | 151 |
| C177 | 3.16 | 0.5 | 6 |
| C178 | >100 | 0.035 | >2843 |
| C180 | 80.76 | 34.8 | 2 |
| C181 | 79.89 | 0.25 | 322 |
| C182 | 36.19 | 21.79 | 2 |
| C183 | 76.98 | 50.19 | 2 |
| C184 | 85.02 | 54.98 | 2 |
| C185 | 19.03 | 10.4 | 2 |
| C186 | 2.79 | 0.04 | 71 |
| C187 | 12.4 | 0.01 | 1054 |
| C188 | 45.05 | 0.1 | 451 |
| C189 | 82.22 | 26.06 | 3 |
| C190 | 75.94 | 0.14 | 559 |
| C191 | 52.5 | 0.013 | 4072 |
| C192 | 0.98 | 0.01 | 99 |
| C194 | 11.6 | 0.019 | 609 |
| C195 | 72.09 | 0.13 | 571 |
| C197 | 78.21 | 4 | 20 |
| C198 | 2.33 | 4 | 0 |
| C199 | 5.97 | 0.12 | 50 |
| C200 | 59.43 | 0.32 | 187 |
| C201 | 19.49 | 0.078 | 249 |
| C202 | 7.89 | 0.17 | 46 |
| C203 | 9.4 | 0.019 | 491 |
| C205 | 38.83 | 0.034 | 1155 |
| C206 | 52.29 | 3.72 | 14 |
| C207 | 72.35 | 27.44 | 3 |
| C208 | 7.94 | 0.39 | 21 |
| C209 | 57.82 | 0.11 | 524 |
| C210 | 15.7 | 0.072 | 218 |
| C211 | 30.14 | 0.1 | 301 |
| C212 | 52.65 | 0.16 | 326 |
| C213 | >100 | 0.036 | >2768 |
| C214 | 85.35 | 5.29 | 16 |
| C215 | 52.9 | 1.04 | 51 |
| C216 | 10.95 | 2.57 | 4 |
| C217 | 83.67 | 0.76 | 110 |
| C218 | 83.83 | 0.1 | 838 |
| C219 | 91.11 | 1.14 | 80 |
| C220 | 81.94 | 2.67 | 31 |
| C221 | 3.52 | 0.36 | 10 |
| C222 | 16.19 | 0.21 | 76 |
| C223 | 79.02 | 1 | 79 |
| C273 | 14.9 | 1.4 | 10 |

IC₅₀: concentration required to inhibit luciferase activity in the replicon system by 50%.
CC₅₀: concentration required to inhibit the proliferation of exponentially growing Huh-5-2 cells by 50%.
SI: selectivity index (=CC₅₀/IC₅₀)

### Example 225

### Effect of Compounds of the Invention on Replicon (RNA) Synthesis in Huh-5-2 Cells

A selection of compounds was also tested for their effect on the synthesis of the HCV replicon as determined by means of real-time quantitative RT-PCR and thereby showed a clear reduction of viral RNA (reduction with more than 80%) at non-cytotoxic or cytostatic concentrations. As an example, the detailed data for compound 2-(2-methoxyethyl)-N-(3-methoxyphenyl)-1-oxo-3-(thiophen-2-yl)-1,2,3,4-tetrahydroisoquinoline-4-carboxamide are given below in table 3 and more results are shown in table 4.

TABLE 3
Effect of compound 2-(2-methoxyethyl)-N-(3-methoxyphenyl)-
1-oxo-3-(thiophen-2-yl)-1,2,3,4-tetrahydroisoquinoline-4-
carboxamide on replicon (RNA) synthesis in Huh-5-2 cells.

| | CT | Delta CT |
|---|---|---|
| 20 ug/ml | 29.24 | 2.97 |
| 10 ug/ml | 29.29 | 3.02 |
| 5 ug/ml | 28.65 | 2.38 |
| 2.5 ug/ml | 27.87 | 1.6 |
| 1.25 ug/ml | 29.08 | 2.81 |
| untreated | 26.27 ± 0.34 | |

Effect of the antivirals on the synthesis of the HCV replicon as determined by means of real-time quantitative RT-PCR. A higher Delta CT value indicates a more profound inhibition of viral RNA synthesis.

TABLE 4
Effect of example compounds on replicon (RNA)
synthesis in Huh-5-2 cells as determined by means
of real-time quantitative RT-PCR.

| | CC50 (μg/mL) | EC50 (μg/mL) |
|---|---|---|
| C1 | >50 | 24.000 |
| C2 | >50 | 17.000 |
| C31 | >50 | 18.000 |
| C41 | >50 | 0.280 |
| C45 | >50 | 12.000 |
| C46 | >50 | 1.190 |
| C47 | >50 | 1.420 |
| C48 | >50 | 0.600 |
| C49 | >50 | 0.850 |
| C51 | >50 | <0.4 |
| C52 | >50 | <0.4 |
| C116 | >50 | 0.062 |
| C128 | >50 | 0.140 |
| C142 | >30 | 0.220 |
| C152 | >30 | 0.200 |
| C153 | >50 | 1.12 |
| C157 | >50 | 0.036 |
| C159 | >50 | 0.064 |
| C168 | >50 | 0.048 |
| C175 | >50 | 0.046 |
| C178 | >50 | 0.036 |
| C186 | >50 | 0.045 |
| C187 | >50 | <0.016 |
| C191 | >50 | 0.013 |

EC₅₀: concentration required to reduce the synthesis of HCV replicon by 50% as determined by real-time quantitative RT-PCR.
CC₅₀: concentration required to inhibit the proliferation of exponentially growing Huh-5-2 cells by 50%.

All publications and patent applications cited herein are incorporated by reference to the same extent as if each individual publication or patent application was specifically and individually indicated to be incorporated by reference. Specifically cited sections or pages of the above cited works are incorporated by reference with specificity. The invention has been described in detail sufficient to allow one of ordinary skill in the art to make and use the subject matter of the following Embodiments. Many modifications are possible in the embodiments without departing from the teachings thereof. All such modifications are intended to be encompassed within the claims of the invention.

1-33. (canceled)

34. A method for the prevention or treatment of a RNA virus infection in an animal or a mammal, comprising the administration to said animal or mammal of a compound according to formula (I), 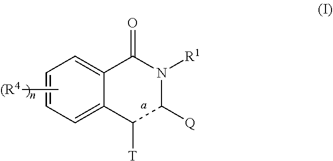
wherein:
the dotted line “a” is selected from a single bond or a double bond,
each of R¹, R², and R³ is independently selected from hydrogen or a C₁₋₁₈ hydrocarbyl group which optionally includes one or more heteroatoms selected from the group consisting of O, S, P, and N, wherein said C₁₋₁₈ hydrocarbyl group can be unsubstituted or substituted,
each R⁴ is independently selected from the group consisting of halogen, —OH, C₁₋₁₈ alkoxy, —SH, C₁₋₁₈ thioalkoxy, trifluoromethyl, trifluoromethoxy, nitro, -cyano, —COOH, —COO—C₁₋₆ alkyl, —COO—C₂₋₆ alkenyl, —COO—C₂₋₆ alkynyl, amino, C₁₋₁₈ heteroalkyl, C₁₋₁₈ alkyl, C₂₋₁₈ alkenyl, C₂₋₁₈ alkynyl, aryl, heterocycle, aryl-C₁₋₁₈ alkyl, aryl-C₂₋₁₈ alkenyl, aryl-C₂₋₁₈ alkynyl, heterocycle-C₁₋₁₈ alkyl, heterocycle-C₂₋₁₈ alkenyl, and heterocycle-C₂₋₁₈-alkynyl,
n is 0, 1, 2, 3, or 4,
Q is -L¹R² or —R²,
T is -L²R³ or —R³,
each of L¹ and L² is independently selected from the group consisting of C₁₋₆ alkylene, C₂₋₆-alkenylene, and C₂₋₆ alkynylene, wherein each of said C₁₋₆ alkylene, C₂₋₆ alkenylene or C₂₋₆ alkynylene optionally includes one or more heteroatoms selected from the group consisting of O, S, P, and N, and wherein each of said C₁₋₆ alkylene, C₂₋₆ alkenylene or C₂₋₆ alkynylene can be unsubstituted or substituted, stereo-isomers, tautomers, hydrates, or pharmaceutically acceptable salts thereof.

35. The method according to claim 34, wherein the compound is according to one of the formulae (Ia), (Ib), (Ic), or (Id)

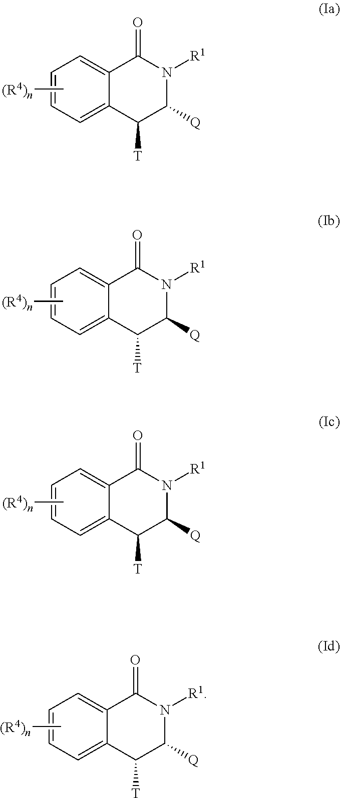

36. The method according to claim 34, wherein
each of R¹ and R² is independently selected from the group consisting of alkyl, alkenyl, alkynyl, aryl, heterocycle, arylalkyl, arylalkenyl, arylalkynyl, heterocycle-alkyl, heterocycle-alkenyl, and heterocycle-alkynyl, and wherein said alkyl, alkenyl, alkynyl, aryl, heterocycle, arylalkyl, arylalkenyl, arylalkynyl, heterocycle-alkyl, heterocycle-alkenyl or heterocycle-alkynyl optionally includes one or more heteroatoms selected from the group consisting of O, S, P, and N, and wherein said alkyl, alkenyl, alkynyl, aryl, heterocycle, arylalkyl, arylalkenyl, arylalkynyl, heterocycle-alkyl, heterocycle-alkenyl or heterocycle-alkynyl can be unsubstituted or substituted with one or more Z¹,
R³ is selected from the group consisting of alkyl, alkenyl, alkynyl, heterocycle, aryl, arylalkyl, arylalkenyl, arylalkynyl, heterocycle-alkyl, heterocycle-alkenyl, and heterocycle-alkynyl, wherein said alkyl, alkenyl, alkynyl, arylalkyl, arylalkenyl, arylalkynyl, heterocycle-alkyl, heterocycle-alkenyl or heterocycle-alkynyl optionally include one or more heteroatoms selected from the group consisting of O, S, P, and N, and wherein said alkyl, alkenyl, alkynyl, aryl, heterocycle, arylalkyl, arylalkenyl, arylalkynyl, heterocycle-alkyl, heterocycle-alkenyl or heterocycle-alkynyl is optionally substituted with one or more Z¹⁶,
each of R⁴ is independently selected from the group consisting of halogen, —OH, alkoxy, —SH, thioalkoxy, trifluoromethyl, trifluoromethoxy, nitro, cyano, —COOH, —COO-alkyl, —COO-alkenyl, —COO-alkynyl, amino, heteroalkyl, alkyl, alkenyl, alkynyl, aryl, heterocycle, arylalkyl, arylalkenyl, arylalkynyl, heterocycle-alkyl, heterocycle-alkenyl, and heterocycle-alkynyl,
n is 0, 1, 2, 3, or 4,
Q is —R²,
T is -L²R³ or —R³,
L² is selected from —CH₂NZ⁶—, —CH₂NH—, —CO—NZ⁶—, —CH₂O—, —COO—, or —CONH—,
each Z¹ is independently selected from the group consisting of halogen, hydroxyl, sulfhydryl, alkoxy, thioalkoxy, ═O, ═S, trifluoromethyl, trifluoromethoxy, nitro, amino, cyano, —COOH, —COO-alkyl, —COO-alkenyl, —COO-alkynyl, —CONH₂, alkyl, alkenyl, alkynyl, aryl, heterocycle, arylalkyl, arylalkenyl, arylalkynyl, heterocycle-alkyl, heterocycle-alkenyl, and heterocycle-alkynyl,
Z⁶ is independently selected from the group consisting of alkyl, alkenyl, alkynyl, heteroalkyl, heterocycle, heterocycle-alkyl, arylalkyl, and aryl,
each Z¹⁶ is independently selected from the group consisting of halogen, hydroxyl, sulfhydryl, alkoxy, thioalkoxy, ═O, ═S, trifluoromethyl, trifluoromethoxy, nitro, amino, cyano, —NH—SO₂—C₁₋₆-alkyl, alkyl, alkenyl, alkynyl, aryl, heterocycle, arylalkyl, arylalkenyl, arylalkynyl, heterocycle-alkyl, heterocycle-alkenyl, and heterocycle-alkynyl, wherein said alkoxy, thioalkoxy, alkyl, alkenyl, alkynyl, aryl, heterocycle, arylalkyl, arylalkenyl, arylalkynyl, heterocycle-alkyl, heterocycle-alkenyl, and heterocycle-alkynyl group which optionally includes one or more heteroatoms selected from the group consisting of O, S, P, and N, and wherein said alkoxy, thioalkoxy, alkyl, alkenyl, alkynyl, aryl, heterocycle, arylalkyl, arylalkenyl, arylalkynyl, heterocycle-alkyl, heterocycle-alkenyl, and heterocycle-alkynyl group is optionally substituted with one or more Z¹⁷,
each Z¹⁷ is independently selected from the group consisting of halogen, —OH, alkoxy, —SH, thioalkoxy, trifluoromethyl, trifluoromethoxy, ═O, ═S, nitro, cyano, —CONH₂, —COCH₃, —COOH, —COO-alkyl, —COO-alkenyl, —COO-alkynyl, amino, heteroalkyl, alkyl, alkenyl, alkynyl, aryl, heterocycle, arylalkyl, arylalkenyl, arylalkynyl, heterocycle-alkyl, heterocycle-alkenyl, and heterocycle-alkynyl.

37. The method according to claim 34, wherein:
each of R¹ and R² is independently selected from the group consisting of C₁₋₁₈-alkyl, C₂₋₁₈-alkenyl, C₂₋₁₈-alkynyl, aryl, heterocycle, aryl-C₁₋₁₈-alkyl, aryl-C₂₋₁₈-alkenyl, aryl-C₂₋₁₈-alkynyl, heterocycle-C₁₋₁₈-alkyl, heterocycle-C₂₋₁₈-alkenyl and heterocycle-C₂₋₁₈-alkynyl, and wherein said C₁₋₁₈-alkyl, C₂₋₁₈-alkenyl, C₂₋₁₈-alkynyl, aryl, heterocycle, aryl-C₁₋₁₈-alkyl, aryl-C₂₋₁₈-alkenyl, aryl-C₂₋₁₈-alkynyl, heterocycle-C₁₋₁₈-alkyl, heterocycle-C₂₋₁₈-alkenyl or heterocycle-C₂₋₁₈-alkynyl, which optionally includes one or more heteroatoms selected from the group consisting of O, S, P, and N, and wherein said C₁₋₁₈-alkyl, C₂₋₁₈-alkenyl, C₂₋₁₈-alkynyl, aryl, heterocycle, aryl-C₁₋₁₈-alkyl, aryl-C₂₋₁₈-alkenyl, aryl-C₂₋₁₈-alkynyl, heterocycle-C₁₋₁₈-alkyl, heterocycle-C₂₋₁₈-alkenyl or heterocycle-C₂₋₁₈-alkynyl can be unsubstituted or substituted with one or more Z¹,
R³ is an C₁₋₁₈-alkyl, heterocyclic group or aryl group optionally substituted with one or more Z¹⁶,
each R⁴ is independently selected from the group consisting of halogen, —OH, C₁₋₁₈-alkoxy, —SH, C₁₋₁₈-thioalkoxy, trifluoromethyl, trifluoromethoxy, nitro, -cyano, —NHSO₂—C₁₋₆-alkyl, —COOH, —COO—C₁₋₆-alkyl, —COO—C₂₋₆-alkenyl, —COO—C₂₋₆-alkynyl, amino, C₁₋₁₈-heteroalkyl, C₁₋₁₈-alkyl, C₂₋₁₈-alkenyl, C₂₋₁₈-alkynyl, aryl, and heterocycle,
n is 0, 1, 2, 3 or 4,
Q is —R²,
T is -L²R³ or —R³,
L² is selected from the group consisting of —CH₂NZ⁶—, CH₂NH—, —CO—NZ⁶—, —C(═O)—, —CH₂O—, —COO—, and —CONH,
each Z¹ is independently selected from the group consisting of hydrogen, halogen, hydroxyl, sulfhydryl, C₁₋₁₈-alkoxy, C₁₋₁₈-thioalkoxy, ═O, trifluoromethyl, trifluoromethoxy, nitro, -amino, C₁₋₁₈-alkyl, C₂₋₁₈-alkenyl, C₂₋₁₈-alkynyl, aryl, heterocycle, aryl-C₁₋₁₈-alkyl, aryl-C₂₋₁₈-alkenyl, aryl-C₂₋₁₈-alkynyl, heterocycle-C₁₋₁₈-alkyl, heterocycle-C₂₋₁₈-alkenyl, and heterocycle-C₂₋₁₈-alkynyl,
each Z⁶ is independently selected from the group consisting of alkyl, heteroalkyl, and aryl, and
each Z¹⁶ is independently selected from the group consisting of halogen, hydroxyl, sulfhydryl, C₁₋₁₈-alkoxy, C₁₋₁₈-thioalkoxy, ═O, ═S, trifluoromethyl, trifluoromethoxy, nitro, -amino, cyano, —NH—SO₂—C₁₋₆-alkyl, —COOH, —COO—C₁₋₆-alkyl, —COO—C₂₋₆-alkenyl, —COO—C₂₋₆-alkynyl, —CONH₂, C₁₋₁₈-alkyl, C₂₋₁₈-alkenyl, C₂₋₁₈-alkynyl, aryl, heterocycle, aryl-C₁₋₁₈-alkyl, aryl-C₂₋₁₈-alkenyl, aryl-C₂₋₁₈-alkynyl, heterocycle-C₁₋₁₈-alkyl, heterocycle-C₂₋₁₈-alkenyl, and heterocycle-C₂₋₁₈-alkynyl, wherein said C₁₋₁₈-alkoxy, C₁₋₁₈-thioalkoxy, C₁₋₁₈-alkyl, C₂₋₁₈-alkenyl, C₂₋₁₈-alkynyl, aryl, heterocycle, aryl-C₁₋₁₈-alkyl, aryl-C₂₋₁₈-alkenyl, aryl-C₂₋₁₈-alkynyl, heterocycle-C₁₋₁₈-alkyl, heterocycle-C₂₋₁₈-alkenyl and heterocycle-C₂₋₁₈-alkynyl group is optionally substituted with a heterocyclic or carbocyclic group.

38. The method according to claim 34, wherein n is 2.

39. The method according to claim 34, wherein the dotted line “a” is a single bond.

40. The method according to claim 34, wherein R¹ is selected from the group consisting of C₁₋₆-alkyl, C₂₋₆-alkenyl, or C₂₋₆-alkynyl, wherein said C₁₋₆-alkyl, C₂₋₆-alkenyl or C₂₋₆-alkynyl optionally include one or more heteroatoms selected from the group consisting of O, S, P, and N, and wherein said C₁₋₆-alkyl, C₂₋₆-alkenyl or C₂₋₆-alkynyl can be unsubstituted or substituted.

41. The method according to claim 40, wherein R¹ is selected from the group consisting of an acyclic alkyl, acyclic alkenyl, and acyclic alkynyl, wherein said acyclic alkyl, acyclic alkenyl or acyclic alkynyl optionally includes one or more heteroatoms selected from the group consisting of O, S, P, and N, and wherein said acyclic alkyl, acyclic alkenyl or acyclic alkynyl can be unsubstituted or substituted.

42. The method according to claim 34, wherein
Q is R²,
R² is selected from the group consisting of aryl, heterocycle, aryl-C₁₋₆-alkyl, aryl-C₂₋₆-alkenyl, aryl-C₂₋₆-alkynyl, heterocycle-C₁₋₆-alkyl, heterocycle-C₂₋₆-alkenyl, and heterocycle-C₂₋₆-alkynyl, wherein said aryl, heterocycle, aryl-C₁₋₆-alkyl, aryl-C₂₋₆-alkenyl, aryl-C₂₋₆-alkynyl, heterocycle-C₁₋₆-alkyl, heterocycle-C₂₋₆-alkenyl or heterocycle-C₂₋₆-alkynyl optionally includes one or more heteroatoms selected from the group consisting of O, S, P, and N, and wherein said aryl, heterocycle, aryl-C₁₋₆-alkyl, aryl-C₂₋₆-alkenyl, aryl-C₂₋₆-alkynyl, heterocycle-C₁₋₆-alkyl, heterocycle-C₂₋₆-alkenyl or heterocycle-C₂₋₆-alkynyl can be unsubstituted or substituted.

43. The method according to claim 42, wherein R² is selected from unsubstituted or substituted thienyl or furanyl.

44. The method according to claim 34, wherein L² is selected from the group consisting of —CH₂NZ⁶—, CH₂NH—, —CO—NZ⁶—, —CONH—, —COO—, —C(═O)—, and —CH₂O—, and wherein each Z⁶ is independently selected from the group consisting of C₁₋₆-alkyl, C₁₋₆-heteroalkyl, and aryl which can be unsubstituted or substituted with one or more Z¹.

45. The method according to claim 34, wherein R³ is selected from the group consisting of aryl, heterocycle, aryl-C₁₋₆-alkyl, aryl-C₂₋₆-alkenyl, aryl-C₂₋₆-alkynyl, heterocycle-C₁₋₆-alkyl, heterocycle-C₂₋₆-alkenyl, and heterocycle-C₂₋₆-alkynyl, and wherein said C₁₋₆-alkyl, C₂₋₆-alkenyl, C₂₋₆-alkynyl, aryl, heterocycle, aryl-C₁₋₆-alkyl, aryl-C₂₋₆-alkenyl, aryl-C₂₋₆-alkynyl, heterocycle-C₁₋₆-alkyl, heterocycle-C₂₋₆-alkenyl or heterocycle-C₂₋₆-alkynyl optionally includes one or more heteroatoms selected from the group consisting of O, S, P, and N, and wherein said C₁₋₆-alkyl, C₂₋₆-alkenyl, C₂₋₆-alkynyl, aryl, heterocycle, aryl-C₁₋₆-alkyl, aryl-C₂₋₆-alkenyl, aryl-C₂₋₆-alkynyl, heterocycle-C₁₋₆-alkyl, heterocycle-C₂₋₆-alkenyl or heterocycle-C₂₋₆-alkynyl can be unsubstituted or substituted.

46. The method according to claim 34, wherein the compound is selected from the following group of compounds and their isomers, pharmaceutically acceptable salts, and hydrates:
2-(2-methoxyethyl)-N-(3-methoxyphenyl)-7-nitro-1-oxo-3-(thiophen-2-yl)-1,2,3,4-tetrahydroisoquinoline-4-carboxamide,
N-(3-methoxyphenyl)-2-(3-methoxypropyl)-1-oxo-3-(thiophen-2-yl)-1,2,3,4-tetrahydroisoquinoline-4-carboxamide,
2-(2-methoxyethyl)-N-(3-methoxyphenyl)-1-oxo-3-(thiophen-2-yl)-1,2,3,4-tetrahydroisoquinoline-4-carboxamide,
Methyl 2-(4-(3-methoxyphenylcarbamoyl)-1-oxo-3-(thiophen-2-yl)-3,4-dihydro-isoquinolin-2(1H)-yl)acetate,
2-(2-(Dimethylamino)ethyl)-N-(3-methoxyphenyl)-1-oxo-3-(thiophen-2-yl)-1,2,3,4-tetrahydroisoquinoline-4-carboxamide,
2-Butyl-N-(3-methoxyphenyl)-1-oxo-3-(thiophen-2-yl)-1,2,3,4-tetrahydroiso-quinoline-4-carboxamide,
2-(2-Methoxyethyl)-N-(3-methoxyphenyl)-5-methyl-1-oxo-3-(thiophen-2-yl)-1,2,3,4-tetrahydroisoquinoline-4-carboxamide,
2-(2-Methoxyethyl)-N-(3-methoxyphenyl)-6-methyl-1-oxo-3-(thiophen-2-yl)-1,2,3,4-tetrahydroisoquinoline-4-carboxamide,
2-(2-Methoxyethyl)-N-(3-methoxyphenyl)-7-methyl-1-oxo-3-(thiophen-2-yl)-1,2,3,4-tetrahydroisoquinoline-4-carboxamide,
2-(2-Methoxyethyl)-N-(3-methoxyphenyl)-8-methyl-1-oxo-3-(thiophen-2-yl)-1,2,3,4-tetrahydroisoquinoline-4-carboxamide,
7-Chloro-2-(2-methoxyethyl)-N-(3-methoxyphenyl)-1-oxo-3-(thiophen-2-yl)-1,2,3,4-tetrahydroisoquinoline-4-carboxamide,
8-Chloro-2-(2-methoxyethyl)-N-(3-methoxyphenyl)-1-oxo-3-(thiophen-2-yl)-1,2,3,4-tetrahydroisoquinoline-4-carboxamide,
7-Iodo-2-(2-methoxyethyl)-N-(3-methoxyphenyl)-1-oxo-3-(thiophen-2-yl)-1,2,3,4-tetrahydroisoquinoline-4-carboxamide,
2-(2-Methoxyethyl)-1-oxo-N-phenyl-3-(thiophen-2-yl)-1,2,3,4-tetrahydroiso-quinoline-4-carboxamide,
2-(2-Methoxyethyl)-1-oxo-3-(thiophen-2-yl)-N-(4-(trifluoromethyl)phenyl)-1,2,3,4-tetrahydroisoquinoline-4-carboxamide,
N-(4-cyanophenyl)-2-(2-methoxyethyl)-1-oxo-3-(thiophen-2-yl)-1,2,3,4-tetra-hydroisoquinoline-4-carboxamide,
N-(biphenyl-3-yl)-2-(2-methoxyethyl)-1-oxo-3-(thiophen-2-yl)-1,2,3,4-tetra-hydroisoquinoline-4-carboxamide,
2-(2-Methoxyethyl)-1-oxo-N-(2-phenoxyethyl)-3-(thiophen-2-yl)-1,2,3,4-tetra-hydroisoquinoline-4-carboxamide,
2-(2-Methoxyethyl)-N-(2-(1-methyl-1H-indol-3-yl)ethyl)-1-oxo-3-(thiophen-2-yl)-1,2,3,4-tetrahydroisoquinoline-4-carboxamide,
Methyl 4-(2-(2-methoxyethyl)-1-oxo-3-(thiophen-2-yl)-1,2,3,4-tetrahydroiso-quinoline-4-carboxamido)benzoate,
Ethyl 3-(2-(2-methoxyethyl)-1-oxo-3-(thiophen-2-yl)-1,2,3,4-tetrahydroiso-quinoline-4-carboxamido)benzoate,
N-(3-fluorophenyl)-2-(2-methoxyethyl)-1-oxo-3-(thiophen-2-yl)-1,2,3,4-tetrahydroisoquinoline-4-carboxamide,
N-(3,4-dimethoxyphenyl)-2-(2-methoxyethyl)-1-oxo-3-(thiophen-2-yl)-1,2,3,4-tetrahydroisoquinoline-4-carboxamide,
N-(4-chlorophenyl)-2-(2-methoxyethyl)-1-oxo-3-(thiophen-2-yl)-1,2,3,4-tetrahydroisoquinoline-4-carboxamide,
2-(Furan-2-ylmethyl)-N-(3-methoxyphenyl)-1-oxo-3-(thiophen-2-yl)-1,2,3,4-tetrahydroisoquinoline-4-carboxamide,
2-Ethyl-N-(3-methoxyphenyl)-1-oxo-3-(thiophen-2-yl)-1,2,3,4-tetrahydroisoquinoline-4-carboxamide,
2-(Cyclohexylmethyl)-N-(3-methoxyphenyl)-1-oxo-3-(thiophen-2-yl)-1,2,3,4-tetrahydroisoquinoline-4-carboxamide,
2-(2-Hydroxyethyl)-N-(3-methoxyphenyl)-1-oxo-3-(thiophen-2-yl)-1,2,3,4-tetrahydroisoquinoline-4-carboxamide,
2-Cyclohexyl-N-(3-methoxyphenyl)-1-oxo-3-(thiophen-2-yl)-1,2,3,4-tetrahydroisoquinoline-4-carboxamide,
N-(3-methoxyphenyl)-1-oxo-2-((tetrahydrofuran-2-yl)methyl)-3-(thiophen-2-yl)-1,2,3,4-tetrahydroisoquinoline-4-carboxamide,
2-Benzyl-N-(3-methoxyphenyl)-1-oxo-3-(thiophen-2-yl)-1,2,3,4-tetrahydroisoquinoline-4-carboxamide,
7-Methoxy-2-(2-methoxyethyl)-N-(3-methoxyphenyl)-1-oxo-3-(thiophen-2-yl)-1,2,3,4-tetrahydroisoquinoline-4-carboxamide,
8-Fluoro-2-(2-methoxyethyl)-N-(3-methoxyphenyl)-1-oxo-3-(thiophen-2-yl)-1,2,3,4-tetrahydroisoquinoline-4-carboxamide,
7-Fluoro-2-(2-methoxyethyl)-N-(3-methoxyphenyl)-1-oxo-3-(thiophen-2-yl)-1,2,3,4-tetrahydroisoquinoline-4-carboxamide,
5-Fluoro-2-(2-methoxyethyl)-N-(3-methoxyphenyl)-1-oxo-3-(thiophen-2-yl)-1,2,3,4-tetrahydroisoquinoline-4-carboxamide,
N-(3-methoxyphenyl)-1-oxo-3-(thiophen-2-yl)-2-(2,2,2-trifluoroethyl)-1,2,3,4-tetrahydroisoquinoline-4-carboxamide,
N-(3-methoxyphenyl)-2-(2-(methylthio)ethyl)-1-oxo-3-(thiophen-2-yl)-1,2,3,4-tetrahydroisoquinoline-4-carboxamide,
N-(3-methoxyphenyl)-2-(3-morpholinopropyl)-1-oxo-3-(thiophen-2-yl)-1,2,3,4-tetrahydroisoquinoline-4-carboxamide,
N-(3-ethoxyphenyl)-2-(2-methoxyethyl)-1-oxo-3-(thiophen-2-yl)-1,2,3,4-tetrahydroisoquinoline-4-carboxamide,
N-(3-chlorophenyl)-2-(2-methoxyethyl)-1-oxo-3-(thiophen-2-yl)-1,2,3,4-tetrahydroisoquinoline-4-carboxamide,
2-(2-Ethoxyethyl)-N-(3-methoxyphenyl)-1-oxo-3-(thiophen-2-yl)-1,2,3,4-tetrahydroisoquinoline-4-carboxamide,
2-(2-Isopropoxyethyl)-N-(3-methoxyphenyl)-1-oxo-3-(thiophen-2-yl)-1,2,3,4-tetrahydroisoquinoline-4-carboxamide,
2-((1-ethylpyrrolidin-2-yl)methyl)-N-(3-methoxyphenyl)-1-oxo-3-(thiophen-2-yl)-1,2,3,4-tetrahydroisoquinoline-4-carboxamide,
2-(((S)-1-ethylpyrrolidin-2-yl)methyl)-N-(3-methoxyphenyl)-1-oxo-3-(thiophen-2-yl)-1,2,3,4-tetrahydroisoquinoline-4-carboxamide,
6,7-dimethoxy-2-(2-methoxyethyl)-N-(3-methoxyphenyl)-1-oxo-3-(thiophen-2-yl)-1,2,3,4-tetrahydroisoquinoline-4-carboxamide,
2-(2-methoxyethyl)-N-(naphthalen-1-ylmethyl)-1-oxo-3-(thiophen-2-yl)-1,2,3,4-tetrahydroisoquinoline-4-carboxamide,
N-(4-methoxybenzyl)-2-(2-methoxyethyl)-1-oxo-3-(thiophen-2-yl)-1,2,3,4-tetrahydroisoquinoline-4-carboxamide,
2-(2-methoxyethyl)-1-oxo-N-(pyridin-4-ylmethyl)-3-(thiophen-2-yl)-1,2,3,4-tetrahydroisoquinoline-4-carboxamide,
N-(benzo[d]thiazol-5-yl)-2-(2-methoxyethyl)-1-oxo-3-(thiophen-2-yl)-1,2,3,4-tetrahydroisoquinoline-4-carboxamide,
N-(biphenyl-2-yl)-2-(2-methoxyethyl)-1-oxo-3-(thiophen-2-yl)-1,2,3,4-tetrahydroisoquinoline-4-carboxamide,
2-(2-methoxyethyl)-1-oxo-3-(thiophen-2-yl)-N-m-tolyl-1,2,3,4-tetrahydroisoquinoline-4-carboxamide,
2-(2-methoxyethyl)-1-oxo-3-(thiophen-2-yl)-N-p-tolyl-1,2,3,4-tetrahydroisoquinoline-4-carboxamide,
N-(3,4-dichlorophenyl)-2-(2-methoxyethyl)-1-oxo-3-(thiophen-2-yl)-1,2,3,4-tetrahydroisoquinoline-4-carboxamide,
N-(3,4-difluorophenyl)-2-(2-methoxyethyl)-1-oxo-3-(thiophen-2-yl)-1,2,3,4-tetrahydroisoquinoline-4-carboxamide,
N-(3-benzylphenyl)-2-(2-methoxyethyl)-1-oxo-3-(thiophen-2-yl)-1,2,3,4-tetrahydroisoquinoline-4-carboxamide,
N-(4-benzylphenyl)-2-(2-methoxyethyl)-1-oxo-3-(thiophen-2-yl)-1,2,3,4-tetrahydroisoquinoline-4-carboxamide,
N-(3-chloro-4-fluorophenyl)-2-(2-methoxyethyl)-1-oxo-3-(thiophen-2-yl)-1,2,3,4-tetrahydroisoquinoline-4-carboxamide,
N-(4-fluorophenyl)-2-(2-methoxyethyl)-1-oxo-3-(thiophen-2-yl)-1,2,3,4-tetrahydroisoquinoline-4-carboxamide,
2-(2-methoxyethyl)-1-oxo-N-(4-(piperidin-1-yl)phenyl)-3-(thiophen-2-yl)-1,2,3,4-tetrahydroisoquinoline-4-carboxamide,
N-(3-acetamidophenyl)-2-(2-methoxyethyl)-1-oxo-3-(thiophen-2-yl)-1,2,3,4-tetrahydroisoquinoline-4-carboxamide,
N-(1,3-dimethyl-1H-pyrazol-5-yl)-2-(2-methoxyethyl)-1-oxo-3-(thiophen-2-yl)-1,2,3,4-tetrahydroisoquinoline-4-carboxamide,
2-(2-methoxyethyl)-1-oxo-3-(thiophen-2-yl)-N-(3-(trifluoromethyl)phenyl)-1,2,3,4-tetrahydroisoquinoline-4 carboxamide,
N-(4-chloro-3-methoxyphenyl)-2-(2-methoxyethyl)-1-oxo-3-(thiophen-2-yl)-1,2,3,4-tetrahydroisoquinoline-4-carboxamide,
2-(2-methoxyethyl)-1-oxo-3-(thiophen-2-yl)-N-(3-(trifluoromethoxy)phenyl)-1,2,3,4-tetrahydroisoquinoline-4-carboxamide,
2-(2-methoxyethyl)-1-oxo-3-(thiophen-2-yl)-N-(4-(trifluoromethoxy)phenyl)-1,2,3,4-tetrahydroisoquinoline-4-carboxamide,
methyl 2-methoxy-4-(2-(2-methoxyethyl)-1-oxo-3-(thiophen-2-yl)-1,2,3,4-tetrahydroisoquinoline-4-carboxamido)benzoate,
N-(3-(1H-pyrrol-1-yl)phenyl)-2-(2-methoxyethyl)-1-oxo-3-(thiophen-2-yl)-1,2,3,4-tetrahydroisoquinoline-4-carboxamide,
N-(4-(1H-pyrrol-1-yl)phenyl)-2-(2-methoxyethyl)-1-oxo-3-(thiophen-2-yl)-1,2,3,4-tetrahydroisoquinoline-4-carboxamide,
N-(2,3-dihydrobenzo[b][1,4]dioxin-6-yl)-2-(2-methoxyethyl)-1-oxo-3-(thiophen-2-yl)-1,2,3,4-tetrahydroisoquinoline-4-carboxamide,
2-(2-methoxyethyl)-N-(4-morpholinophenyl)-1-oxo-3-(thiophen-2-yl)-1,2,3,4-tetrahydroisoquinoline-4-carboxamide,
2-(2-methoxyethyl)-1-oxo-N-(3-(pyrrolidin-1-yl)phenyl)-3-(thiophen-2-yl)-1,2,3,4-tetrahydroisoquinoline-4-carboxamide,
2-(2-methoxyethyl)-N-(1-methyl-1H-indol-5-yl)-1-oxo-3-(thiophen-2-yl)-1,2,3,4-tetrahydroisoquinoline-4-carboxamide,
2-(2-methoxyethyl)-N-((1R,4Rr)-4-methylcyclohexyl)-1-oxo-3-(thiophen-2-yl)-1,2,3,4-tetrahydroisoquinoline-4-carboxamide,
N-(5-ethyl-1,3,4-oxadiazol-2-yl)-2-(2-methoxyethyl)-1-oxo-3-(thiophen-2-yl)-1,2,3,4-tetrahydroisoquinoline-4-carboxamide,
2-(2-methoxyethyl)-N-(5-methylisoxazol-3-yl)-1-oxo-3-(thiophen-2-yl)-1,2,3,4-tetrahydroisoquinoline-4-carboxamide,
2-(2-methoxyethyl)-N-(3-methylisoxazol-5-yl)-1-oxo-3-(thiophen-2-yl)-1,2,3,4-tetrahydroisoquinoline-4-carboxamide,
N-(4-acetamidophenyl)-2-(2-methoxyethyl)-1-oxo-3-(thiophen-2-yl)-1,2,3,4-tetrahydroisoquinoline-4-carboxamide,
N-(3,5-dimethoxyphenyl)-2-(2-methoxyethyl)-1-oxo-3-(thiophen-2-yl)-1,2,3,4-tetrahydroisoquinoline-4-carboxamide,
2-(2-methoxyethyl)-N-(2-methyl-1H-indol-5-yl)-1-oxo-3-(thiophen-2-yl)-1,2,3,4-tetrahydroisoquinoline-4-carboxamide,
N-(1H-indazol-5-yl)-2-(2-methoxyethyl)-1-oxo-3-(thiophen-2-yl)-1,2,3,4-tetrahydroisoquinoline-4-carboxamide,
2-(2-methoxyethyl)-N,3-bis(3-methoxyphenyl)-1-oxo-1,2,3,4-tetrahydroisoquinoline-4-carboxamide,
2-(2-methoxyethyl)-3-(2-methoxyphenyl)-N-(3-methoxyphenyl)-1-oxo-1,2,3,4-tetrahydroisoquinoline-4 carboxamide,
ethyl 1-(2-(2-methoxyethyl)-1-oxo-3-(thiophen-2-yl)-1,2,3,4-tetrahydroiso-quinoline-4-carbonyl)piperidine-4-carboxylate,
N-(5-tert-butylisoxazol-3-yl)-2-(2-methoxyethyl)-1-oxo-3-(thiophen-2-yl)-1,2,3,4-tetrahydroisoquinoline-4-carboxamide,
5,8-difluoro-2-(2-methoxyethyl)-N-(3-methoxyphenyl)-1-oxo-3-(thiophen-2-yl)-1,2,3,4-tetrahydroisoquinoline-4-carboxamide,
8-fluoro-7-methoxy-2-(2-methoxyethyl)-N-(3-methoxyphenyl)-1-oxo-3-(thiophen-2-yl)-1,2,3,4-tetrahydroisoquinoline-4-carboxamide,
2-(2-methoxyethyl)-N-(3-methoxyphenyl)-1-oxo-3-(thiophen-2-yl)-7-(trifluoromethyl)-1,2,3,4-tetrahydroisoquinoline-4-carboxamide,
8-fluoro-2-(2-methoxyethyl)-N-(3-methoxyphenyl)-7-methyl-1-oxo-3-(thiophen-2-yl)-1,2,3,4-tetrahydroisoquinoline-4-carboxamide,
5-methoxy-2-(2-methoxyethyl)-N-(3-methoxyphenyl)-1-oxo-3-(thiophen-2-yl)-1,2,3,4-tetrahydroisoquinoline-4-carboxamide,
3-(2,4-dimethylthiazol-5-yl)-2-(2-methoxyethyl)-N-(3-methoxyphenyl)-1-oxo-1,2,3,4-tetrahydroisoquinoline-4-carboxamide,
2-(2-methoxyethyl)-N-(3-methoxyphenyl)-1-oxo-3-((tetrahydro-2H-pyran-4-yl)methyl)-1,2,3,4-tetrahydroisoquinoline-4-carboxamide,
2-(2-methoxyethyl)-N-(3-methoxyphenyl)-3-(2-methylthiazol-4-yl)-1-oxo-1,2,3,4-tetrahydroisoquinoline-4-carboxamide,
3-(3,5-dimethylisoxazol-4-yl)-2-(2-methoxyethyl)-N-(3-methoxyphenyl)-1-oxo-1,2,3,4-tetrahydroisoquinoline-4-carboxamide,
3-(1,3-dimethyl-1H-pyrazol-5-yl)-2-(2-methoxyethyl)-N-(3-methoxyphenyl)-1-oxo-1,2,3,4-tetrahydroisoquinoline-4-carboxamide,
3-(1,5-dimethyl-1H-pyrazol-3-yl)-2-(2-methoxyethyl)-N-(3-methoxyphenyl)-1-oxo-1,2,3,4-tetrahydroisoquinoline-4-carboxamide,
2-(2-methoxyethyl)-N-(3-methoxyphenyl)-1-oxo-3-(thiazol-2-yl)-1,2,3,4-tetrahydroisoquinoline-4-carboxamide,
2-(2-methoxyethyl)-N-(3-methoxyphenyl)-3-(3-methylisoxazol-5-yl)-1-oxo-1,2,3,4-tetrahydroisoquinoline-4-carboxamide,
2-(2-methoxyethyl)-N-(3-methoxyphenyl)-3-(1-methyl-1H-pyrrol-2-yl)-1-oxo-1,2,3,4-tetrahydroisoquinoline-4-carboxamide,
2-(2-methoxyethyl)-N-(3-methoxyphenyl)-1-oxo-3-(phenylethynyl)-1,2,3,4-tetrahydroisoquinoline-4-carboxamide,
3-(5-chlorothiophen-2-yl)-2-(2-methoxyethyl)-N-(3-methoxyphenyl)-1-oxo-1,2,3,4-tetrahydroisoquinoline-4-carboxamide,
3-(benzyloxymethyl)-2-(2-methoxyethyl)-N-(3-methoxyphenyl)-1-oxo-1,2,3,4-tetrahydroisoquinoline-4-carboxamide,
3-(hydroxymethyl)-2-(2-methoxyethyl)-N-(3-methoxyphenyl)-1-oxo-1,2,3,4-tetrahydroisoquinoline-4-carboxamide,
3-cyano-2-(2-methoxyethyl)-N-(3-methoxyphenyl)-1-oxo-1,2,3,4-tetrahydroisoquinoline-4-carboxamide,
2-(2-methoxyethyl)-N4-(3-methoxyphenyl)-1-oxo-1,2,3,4-tetrahydroisoquinoline-3,4-dicarboxamide,
2-(2-methoxyethyl)-N-(4-methyloxazol-2-yl)-1-oxo-3-(thiophen-2-yl)-1,2,3,4-tetrahydroisoquinoline-4-carboxamide,
N-(4-cyano-3-methylisoxazol-5-yl)-2-(2-methoxyethyl)-1-oxo-3-(thiophen-2-yl)-1,2,3,4-tetrahydroisoquinoline-4-carboxamide,
N-(4,5-dimethylthiazol-2-yl)-2-(2-methoxyethyl)-1-oxo-3-(thiophen-2-yl)-1,2,3,4-tetrahydroisoquinoline-4-carboxamide,
2-(2-methoxyethyl)-N-(4-(2-methylthiazol-4-yl)phenyl)-1-oxo-3-(thiophen-2-yl)-1,2,3,4-tetrahydroisoquinoline-4-carboxamide,
2-(2-methoxyethyl)-1-oxo-N-(6-phenoxypyridin-3-yl)-3-(thiophen-2-yl)-1,2,3,4-tetrahydroisoquinoline-4-carboxamide,
N-(4-(furan-2-yl)phenyl)-2-(2-methoxyethyl)-1-oxo-3-(thiophen-2-yl)-1,2,3,4-tetrahydroisoquinoline-4-carboxamide,
N-(5-(4-fluorophenyl)isoxazol-3-yl)-2-(2-methoxyethyl)-1-oxo-3-(thiophen-2-yl)-1,2,3,4-tetrahydroisoquinoline-4-carboxamide,
2-(2-methoxyethyl)-1-oxo-N-(1H-pyrazol-3-yl)-3-(thiophen-2-yl)-1,2,3,4-tetrahydroisoquinoline-4-carboxamide,
N-(5-tert-butyl-1H-pyrazol-3-yl)-2-(2-methoxyethyl)-1-oxo-3-(thiophen-2-yl)-1,2,3,4-tetrahydroisoquinoline-4-carboxamide,
2-(2-methoxyethyl)-1-oxo-N-(6-phenylpyridin-3-yl)-3-(thiophen-2-yl)-1,2,3,4-tetrahydroisoquinoline-4-carboxamide,
2-(2-methoxyethyl)-N-(4-(5-methyl-1,2,4-oxadiazol-3-yl)phenyl)-1-oxo-3-(thiophen-2-yl)-1,2,3,4-tetrahydroisoquinoline-4-carboxamide,
2-(2-methoxyethyl)-N-(4-(morpholinomethyl)phenyl)-1-oxo-3-(thiophen-2-yl)-1,2,3,4-tetrahydroisoquinoline-4-carboxamide,
N-(1,3-dihydroisobenzofuran-5-yl)-2-(2-methoxyethyl)-1-oxo-3-(thiophen-2-yl)-1,2,3,4-tetrahydroisoquinoline-4-carboxamide,
2-(2-methoxyethyl)-N-(4-(6-methylpyrazin-2-yloxy)phenyl)-1-oxo-3-(thiophen-2-yl)-1,2,3,4-tetrahydroisoquinoline-4-carboxamide,
N-(6-chlorobenzo[d]thiazol-2-yl)-2-(2-methoxyethyl)-1-oxo-3-(thiophen-2-yl)-1,2,3,4-tetrahydroisoquinoline-4-carboxamide,
2-(2-methoxyethyl)-N-(4-(1-methyl-1H-pyrazol-3-yl)phenyl)-1-oxo-3-(thiophen-2-yl)-1,2,3,4-tetrahydroisoquinoline-4-carboxamide,
N-(1,3-dimethyl-1H-pyrazol-5-yl)-2-(2-methoxyethyl)-1-oxo-3-(thiophen-2-yl)-1,2,3,4-tetrahydroisoquinoline-4-carboxamide,
2-(2-methoxyethyl)-N-(4-(5-methyl-1,3,4-oxadiazol-2-yl)phenyl)-1-oxo-3-(thiophen-2-yl)-1,2,3,4-tetrahydroisoquinoline-4-carboxamide,
2-(2-methoxyethyl)-N-(1-methyl-2-oxoindolin-5-yl)-1-oxo-3-(thiophen-2-yl)-1,2,3,4-tetrahydroisoquinoline-4-carboxamide,
5-amino-3-(-2-(2-methoxyethyl)-1-oxo-3-(thiophen-2-yl)-1,2,3,4-tetrahydroisoquinoline-4-carbonyl)benzo[d]oxazol-2(3H)-one,
2-(2-methoxyethyl)-N-(1-methyl-3-phenyl-1H-pyrazol-5-yl)-1-oxo-3-(thiophen-2-yl)-1,2,3,4-tetrahydroisoquinoline-4-carboxamide,
Methyl 4-(2-(2-methoxyethyl)-1-oxo-3-(thiophen-2-yl)-1,2,3,4-tetrahydroiso-quinoline-4-carboxamido)-1-methyl-1H-pyrrole-2-carboxylate,
2-(2-methoxyethyl)-N-(3-(oxazol-5-yl)phenyl)-1-oxo-3-(thiophen-2-yl)-1,2,3,4-tetrahydroisoquinoline-4-carboxamide,
2-(2-methoxyethyl)-N-(3-methoxyphenyl)-N-methyl-1-oxo-3-(thiophen-2-yl)-1,2,3,4-tetrahydroisoquinoline-4-carboxamide,
N-(4-(1H-pyrrol-1-yl)phenyl)-8-fluoro-7-methoxy-2-(2-methoxyethyl)-1-oxo-3-(thiophen-2-yl)-1,2,3,4-tetrahydroisoquinoline-4-carboxamide,
N-(4-(1H-pyrrol-1-yl)phenyl)-3-(5-chlorothiophen-2-yl)-2-(2-methoxyethyl)-1-oxo-1,2,3,4-tetrahydroisoquinoline-4-carboxamide,
N-(3-methoxyphenyl)-1-oxo-3-(thiophen-2-yl)-1,2,3,4-tetrahydroisoquinoline-4-carboxamide,
2-(2-methoxyethyl)-1-oxo-N-(pyrimidin-4-yl)-3-(thiophen-2-yl)-1,2,3,4-tetrahydroisoquinoline-4-carboxamide,
2-(2-methoxyethyl)-1-oxo-N-(pyrazin-2-yl)-3-(thiophen-2-yl)-1,2,3,4-tetrahydroisoquinoline-4-carboxamide,
N-(benzo[d]thiazol-5-yl)-2-(2-methoxyethyl)-1-oxo-3-(thiophen-2-yl)-1,2,3,4-tetrahydroisoquinoline-4-carboxamide,
N-(4-isopropylphenyl)-2-(2-methoxyethyl)-1-oxo-3-(thiophen-2-yl)-1,2,3,4-tetrahydroisoquinoline-4-carboxamide,
N-(3-(hydroxymethyl)phenyl)-2-(2-methoxyethyl)-1-oxo-3-(thiophen-2-yl)-1,2,3,4-tetrahydroisoquinoline-4-carboxamide,
N-(4-(1H-1,2,4-triazol-1-yl)phenyl)-2-(2-methoxyethyl)-1-oxo-3-(thiophen-2-yl)-1,2,3,4-tetrahydroisoquinoline-4-carboxamide,
2-(2-methoxyethyl)-N-(2-methylquinolin-6-yl)-1-oxo-3-(thiophen-2-yl)-1,2,3,4-tetrahydroisoquinoline-4-carboxamide,
N-(3,5-dimethylisoxazol-4-yl)-2-(2-methoxyethyl)-1-oxo-3-(thiophen-2-yl)-1,2,3,4-tetrahydroisoquinoline-4-carboxamide,
N-(benzo[d]thiazol-6-yl)-2-(2-methoxyethyl)-1-oxo-3-(thiophen-2-yl)-1,2,3,4-tetrahydroisoquinoline-4-carboxamide,
2-(2-methoxyethyl)-1-oxo-N-(quinoxalin-6-yl)-3-(thiophen-2-yl)-1,2,3,4-tetrahydroisoquinoline-4-carboxamide,
N-(3-(1H-pyrazol-1-yl)phenyl)-2-(2-methoxyethyl)-1-oxo-3-(thiophen-2-yl)-1,2,3,4-tetrahydroisoquinoline-4-carboxamide,
N-(4-(1H-pyrazol-1-yl)phenyl)-2-(2-methoxyethyl)-1-oxo-3-(thiophen-2-yl)-1,2,3,4-tetrahydroisoquinoline-4-carboxamide,
2-(2-methoxyethyl)-1-oxo-N-(4-(pyridin-3-yl)phenyl)-3-(thiophen-2-yl)-1,2,3,4-tetrahydroisoquinoline-4-carboxamide,
N-(4-((1H-pyrazol-1-yl)methyl)phenyl)-2-(2-methoxyethyl)-1-oxo-3-(thiophen-2-yl)-1,2,3,4-tetrahydroisoquinoline-4-carboxamide,
2-(2-methoxyethyl)-1-oxo-N-(4-(pyrimidin-2-yl)phenyl)-3-(thiophen-2-yl)-1,2,3,4-tetrahydroisoquinoline-4-carboxamide,
2-(2-methoxyethyl)-N-(1-methylindolin-5-yl)-1-oxo-3-(thiophen-2-yl)-1,2,3,4-tetrahydroisoquinoline-4-carboxamide,
N-(4-tert-butylthiazol-2-yl)-2-(2-methoxyethyl)-1-oxo-3-(thiophen-2-yl)-1,2,3,4-tetrahydroisoquinoline-4-carboxamide,
2-(2-methoxyethyl)-N-(4-(oxazol-5-yl)phenyl)-1-oxo-3-(thiophen-2-yl)-1,2,3,4-tetrahydroisoquinoline-4-carboxamide,
2-(2-methoxyethyl)-1-oxo-3-(thiophen-2-yl)-N-(3-(trifluoromethyl)-1,2,4-thiadiazol-5-yl)-1,2,3,4-tetrahydroisoquinoline-4-carboxamide,
2-(2-methoxyethyl)-N-(1-methyl-1H-pyrazol-3-yl)-1-oxo-3-(thiophen-2-yl)-1,2,3,4-tetrahydroisoquinoline-4-carboxamide,
N-(4-(hydroxymethyl)phenyl)-2-(2-methoxyethyl)-1-oxo-3-(thiophen-2-yl)-1,2,3,4-tetrahydroisoquinoline-4-carboxamide,
2-(2-methoxyethyl)-1-oxo-N-(5-phenyl-1H-pyrazol-3-yl)-3-(thiophen-2-yl)-1,2,3,4-tetrahydroisoquinoline-4-carboxamide,
N-(5-(furan-2-yl)-1H-pyrazol-3-yl)-2-(2-methoxyethyl)-1-oxo-3-(thiophen-2-yl)-1,2,3,4-tetrahydroisoquinoline-4-carboxamide,
2-(2-methoxyethyl)-1-oxo-N-(4-phenoxyphenyl)-3-(thiophen-2-yl)-1,2,3,4-tetrahydroisoquinoline-4-carboxamide,
ethyl 5-(-2-(2-methoxyethyl)-1-oxo-3-(thiophen-2-yl)-1,2,3,4-tetrahydroiso-quinoline-4-carboxamido)-1,3,4-oxadiazole-2-carboxylate,
2-(2-methoxyethyl)-1-oxo-N-(3-phenyl-1,2,4-thiadiazol-5-yl)-3-(thiophen-2-yl)-1,2,3,4-tetrahydroisoquinoline-4-carboxamide,
N-(benzofuran-5-yl)-2-(2-methoxyethyl)-1-oxo-3-(thiophen-2-yl)-1,2,3,4-tetrahydroisoquinoline-4-carboxamide,
2-(2-methoxyethyl)-1-oxo-N-(5-methyl-1H-pyrazol-3-yl)-3-(thiophen-2-yl)-1,2,3,4-tetrahydroisoquinoline-4-carboxamide,
N-(3-cyclopropyl-1-methyl-1H-pyrazol-5-yl)-2-(2-methoxyethyl)-1-oxo-3-(thiophen-2-yl)-1,2,3,4-tetrahydroisoquinoline-4-carboxamide,
N-(1,1-dioxobenzo[b]thiophen-6-yl)-2-(2-methoxyethyl)-1-oxo-3-(thiophen-2-yl)-1,2,3,4-tetrahydroisoquinoline-4-carboxamide,
N-(6-fluorobenzo[d]thiazol-2-yl)-2-(2-methoxyethyl)-1-oxo-3-(thiophen-2-yl)-1,2,3,4-tetrahydroisoquinoline-4-carboxamide,
N-(3-(1H-1,2,4-triazol-1-yl)phenyl)-2-(2-methoxyethyl)-1-oxo-3-(thiophen-2-yl)-1,2,3,4-tetrahydroisoquinoline-4-carboxamide,
N-(4-(3,5-dimethyl-1H-pyrazol-1-yl)phenyl)-2-(2-methoxyethyl)-1-oxo-3-(thiophen-2-yl)-1,2,3,4-tetrahydroisoquinoline-4-carboxamide,
2-(2-methoxyethyl)-N-(4-(2-methyl-1H-imidazol-1-yl)phenyl)-1-oxo-3-(thiophen-2-yl)-1,2,3,4-tetrahydroisoquinoline-4-carboxamide,
N-(3-((1H-imidazol-1-yl)methyl)phenyl)-2-(2-methoxyethyl)-1-oxo-3-(thiophen-2-yl)-1,2,3,4-tetrahydroisoquinoline-4-carboxamide,
2-(2-methoxyethyl)-N-(4-methyl-3-(methylsulfonamido)phenyl)-1-oxo-3-(thiophen-2-yl)-1,2,3,4-tetrahydroisoquinoline-4-carboxamide,
2-(2-methoxyethyl)-N-(4-(4-methyl-4H-1,2,4-triazol-3-yl)phenyl)-1-oxo-3-(thiophen-2-yl)-1,2,3,4-tetrahydroisoquinoline-4-carboxamide,
N-(4-((1H-imidazol-1-yl)methyl)phenyl)-2-(2-methoxyethyl)-1-oxo-3-(thiophen-2-yl)-1,2,3,4-tetrahydroisoquinoline-4-carboxamide,
2-(2-methoxyethyl)-1-oxo-N-(4-(piperidin-1-ylmethyl)phenyl)-3-(thiophen-2-yl)-1,2,3,4-tetrahydroisoquinoline-4-carboxamide,
N-(4-((1H-1,2,4-triazol-1-yl)methyl)phenyl)-2-(2-methoxyethyl)-1-oxo-3-(thiophen-2-yl)-1,2,3,4-tetrahydroisoquinoline-4-carboxamide,
2-(2-methoxyethyl)-1-oxo-N-(4-(tetrahydro-2H-pyran-4-yloxy)phenyl)-3-(thiophen-2-yl)-1,2,3,4-tetrahydroisoquinoline-4-carboxamide,
N-(benzo[d]oxazol-6-yl)-2-(2-methoxyethyl)-1-oxo-3-(thiophen-2-yl)-1,2,3,4-tetrahydroisoquinoline-4-carboxamide,
N-(3,4-dimethylisoxazol-5-yl)-2-(2-methoxyethyl)-1-oxo-3-(thiophen-2-yl)-1,2,3,4-tetrahydroisoquinoline-4-carboxamide,
3-(furan-2-yl)-2-(2-methoxyethyl)-N-(3-methoxyphenyl)-1-oxo-1,2,3,4-tetrahydroisoquinoline-4-carboxamide,
3-(2-fluorophenyl)-2-(2-methoxyethyl)-N-(3-methoxyphenyl)-1-oxo-1,2,3,4-tetrahydroisoquinoline-4-carboxamide,
3-(3-Fluorophenyl)-2-(2-methoxyethyl)-N-(3-methoxyphenyl)-1-oxo-1,2,3,4-tetrahydroisoquinoline-4-carboxamide,
3-(4-Fluorophenyl)-2-(2-methoxyethyl)-N-(3-methoxyphenyl)-1-oxo-1,2,3,4-tetrahydroisoquinoline-4-carboxamide,
3-(2,6-Difluorophenyl)-2-(2-methoxyethyl)-N-(3-methoxyphenyl)-1-oxo-1,2,3,4-tetrahydroisoquinoline-4-carboxamide,
2-(2-Methoxyethyl)-N-(3-methoxyphenyl)-1-oxo-3-(thiophen-3-yl)-1,2,3,4-tetrahydroisoquinoline-4-carboxamide,
2-(2-Methoxyethyl)-N-(3-methoxyphenyl)-3-(5-methylthiophen-2-yl)-1-oxo-1,2,3,4-tetrahydroisoquinoline-4-carboxamide,
3-(3-Chlorothiophen-2-yl)-2-(2-methoxyethyl)-N-(3-methoxyphenyl)-1-oxo-1,2,3,4-tetrahydroisoquinoline-4-carboxamide,
2-(2-Methoxyethyl)-N-(3-methoxyphenyl)-1-oxo-3-(pyridin-2-yl)-1,2,3,4-tetrahydroisoquinoline-4-carboxamide,
2-(2-Methoxyethyl)-N-(3-methoxyphenyl)-1-oxo-3-(pyridin-4-yl)-1,2,3,4-tetrahydroisoquinoline-4-carboxamide,
2-(2-Methoxyethyl)-N-(3-methoxyphenyl)-1-oxo-3-phenyl-1,2,3,4-tetrahydroisoquinoline-4-carboxamide,
2-(2-Methoxyethyl)-N-(3-methoxyphenyl)-1-oxo-3-(1H-pyrrol-2-yl)-1,2,3,4-tetrahydroisoquinoline-4-carboxamide,
3-(1H-imidazol-2-yl)-2-(2-methoxyethyl)-N-(3-methoxyphenyl)-1-oxo-1,2,3,4-tetrahydroisoquinoline-4-carboxamide,
2-(2-Methoxyethyl)-N-(3-methoxyphenyl)-1-oxo-3-(pyridin-3-yl)-1,2,3,4-tetrahydroisoquinoline-4-carboxamide,
2-(2-Methoxyethyl)-N-(3-methoxyphenyl)-3-(4-methyl-1H-imidazol-5-yl)-1-oxo-1,2,3,4-tetrahydroisoquinoline-4-carboxamide,
3-(Benzo[b]thiophen-2-yl)-2-(2-methoxyethyl)-N-(3-methoxyphenyl)-1-oxo-1,2,3,4-tetrahydroisoquinoline-4-carboxamide,
3-tert-Butyl-2-(2-methoxyethyl)-N-(3-methoxyphenyl)-1-oxo-1,2,3,4-tetrahydroisoquinoline-4-carboxamide,
3-Ethyl-2-(2-methoxyethyl)-N-(3-methoxyphenyl)-1-oxo-1,2,3,4-tetrahydroisoquinoline-4-carboxamide,
3-Benzyl-2-(2-methoxyethyl)-N-(3-methoxyphenyl)-1-oxo-1,2,3,4-tetrahydroisoquinoline-4-carboxamide,
2-(2-methoxyethyl)-N-(3-methoxyphenyl)-3-methyl-1-oxo-1,2,3,4-tetrahydroisoquinoline-4-carboxamide,
2-(2-methoxyethyl)-N-(3-methoxyphenyl)-1-oxo-3-((trimethylsilyl)ethynyl)-1,2,3,4-tetrahydroisoquinoline-4-carboxamide,
3-ethynyl-2-(2-methoxyethyl)-N-(3-methoxyphenyl)-1-oxo-1,2,3,4-tetrahydroisoquinoline-4-carboxamide,
2-(2-methoxyethyl)-N-(3-methoxyphenyl)-3-(5-methylisoxazol-3-yl)-1-oxo-1,2,3,4-tetrahydroisoquinoline-4-carboxamide,
2-(2-methoxyethyl)-N-(3-methoxyphenyl)-3-(1-methyl-1H-pyrazol-5-yl)-1-oxo-1,2,3,4-tetrahydroisoquinoline-4-carboxamide,
3-cyclopentyl-2-(2-methoxyethyl)-N-(3-methoxyphenyl)-1-oxo-1,2,3,4-tetrahydroisoquinoline-4-carboxamide,
2-(2-methoxyethyl)-N-(3-methoxyphenyl)-3-(2-methylprop-1-enyl)-1-oxo-1,2,3,4-tetrahydroisoquinoline-4-carboxamide,
2-(2-methoxyethyl)-N-(3-methoxyphenyl)-1-oxo-3-(2,2,2-trifluoroethyl)-1,2,3,4-tetrahydroisoquinoline-4-carboxamide,
2-(2-methoxyethyl)-N-(3-methoxyphenyl)-3-(methylthiomethyl)-1-oxo-1,2,3,4-tetrahydroisoquinoline-4-carboxamide,
2-(2-methoxyethyl)-N-(3-methoxyphenyl)-3-(methylsulfonylmethyl)-1-oxo-1,2,3,4-tetrahydroisoquinoline-4-carboxamide,
N-(4-(benzylcarbamoyl)phenyl)-2-(2-methoxyethyl)-1-oxo-3-(thiophen-2-yl)-1,2,3,4-tetrahydroisoquinoline-4-carboxamide,
N-(4-(dimethylcarbamoyl)phenyl)-2-(2-methoxyethyl)-1-oxo-3-(thiophen-2-yl)-1,2,3,4-tetrahydroisoquinoline-4-carboxamide,
2-(2-methoxyethyl)-N-(4-(methylcarbamoyl)phenyl)-1-oxo-3-(thiophen-2-yl)-1,2,3,4-tetrahydroisoquinoline-4-carboxamide,
7-amino-2-(2-methoxyethyl)-N-(3-methoxyphenyl)-1-oxo-3-(thiophen-2-yl)-1,2,3,4-tetrahydroisoquinoline-4-carboxamide,
2-(2-methoxyethyl)-4-((3-methoxyphenylamino)methyl)-3-(thiophen-2-yl)isoquinolin-1(2H)-one,
2-(2-methoxyethyl)-1-oxo-3-(thiophen-2-yl)-1,2,3,4-tetrahydroisoquinoline-4-carboxylic acid,
2-(2-methoxyethyl)-N-(3-methoxyphenyl)-1-oxo-3-(thiophen-2-yl)-1,2-dihydroisoquinoline-4-carboxamide,
3-(furan-2-yl)-2-(2-methoxyethyl)-N-(3-methoxyphenyl)-1-oxo-1,2-dihydroisoquinoline-4-carboxamide,
4-(hydroxymethyl)-2-(2-methoxyethyl)-3-(thiophen-2-yl)-3,4-dihydroisoquinolin-1(2H)-one,
4-(benzyloxymethyl)-2-(2-methoxyethyl)-3-(thiophen-2-yl)-3,4-dihydroisoquinolin-1 (2H)-one,
6-fluoro-2-(2-methoxyethyl)-N-(3-methoxyphenyl)-1-oxo-3-(thiophen-2-yl)-1,2,3,4-tetrahydroisoquinoline-4-carboxamide,
2-(2-methoxyethyl)-4-((3-methoxyphenylamino)methyl)-3-(thiophen-2-yl)-3,4-dihydroisoquinolin-1(2H)-one,
4-((4-chlorophenylamino)methyl)-2-(2-methoxyethyl)-3-(thiophen-2-yl)-3,4-dihydroisoquinolin-1(2H)-one,
N-(4-cyano-3-(trifluoromethyl)phenyl)-2-(2-methoxyethyl)-1-oxo-3-(thiophen-2-yl)-1,2,3,4-tetrahydroisoquinoline-4-carboxamide,
N-cyclohexyl-2-(2-methoxyethyl)-1-oxo-3-(thiophen-2-yl)-1,2,3,4-tetrahydroisoquinoline-4-carboxamide,
2-(2-methoxyethyl)-4-(4-methylpiperidine-1-carbonyl)-3-(thiophen-2-yl)-3,4-dihydroisoquinolin-1(2H)-one,
2-(2-methoxyethyl)-N-(3-(4-methylpiperidin-1-yl)propyl)-1-oxo-3-(thiophen-2-yl)-1,2,3,4-tetrahydroisoquinoline-4-carboxamide,
N-(2,5-dimethylphenyl)-N-ethyl-2-(2-methoxyethyl)-1-oxo-3-(thiophen-2-yl)-1,2,3,4-tetrahydroisoquinoline-4-carboxamide,
2-(2-methoxyethyl)-1-oxo-N-(pyridin-3-yl)-3-(thiophen-2-yl)-1,2,3,4-tetrahydroisoquinoline-4-carboxamide,
2-cyclohexyl-N-(3-methoxyphenyl)-3-(4-methoxyphenyl)-1-oxo-1,2,3,4-tetrahydroisoquinoline-4-carboxamide,
3-(4-fluorophenyl)-N-(6-methoxybenzo[d]thiazol-2-yl)-2-(2-methoxyethyl)-1-oxo-1,2,3,4-tetrahydroisoquinoline-4-carboxamide,
3-(4-fluorophenyl)-2-(2-methoxyethyl)-1-oxo-N-(quinolin-3-yl)-1,2,3,4-tetrahydroisoquinoline-4-carboxamide,
3-(4-fluorophenyl)-N-(furan-2-ylmethyl)-2-(2-methoxyethyl)-1-oxo-1,2,3,4-tetrahydroisoquinoline-4-carboxamide,
N-((1-ethylpyrrolidin-2-yl)methyl)-3-(4-fluorophenyl)-2-(2-methoxyethyl)-1-oxo-1,2,3,4-tetrahydroisoquinoline-4-carboxamide,
3-(4-fluorophenyl)-2-(2-methoxyethyl)-1-oxo-N-(pyridin-2-ylmethyl)-1,2,3,4-tetrahydroisoquinoline-4-carboxamide,
3-(3,4-dimethoxyphenyl)-N-(3-methoxyphenyl)-2-methyl-1-oxo-1,2,3,4-tetrahydroisoquinoline-4-carboxamide,
2-(4-fluorobenzyl)-N-(3-methoxyphenyl)-3-(4-methoxyphenyl)-1-oxo-1,2,3,4-tetrahydroisoquinoline-4-carboxamide,
3-(3,4-dimethoxyphenyl)-6,7-dimethoxy-N-(3-methoxyphenyl)-2-methyl-1-oxo-1,2,3,4-tetrahydroisoquinoline-4-carboxamide,
6,7-dimethoxy-N-(3-methoxyphenyl)-3-(4-methoxyphenyl)-2-methyl-1-oxo-1,2,3,4-tetrahydroisoquinoline-4-carboxamide,
6,7-dimethoxy-N-(3-methoxyphenyl)-2-methyl-1-oxo-3-(thiophen-2-yl)-1,2,3,4-tetrahydroisoquinoline-4-carboxamide,
3-(2-fluorophenyl)-6,7-dimethoxy-N-(3-methoxyphenyl)-2-methyl-1-oxo-1,2,3,4-tetrahydroisoquinoline-4-carboxamide,
N-(3-methoxyphenyl)-2-methyl-1-oxo-3-(thiophen-2-yl)-1,2,3,4-tetrahydroisoquinoline-4-carboxamide,
2-(2-methoxyethyl)-1-oxo-N-((tetrahydrofuran-2-yl)methyl)-3-(thiophen-2-yl)-1,2,3,4-tetrahydroisoquinoline-4-carboxamide,
N,2-bis(2-methoxyethyl)-1-oxo-3-(thiophen-2-yl)-1,2,3,4-tetrahydroiso-quinoline-4-carboxamide,
N-(furan-2-ylmethyl)-2-(2-methoxyethyl)-1-oxo-3-(thiophen-2-yl)-1,2,3,4-tetrahydroisoquinoline-4-carboxamide,
N-((1-ethylpyrrolidin-2-yl)methyl)-2-(2-methoxyethyl)-1-oxo-3-(thiophen-2-yl)-1,2,3,4-tetrahydroisoquinoline-4-carboxamide,
2-(2-methoxyethyl)-1-oxo-N-(pyridin-2-ylmethyl)-3-(thiophen-2-yl)-1,2,3,4-tetrahydroisoquinoline-4-carboxamide,
N-(2-chloropyridin-3-yl)-2-(2-methoxyethyl)-1-oxo-3-(thiophen-2-yl)-1,2,3,4-tetrahydroisoquinoline-4-carboxamide,
N-(2-carbamoylphenyl)-2-(2-methoxyethyl)-1-oxo-3-(thiophen-2-yl)-1,2,3,4-tetrahydroisoquinoline-4-carboxamide,
2-(2-methoxyethyl)-4-(4-(3-methoxyphenyl)piperazine-1-carbonyl)-3-(thiophen-2-yl)-3,4-dihydroisoquinolin-1(2H)-one,
N-(3-acetylphenyl)-2-(2-methoxyethyl)-1-oxo-3-(thiophen-2-yl)-1,2,3,4-tetra-hydroisoquinoline-4-carboxamide,
N-benzyl-2-(2-methoxyethyl)-1-oxo-3-(thiophen-2-yl)-1,2,3,4-tetrahydro-isoquinoline-4-carboxamide,
2-isobutyl-N-(3-methoxyphenyl)-1-oxo-3-(thiophen-2-yl)-1,2,3,4-tetrahydroiso-quinoline-4-carboxamide,
2-cyclopentyl-N-(3-methoxyphenyl)-1-oxo-3-(thiophen-2-yl)-1,2,3,4-tetra-hydroisoquinoline-4-carboxamide,
N-(3-cyano-4-(1H-pyrrol-1-yl)phenyl)-2-(2-methoxyethyl)-1-oxo-3-(thiophen-2-yl)-1,2,3,4-tetrahydroisoquinoline-4-carboxamide, and
N-(5-chloropyridin-2-yl)-2-(2-methoxyethyl)-1-oxo-3-(thiophen-2-yl)-1,2,3,4-tetrahydroisoquinoline-4-carboxamide.

47. The method of claim 34, wherein the RNA-virus is a Flavivirus.

48. The method of claim 34, wherein the RNA virus is the Hepatitis C virus.

49. A compound according to formula (II), 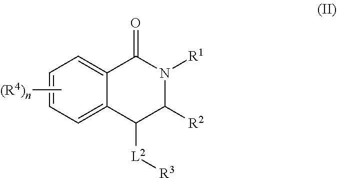
wherein
R¹ is C₁₋₁₈-alkyl, C₁₋₁₈-heteroalkyl or trialkylsilyl, which can be unsubstituted or substituted with one or more Z¹,
each of R² and R³ is independently selected from the group consisting of aryl, heterocycle, aryl-C₁₋₁₈-alkyl, aryl-C₂₋₁₈-alkenyl, aryl-C₂₋₁₈-alkynyl, heterocycle-C₁₋₁₈-alkyl, heterocycle-C₂₋₁₈-alkenyl, and heterocycle-C₂₋₁₈-alkynyl, wherein said aryl, heterocycle, aryl-C₁₋₁₈-alkyl, aryl-C₂₋₁₈-alkenyl, aryl-C₂₋₁₈-alkynyl, heterocycle-C₁₋₁₈-alkyl, heterocycle-C₂₋₁₈-alkenyl or heterocycle-C₂₋₁₈-alkynyl optionally includes one or more heteroatoms selected from the group consisting of O, S, P, and N, and wherein said aryl, heterocycle, aryl-C₁₋₁₈-alkyl, aryl-C₂₋₁₈-alkenyl, aryl-C₂₋₁₈-alkynyl, heterocycle-C₁₋₁₈-alkyl, heterocycle-C₂₋₁₈-alkenyl or heterocycle-C₂₋₁₈-alkynyl can be unsubstituted or substituted with one or more Z¹,
L² is selected from the group consisting of —CH₂NZ⁶—, CH₂NH—, —CH₂O—, —CO—NZ⁶—, and —CONH—,
each R⁴ is independently selected from the group consisting of halogen, —OH, C₁₋₁₈-alkoxy, —SH, C₁₋₁₈-thioalkoxy, trifluoromethyl, trifluoromethoxy, nitro, -cyano, —NHSO₂—C₁₋₆-alkyl, —COOH, —COO—C₁₋₆-alkyl, —COO—C₂₋₆-alkenyl, —COO—C₂₋₆-alkynyl, amino, C₁₋₁₈-heteroalkyl, C₁₋₁₈-alkyl, C₂₋₁₈-alkenyl, C₂₋₁₈-alkynyl, aryl, and heteroaryl,
n is 0, 1, 2, 3, or 4,
Z¹ is independently selected from the group consisting of halogen, hydroxyl, C₁₋₁₈-alkoxy, C₁₋₁₈-thioalkoxy, trifluoromethyl, trifluoromethoxy, nitro, amino, cyano, —NHSO₂—C₁₋₆-alkyl, —COOH, —COO—C₁₋₆-alkyl, —COO—C₂₋₆-alkenyl, —COO—C₂₋₆-alkynyl, C₁₋₁₈-alkyl, C₂₋₁₈-alkenyl, C₂₋₁₈-alkynyl, and aryl,
Z⁶ is independently selected from the group consisting of C₁₋₁₈-alkyl, C₁₋₁₈-heteroalkyl, and aryl,
and stereo-isomers, tautomers, hydrates or pharmaceutically acceptable salts thereof.

50. The compound according to claim 49, wherein said compound has a structure according to formula (IIIa) 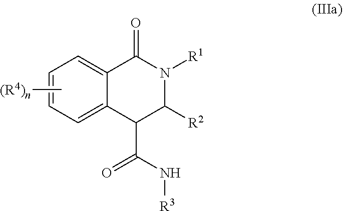
wherein —R¹ is a C₁₋₁₈-heteroalkyl group or a C₁₋₁₈-alkyl group optionally substituted with a halogen, hydroxyl, carboxy, —COO—C₁₋₆ alkyl, —COO—C₂₋₆ alkenyl, —COO—C₂₋₆alkynyl, aryl or an optionally alkyl group-substituted heterocyclic group,
R² is a C₁₋₁₈ alkyl, aryl or heteroaryl group optionally substituted with a halogen or C₁₋₁₈-alkyl group,
R³ is a C₁₋₁₈ alkyl, heterocyclic or aryl group optionally substituted with one or more Z¹,
R⁴ is a halogen atom, trifluoromethyl, trifluoromethoxy, —NHSO₂—C₁₋₆ alkyl, a C₁₋₁₈ alkyl group, a C₁₋₁₈ alkoxy group, a nitro group or an amino group,
n is 0, 1, 2, 3, or 4, and
each Z¹ is independently selected from the group consisting of halogen, hydroxyl, C₁₋₁₈ alkoxy, C₁₋₁₈ thioalkoxy, trifluoromethyl, nitro, amino, cyano, —NHSO₂—C₁₋₆alkyl, —COOH, —COO—C₁₋₆ alkyl, —COO—C₂₋₆ alkenyl, —COO—C₂₋₆ alkynyl, C₁₋₁₈ alkyl, C₂₋₁₈ alkenyl, C₂₋₁₈ alkynyl, and aryl,
and stereo-isomers, tautomers, hydrates and pharmaceutically acceptable salts thereof.

51. The compound according to claim 49, wherein said compound has a structure according to formula (IVa): 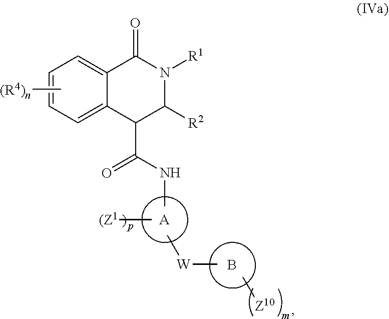
wherein
R¹ is selected from the group consisting of alkyl, alkenyl, alkynyl, aryl, heterocycle, arylalkyl, arylalkenyl, arylalkynyl, heterocycle-alkyl, heterocycle-alkenyl, and heterocycle-alkynyl, and wherein said alkyl, alkenyl, alkynyl, aryl, heterocycle, arylalkyl, arylalkenyl, arylalkynyl, heterocycle-alkyl, heterocycle-alkenyl or heterocycle-alkynyl optionally includes one or more heteroatoms selected from the group consisting of O, S, P, and N, and wherein said alkyl, alkenyl, alkynyl, aryl, heterocycle, arylalkyl, arylalkenyl, arylalkynyl, heterocycle-alkyl, heterocycle-alkenyl or heterocycle-alkynyl can be unsubstituted or substituted with one or more Z¹,
R² is selected from the group consisting of aryl, heterocycle, arylalkyl, arylalkenyl, arylalkynyl, heterocycle-alkyl, heterocycle-alkenyl, and heterocycle-alkynyl, and wherein said arylalkyl, arylalkenyl, arylalkynyl, heterocycle-alkyl, heterocycle-alkenyl or heterocycle-alkynyl optionally includes one or more heteroatoms selected from the group consisting of O, S, P, and N, and wherein said aryl, heterocycle, arylalkyl, arylalkenyl, arylalkynyl, heterocycle-alkyl, heterocycle-alkenyl or heterocycle-alkynyl can be unsubstituted or substituted with one or more Z¹,
each R⁴ is independently selected from the group consisting of halogen, —OH, alkoxy, —SH, thioalkoxy, trifluoromethyl, trifluoromethoxy, nitro, cyano, —COOH, —COO-alkyl, —COO-alkenyl, —COO-alkynyl, amino, heteroalkyl, alkyl, alkenyl, alkynyl, aryl, heterocycle, arylalkyl, arylalkenyl, arylalkynyl, heterocycle-alkyl, heterocycle-alkenyl, and heterocycle-alkynyl,
n is 0, 1, 2, 3, or 4,
each of cycle A and cycle B is independently selected from aryl or heterocycle,
“—W—” is a single bond, —O—, —S—, an alkylene group, a —C(═O)O— group, a —NH—C(═O)— group, a —C(═O)— group or a —C(═O)—NH— group, wherein said alkylene optionally includes one or more heteroatoms selected from the group consisting of O, S, P, and N,
each Z¹ and each Z¹⁰ are independently selected from the group consisting of halogen, hydroxyl, sulfhydryl, alkoxy, thioalkoxy, ═O, ═S, trifluoromethyl, trifluoromethoxy, nitro, amino, cyano, —COOH, —COO-alkyl, —COO-alkenyl, —COO-alkynyl, —CONH₂, alkyl, alkenyl, alkynyl, aryl, heterocycle, arylalkyl, arylalkenyl, arylalkynyl, heterocycle-alkyl, heterocycle-alkenyl, and heterocycle-alkynyl,
m is 0, 1, 2, 3, or 4,
n is 0, 1, 2, 3, or 4,
p is 0, 1, 2, or 3,
and stereo-isomers, tautomers, hydrates, and pharmaceutically acceptable salts thereof.

52. The compound according to claim 51 wherein:
R¹ is a C₁₋₁₈-heteroalkyl group or a C₁₋₁₈-alkyl group optionally substituted with an optionally C₁₋₆ alkyl group-substituted heterocyclic group,
R² is a C₁₋₁₈ alkyl, aryl or heteroaryl group optionally substituted with a halogen or C₁₋₁₈ alkyl group,
each of cycle A and cycle B is independently selected from aryl or heterocyclic groups,
—W— is a single bond, —O—, —S—, a C₁₋₆-alkylene group, a —C(═O)O— group, a NH—C(═O)— group, a —C(═O)— group or a —C(═O)—NH— group, wherein said C₁₋₆ alkylene optionally includes one or more heteroatoms selected from the group consisting of O, S, P, and N,
R⁴ is selected from the group consisting of halogen, C₁₋₁₈-alkyl, C₁₋₁₈ alkoxy, nitro, and amino,
Z¹ and Z¹⁰ are independently selected from the group consisting of halogen, hydroxyl, C₁₋₆ alkoxy, C₁₋₆-thioalkoxy, trifluoromethyl, nitro, amino, cyano, —COO—C₁₋₆ alkyl, —COO—C₂₋₆ alkenyl, —COO—C₂₋₆ alkynyl, C₁₋₆ alkyl, C₂₋₆ alkenyl, C₂₋₆ alkynyl, and aryl,
m is 0, 1, 2, 3 or 4,
n is 0, 1, 2, 3 or 4,
p=0, 1, 2 or 3,
and stereo-isomers, tautomers, hydrates, and pharmaceutically acceptable salts thereof.

53. The compound according to claim 49, wherein said compound has a structure according to formula (V): 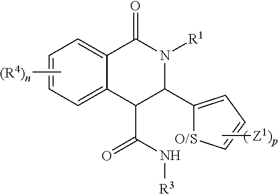
wherein
R¹ is a heteroalkyl which can be unsubstituted or substituted with one or more Z¹,
R³ is selected from the group consisting of heterocycle and aryl, wherein said aryl or heterocycle is optionally substituted with one or more Z¹⁶,
each R⁴ is independently selected from the group consisting of halogen, —OH, alkoxy, —SH, thioalkoxy, trifluoromethyl, trifluoromethoxy, nitro, cyano, —COOH, COO-alkyl, —COO-alkenyl, —COO-alkynyl, amino, heteroalkyl, alkyl, alkenyl, alkynyl, aryl, heterocycle, arylalkyl, arylalkenyl, arylalkynyl, heterocycle-alkyl, heterocycle-alkenyl, and heterocycle-alkynyl,
n is 0, 1, 2, 3, or 4,
p is 0, 1, 2, or 3,
each Z¹ is independently selected from the group consisting of halogen, hydroxyl, sulfhydryl, alkoxy, thioalkoxy, ═O, ═S, trifluoromethyl, trifluoromethoxy, nitro, amino, cyano, —COOH, —COO-alkyl, —COO-alkenyl, —COO-alkynyl, —CONH₂, alkyl, alkenyl, alkynyl, aryl, heterocycle, arylalkyl, arylalkenyl, arylalkynyl, heterocycle-alkyl, heterocycle-alkenyl, and heterocycle-alkynyl,
each Z¹⁶ is independently selected from the group consisting of halogen, hydroxyl, sulfhydryl, alkoxy, thioalkoxy, ═O, ═S, trifluoromethyl, trifluoromethoxy, nitro, amino, cyano, alkyl, alkenyl, alkynyl, aryl, heterocycle, arylalkyl, arylalkenyl, arylalkynyl, heterocycle-alkyl, heterocycle-alkenyl, and heterocycle-alkynyl, wherein said alkoxy, thioalkoxy, alkyl, alkenyl, alkynyl, aryl, heterocycle, arylalkyl, arylalkenyl, arylalkynyl, heterocycle-alkyl, heterocycle-alkenyl, and heterocycle-alkynyl group optionally include one or more heteroatoms selected from the group consisting of O, S, P, and N, and wherein said alkoxy, thioalkoxy, alkyl, alkenyl, alkynyl, aryl, heterocycle, arylalkyl, arylalkenyl, arylalkynyl, heterocycle-alkyl, heterocycle-alkenyl, and heterocycle-alkynyl group is optionally substituted with one or more Z¹⁷,
each Z¹⁷ is independently selected from the group consisting of halogen, —OH, alkoxy, —SH, thioalkoxy, trifluoromethyl, trifluoromethoxy, ═O, ═S, nitro, cyano, —COOH, —COO-alkyl, —COO-alkenyl, —COO-alkynyl, amino, heteroalkyl, alkyl, alkenyl, alkynyl, aryl, heterocycle, arylalkyl, arylalkenyl, arylalkynyl, heterocycle-alkyl, heterocycle-alkenyl, and heterocycle-alkynyl,
and stereo-isomers, tautomers, hydrates and pharmaceutically acceptable salts thereof.

54. The compound according to claim 53, wherein said compound has a structure according to formula (Va) 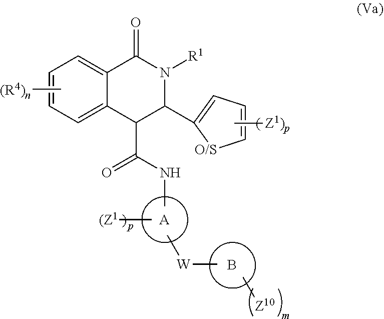
wherein
each of cycle A and cycle B is independently selected from the group consisting of aryl and heterocycle,
R¹ is C₁₋₁₈ heteroalkyl or C₁₋₁₈ alkyl optionally substituted with an optionally C₁₋₆ alkyl-substituted heterocyclic,
W is selected from the group consisting of a single bond, —O—, —S—, alkylene, —C(═O)O—, —NH—C(═O)—, —C(═O)—, and —C(═O)—NH—, wherein said alkylene optionally includes one or more heteroatoms selected from the group consisting of O, S, P, and N,
each R⁴ is independently selected from the group consisting of halogen, —OH, alkoxy, —SH, thioalkoxy, trifluoromethyl, trifluoromethoxy, nitro, cyano, —COOH, —COO-alkyl, —COO-alkenyl, —COO-alkynyl, amino, heteroalkyl, alkyl, alkenyl, alkynyl, aryl, heterocycle, arylalkyl, arylalkenyl, arylalkynyl, heterocycle-alkyl, heterocycle-alkenyl, and heterocycle-alkynyl,
each Z¹ and each Z¹⁰ are independently selected from the group consisting of hydrogen, halogen, hydroxyl, sulfhydryl, alkoxy, thioalkoxy, ═O, ═S, trifluoromethyl, trifluoromethoxy, nitro, amino, cyano, —COOH, —COO-alkyl, —COO-alkenyl, —COO-alkynyl, —CONH₂, alkyl, alkenyl, alkynyl, aryl, heterocycle, arylalkyl, arylalkenyl, arylalkynyl, heterocycle-alkyl, heterocycle-alkenyl, and heterocycle-alkynyl,
m is 0, 1, 2, 3, or 4,
n is 0, 1, 2, 3, or 4,
p is 0, 1, 2, or 3,
and stereo-isomers, tautomers, hydrates and pharmaceutically acceptable salts thereof.

55. A pharmaceutical composition comprising a compound according to claim 49 in combination with a pharmaceutically acceptable carrier.

56. A method for the prevention or treatment of an infection of an animal, mammal or human with a RNA virus, comprising the administration to said animal, mammal or human of a compound according to claim 49.

57. The method according to claim 56, wherein the RNA virus is the Hepatitis C virus.

58. A method for the preparation of an 1-oxo-1,2,3,4-tetrahydroisoquinoline-4-carboxylic acid, comprising the steps of
reacting an aromatic aldehyde with a primary amine to obtain an azomethine,
reacting a homophthalic acid optionally substituted in the benzene ring with an anhydride to obtain the corresponding homophthalic anhydride, and
reacting the homophthalic anhydride with the azomethine in a polar or apolar solvent to obtain a 1-oxo-1,2,3,4-tetrahydroisoquinoline-4-carboxylic acid.

59. A pharmaceutical composition comprising a compound according to formula (II) 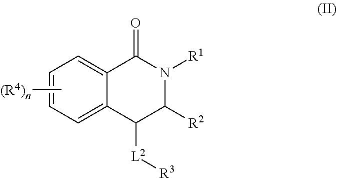
wherein
R¹ is selected from the group consisting of alkyl, alkenyl, alkynyl, aryl, heterocycle, arylalkyl, arylalkenyl, arylalkynyl, heterocycle-alkyl, heterocycle-alkenyl, and heterocycle-alkynyl, wherein said alkyl, alkenyl, alkynyl, aryl, heterocycle, arylalkyl, arylalkenyl, arylalkynyl, heterocycle-alkyl, heterocycle-alkenyl or heterocycle-alkynyl optionally includes one or more heteroatoms selected from the group consisting of O, S, P, and N, and wherein said alkyl, alkenyl, alkynyl, aryl, heterocycle, arylalkyl, arylalkenyl, arylalkynyl, heterocycle-alkyl, heterocycle-alkenyl or heterocycle-alkynyl can be unsubstituted or substituted with one or more Z¹,
R² is thienyl or furanyl, wherein said thienyl or furanyl can be unsubstituted or substituted with one or more Z¹,
R³ is selected from the group consisting of alkyl, alkenyl, alkynyl, heterocycle, aryl, arylalkyl, arylalkenyl, arylalkynyl, heterocycle-alkyl, heterocycle-alkenyl, and heterocycle-alkynyl, wherein said alkyl, alkenyl, alkynyl, arylalkyl, arylalkenyl, arylalkynyl, heterocycle-alkyl, heterocycle-alkenyl or heterocycle-alkynyl optionally includes one or more heteroatoms selected from the group consisting of O, S, P, and N, and wherein said alkyl, alkenyl, alkynyl, aryl, heterocycle, arylalkyl, arylalkenyl, arylalkynyl, heterocycle-alkyl, heterocycle-alkenyl or heterocycle-alkynyl is optionally substituted with one or more Z¹⁶,
L² is selected from the group consisting of —CH₂NZ⁶—, CH₂NH—, —CH₂O—, —CO—NZ⁶—, and —CONH—,
each R⁴ is independently selected from the group consisting of halogen, —OH, alkoxy, —SH, thioalkoxy, trifluoromethyl, trifluoromethoxy, nitro, cyano, —COOH, COO-alkyl, —COO-alkenyl, —COO-alkynyl, amino, heteroalkyl, alkyl, alkenyl, alkynyl, aryl, heterocycle, arylalkyl, arylalkenyl, arylalkynyl, heterocycle-alkyl, heterocycle-alkenyl, and heterocycle-alkynyl,
n is 0, 1, 2, 3, or 4,
each Z¹ is independently selected from the group consisting of halogen, hydroxyl, sulfhydryl, alkoxy, thioalkoxy, ═O, trifluoromethyl, trifluoromethoxy, nitro, amino, cyano, —COOH, —COO-alkyl, —COO-alkenyl, —COO-alkynyl, —CONH₂, alkyl, alkenyl, alkynyl, aryl, heterocycle, arylalkyl, arylalkenyl, arylalkynyl, heterocycle-alkyl, heterocycle-alkenyl, and heterocycle-alkynyl,
each Z⁶ is independently selected from the group consisting of alkyl, alkenyl, alkynyl, heteroalkyl, heterocycle, and aryl,
each Z¹⁶ is independently selected from the group consisting of halogen, hydroxyl, sulfhydryl, alkoxy, thioalkoxy, ═O, ═S, trifluoromethyl, trifluoromethoxy, nitro, amino, cyano, alkyl, alkenyl, alkynyl, aryl, heterocycle, arylalkyl, arylalkenyl, arylalkynyl, heterocycle-alkyl, heterocycle-alkenyl, and heterocycle-alkynyl, wherein said alkoxy, thioalkoxy, alkyl, alkenyl, alkynyl, aryl, heterocycle, arylalkyl, arylalkenyl, arylalkynyl, heterocycle-alkyl, heterocycle-alkenyl, and heterocycle-alkynyl group optionally includes one or more heteroatoms selected from the group consisting of O, S, P, and N, and wherein said alkoxy, thioalkoxy, alkyl, alkenyl, alkynyl, aryl, heterocycle, arylalkyl, arylalkenyl, arylalkynyl, heterocycle-alkyl, heterocycle-alkenyl, and heterocycle-alkynyl group is optionally substituted with one or more Z¹⁷,
each Z¹⁷ is independently selected from halogen, —OH, alkoxy, —SH, thioalkoxy, trifluoromethyl, trifluoromethoxy, ═O, ═S, nitro, cyano, —COOH, —COO-alkyl, —COO-alkenyl, —COO-alkynyl, amino, heteroalkyl, alkyl, alkenyl, alkynyl, aryl, heterocycle, arylalkyl, arylalkenyl, arylalkynyl, heterocycle-alkyl, heterocycle-alkenyl, and heterocycle-alkynyl,
or a stereo-isomer, tautomer, hydrate, or pharmaceutically acceptable salt thereof.

60. The pharmaceutical composition according to claim 59, wherein R¹ is a heteroalkyl which can be unsubstituted or substituted with one or more Z¹, and wherein L² is —CONH—.

61. The pharmaceutical composition according to claim 59, wherein R³ is selected from the group consisting of heterocycle and aryl, wherein said aryl or heterocycle is optionally substituted with one or more Z¹⁶.
